(19) United States
(12) Patent Application Publication
(10) Pub. No.: US 2016/0221992 A1
(43) Pub. Date: Aug. 4, 2016

(54) DENDRITIC COMPOUNDS INCLUDING A CHELATING, FLUOROCHROME OR RECOGNITION AGENT, COMPOSITIONS INCLUDING SAME AND USES THEREOF

(71) Applicants: UNIVERSITE DE STRASBOURG, Strasbourg (FR); UNIVERSITE CLAUDE BERNARD LYON 1, Villeurbanne (FR); HOSPICES CIVILS DE LYON, Lyon (FR); CENTRE NATIONAL DE LA RECHERCHE SCIENTIFIQUE -CNRS, Paris Cedexc (FR)

(72) Inventors: Delphine FELDER-FLESCH, Hattstatt (FR); Claire BILLOTEY, Lyon (FR); Audrey PARAT, Strasbourg (FR); Antonio GAROFALO, Lipsheim (FR); David KRYZA, Lyon (FR); Marc JANIER, Limas (FR)

(21) Appl. No.: 14/891,443

(22) PCT Filed: May 16, 2014
(86) PCT No.: PCT/FR2014/051143

§ 371 Date: Apr. 8, 2016

(30) Foreign Application Priority Data
May 16, 2013 (FR) 1354420
May 23, 2013 (FR) 1354668
May 23, 2013 (FR) 1354968

(51) Int. Cl. C07D 403/14; A61K 49/04; A61K 51/04; C07F 13/00; C07F 5/00; C07D 403/12; C07D 241/44

(52) U.S. Cl. CPC C07D 403/14; C07D 403/12; A61K 49/04; C07D 241/44; C07F 13/00; C07F 5/00; A61K 51/0459

(57) ABSTRACT

The invention relates to dendritic compounds comprising a chelating, fluorochrome or recognition agent of formula (I), to compositions comprising same, and to uses thereof, wherein in said formula (I): T, L1, D, R, L2, V and n are as defined in the description.

### FIELD OF THE INVENTION

The present invention relates to dendritic compounds comprising a chelating agent, fluorochrome or recognition agent, compositions comprising them and uses thereof.

In the following description, the references in square brackets ([ ]) refer to the list of references given at the end of the text.

### BACKGROUND

The use of dendrimers or of dendritic compounds for biomedical applications is a flourishing area of research, mainly on account of their precisely defined structure that leads to systems that are biocompatible, polyfunctional and water-soluble (S. E. Stiriba, H. Frey and R. Haag, Angew. Chem. Int. Ed., 2002, 41, 1329-1334 [1]; M. J. Cloninger, Curr. Opin. Chem. Biol., 2002, 6, 742-748 [2]: R Duncan and L. Izzo, Adv. Drug Delivery Rev., 2005, 57, 2215-2237 [3]; C. C. Lee, J. A. MacKay, J. M. J. Fréchet and F. C. Szoka, Nat. Biotechnol., 2005, 23, 1517-1526 [4]; O. Rolland, C-O. Turrin. A-M. Caminade, J-P. Majoral, New J. Chem., 2009, 33, 1809-1824 [5]).

In recent years several research teams have explored the use of dendrimers as a new class of macromolecular contrast agents for magnetic resonance imaging (MRI). The efficiency of MRI contrast agents is often expressed in terms of longitudinal relaxivity (r₁/mM⁻¹s⁻¹), relating to their ability to shorten the longitudinal relaxation time of the protons of the water molecules (T₁/s).

In earlier works, Wiener et al. (E. C. Wiener, M. W. Brechbiel, H. Brothers. R. L. Magin, O. A. Gansow, D. A. Tomalia and P. C. Lauterbur, Magn. Reson. Med., 1994, 31, 1 [6]) described the synthesis of different generations of PAMAM dendrimers bound to the Gd(III)-DTPA chelate. The sixth-generation dendritic MRI contrast agent (MW=139 kg·mol⁻¹) has a relaxation rate r₁ of 34 mM⁻¹s⁻¹ (0.6 T, 20° C.), which is six times higher than the relaxation rate r₁ of the Gd(III)-DTPA chelate alone (MW=0.55 kg·mol⁻¹, r₁=5.4 mM⁻¹s⁻¹). This strong increase in the rate r₁ was attributed to the lowering of the kinetics of inversion of the Gd(III)-DTPA chelate at the periphery of the dendrimer, as shown by the increase in the rotational correlation times (E. C. Wiener, F. P. Auteri, J. W. Chen, M. W. Brechbiel, O. A. Gansow, D. S. Schneider, R L. Belford, R. B. Clarkson and P. C. Lauterbur, J. Am. Chem. Soc., 1996, 118, 7774 [7]). Interestingly, no increase in the value of r₁ was observed for the flexible macromolecular polymers of comparable molecular weight (V. S. Vexler, O. Clement, H. Schmitt-Willich and R C. Brasch, J. Magn. Reson. Imaging, 1994, 4, 381 [8]; T. S. Desser. D. L. Rubin. H. H. Muller, F. Qing, S. Khodor, G. Zanazzi, S. W. Young, D. L. Ladd, J. A. Wellons and K. E. Kellar, J. Magn. Reson. Imaging, 1994, 4, 467 [9]), which implies that segmental mobility predominates over the rotational correlation time. Bryant et al. studied the relation between r, and the molecular weight of different generations of PAMAM dendrimers bound to the Gd(III)-DOTA chelate (L. H. Bryant, Jr, M. W. Brechbiel, C. Wu, J. W. Bulte. V. Hervnek and J. A. Frank, J. Magn. Reson. Imaging, 1999, 9, 348 [10]): a plateau value for r₁ of 36 mM⁻¹s⁻¹ (0.47 T, 20° C.) was reached for the seventh generation of dendrimer (MW=375 kg·mol⁻¹). Moreover, it was demonstrated that the value of r₁ relating to this seventh generation of dendrimer increases when the temperature increases, which indicates that a slow exchange of water molecules limits the relaxivity (E. Toth, D. Pubanz, S. Vauthey, L. Helm and A. E. Merbach, Chem. Eur. J., 1996, 2, 1607 [11]). Rudovsky et al. studied the effect on r₁ of the ionic interactions between negatively-charged PAMAM-Gd(III) dendrimers and positively-charged polyamino acids (J. Rudovsky, P. Hermann, M. Botta, S. Aime and I. Lukes, Chem. Commun., 2005, 2390 [12]). Titration experiments on a second-generation dendritic contrast agent in the presence of polyarginine showed an increase in r₁ from 20 to 28 mM⁻¹s⁻¹ (0.47 T, 20° C.). This effect was attributed to a decrease in the mobility of the complex comprising Gd(III), induced by the interactions between the anionic dendrimer and the cationic polyarginine. A series of PPI dendrimers functionalized with a chelate of Gd(III)-DTPA was described by Kobayashi et al. (H. Kobayashi, S. Kawamoto, S.-K. Jo, H. L. Bryant, Jr, M. W. Brechbiel, Jr and R. A. Star, Bioconjugate Chem., 2003, 14, 388 [13]). The authors demonstrated that r₁ increases almost linearly with the increase in the molecular weight of the dendrimer without reaching a plateau value, a value of r₁ of 29 mM⁻¹s⁻¹ (1.5 T, 20° C.) for example being reached for the fifth generation of dendritic contrast agent. Later, E. W. Meijer et al. proposed a new series of PPI dendrimers functionalized with a Gd(III)-DTPA chelate using a different spacer between the Gd(III)-chelating agent complex and the dendrimer (S. Langereis, Q. G. de Lussanet, M. H. P. van Genderen, W. H. Backes and E. W. Meijer, Macromolecules, 2004, 37, 3084 [14]). For these last-mentioned dendrimers, a significant increase in r₁, but less pronounced than for the dendritic MRI contrast agents described by Kobayashi et al., was observed, whereas the molecular weights of the two systems were comparable (fifth generation: r₁=20 mM⁻¹s⁻¹, 1.5 T and 20° C.). Researchers at Schering AG (Berlin, Germany) developed a class of dendritic contrast agents constructed from lysine: Gadomer-17® (r₁=15.2 mM⁻¹s⁻¹, 1.5 T and 37° C.) (C. Fink, F. Kiessling, M. Bock, M. P. Lichy, B. Misselwitz, P. Peschke, N. E. Fusenig, R. Grobholz and S. Delorme, J. Magn. Reson. Imaging, 2003, 18, 59 [15]; G. M. Nicolle, E. Toth, H. Schmitt-Willich, B. Raduchel and A. E. Merbach, Chem. Eur. J., 2002, 8, 1040 [16]: G. Adam, J. Neuerburg, E. Spuntrup, A. Muhler, K. Scherer and R. W. Gunther, J. Magn. Reson. Imaging, 1994, 4, 462 [17]; G. Adam, J. Neuerburg, E. Spuntrup, A. Muhler, K. Scherer and R W. Gunther, Magn. Reson. Med., 1994, 32, 622 [18]; H. C. Schwickert, T. P. Roberts, A. Muhler, M. Stiskal, F. Demsar and R. C. Brasch, Eur. J. Radiol., 1995, 20, 144 [19]: H. C. Roberts, M. Saeed, T. P. Roberts, A. Muhler, D. M. Shames, J. S. Mann, M. Stiskal, F. Demsar and R C. Brasch, J. Magn. Reson. Imaging, 1997, 7, 331 [20]). These macromolecular MRI contrast agents were synthesized starting from a trimesoyltriamide central nucleus, onto which 18 lysine amino acid residues were introduced.

In the above examples, the dendrimers proved to be suitable synthetic platforms for incorporating several groups comprising Gd(III), leading to an improvement in sensitivity in MRI in terms of r₁ value. These conclusions are based on measurements at existing magnetic fields, of 0.5-1.5 T. However, at magnetic fields of 10 T, the r₁ values of dendritic contrast agents are far lower, not exceeding the r₁ values of low molecular weight complexes based on Gd(III). The dendrimers also improve the protection of the gadolinium and its stability and therefore make it possible to reduce the risks of toxicity.

The dendritic MRI contrast agents are excellent intravascular contrast agents. However, these structures do not have the specificity required for molecular MRI (D. Artemov, J. Cell. Biochem., 2003, 90, 518 [21]), owing to lack of accumulation of the MRI contrast agents in the regions of interest, for example a tumor.

Moreover, to date, there has been little description of the use of dendrimers for complexation of 99 mTc in the literature: in 2001, F. Vögtle et al. (H. Stephan. H. Spies, B. Johannsen, K. Gloe, M. Gorka, F. Vögtle, Eur. J Inorg. Chem., 2001, 2957-2963 [22]) reported the host-guest properties of multi-crown dendrimers of four different generations with respect to sodium pertechnetate. Extraction studies showed that the host molecules are essentially bound to the interior of the polyamine skeleton. The same year, H. Mukhtar et al. (M. Subbarayan, S. J. Shetty, T. S. Srivastava, O. P. D. Noronha, A. M. Samuel, H. Mukhtar, Biochem. and Biophys. Res. Commun., 2001, 281, 32-36 [23]) described the synthesis and distribution in vivo of 99 mTc-labeled dendritic porphyrins for imaging of tumors and for diagnostics: these dendritic systems were administered to Wistar rats with glial tumors, and scintigraphic imaging studies have shown their potential for the early detection of tumors. Finally, A. Adronov, J F Valliant et al. (M. C. Parrott, S. R. Benhabbour, C. Saab, J. A. Lemon, S. Parker, J. F. Valliant, A. Adronov, J. Am. Chem. Soc., 2009, 131, 2906-2916 [24]) described the use of high-generation polyester dendrimers for complexing 99 mTc and their subsequent use for SPECT imaging: it was found that the three generations of dendrimer (G5 to G7) were eliminated from the blood circulation quickly and efficiently via the kidneys and were excreted by the bladder within 15 min after injection. The SPECT-CT data were confirmed by a quantitative biodistribution study involving taking samples from various organs ex vivo and determining the radioactivity in the organs as a function of time.

However, the uptake rate of said dendrimer-99 mTc complexes is too low to allow reliable detection of tumors, especially micrometastases. The documents mentioned above do not show targeting of micrometastases by the compounds that are described there.

There is therefore a real need to supply compounds allowing targeting of tumor cells, in particular in the form of micrometastases.

Moreover, there is a real need to supply compounds used as tools for detecting tumors, notably in the form of micrometastases.

Furthermore, there is a real need to supply compounds used as radiotherapeutic medicaments in the treatment of tumors, notably in the form of micrometastases.

Moreover, there is a real need to supply pharmaceutical or diagnostic compositions comprising said compounds.

### DESCRIPTION

The present invention specifically aims at responding to these needs by supplying a compound of the following Formula I:

T-L₁-D-R-L₂V)ₙ (I)

wherein:

T is a chelating agent, a fluorochrome or a recognition agent
[symbol] the chelating agent being able to fix;
a gadolinium or manganese metal ion, or
a radioelement that emits gamma radiation, or emits positrons, and/or emits particle rays, beta minus, Auger electron or alpha particles such as gold-33, copper-61, copper-62, copper-64, copper-67, gallium-67, gallium-68, zirconium-89, yttrium-90, technetium-99m, indium-111 iodine-123, iodine-124, iodine-125, iodine-131, holmium-166, lutetium-177, rhenium-186, astatine-211, actinium-89, bismuth-213;
[symbol] the recognition agent being able to form a complex with a specific molecule, optionally bound to another dendrimer, said complex being selected from the biotin-avidin complex, the biotin-streptavidin complex, an antibody-antigen complex, a ligand-receptor complex, a double-stranded oligonucleotide, or an adamantane-cyclodextrin complex,

L₁ represents Ø (i.e. a covalent bond), or a spacer —C(═O)NH—, —NHC(═O)—, —NHC(═S)NH— or triazolyl of formula:

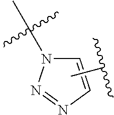

wherein one of the points of attachment is bound to T and the other to D directly or via a heteroalkyl chain of formula:

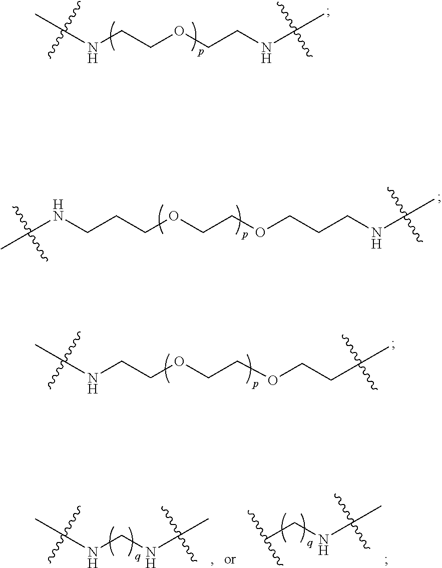

wherein p is an integer from 1 to 10, and q is an integer from 1 to 12;

D is a PAMAM, gallate or aspartate dendrimer of formula (DD):

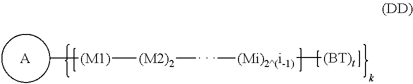

wherein:
(a) A is the dendrimer nucleus, of multivalence k, where:
   k represents the number of dendrons and is an integer ranging from 2 to 8;
   A is a synthon of structure:

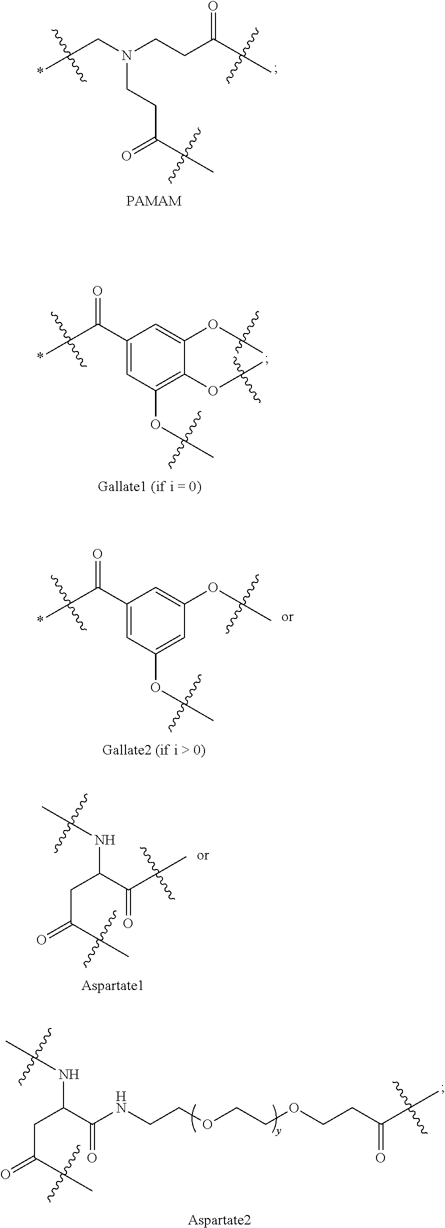

where * indicates the point of attachment to the spacer L₁; and y represents an integer from 1 to 10, preferably from 1 to 6, preferably from 1 to 4;
(b) Mi is a monomer of generation i, where:
   i is an integer ranging from 0 to g, g being the generation number of the dendrimer;
   when i=0, Mi is absent, and the terminal branch TB is then bound directly to the nucleus A;
   when i>0, Mi represents:

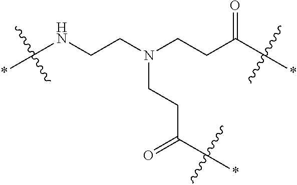

when D is PAMAM;

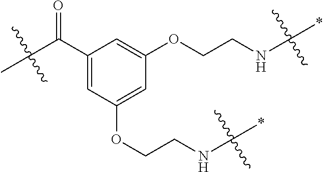

when D is gallate

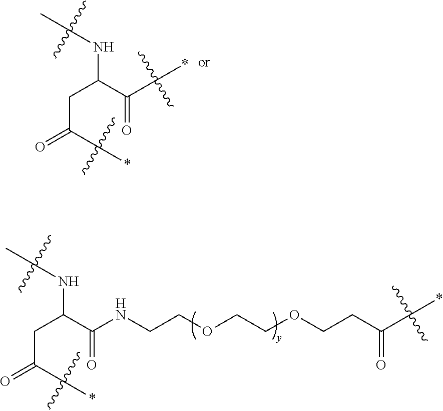

when D is aspartate
where the symbol * denotes the point of attachment of the monomer Mi to the monomer of higher generation; and y represents an integer from 1 to 10, preferably from 1 to 6, preferably from 1 to 4;
(c) TB is the terminal branch, and t is the number of terminal units from the monomer of lower generation, where:
   t is an integer ranging from 1 to 3; preferably t is 2 or 3;
TB is a group selected from the group comprising a hydrogen, an —NH₂ group, an —OH group, a C₁ to C₆ alkyl, a polyethylene glycol chain of formula

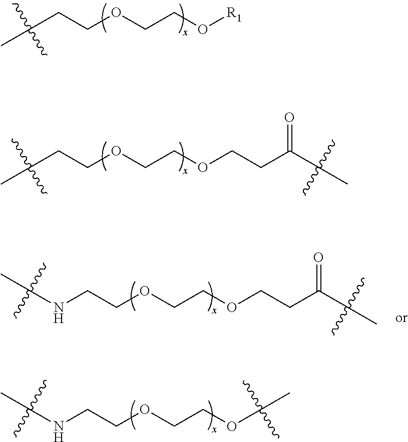

wherein each occurrence of x is independently an integer from 1 to 10;
R₁ represents C₁₋₆ alkyl or —C(═O)OR₂ where R₂ represents C₁₋₆ alkyl, and preferably R₁ represents —OMe or —COOtBu;
and at least one occurrence of TB is covalently bound to a targeting agent V;

R represents Ø (i.e. a covalent bond), or represents a radiolabelable moiety or group such as L-thyroxine or dinitrobenzoate:

L₂ represents Ø (i.e. a covalent bond), or a heteroalkyl chain of formula:

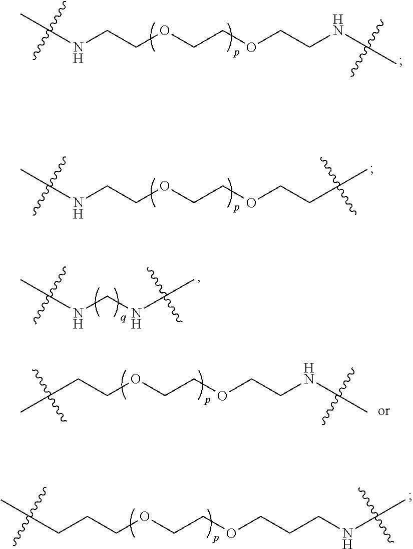

wherein p is an integer from 1 to 10, and q is an integer from 1 to 12;

V is a targeting agent; and

n is an integer greater than or equal to 1, and represents the number of targeting agents attached to the compound formula I;

provided that when D is a gallate dendrimer, V does not comprise a DOPA structure of formula

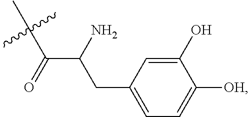 [SMILES: *C(=O)C(N)Cc1ccc(O)c(O)c1]

and T does not comprise a catechol structure of the following formula:

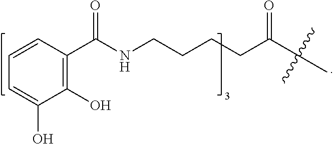

The present invention also provides a compound of Formula I wherein L₂ is Ø (i.e. a covalent bond), and the compound has the following formula II:

T-L₁-D-RV)ₙ (II)

wherein:

T, L₁, D, V and n are as defined above; and

R is a radiolabelable moiety selected from:

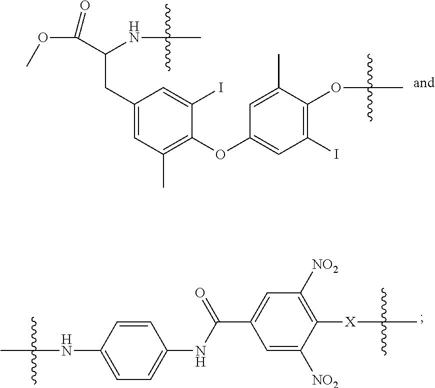

where X represents O or NH.

The present invention also provides a compound of Formula I wherein R and L₂ each represent Ø (i.e. a covalent bond), and the compound has the following formula III:

T-L₁-DV)ₙ (III)

wherein T, L₁, D, V and n are as defined above.

Thus, the compounds of the invention of formula (I), as well as of the subclasses (II) and (III), comprise at least one targeting agent V.

Chelating Agent, Fluorochrome or Recognition Agent T
i. T may represent a chelating agent of formula:

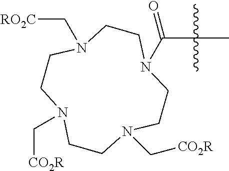

where each occurrence of R represents independently H or a protective group such as t-Bu; preferably all the occurrences of R are identical, preferably each R represents H:
ii. T may represent a chelating agent of formula:

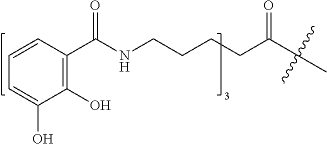

except in the case when D is a gallate dendrimer and V represents a DOPA targeting agent comprising the structure of formula

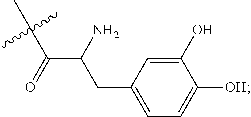 [SMILES: *C(=O)C(N)Cc1ccc(O)c(O)c1]

iii. T may represent a fluorescein fluorochrome of formula (isomer 5 or 6):

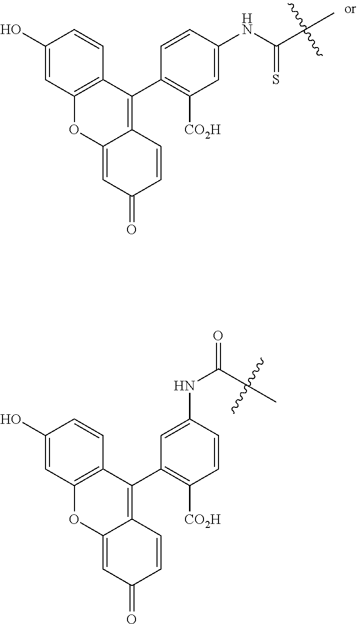 [SMILES: *C(=S)Nc1ccc(-c2c3ccc(=O)cc-3oc3cc(O)ccc23)c(C(=O)O)c1 ; *C(=O)Nc1ccc(C(=O)O)c(-c2c3ccc(=O)cc-3oc3cc(O)ccc23)c1]

iv. T may represent a recognition agent such as cyclodextrin, adamantane, biotin, avidin or streptavidin:

Spacer L₁
v. L₁ may represent Ø (i.e. a covalent bond), or a spacer —C(═O)NH—, —NHC(═O)—, —NHC(═S)NH—, or triazolyl of formula:

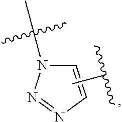

preferably

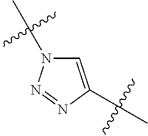 [SMILES: *c1cn(*)nn1]

vi. L₁ may advantageously represent Ø (i.e. a covalent bond), or a triazolyl spacer of formula:

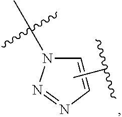

preferably

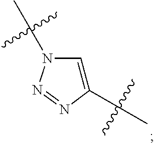 [SMILES: *c1cn(*)nn1]

vii. L₁ as defined at v and vi may advantageously be bound to T via a heteroalkyl chain of formula:

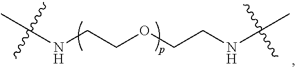

wherein p is an integer from 1 to 10, preferably from 1 to 6, preferably 2, 3, 4, 5 or 6;
viii. L₁ as defined at v and vi may advantageously be bound to T via a heteroalkyl chain of formula:

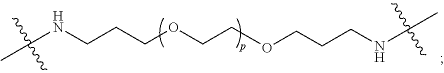

wherein p is an integer from 1 to 10; preferably from 1 to 6; preferably from 1 to 4, preferably from 1 to 2; preferably 2;
ix. L₁ as defined at v and vi may advantageously be bound to T via a heteroalkyl chain of formula:

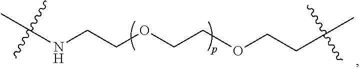

wherein p is an integer from 1 to 10, preferably from 1 to 6, preferably 2, 3, 4, 5 or 6. For example, L₁ may represent a triazolyl spacer of formula

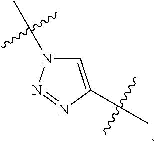 [SMILES: *c1cn(*)nn1]

advantageously with p=6 so that T and D are bound by the chain:

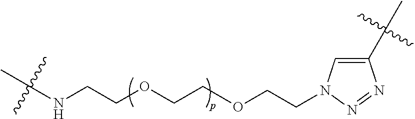

wherein p is an integer from 1 to 10, preferably from 1 to 6, preferably 2, 3, 4, 5 or 6, advantageously p is 6;
x. L₁ as defined at v and vi may advantageously be bound to T via a heteroalkyl chain of formula:

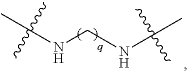

wherein q is an integer from 1 to 12, preferably from 1 to 6, preferably 2, 3, 4, 5 or 6; preferably 3; For example, L₁ may represent Ø (i.e. a covalent bond) so that T and D are bound by the chain:

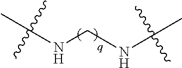

wherein q is an integer from 1 to 12, preferably from 1 to 6, preferably 2, 3, 4, 5 or 6; preferably 3;
xi. L₁ as defined at v and vi may advantageously be bound to T via a heteroalkyl chain of formula:

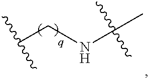

wherein q is an integer from 1 to 12, preferably from 1 to 6, preferably 2, 3, 4, 5 or 6; preferably 3. For example, L₁ may represent a spacer —NHC(═S)NH—, advantageously with q=3 so that T and D are bound by the chain:

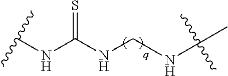

wherein q is an integer from 1 to 12, preferably from 1 to 6, preferably 2, 3, 4, 5 or 6; preferably 3;

### Dendrimer D

xii. D may represent a PAMAM dendrimer of formula (DD) wherein:

A is a synthon of structure

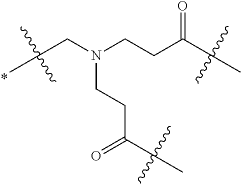 [SMILES: *CN(CCC(*)=O)CCC(*)=O]

where * indicates the point of attachment to the spacer L₁;
(b) Mi is a monomer of generation i, where:
   i is an integer ranging from 0 to 3, preferably from 0 to 2, preferably 0 or 1; and when i=0. Mi is absent, and the terminal branch TB is then bound directly to the nucleus A;
   when i>0, Mi represents:

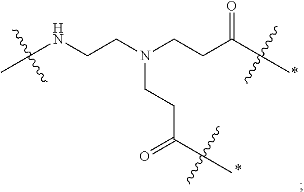 [SMILES: *NCCN(CCC(*)=O)CCC(*)=O]

where the symbol * denotes the point of attachment of the monomer Mi to the monomer of higher generation;
(c) TB is the terminal branch, and t is the number of terminal units from the monomer of lower generation, where:
   t is an integer ranging from 1 to 2, preferably t is 2;
   TB is a radical selected from the group comprising a hydrogen, an —NH₂ group, a —COOH group, an —OH group, a C₁ to C₆ alkyl, a polyethlene glycol chain of formula

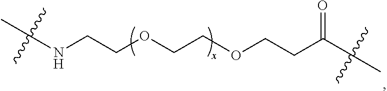

wherein each occurrence of x is independently an integer from 1 to 10, preferably from 1 to 6, preferably from 1 to 4, preferably 3; and at least one occurrence of TB is covalently bound to a targeting agent V or a radiolabelable moiety R, preferably at least one occurrence of TB per monomer Mi is covalently bound to a targeting agent V or a radiolabelable moiety R, preferably each occurrence of TB per monomer Mi is covalently bound to a targeting agent V or a radiolabelable moiety R; the occurrences of TB not bound to V or a radiolabelable moiety R are terminated with H, or a C₁ to C₆ alkyl such as methyl or ethyl;
xiii. D may represent a gallate dendrimer of formula (DD) wherein:

A is a synthon of structure

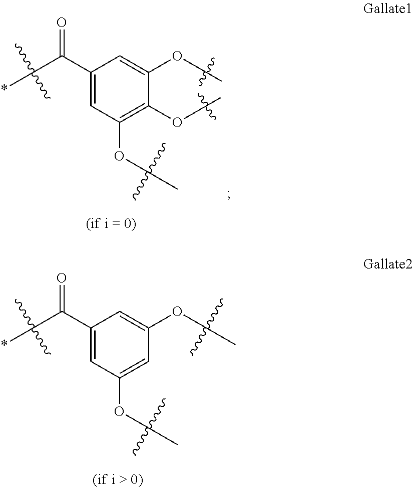

where * indicates the point of attachment to the spacer L₁;
(b) Mi is a monomer of generation i, where:
   i is an integer ranging from 0 to 3, preferably from 0 to 2, preferably 0 or 1; and when i=0, Mi is absent, and the terminal branch TB is then bound directly to the nucleus A;
   when i>0, Mi represents:

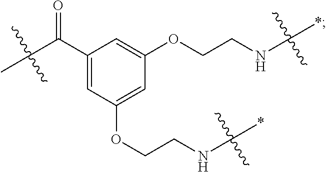

where the symbol * denotes the point of attachment of the monomer Mi to the monomer of higher generation;
(c) TB is the terminal branch, and t is the number of terminal units from the monomer of lower generation, where:
   t is an integer ranging from 1 to 3, preferably t is 3;
   TB is a radical selected from the group comprising a hydrogen, an —NH₂ group, an —OH group, a C₁ to C₆ alkyl, a polyethylene glycol chain of formula

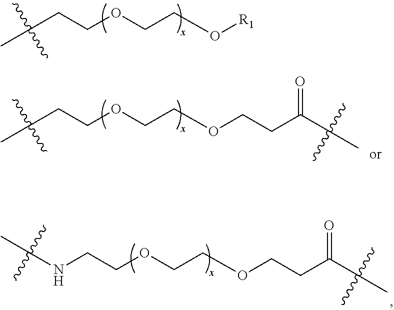

wherein each occurrence of x is independently an integer from 1 to 10, preferably from 1 to 8, preferably from 2 to 8, preferably 7;
R₁ represents C₁₋₆alkyl or —C(═O)OR₂ where R₂ represents C₁₋₆ alkyl, preferably R₁ represents —OMe or —COOtBu;
and at least one occurrence of TB is covalently bound to a targeting agent V, preferably at least one occurrence of TB per monomer Mi is covalently bound to a targeting agent V or a radiolabelable moiety R, preferably each occurrence of TB per monomer Mi is covalently bound to a targeting agent V or a radiolabelable moiety R; and the occurrences of TB not bound to V or a radiolabelable moiety R are terminated by a C₁ to C₆ alkyl such as methyl or ethyl;
xiv. D may represent an aspartate dendrimer of formula (DD) wherein:

A is a synthon of structure

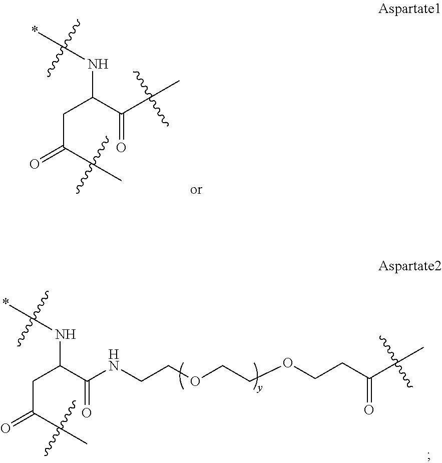

where * indicates the point of attachment to the spacer L₁; and y represents an integer from 1 to 10, preferably from 1 to 6, preferably from 1 to 4;
(b) Mi is a monomer of generation i, where:
   i is an integer ranging from 0 to 3, preferably from 0 to 2, preferably 0 or 1; and when i=0, Mi is absent, and the terminal branch TB is then bound directly to the nucleus A;
   when i>0, Mi represents:

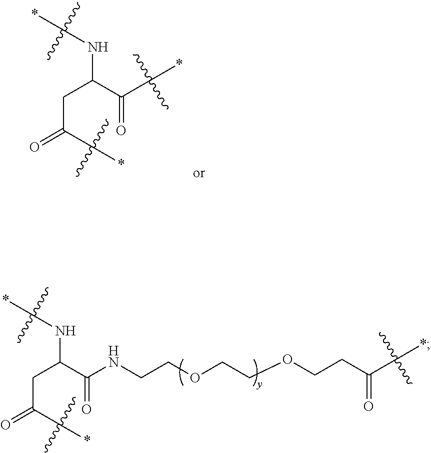

where the symbol * denotes the point of attachment of the monomer Mi to the monomer of higher generation; and y represents an integer from 1 to 10, preferably from 1 to 6, preferably from 1 to 4;
(c) TB is the terminal branch, and t is the number of terminal units from the monomer of lower generation, where:
   t is an integer ranging from 1 to 2, preferably t is 2;
   TB is a radical selected from the group comprising a hydrogen, an —NH₂ group, an —OH group, a C₁ to C₆ alkyl, a polyethylene glycol chain of formula

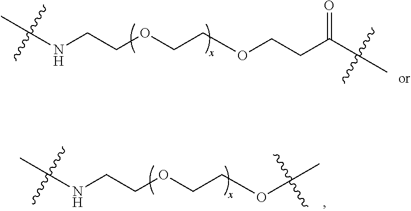

wherein each occurrence of x is independently an integer from 1 to 10, preferably from 1 to 8, preferably from 2 to 8, preferably 7; and at least one occurrence of TB is covalently bound to a targeting agent V or a radiolabelable moiety R, preferably at least one occurrence of TB per monomer Mi is covalently bound to a targeting agent V or a radiolabelable moiety R, preferably each occurrence of TB per monomer Mi is covalently bound to a targeting agent V or a radiolabelable moiety R, preferably each occurrence of TB per monomer Mi is covalently bound to a radiolabelable moiety R; the occurrences of TB not bound to V or a radiolabelable moiety R are terminated with H, or a C₁ to C₆ alkyl such as methyl or ethyl; advantageously, the radiolabelable moiety corresponds to the structure:

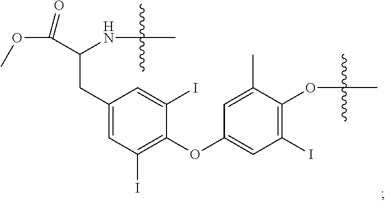 [SMILES: *NC(Cc1cc(I)c(Oc2cc(C)c(O*)c(I)c2)c(I)c1)C(=O)OC]

Radiolabelable Moiety R
xv. R may advantageously represent Ø (i.e. a covalent bond);
xvi. R may advantageously represent a labelable moiety such as L-thyroxine of formula:

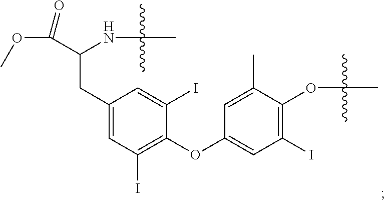 [SMILES: *NC(Cc1cc(I)c(Oc2cc(C)c(O*)c(I)c2)c(I)c1)C(=O)OC]

xvii. R may advantageously represent a labelable moiety such as the dinitrobenzoate of formula:

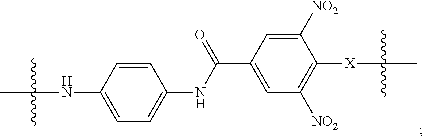

where X represents O or NH;

Spacer L₂
xviii. L₂ may advantageously represent Ø (i.e. a covalent bond);
xix. L₂ may advantageously represent a heteroalkyl chain of formula:

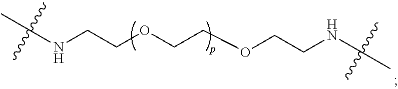

wherein p is an integer from 1 to 10; preferably from 1 to 6;
xx. L₂ may advantageously represent a heteroalkyl chain of formula:

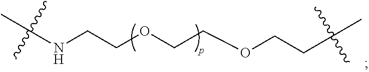

wherein p is an integer from 1 to 10; preferably from 1 to 6;

L₂ may advantageously represent a heteroalkyl chain of formula:

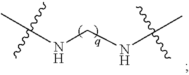

wherein q is an integer from 1 to 12; preferably from 1 to 6; preferably from 1 to 4; preferably from 1 to 3; preferably 3;
xxi. L₂ may advantageously represent a heteroalkyl chain of formula:

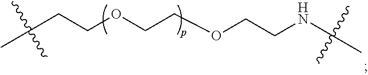

wherein p is an integer from 1 to 10; preferably from 1 to 6;
xxii. L₂ may advantageously represent a heteroalkyl chain of formula:

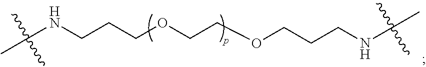

wherein p is an integer from 1 to 10; preferably from 1 to 6; preferably from 1 to 4; preferably from 1 to 2; preferably 2;
xxiii. L₂ may advantageously represent a heteroalkyl chain of formula:

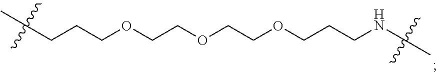 [SMILES: *CCCOCCOCCOCCCN*]

advantageously, this variant of L₂ is used in the compound of formula I where R represents advantageously a labelable moiety such as the dinitrobenzoate of formula:

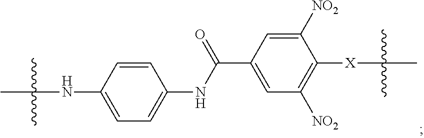

where X represents NH, to form a structure of formula:

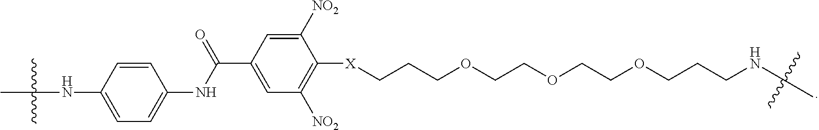

### Targeting Agent V

xxiv. V may represent a melanoma targeting agent corresponding to the formula:

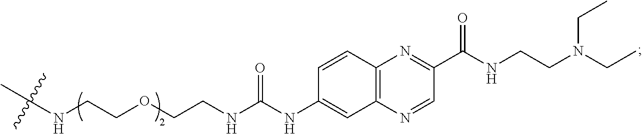

xxv. V may represent a tripeptide targeting agent of formula:

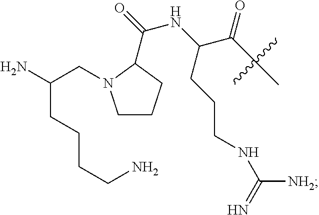 [SMILES: *C(=O)C(CCCNC(=N)N)NC(=O)C1CCCN1CC(N)CCCCN]

xxvi. V may represent an amino-metronidazolyl targeting agent of formula:

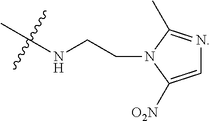 [SMILES: *NCCn1c([N+](=O)[O-])cnc1C]

The compounds of the invention comprise all possible combinations of the variables T, L₁, D, R, L₂ and V, as described at points (i) to (xxvi) above. These variants are applicable to any one of the formulas of the compounds described in the present document, when the variable in question (i.e., T, L₁, D, R, L₂ and V) is present in the formula, including the compounds of formula I, II, III, IV and I^{A} to I^{E}. Out of concerns for temperance in the drafting, each variant is not described explicitly in the present text, but the reader will readily understand that all of these variants are included within the scope of the invention, as well as how to bind T, L₁, D, R, L₂ and V chemically in light of the Examples. Advantageously, the compounds of formula I have one of the following structures:

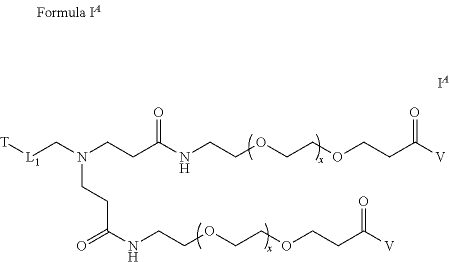

Advantageously, x is an integer from 1 to 10, preferably from 1 to 6, preferably from 1 to 3, for reference 1, 2 or 3. Advantageously, L₁ represents:

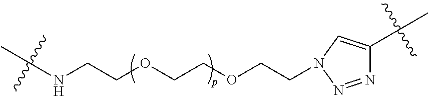

wherein p is an integer from 1 to 10, preferably from 1 to 6, preferably 2, 3, 4, 5 or 6. Advantageously p is 6. Advantageously, T represents either one of the chelating agents i or ii. Advantageously, T represents the fluorochrome iii. Advantageously, T represents one of the recognition agents iv. Advantageously, V represents any one of the targeting agents xxiv to xxvi.

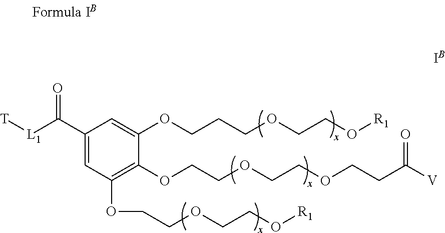

Advantageously, each occurrence of R₁ is independently C₁-C₆ alkyl, preferably methyl or t-butyl, preferably each occurrence of R₁ is methyl. Advantageously, each occurrence of x is independently an integer from 1 to 10, preferably from 1 to 6, preferably from 1 to 3, for reference 1, 2 or 3. Advantageously, L₁ represents:

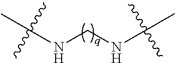

wherein q is an integer from 1 to 12, preferably from 1 to 6, preferably 2, 3, 4, 5 or 6; preferably 3. Advantageously, L₁ may represent: a heteroalkyl chain of formula:

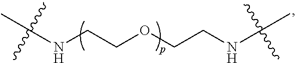

wherein p is an integer from 1 to 10, preferably from 1 to 6, preferably 2, 3, 4, 5 or 6. Advantageously, L₁ may also represent: a spacer of formula

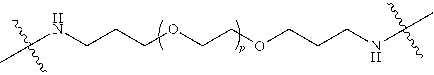

where p=2. Advantageously, T represents either one of the chelating agents i or ii. Advantageously, T represents the fluorochrome iii. Advantageously, T represents one of the recognition agents iv. Advantageously, V represents any one of the targeting agents xxiv to xxvi.

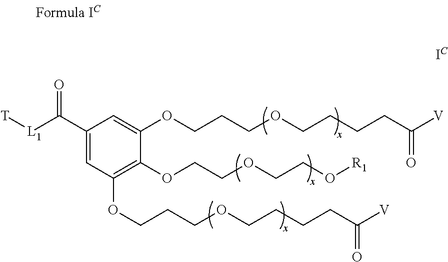

Advantageously, R₁ is C₁-C₆ alkyl, preferably methyl or t-butyl, preferably R₁ is methyl. Advantageously, each occurrence of x is independently an integer from 1 to 10, preferably from 1 to 6, preferably from 1 to 3, for reference 1, 2 or 3. Advantageously, L₁ represents:

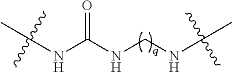

wherein q is an integer from 1 to 12, preferably from 1 to 6, preferably 2, 3, 4, 5 or 6; preferably 3. Advantageously, L₁ may represent: a heteroalkyl chain of formula:

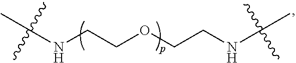

wherein p is an integer from 1 to 10, preferably from 1 to 6, preferably 2, 3, 4, 5 or 6. Advantageously, L₁ may also represent: a spacer of formula

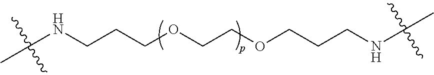

where p=2. Advantageously, T represents either one of the chelating agents i or ii. Advantageously, T represents the fluorochrome iii. Advantageously, T represents one of the recognition agents iv. Advantageously, V represents any one of the targeting agents xxiv to xxvi.

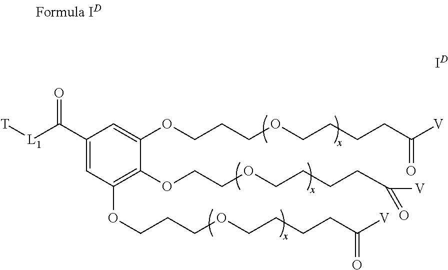

Advantageously, L₁ may represent: a heteroalkyl chain of formula:

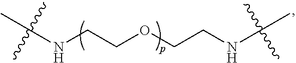

wherein p is an integer from 1 to 10, preferably from 1 to 6, preferably 2, 3, 4, 5 or 6. Advantageously, L₁ may also represent: a spacer of formula

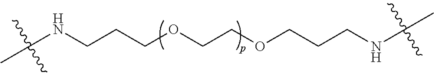

where p=2. Advantageously, T represents either one of the chelating agents i or ii. Advantageously, T represents the fluorochrome iii. Advantageously, T represents one of the recognition agents iv. Advantageously, V represents any one of the targeting agents xxiv to xxvi.

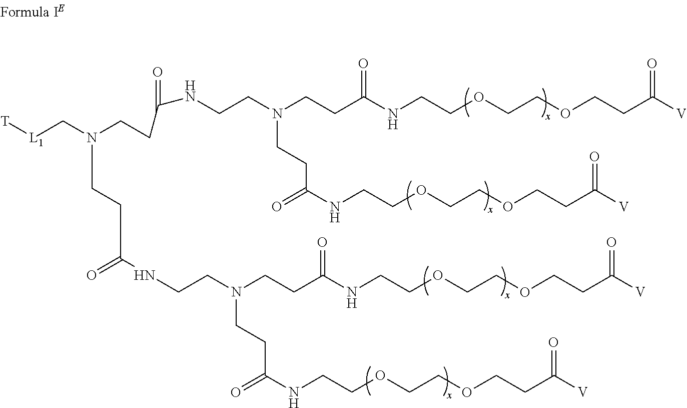

Advantageously, x is an integer from 1 to 10, preferably from 1 to 6, preferably from 1 to 3, for reference 1, 2 or 3. Advantageously, L₁ represents:

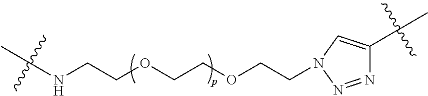

wherein p is an integer from 1 to 10, preferably from 1 to 6, preferably 2, 3, 4, 5 or 6, advantageously p is 6. Advantageously, T represents either one of the chelating agents i or ii. Advantageously, T represents the fluorochrome iii. Advantageously, T represents one of the recognition agents iv. Advantageously, V represents any one of the targeting agents xxiv to xxvi.
The radioelement that may be fixed by the chelating agent may also be any radioelement that can be detected by an imaging or radioactivity counting system or has a radiotoxic effect.

Advantageously, V may be an agent targeting tumor cells.

The inventors noted that the rate of uptake of compounds of the invention by tumors was from 5 to 39%, per gram of tumor, at 4 hours.

“Chelating agent” refers to a group having the property of binding to a species with which it forms a complex called a chelate, wherein said species is bound to said chelating agent by at least two coordination bonds; said species is in particular an ion, more particularly a metal ion, or a metal, more particularly a metallic radioelement. For example, the chelating agent may represent a group that forms a chelate via at least two coordination bonds with a metal ion of gadolinium or manganese, or a radioelement that emits gamma radiation, or emits positrons, and/or emits particle rays, beta minus, Auger electron or alpha particles such as gold-33, copper-61, copper-62, copper-64, copper-67, gallium-67, gallium-68, zirconium-89, yttrium-90, technetium-99m, indium-111, iodine-123, iodine-124, iodine-125, iodine-131, holmium-166, lutetium-177, rhenium-186, astatine-211, actinium-89, bismuth-213. For example, it may be a group of formula:

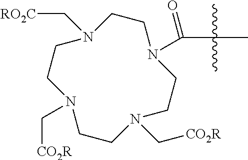

where each occurrence of R represents independently H or a protective group such as t-Bu; preferably all the occurrences of R are identical, preferably each R represents H; or it may be a group of formula:

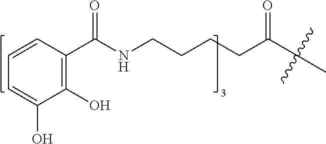

except in the case when D is a gallate dendrimer and V is a DOPA targeting agent comprising the structure of formula

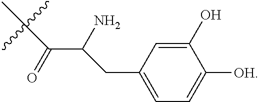 [SMILES: *C(=O)C(N)Cc1ccc(O)c(O)c1]

“Targeting agent” refers to an agent that can be recognized by specific, highly differentiated cells such as the cells of the central nervous system, tumor cells and/or cells in a particular metabolic state, for example hypoxic cells, apoptotic cells, or tumor cells having a particular phenotype giving rise to great aggressiveness and considerable cellular proliferation such as human growth factor receptor 2. The targeting agent may be an organic or inorganic group allowing a compound according to the present invention to be recognized by specific cells at the level of their membrane, of their cytoplasm or of their nucleus, and interacting with these cells preferentially.

The targeting agent may be a biological molecule produced by a living organism, or a molecule produced chemically, or any other molecule that can be recognized by specific cells, or by an extracellular matrix, notably a DNA, an RNA or a cellular, membrane or intracellular macromolecule, in particular a protein.

In particular, a targeting agent carried by a compound according to the present invention may be a biological molecule such as a lipid, phospholipid, glycolipid, sterol, glycerolipid, or a vitamin, a hormone, a neurotransmitter, an amino acid, a peptide, a saccharide, an oligonucleotide, an antibody, an antibody fragment, a nano-antibody, a ligand of a transmembrane transporter or of a membrane receptor, or a nucleic or peptide aptamer.

“Antibody” refers to an immunoglobulin capable of recognizing an antigen and neutralizing the function of said antigen. A complete antibody comprises two light chains and two heavy chains bound together by disulfide bridges.

“Antibody fragment” refers to an immunoglobulin comprising only scFv, divalent scFv, Fab, Fab′ or F(ab′)2 fragments.

“Nano-antibody” refers to a structure having the same structural and functional properties as a complete antibody, for example comprising only two immunoglobulin heavy chains.

Said antibody may be a natural antibody, in other words secreted by the B lymphocytes, or produced by hybridomas, or a recombinant antibody produced by a cell line. They may be monoclonal or polyclonal, of animal or human origin.

An antibody carried by a compound according to the present invention may enable said compound to reach a specific type of target cells, for example a type of tumor cells.

More particularly, said antibody may be an antibody directed against an antigen expressed by a type of tumor cells, such as the carcinoembryonic antigen, overexpressed by the cells of colorectal, thyroid medullary, lung cancers etc., or antibodies more specific of certain types of tumor cells such as those targeting the CA-15.3 antigen overexpressed by breast cancer cells, those targeting the CA125 antigen overexpressed by ovarian cancer cells, those targeting the Ca-19.9 antigen overexpressed by the cells of cancers of the gastrointestinal tract, and in particular pancreatic carcinomas, those targeting epithelial antigen overexpressed by chondrosarcoma cells, those targeting the PSMA antigen overexpressed by prostate cancer cells, those targeting the VEGF overexpressed by the endothelial cells of tumoral neovascularization, or those targeting the CD20 antigen expressed by normal or tumoral lymphocytes such as those proliferating in lymphomas, or an antibody directed against a protein expressed in the extracellular matrix.

As examples, and nonexhaustively, the antibody carried by a compound of the invention may be an anti-ERBB2, anti-CA-15.3, anti-CA-19.9, anti-PSMA, anti-VEGF, anti-CTLA-4, anti-CD20, anti-CD22, anti-CD19, anti-CD33, anti-CEA, anti-MUC1, or anti-tenascin antibody.

The targeting agent may be a peptide, a small chemical molecule, such as a neurotransmitter or a hormone.

As an example, and nonexhaustively, the ligand carried by a compound of the invention may be the peptide RGD (cyclic or not), the peptide NGR, GM-CSF, transferrin or galactosamine, the peptide HB-19, a fragment or a multimer of this peptide, a peptide targeting the melanocortin receptor, any other peptide targeting nucleolin, endostatin or angiostatin, or any other ligand of a receptor known by a person skilled in the art that is overexpressed on tumor cells.

The targeting agent may be an aptamer.

A nucleic aptamer may be a DNA or an RNA, produced by a combinatorial method of selection in vitro called SELEX (systematic evolution of ligands by exponential enrichment) (Ellington and Szostak, “In vitro selection of RNA molecules that bind specific ligands.”, Nature, Vol. 346, 1990, p. 818-822).

A target molecule of an aptamer may be proteins, nucleic acids, small organic molecules or whole cells.

An aptamer carried by a compound according to the present invention may be an aptamer targeting a receptor or a transporter, a transmembrane, intracytoplasmic or intranucleic protein present in normal cells, and overexpressed in tumor cells, such as the cells of acute myeloblastic leukemia (Sefah, Kwame, et al. “Molecular recognition of acute myeloid leukemia using aptamers.” Leukemia, 23 (2009); 235-244 [25]), or the extracellular matrix, for example an anti-MMP9 aptamer, targeting type 9 metalloproteinase secreted by certain types of tumor cells, notably prostatic or melanoma cells. It may also be a nucleic acid or protein aptamer such as AS1411 or derivatives thereof, targeting with strong affinity nucleolin, a protein overexpressed in the nucleus, cytoplasm and membrane of many types of tumor cells (a) Z. Cao, R. Tong, A. Mishra, W. Xu. G. C. L. Wong, J. Cheng, Y. Lu, Angew. Chem. 2009, 121, 6616-6620; Angew. Chem. Int. Ed. 2009, 48, 6494-6498; b) S. Christian, J. Pilch, M. E. Akerman. K. Porkka, P. Laakkonen, E. Ruoslahti, J. Cell Biol. 2003).

In particular, the targeting agent may be any other biological molecule, such as 2-oxoglutarate or metronidazole derivatives (MISO), for targeting hypoxic cells, or a molecule produced chemically, such as the pentavalent DMSA for targeting the overexpressed proteins involved in the calcium metabolism of tumor cells, such as DOPA, targeting (J Neurooncol DOI 10.1007/s11060-012-0986-1) the overexpression of the LAT1 transporters in aggressive glial tumors (J Neurooncol 99:217-225) or neuroendocrine tumors.

The targeting agent may also be selected from the chemical compounds described in Maisonial et al. J. Med. Chem. 2011, 54, 2745 [26]. Rbah-Vidal et al. Eur. J. Med. Mol. Imaging 2012, 39, 1449 [27]. Vivier et al. Eur. J. Med Chem. 2011, 46, 5705 [28] and WO2009/095872 [29], targeting melanoma cells, as well as their analogs and derivatives. Such a targeting agent has for example the structure:

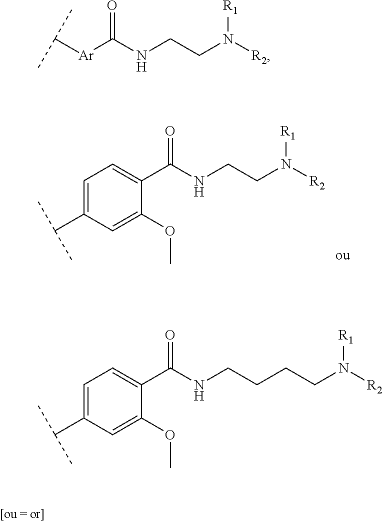

wherein R₁ and R₂ represent, independently of one another, a linear, branched or cyclic (C₁-C₆)-alkyl chain, in particular a methyl, an ethyl, a propyl, an isopropyl, a butyl, more particularly an ethyl, where R, and R₂ may be bound to form a ring, R₁ and R₂ representing in particular 2-azanorborn-2-yl, Ar represents a quinoxalinyl group optionally substituted with a heteroalkyl chain.
Advantageously, the targeting agent may have the following structure V1:

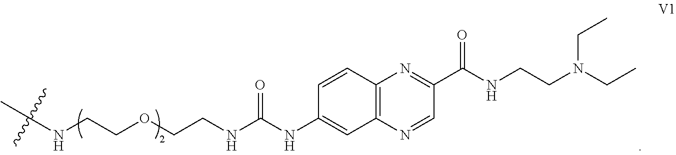

Advantageously, when the compounds according to the invention comprise at least two groups V, said groups V may have the same structure.
According to one embodiment, the following compounds are excluded from the invention:

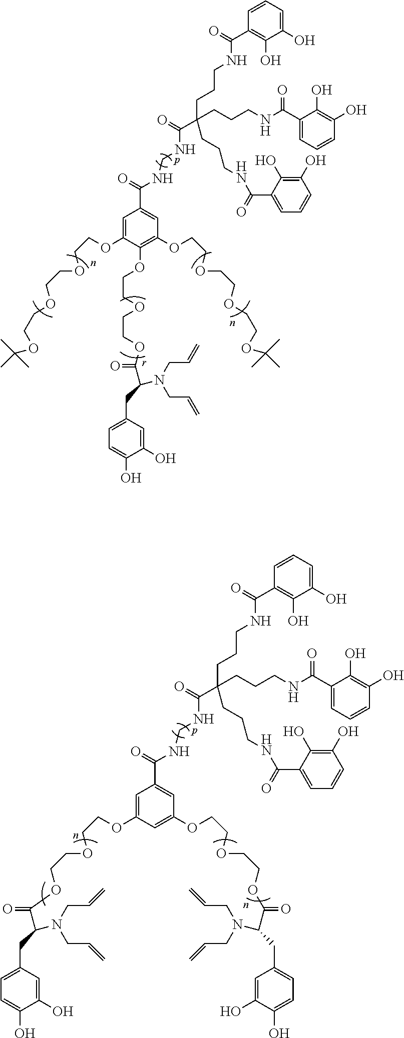

n, p and r being, for these two formulas, between 1 and 10, between 3 and 6, and between 1 and 20, respectively.

According to one embodiment, the compounds specifically disclosed in WO 2008/043911 are excluded.

“Recognition agent” or “specific molecule” refers to a small organic molecule, such as biotin, a single-stranded oligonucleotide, a hormone, or a neurotransmitter; or a macromolecule, such as an antibody, a transmembrane protein that recognizes a receptor or a protein antigen.
The recognition agent and the specific molecule may form a complex, notably the “avidin-biotin” complex, the “streptavidin-biotin” complex, a complex of a double-stranded oligonucleotide, an “antibody and antigen” complex, a “ligand and receptor” complex, or an “adamantane-cyclodextrin” complex.

### Click-Clack System

According to one variant, the group T of the compound of formula I may be avidin, streptavidin, or biotin, which allows said compound (click system) to couple to another therapeutic or diagnostic molecule (clack system), in particular another dendrimer, comprising
biotin, when group T of the compound of formula I is avidin or streptavidin,
avidin or streptavidin, when group T of the compound of formula I is biotin.
adamantane. when group T of the compound of formula I is cyclodextrin, or
cyclodextrin. when group T of the compound of formula I is adamantane.

For example, the “click system” may be a compound of formula III

T-L₁-DV)ₙ (III)

wherein:

T is biotin, avidin, streptavidin, cyclodextrin or adamantane;

L₁ is a heteroalkyl chain of structure:

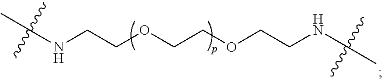

wherein p is an integer from 1 to 10; preferably 4 or 6

or else T is absent and L₁ corresponds to one of the following azide structures:

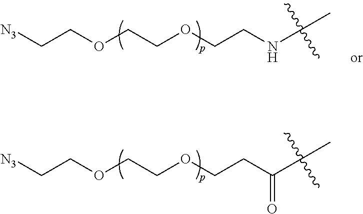

wherein p is an integer from 1 to 10; preferably from 1 to 8; preferably 3 or 7;

D is a PAMAM dendrimer of formula (DD):

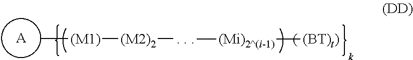

wherein:
(a) A is the dendrimer nucleus, of multivalence 2;
   A is a synthon of structure:

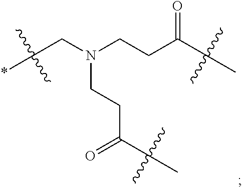

where * indicates the point of attachment to the spacer L₁;
(b) Mi is a monomer of generation i, where:
   i is an integer ranging from 0 to g, g being the generation number of the dendrimer;
   when i=0. Mi is absent, and the terminal branch TB is then bound directly to the nucleus A;
   when i>0, Mi represents:

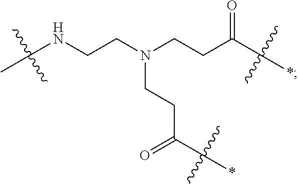

with i=0 or 1; preferably i=1;
where the symbol * denotes the point of attachment of the monomer Mi to the monomer of higher generation;
(c) TB is the terminal branch, and t is the number of terminal units from the monomer of lower generation, where:
   t is 2;
   TB is a radical selected from the group comprising a polyethylene glycol chain of formula:

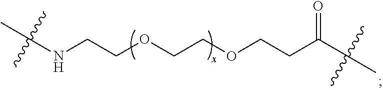

wherein each occurrence of x is independently an integer from 1 to 10; and at least one occurrence of TB is covalently bound to a targeting agent V:

V represents a melanoma targeting agent corresponding to the formula:

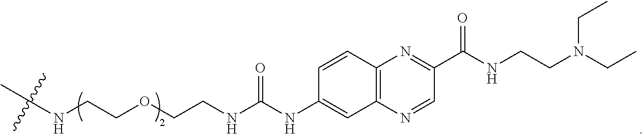

For example, the “clack system” may correspond to formula IV:

T′-L₁-R IV

wherein

T′ is adamantane, cyclodextrin, biotin, avidin or streptavidin;

L₁ is a heteroalkyl chain of structure:

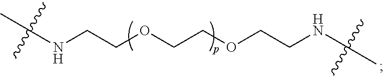

wherein p is an integer from 1 to 10; preferably 4 or 6;

or else T′ is absent and L₁ corresponds to one of the following azide structures:

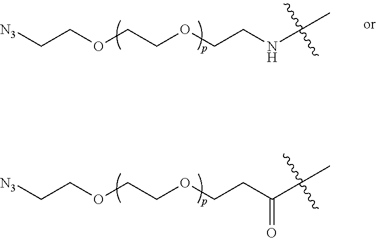

wherein p is an integer from 1 to 10; preferably from 1 to 8; preferably 3 or 7;

R is a labelable moiety such as L-thyroxine or dinitrobenzoate:

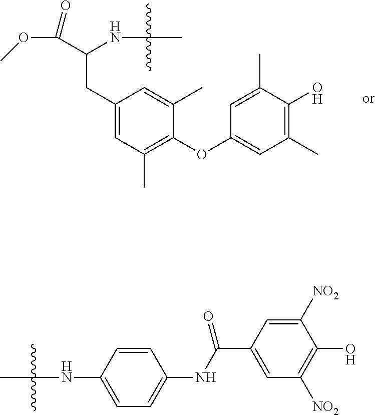

Advantageously, a “click system” compound according to the present invention, comprising avidin, streptavidin, biotin or adamantane or a cyclodextrin as recognition agent T, may have, as “clack system”, a compound of formula IV, where the group T′ may be conjugated to a second dendrimer of formula DD^{A}, to form a complex.
This system makes it possible to combine the properties conferred by different dendrimers.
As an example, but not exhaustively, the second dendrimer may be any dendrimer known by a person skilled in the art.
Advantageously, the second dendrimer is of the following formula (DD^{A}):

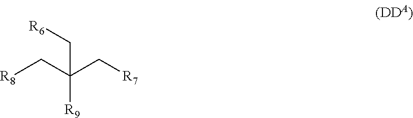

wherein:

R9 represents said specific molecule.

R6, R7, R8 represent, independently of one another, a dendrimeric group of generation 1≦n≦7, said dendrimeric group comprising:

(a) a core having an amine group and two carbonyl groups, and represented by the formula A:

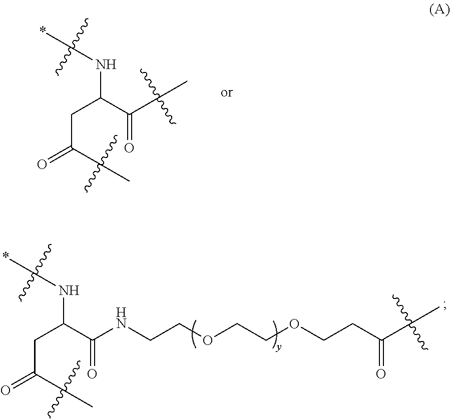

(b) end groups Ra selected from
(i) the end group of formula Ra1

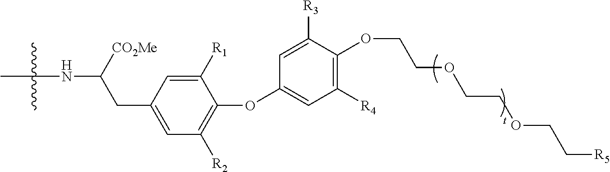

wherein:
R1, R2, R3 and R4, represent I,
R5 represents —NHBoc,
t represents a natural integer greater than 0 and less than 7, or
(ii) the end group of formula Ra2

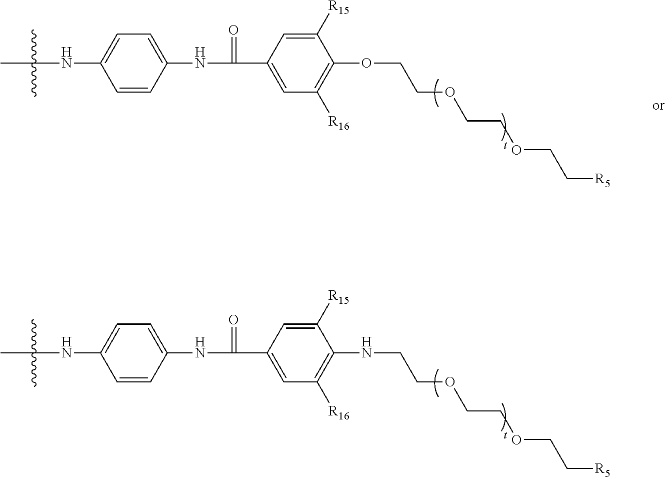

wherein:

R₁, R₁₆ represent F or —NO₂, Cl, Br, CH₂OMs, CH₂OTs, CH₂Br or CH₂Cl; preferably R₁ and R₁₆ both represent —NO₂;

R₅ represents —NHBoc,

t represents a natural integer greater than 0 and less than 7:
(c) and when said dendrimeric group is not a dendrimeric group of generation one, said dendrimeric group also bearing dendrons represented by the formula B:

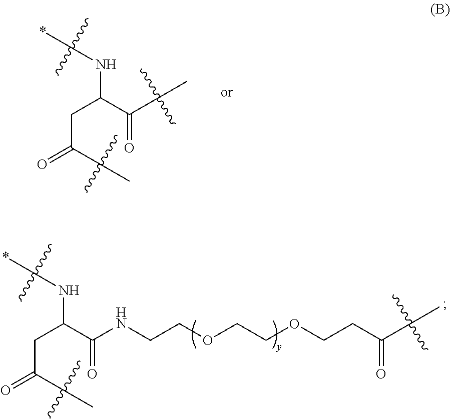

said dendrimeric group of generation n, n≧1, comprising n−1 ranks of dendrons,

the core of formula A being such that:

the amine group of said core is bound to the carbon chain of formula I,

the two carbonyl groups of said core are bound:

(i) either to the amine group of an end group represented by Ra, when said dendrimeric group is of generation one;
(ii) or to the amine group of a first rank dendron of said dendrimeric group, when said dendrimeric group is not of generation one, or
(iii) to an —OH group, provided that the two carbonyl groups of said core are not both bound to an —OH group, and

the end group Ra being such that:

the amine group of said end group is bound:

(i) either to a carbonyl group of the core, when said dendrimeric group is of generation one; or
(ii) to a carbonyl group of a dendron of the last rank (rank n−1) of said dendrimeric group, when said dendrimeric group is not of generation one, and

a dendron of formula B and of rank m, 1≦m≦n−1, being such that:

the amine group of said dendron of rank m is bound:

either to a carbonyl group of the core, when said dendron is of first rank

or to a carbonyl group of a dendron of the preceding rank m−1, when said dendron is of rank greater than 1;

the two carbonyl groups of said dendron are bound:

either to the amine group of an end group Ra, when said dendron is of last rank,

or to the amine group of a dendron of the next rank m+1, when said dendron is not of last rank

or to an —OH group, provided that the carbonyl groups of said dendrimeric group are not all bound to an —OH group.

Such a complex, whether it is with a dendrimer DD^{A} or a labelable moiety such as L-thyroxine makes it possible to combine the high vectoring power supplied by the compound of formula (I) according to the present invention with the signal amplification properties supplied by the compounds of formula DD^{A} or the labelable molecules.

According to one aspect, the present invention supplies the following compounds:

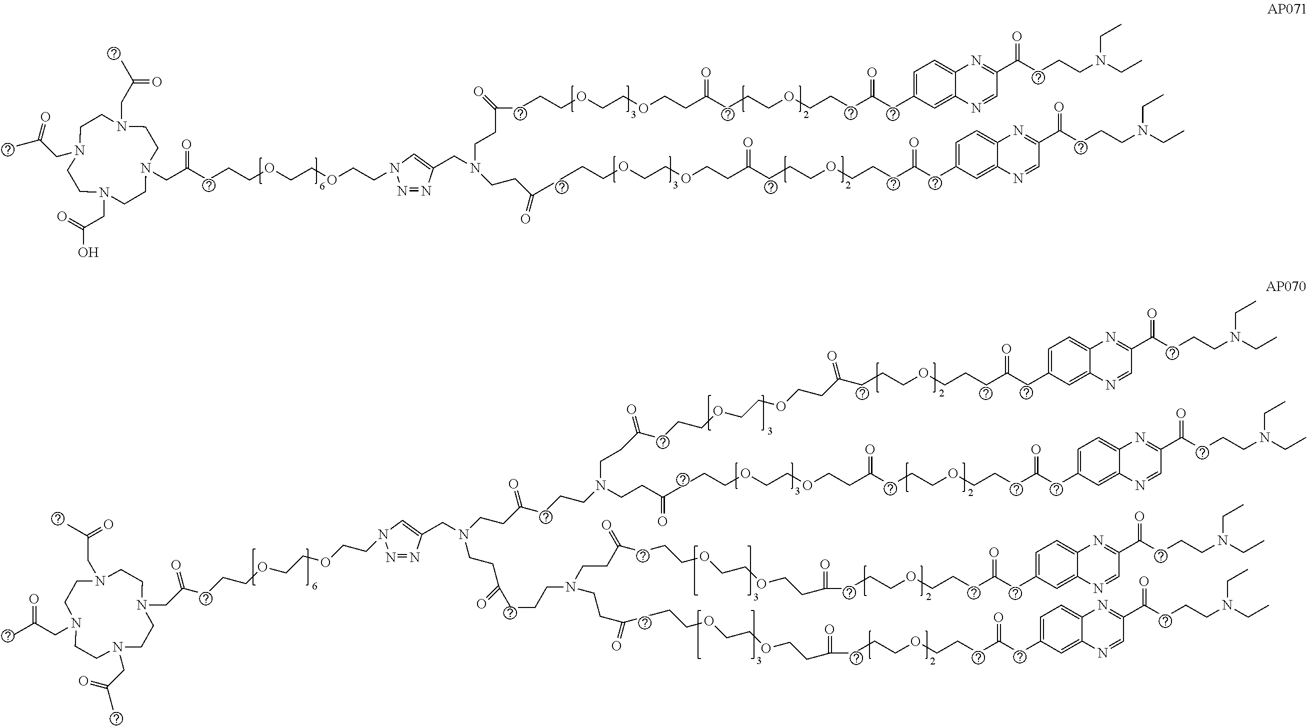

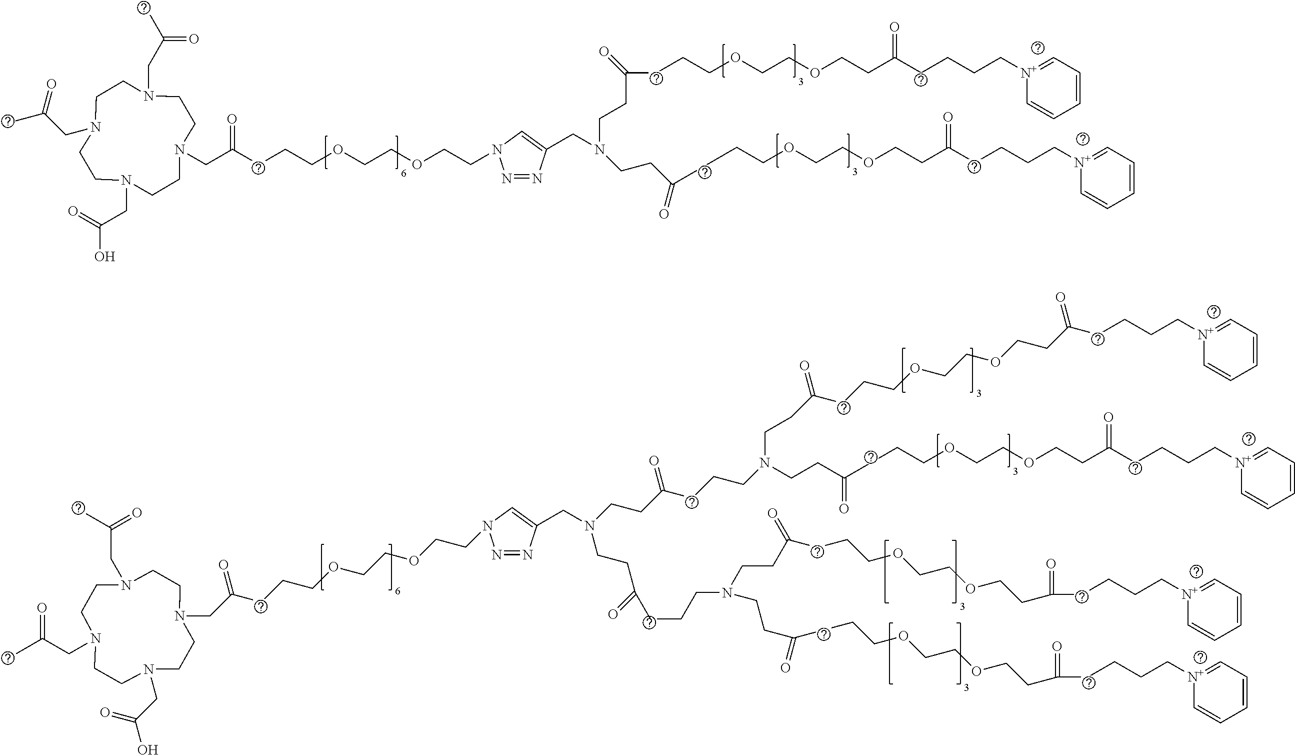

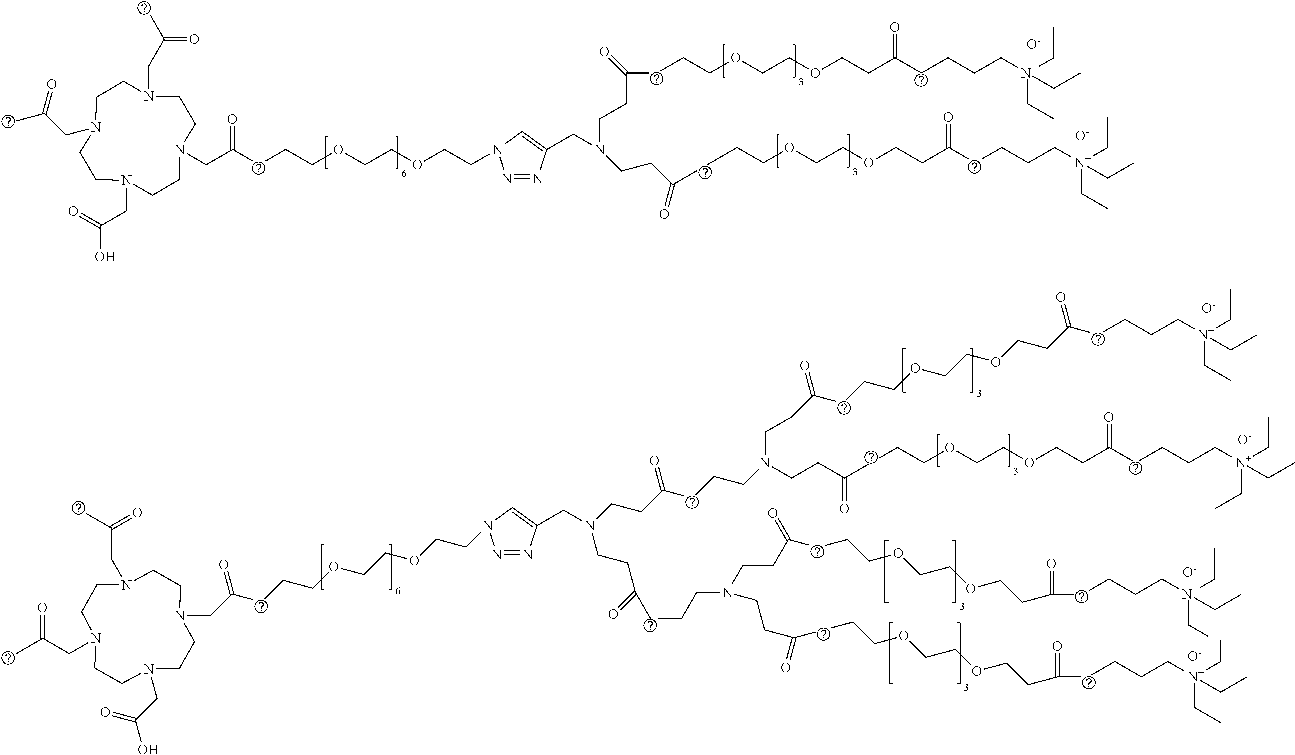

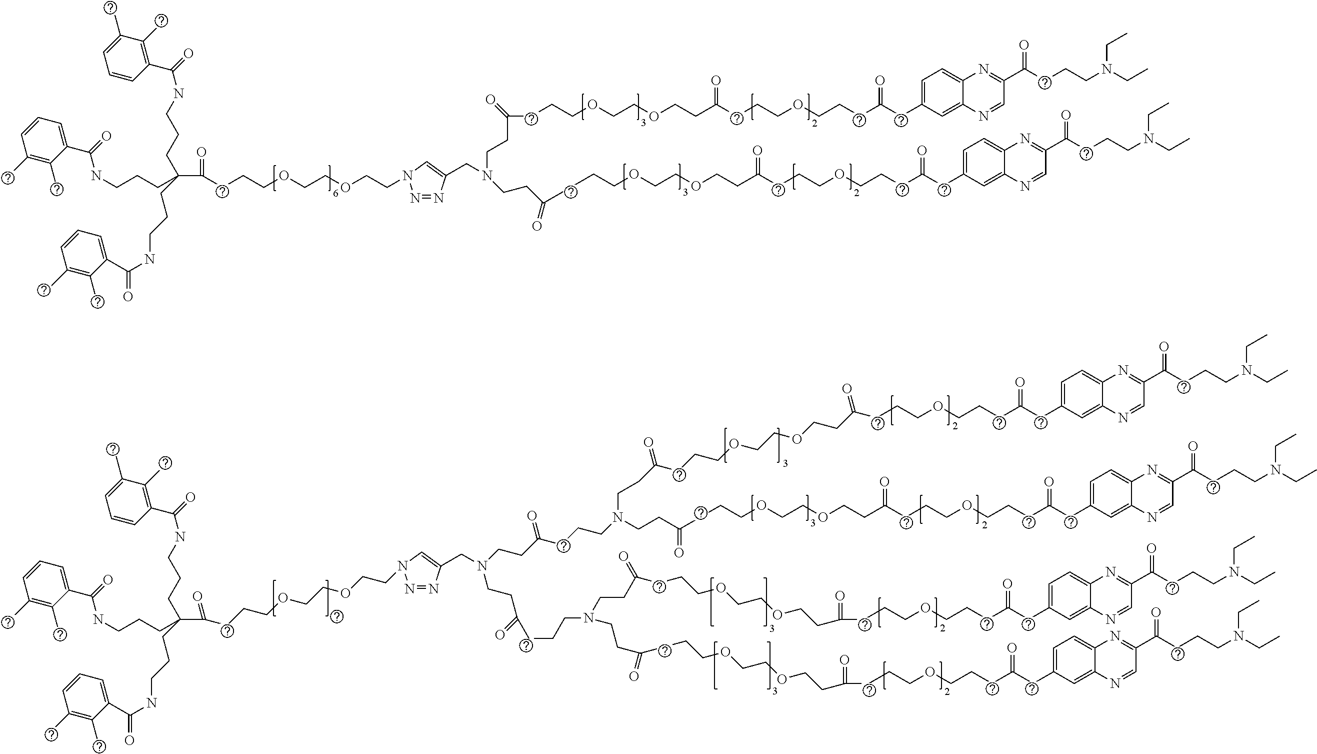

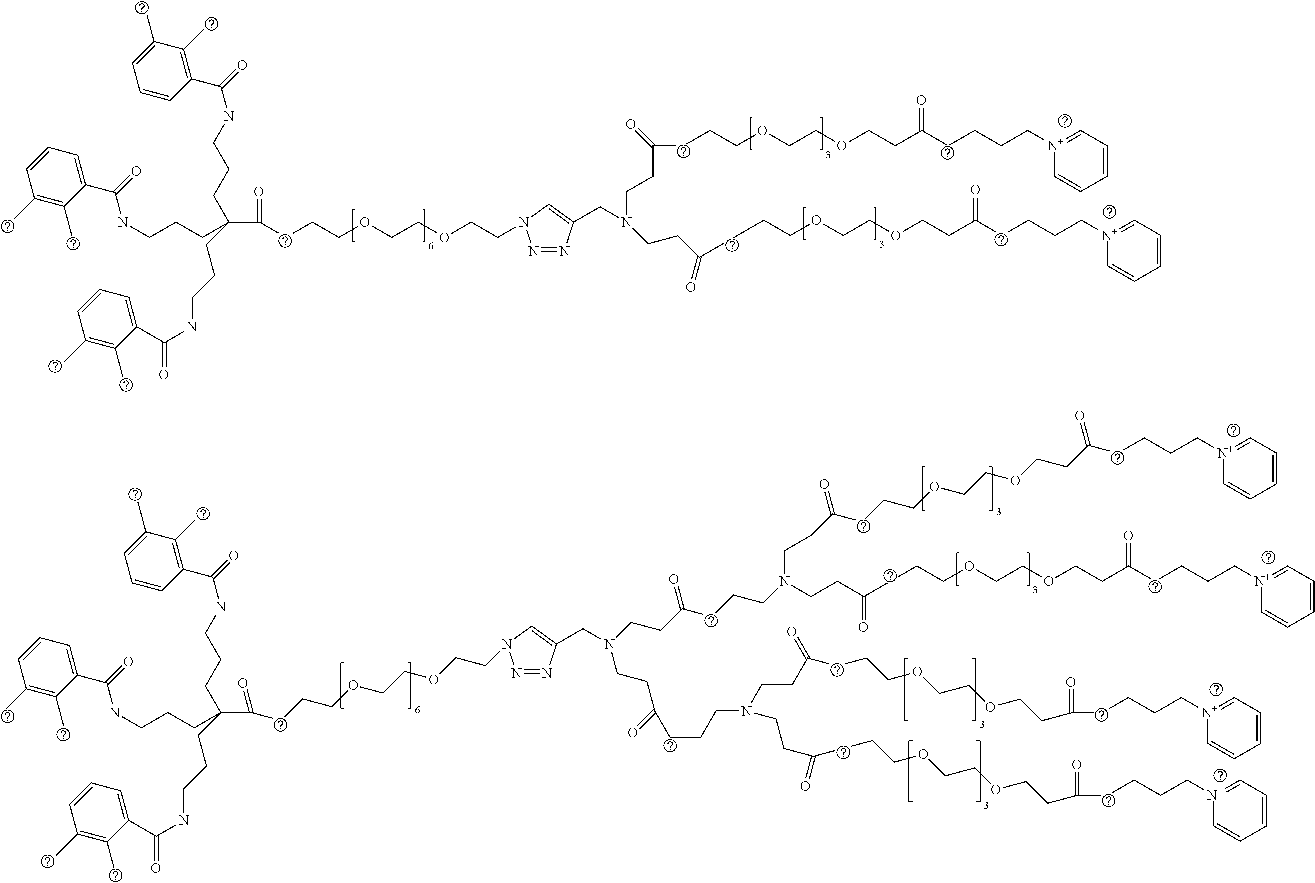

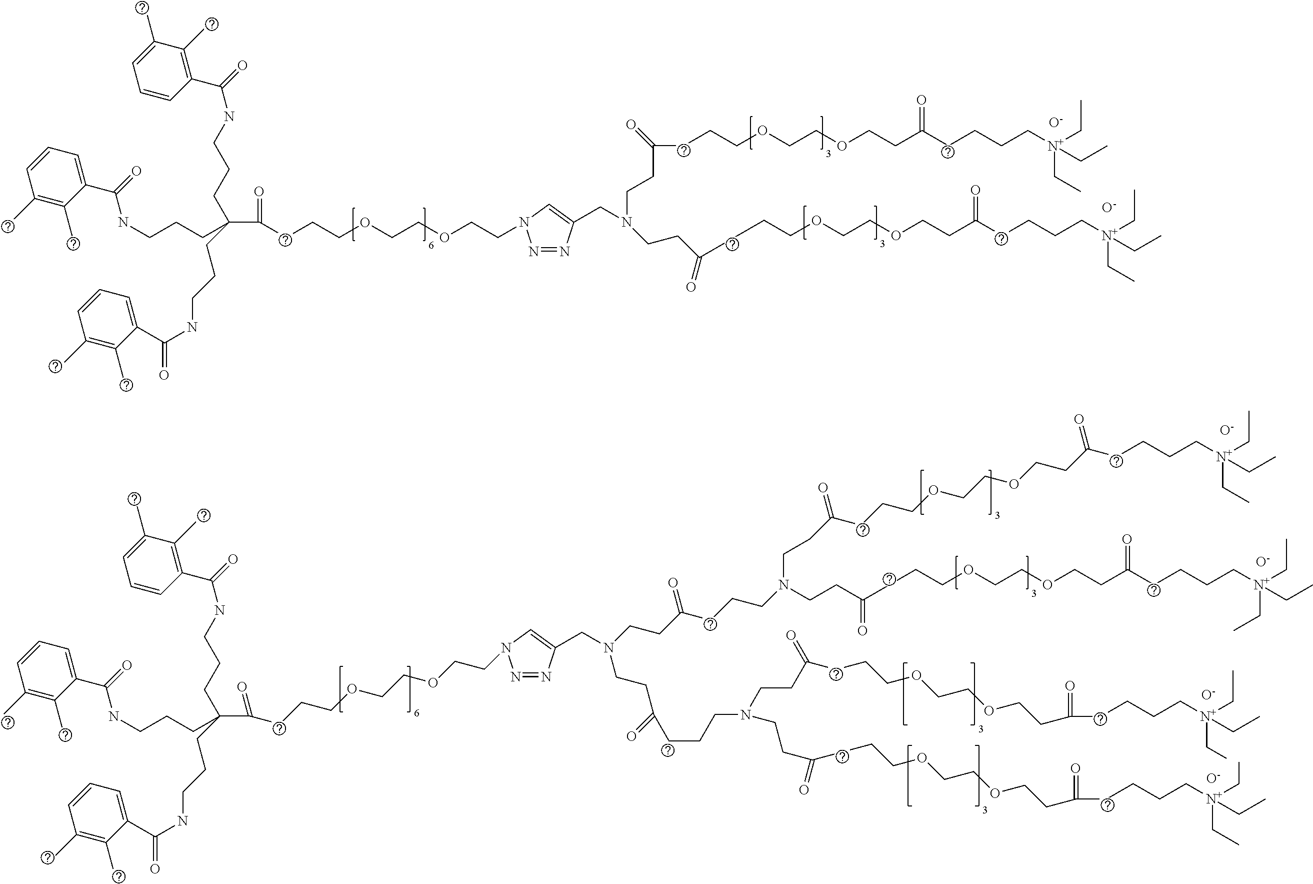

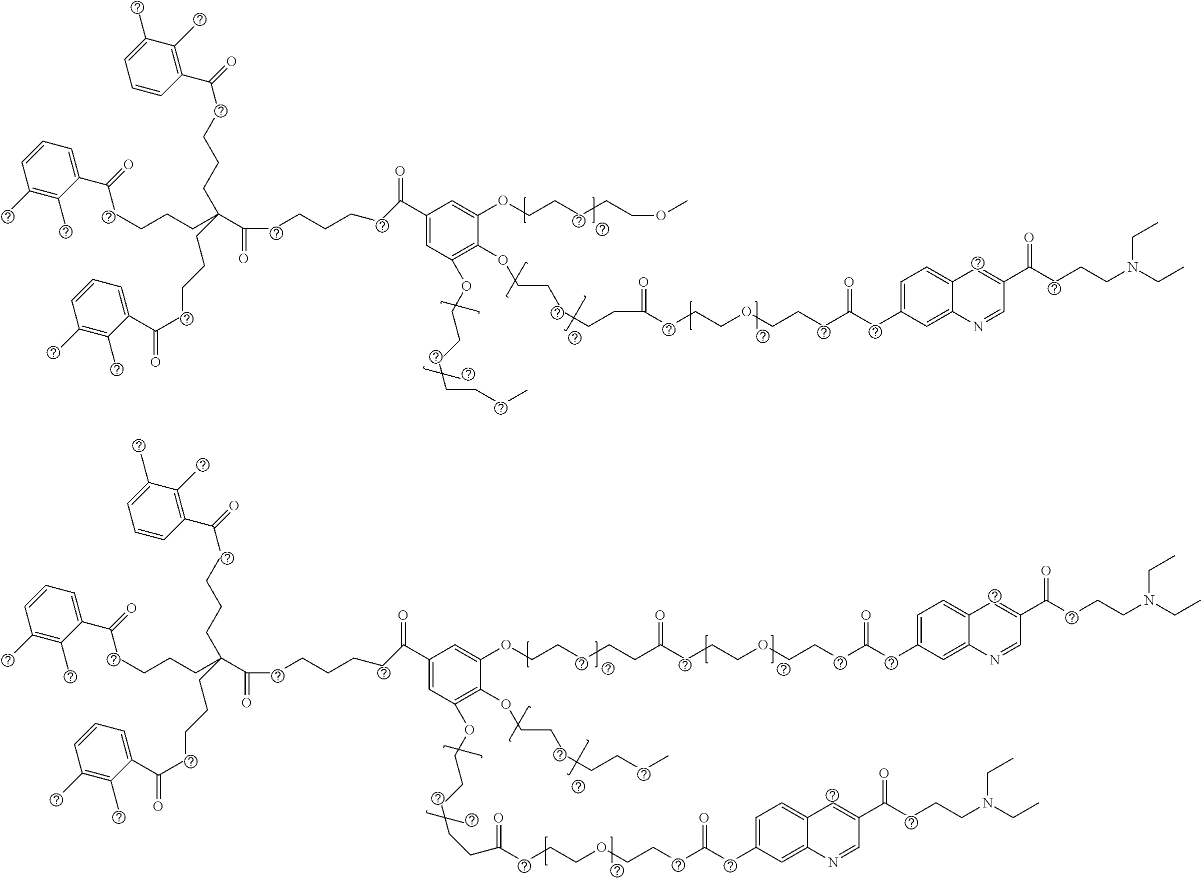

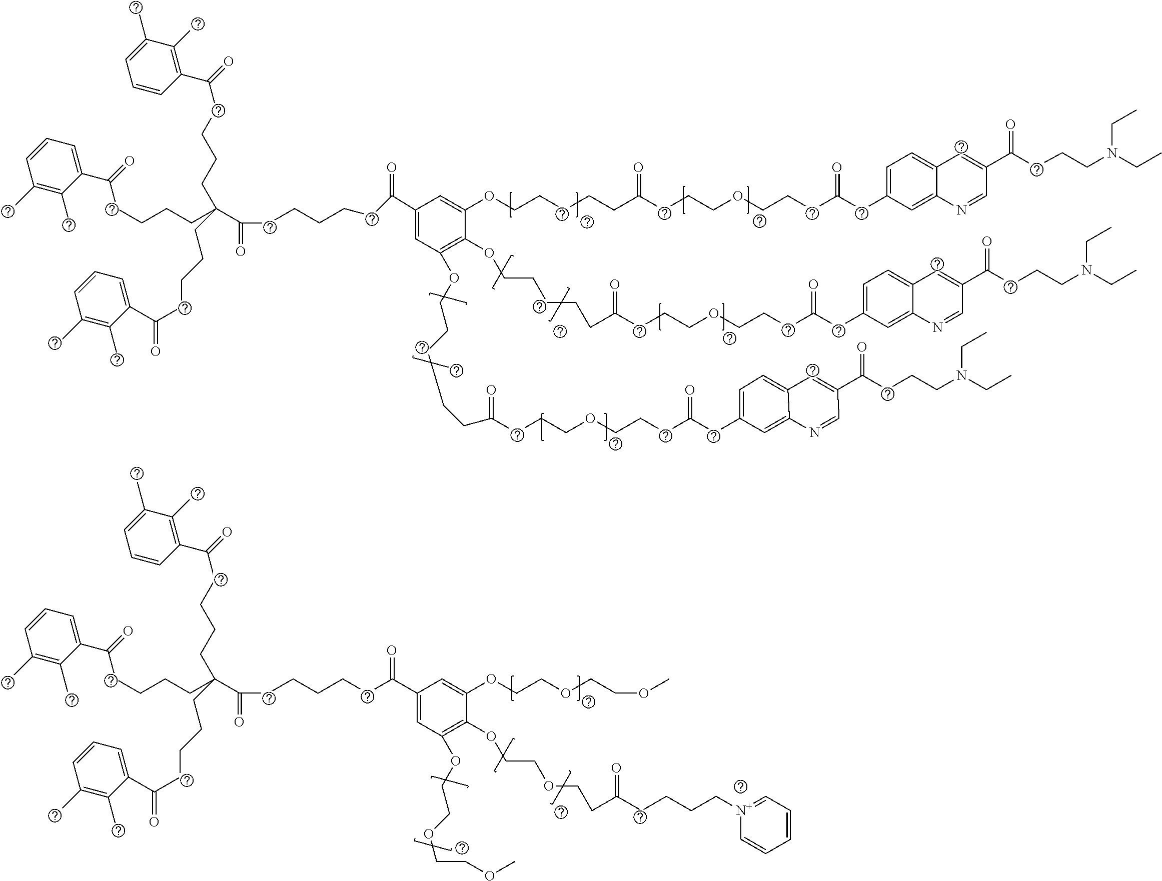

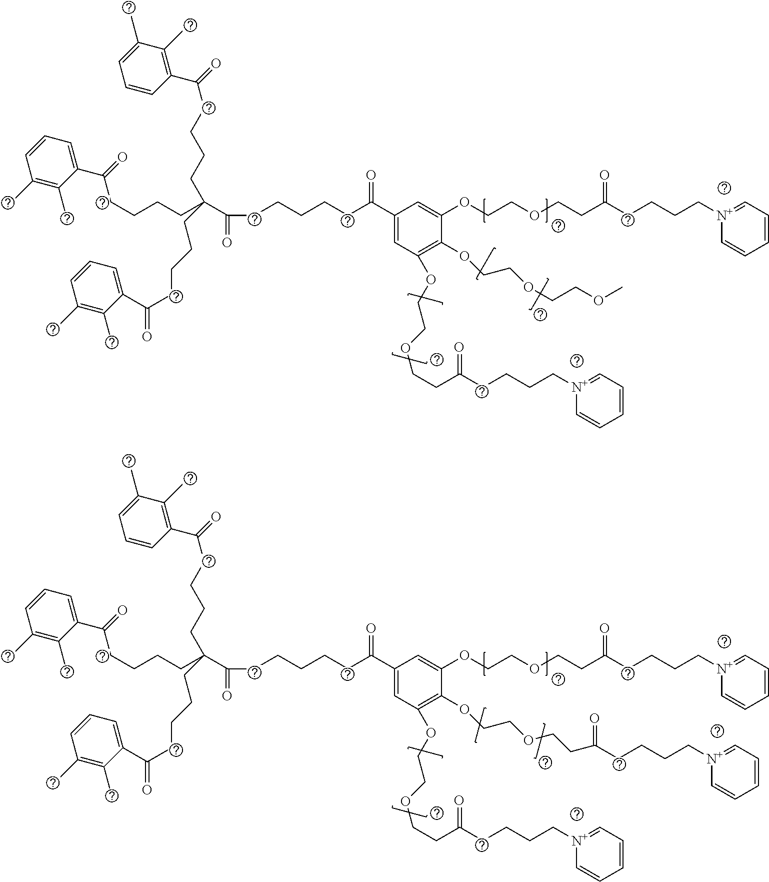

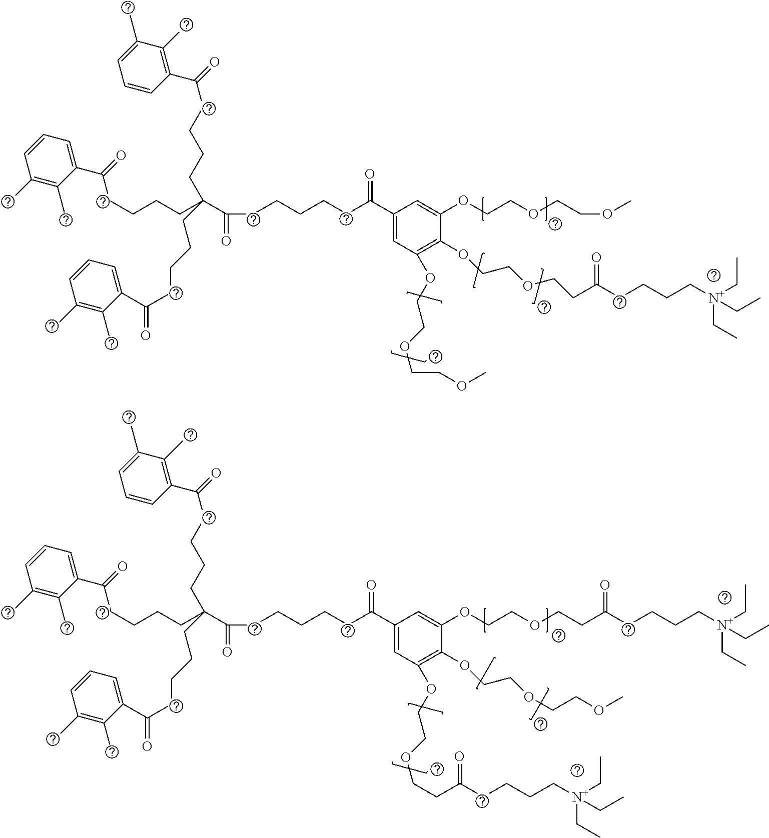

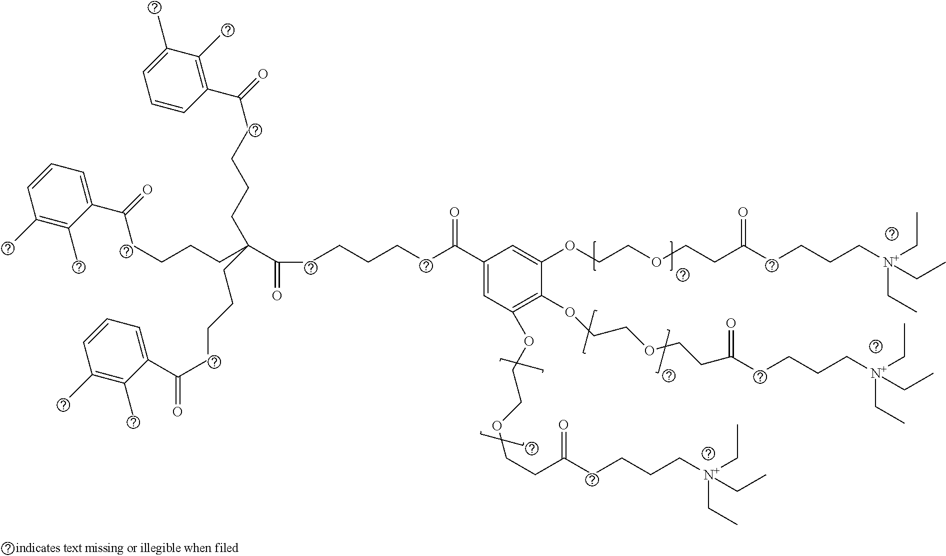

For the gallate units depicted below, the spacer L₁ of formula —NH(CH₂CH₂O)₆CH₂CH₂NH— between the chelating agent and the dendrimer may be replaced by the spacer of formula:

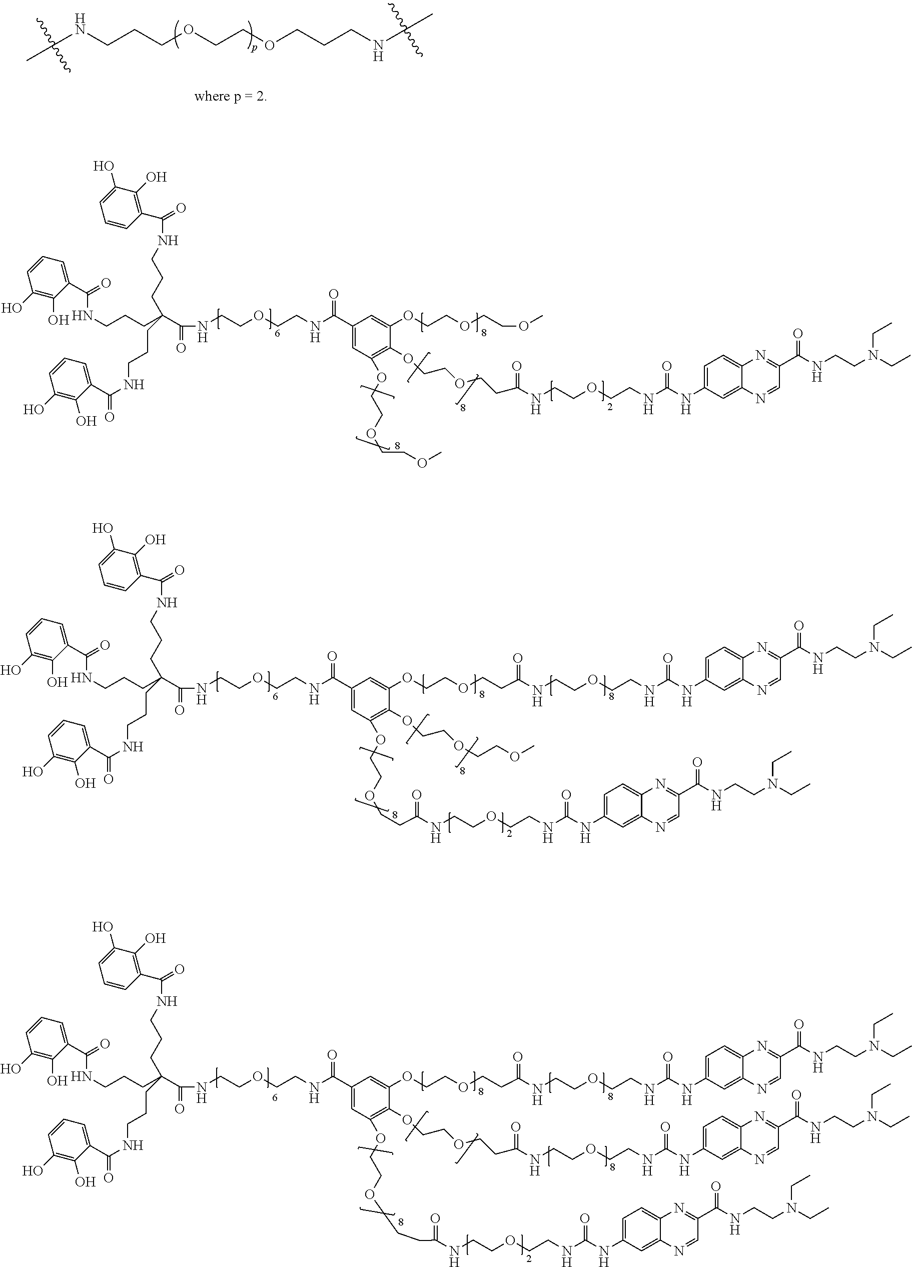

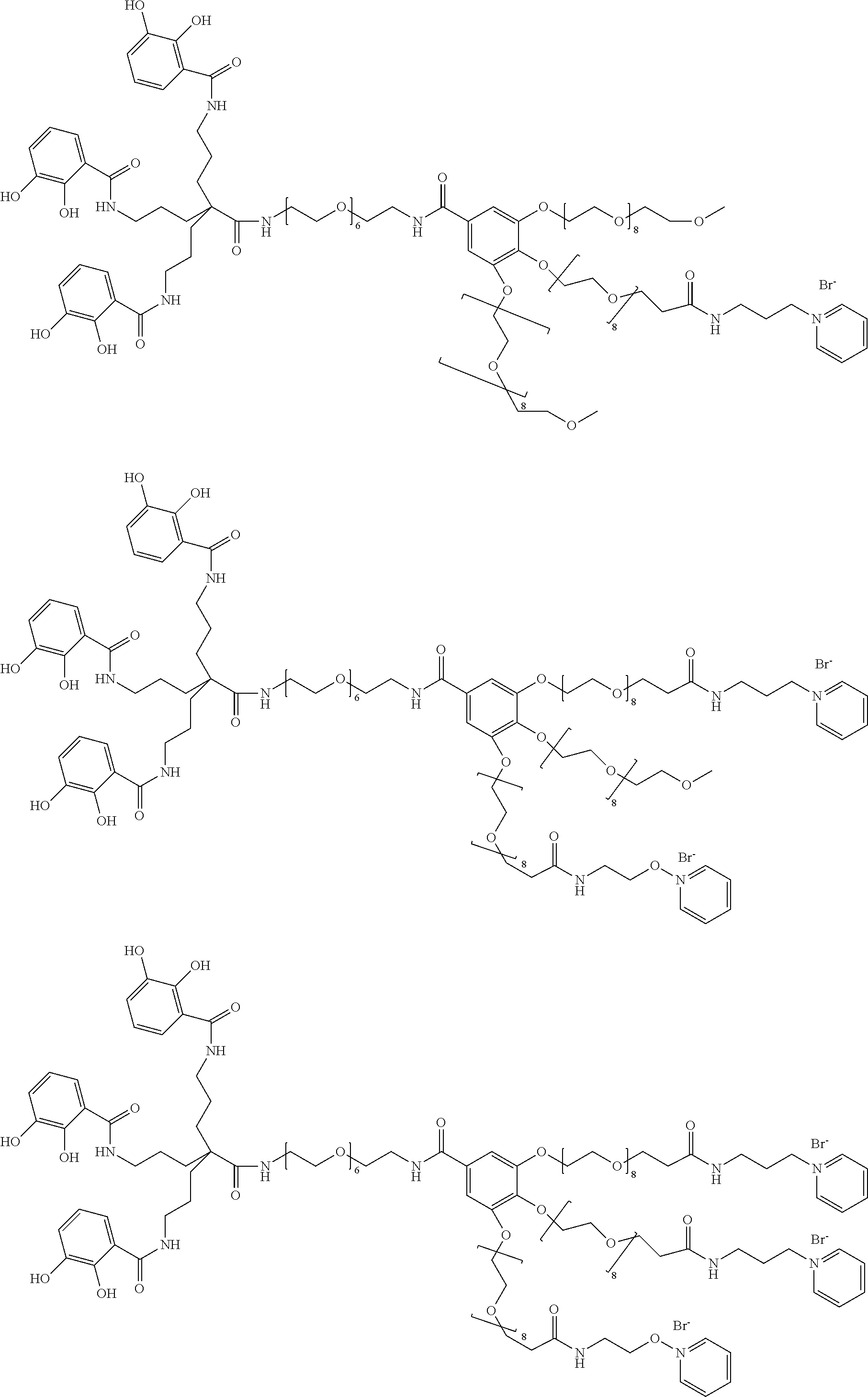

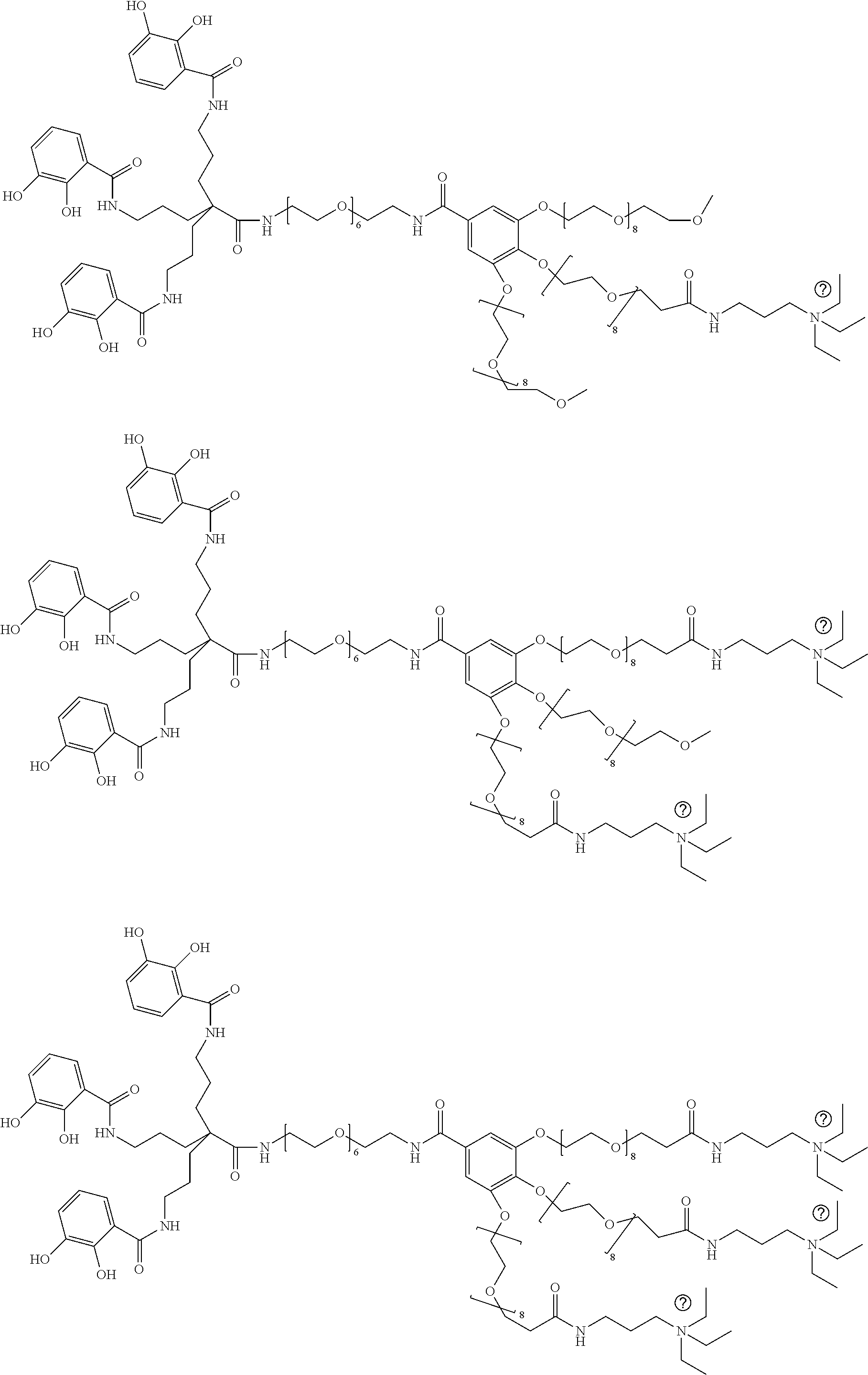

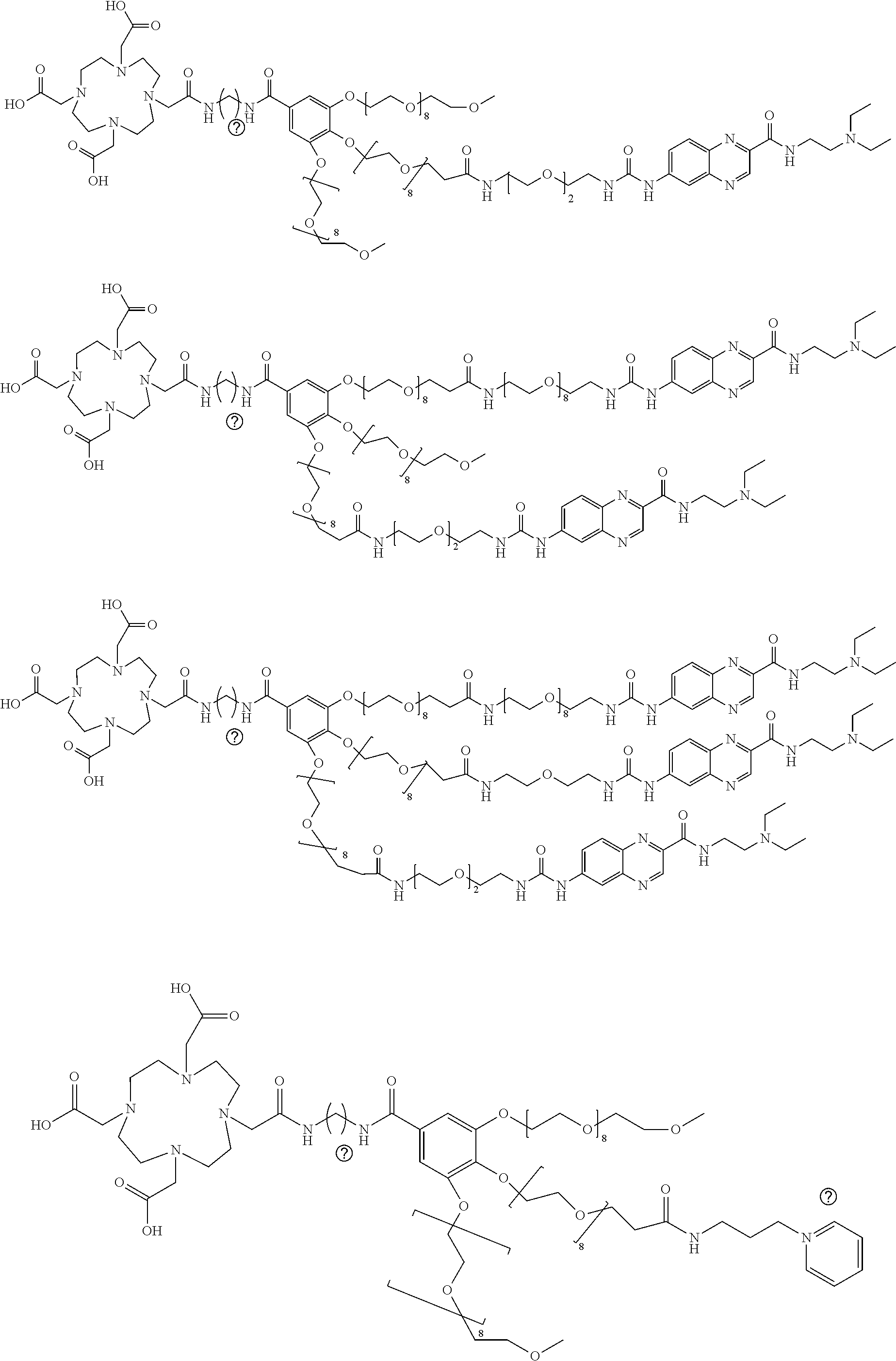

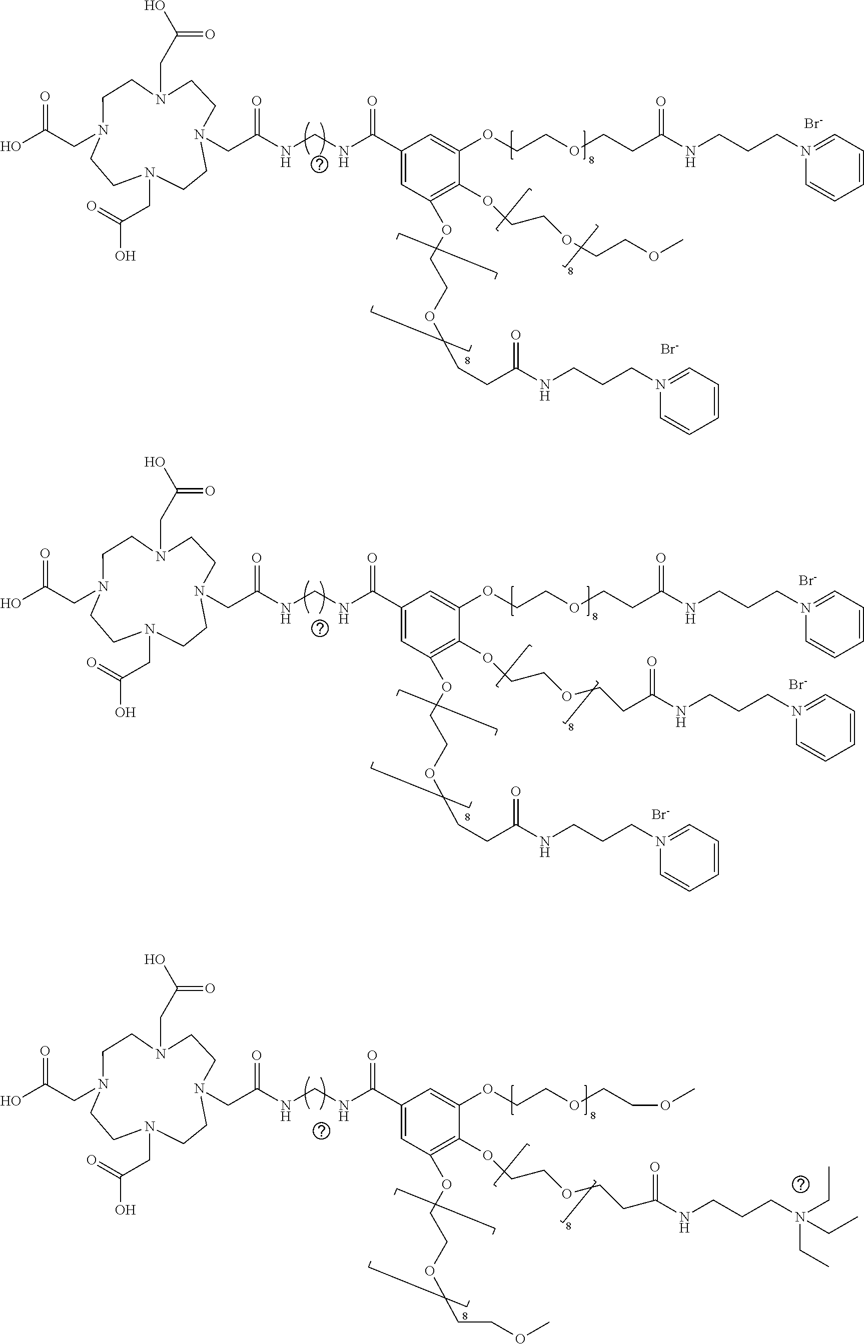

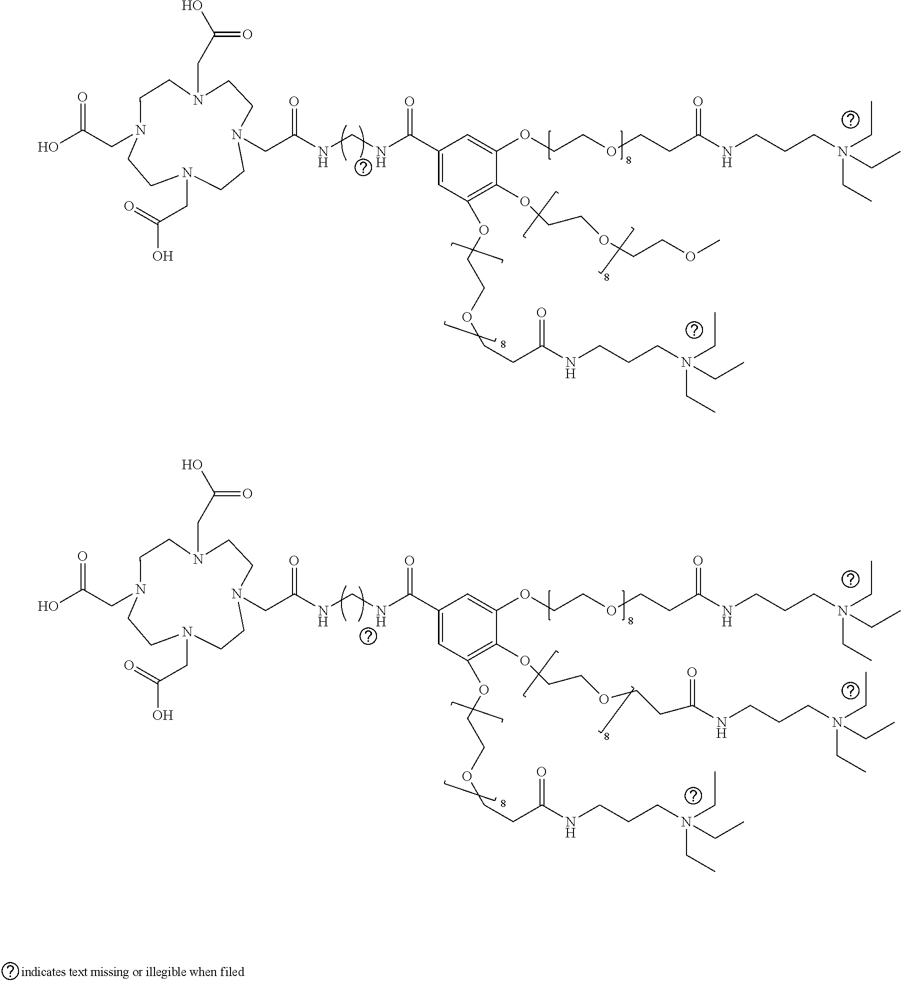

For the gallate units depicted below, the spacer L₁ of formula —NH(CH₂CH₂O)₆CH₂CH₂NH— between the chelating agent and the dendrimer may be replaced by the spacer of formula:

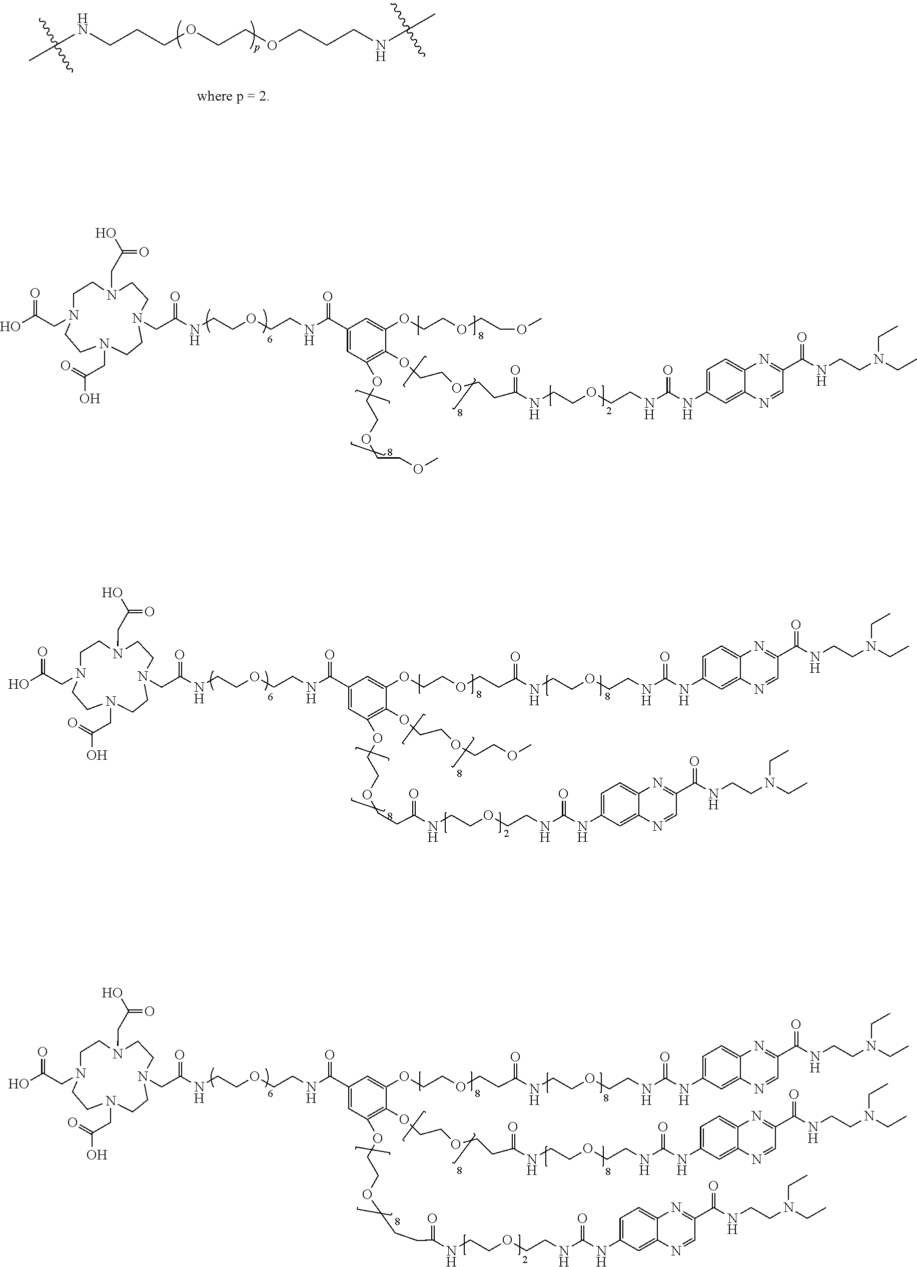

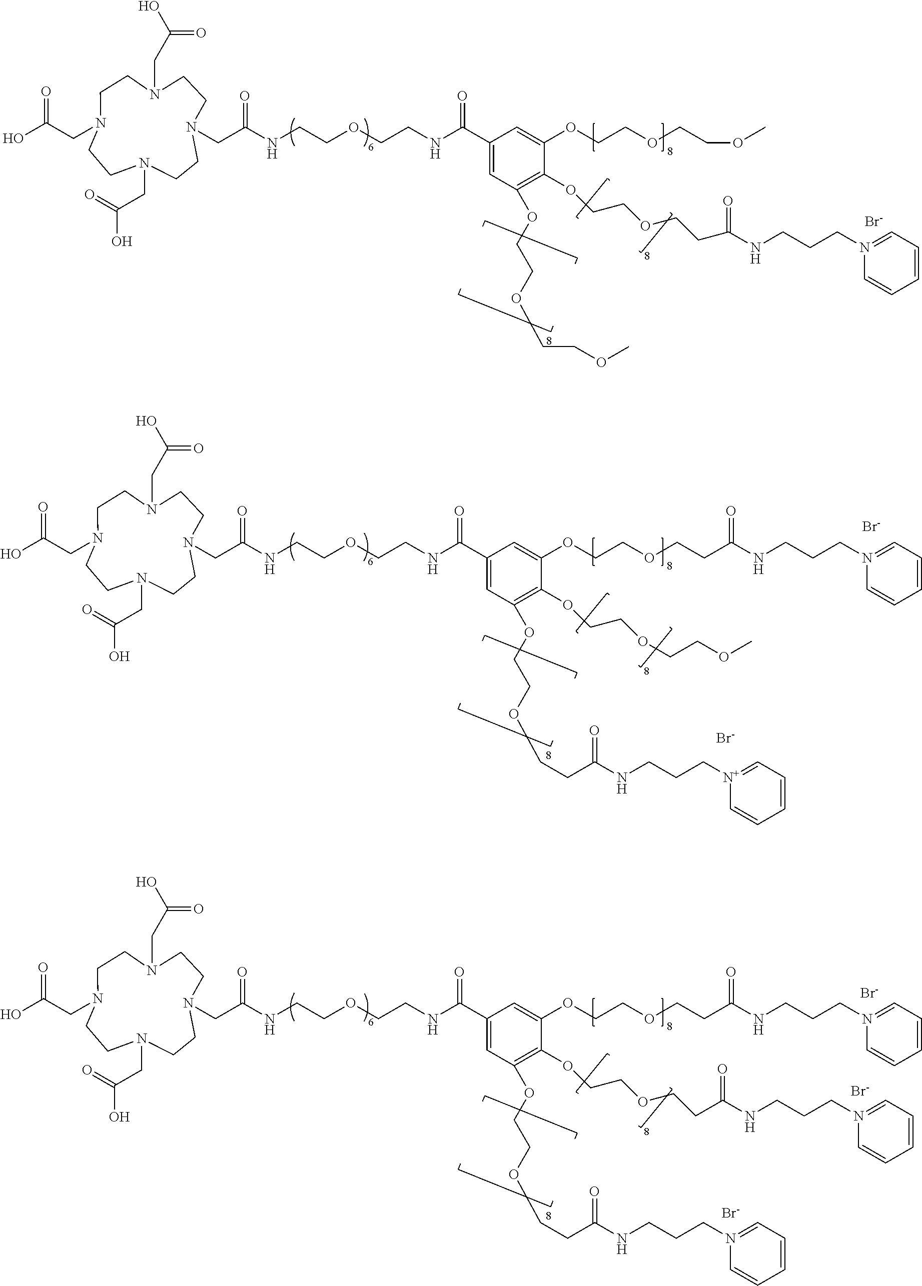

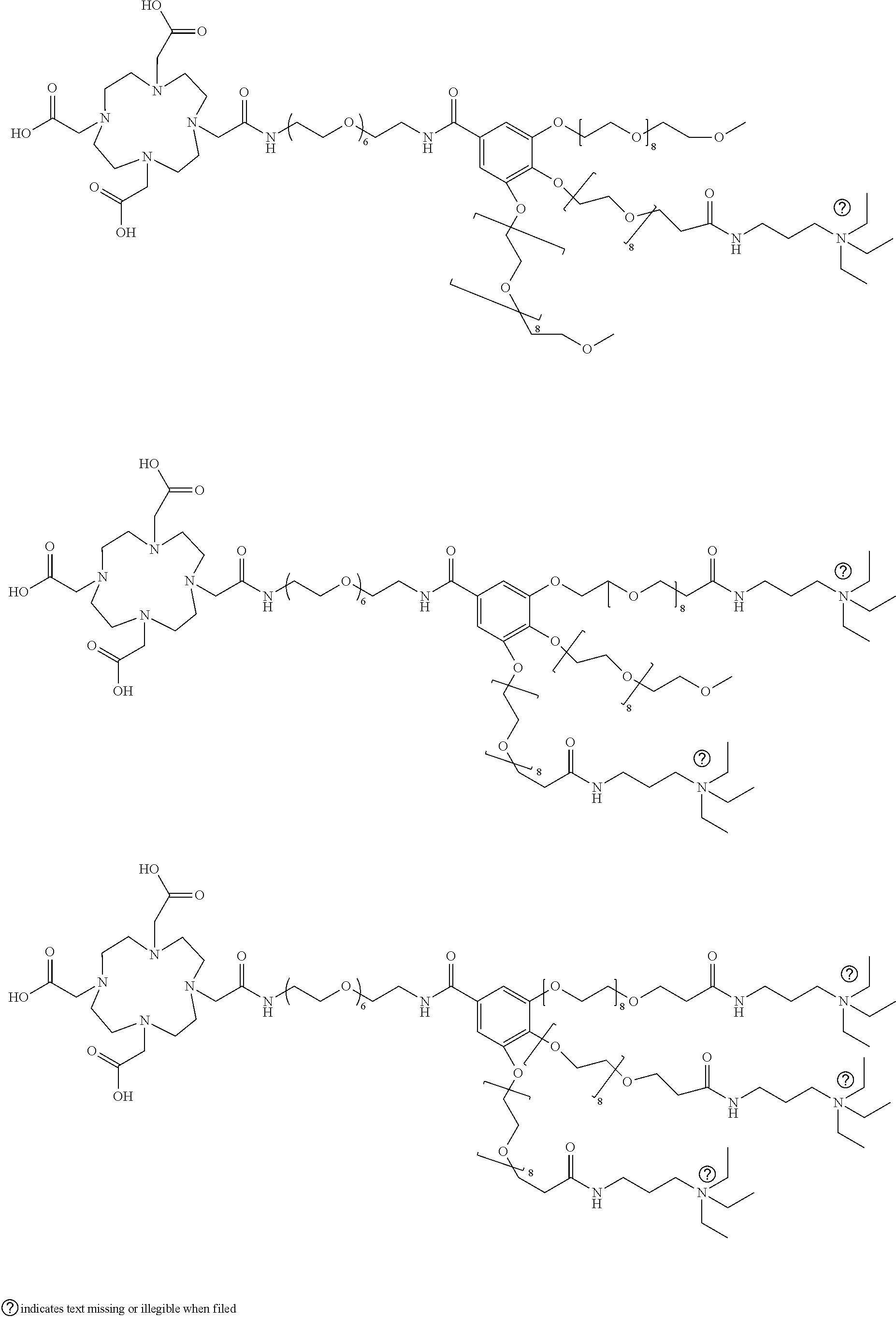

### Uses

The invention also relates to a complex as described above, for use as a tool for detecting tumors, notably in the form of micrometastases, and/or cells in a particular metabolic state, notably hypoxic cells or apoptotic cells.
The invention also relates to a complex as described above, for use as a tool for characterizing the sentinel lymph node.
Currently, determination of the tumoral status of the sentinel lymph node (SLN) of any type of tumors requires a surgical biopsy for histopathological analysis. In melanoma, a method of low invasiveness guided by lymphoscintigraphy is required. In melanoma, minimally invasive surgical biopsy, guided by lymphoscintigraphy, is currently carried out in order to prevent the risk of underestimating the tumor grade by failing to recognize the existence of clinically occult lymph node metastases, thus compromising the choice of adjuvant treatment such as chemotherapy or immunotherapy.
With the aim of optimizing the carcinologic quality of exeresis in Dubreuilh melanosis, the boundaries of which are often poorly definable, a larger exeresis is required with a margin of 5-10 mm at the price of aesthetic sequelae and greater complexity of the reconstructive surgery. Mohs micrographic surgery, allowing restriction of the resection zone, is very rarely applied owing to the extra expense and the time required for exeresis. Optical imaging has already been evaluated by several teams, who perform ex-vivo examination in confocal microscopy, of samples from a 2 mm strip around the lesion thanks to nonspecific fluorescent labeling of the nuclei.
Despite the development of targeted therapies and immunotherapy (anti CTLA4), high-grade melanoma with distant metastases still has a poor prognosis.
Metastatic cutaneous lesions in the elderly are difficult to manage, as the use of chemotherapy, radiotherapy or targeted therapies is contraindicated.
Knowledge of the metastatic tumoral status of the sentinel lymph node with a high negative predictive value would make it possible to avoid an unnecessary lymphadenectomy. An efficient and specific system for in vivo labeling of the tumor cells would make it possible to determine the tumoral limits of lesion in Dubreuilh's disease; this determination can be more accurate if it is based on Raman spectroscopy. Targeted internal radiotherapy (IRT), notably alpha therapy, could be effective against micrometastases or in-transit metastases.
By combining alpha and/or beta IRT and radiosensitization by external radiotherapy, great efficacy is expected on metastases, notably cerebral metastases. Internal radiotherapy with beta emitters delivered by topical application could be interesting as alternative, palliative treatment of metastatic cutaneous lesions in the elderly.
Advantageously, the use of a compound of formula I according to the invention (dendrimeric theragnostic nanoplatform (DNP)) having very effective targeting properties, and high efficiency in terms of diagnosis and/or treatment, makes it possible to respond to several challenges: i) owing to lymphatic drainage, to determine by SPECT or PET with a high negative predictive value, the tumoral status of the lymph nodes, in particular of the SN, ii) owing to Raman spectroscopy or optical fluorescent imaging, to delimit the tumor margins; iii) IV injection of DNP heavily laden with iodine 131 and/or astatine 211 will reinforce therapeutic efficacy of external radiotherapy (ERT) owing to the radiosensitizing effect of the DNP on account of their high electron density, iiii) on the nanoplatform, incorporation of iodine 131 of high specific activity in a hydrogel will allow beta therapy to be delivered by the cutaneous route.
Proof of concept was performed on a preclinical mouse model and may also be performed on human metastatic tumors on the same principle.
Advantageously, the use of the compounds and complexes described in the present text, in melanoma, may have an impact on:
i) biopsy of the sentinel lymph node (SLN), by limiting the number and the associated morbidity to regional lymph node dissection;
ii) an impact on determination of the margins for exeresis of Dubreuilh melanosis
iii) improvement of the therapeutic efficacy and of the prognosis of high grade melanoma:
a) by improving the treatment of cerebral metastases
b) by an impact on micrometastases owing to alpha IRT.
iv) availability of treatment of metastatic cutaneous lesions in the elderly owing to beta-ITR included in a gel forming a patch.
Taking into account the purely organic nature of compositions of the compounds and complexes according to the invention, suggesting low toxicity, short-term clinical transfer seems realistic. The impacts described above on the management of melanomas are also applicable to many types of cancer. This use therefore offers many advantages.
An example of the use of the “click-clack” system according to the invention is illustrated in FIGS. 14 and 15.
“Micrometastases” refer to the initial stage of metastasis, where only a few tumor cells are present, which are not manifested by any clinical, physical or systemic biological sign. Detection of micrometastases thus allows detection of a metastatic process at an early stage.
In certain embodiments, the invention relates to a complex comprising a compound of formula (I), wherein T represents a chelating agent, and a gadolinium or manganese metal ion, for use as a tool for detecting tumors by manganese-enhanced magnetic resonance imaging.

“Tool for detecting tumors” refers to for example a contrast agent, i.e. an agent that makes it possible to increase artificially the contrast between tissues or cells of different kinds, in order to visualize a tumor, which naturally has little or no contrast, and is difficult to distinguish from the neighbouring tissues.

In certain other embodiments, the invention relates to a complex comprising a compound of formula (I), wherein T represents a chelating agent, and a radioelement that emits gamma radiation such as gallium-67, technetium-99 m, indium-111, iodine-123, iodine-125, iodine-131, or holmium-166, for use as a tool for detecting tumors by gamma scintigraphy (GSc) or by single-photon emission tomography (SPET).

In certain other embodiments, the invention relates to a complex comprising a compound of formula (I), wherein T represents a chelating agent, and a radioelement that emits positrons such as scandium-44, copper-64, gallium-68, rubidium-82, zirconium-89, iodine-124 for use as a tool for detecting tumors by positron emission tomography (PET).

Hypoxia is a metabolic state that is responsible, in the case of tumor cells, for resistance of certain tumors to chemotherapy or to external radiotherapy.

The term apoptosis refers to a specific state of cellular death, called “programmed cell death”. This process may be spontaneous and physiological, but may also be induced by treatments such as chemotherapy or external or internal radiotherapy.

Identification and detection of these metabolic states is therefore important for adapting treatments, for example increasing the intensity of radiation in external radiotherapy (ERT), to have sufficient antitumor efficacy, the aim being to “over-irradiate” the hypoxic tumor cells relative to the normoxic tumor cells. Detection of apoptosis of the tumor cells after a first chemotherapy treatment makes it possible to evaluate very quickly whether this chemotherapy is effective. If not, the type of chemotherapy can be modified quickly without waiting several months and confirming absence of efficacy clinically and on the basis of morphological imaging criteria.

In certain other embodiments, the invention relates to a complex comprising a compound of formula (I), wherein T represents a recognition agent of a specific molecule, and said specific molecule, bound to another dendrimer, of formula (DD^{A}), for use as a tool for detecting tumors and/or for characterization of the sentinel lymph node.

In certain other embodiments, the invention relates to a complex comprising a compound of formula (I), wherein T represents a recognition agent of a specific molecule, and said specific molecule, bound to another dendrimer, of formula (DD^{A}), wherein the end groups Ra are such that R1, R2, R3 and R4 are nonradioactive iodine and/or R15 and R16 are nonradioactive fluorine, for use as a tool for detecting tumors and/or for characterization of the sentinel lymph node.

The invention also relates to a complex as described above, for use as a radiotherapeutic medicament in the treatment of tumors, notably in the form of micrometastases.

In certain embodiments, the invention relates to a complex comprising a compound of formula (I), wherein T represents a chelating agent, and a radioelement that emits particle rays, beta minus, Auger electron, or alpha particles, such as copper-67, yttrium-90, indium-ill, iodine-125, iodine-131, holmium-166, lutetium-177, rhenium-186, astatine-211, lead-212, bismuth-212, bismuth-213, actinium-225, for use as a radiotherapeutic medicament in the treatment of tumors, notably in the form of micrometastases.

In a particularly advantageous embodiment, the invention relates to a complex comprising a compound of formula (I), wherein T represents a chelating agent, and a radioelement that emits particle rays, beta minus. Auger electron, or alpha particles, such as copper-67, yttrium-90, indium-111, iodine-125, iodine-131, holmium-166, lutetium-177, rhenium-186, astatine-211, lead-212, bismuth-212, bismuth-213, actinium-225, for use as a medicament in curietherapy or internal or targeted, or vectorized radiotherapy.

“Curietherapy” refers to therapy carried out by introducing a radioactive source within the zone to be treated, or bringing it in contact with the latter.

“Internal or targeted radiotherapy” refers to therapy wherein the unsealed radioactive source, in liquid form or in the form of a capsule, is injectable or is administered by the oral route and will accumulate preferentially on the target tissues to be treated, whether or not tumoral, such as in inflammatory disorders or hyperthyroidism.

Said complex makes it possible to improve treatment specificity and reduce the side-effects, because the complex of the invention is fixed predominantly on the target tumor cells, or by vascular structures present in the tumoral tissues or proliferative lesions, and micrometastases.

According to an advantageous embodiment, the tumor and/or the cells in a particular metabolic state belong to the group comprising:
melanomas, V notably being of formula VI:

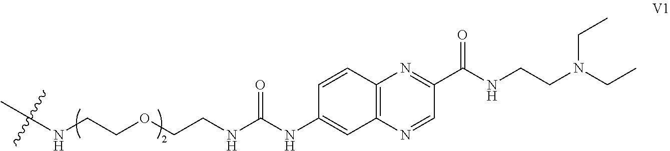

chondrosarcomas, Y notably being of formula V2 or V3:

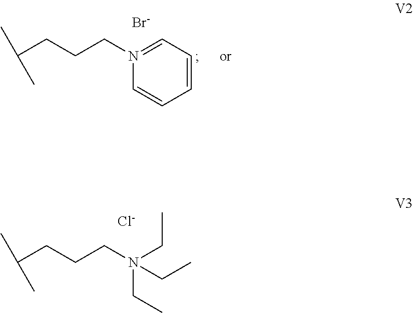

glioblastomas or neuroendocrine tumors, V notably being L-DOPA, or a derivative thereof,
glioblastomas or mammary tumors, V being a ligand of the Human Epidermal Growth Factor Receptor 2 (HER2) tumor receptor, in particular a specific antibody;
prostate tumors, V being a specific antibody targeting the PSMA receptor;
breast cancer tumors, V being an aptamer or a tripeptide targeting nucleolin, in particular of formula V4:

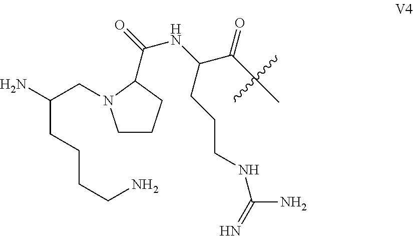 [SMILES: *C(=O)C(CCCNC(=N)N)NC(=O)C1CCCN1CC(N)CCCCN]

hypoxic cells. V being a nidazole derivative (misonidazole (MISO) or metronidazole (METRO))
apoptotic cells, V comprising reactive oxygens, notably capable of reacting with caspase.

Examples of compounds comprising reactive oxygens capable of reacting with caspase are described by Soundararajan et al. Cancer Res. 2008, 68, 2358.[30]

### Combination Products

The present invention also relates to a product containing:
(i) a compound of formula I, II or III, as described in any one of the above variants, wherein T represents a recognition agent of a specific molecule, and
(ii) a compound corresponding to formula IV

T′-L₁-R IV

or a dendrimer, in particular a dendrimer of formula DD^{A}, comprising said specific molecule,
and in said formula IV, T′ comprises said specific molecule and R and L₁ are as described in any one of the above variants.
as a combination product, for simultaneous use, separate use or use spread over time, in the diagnosis of cancers, notably of micrometastases.
According to an advantageous embodiment, the present invention relates to a product containing:
(i) a compound of formula I, II or III, as described in any one of the above variants, wherein T represents avidin, streptavidin, biotin, cyclodextrin or adamantane; and
(ii) a compound corresponding to formula IV

T′-L₁-R IV
or a dendrimer, in particular a dendrimer of formula DD^{A}, comprising said specific molecule T′,
where T′ represents:
[symbol] biotin, when group T of the compound of formula I, II or III is avidin or streptavidin, or
[symbol] avidin or streptavidin, when group T of the compound of formula I, II or III is biotin.
[symbol] adamantane, when group T of the compound of formula I, II or III is a cyclodextrin, or
[symbol] cyclodextrin, when group T of the compound of formula I, II or III is adamantane,
L₁ is a heteroalkyl chain of structure:

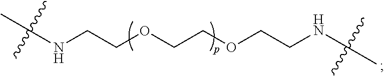

wherein p is an integer from 1 to 10;
   or else T′ is absent and L₁ corresponds to one of the following azide structures:

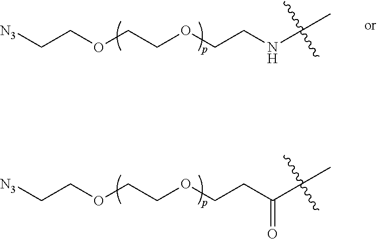

wherein p is an integer from 1 to 10; preferably from 1 to 8; preferably 3 or 7;
R is a labelable moiety such as L-thyroxine or dinitrobenzoate:

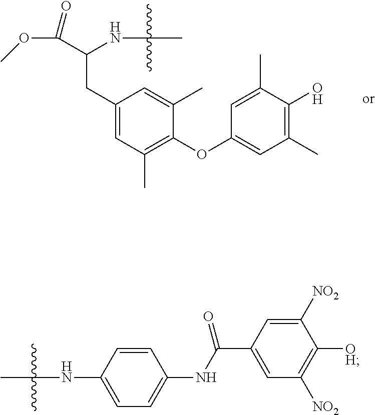

as a combination product, for simultaneous use, separate use or use spread over time in the diagnosis of cancers, notably of micrometastases.
The compounds of formula I, II, III, and IV described in the present text may be prepared using chemical transformations that are familiar to a person skilled in the art. In fact, the various constituent elements of these compounds (T, T′, L₁, D, R, L₂ and V) are bound together by chemical functions whose synthetic chemistry is well known: amide, ether, heterocycles (triazole), thiourea (—NHC(═S)NH—).
The general principle is based on the reaction of a first constituent of the compound (T for example) bearing a reactive group, any other reactive groups carried by the first constituent optionally being protected by ad hoc protective groups, with a second constituent (L₁ for example) bearing a suitable reactive group allowing formation of the desired chemical bond (e.g. amide, ether, heterocycles (triazole), thiourea (—NHC(═S)NH—)) by reaction with the reactive group of the first constituent. Any other reactive groups carried by the second constituent may also optionally be protected by ad hoc protective groups.
As an example, when T represents a recognition agent such as adamantane, T may have the following structure:

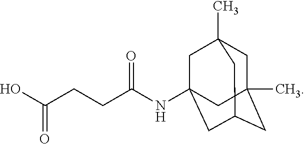 [SMILES: CC12CC3CC(C)(C1)CC(NC(=O)CCC(=O)O)(C3)C2]

which may be bound covalently to a spacer L1, or a radiolabelable moiety or group R, or a dendrimer D, etc. bearing an amine function to form a compound of formula I according to the invention where T, as defined above, is bound to the rest of the molecule by an amide bond. For example, a person skilled in the art will be able to take inspiration from the synthesis techniques described in WO 2008/043911. For example, T as defined above may be prepared as follows:

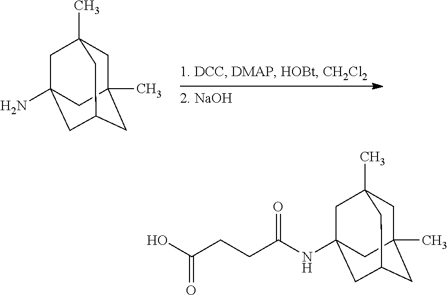 [SMILES: CC12CC3CC(C)(C1)CC(N)(C3)C2 ; CC12CC3CC(C)(C1)CC(NC(=O)CCC(=O)O)(C3)C2]

The various chemical transformations for preparing compounds illustrated in the examples given below will be apparent to a person skilled in the art on reading the text of the application, and in light of all the knowledge that is available in the field of organic chemistry in general, and more particularly synthetic transformations as listed in many reference works, for example:
1. “Advanced Organic Chemistry—Reactions, mechanisms and structure”, Jerry March, John Wiley & Sons, 5th edition, 2001;
2. “Comprehensive Organic Transformations, a guide to functional group preparations”.

Richard C. Larock. VCH Publishers, 2nd edition, 1999;

3. “Protective Groups in Organic Synthesis” by T. W. Greene, P. G. M. Wuts. Wiley-Interscience, New York, 4th edition, 2007.
The examples given below illustrate embodiments for preparing compounds according to the invention. A person skilled in the art will know without difficulty how to adapt these embodiments to the preparation of other compounds according to the invention, based on the teaching in the present text, the works on organic synthetic chemistry cited above, and general knowledge in this area. A person skilled in the art will also know how to adapt the methods of synthesis described in document WO 2008/043911 for preparing all the variants of the compounds according to the invention described in the present text.
The invention also relates to a pharmaceutical composition comprising a complex as described above, as active substance, and a pharmaceutically acceptable vehicle.
The invention also relates to a diagnostic composition comprising a complex as described above, as active substance, and a pharmaceutically acceptable vehicle.
Said pharmaceutically acceptable vehicle may be selected from pharmaceutically acceptable vehicles known by a person skilled in the art for pharmaceutical or diagnostic compositions, comprising in particular a species selected from:
[symbol] a gadolinium or manganese metal ion, or
[symbol] a radioelement that emits gamma radiation, or emits positrons, and/or emits particle rays, beta minus, Auger electron or alpha particles such as gold-33, copper-61, copper-62, copper-64, copper-67, gallium-67, gallium-68, zirconium-89, yttrium-90, technetium-99m, indium-111, iodine-123, iodine-124, iodine-125, iodine-131, holmium-166, lutetium-177, rhenium-186, astatine-211, actinium-89, bismuth-213, or some other radioelement that can be detected by an imaging or radioactivity counting system or having a radiotoxic effect.
   Pharmaceutical or diagnostic compositions according to the invention may be formulated in liquid form, such as injectable solutions or oral solutions, injectable suspensions or oral suspensions, or solutions that can be absorbed by inhalation. They may also be in solid form, such as tablets, capsules, pills, suppositories or pessaries, in particular for diagnosing, in the case of diagnostic compositions, digestive or gynecological abnormalities.

A pharmaceutical composition or a diagnostic composition according to the present invention may be administered by the intravenous, intradermal, intraarterial, intralymphatic or oral route.

When, in the pharmaceutical or diagnostic composition, the complex comprising a compound of formula (I) also comprises a radioelement, said composition is radioactive.
A radioactive pharmaceutical composition according to the present invention may be administered at one or more doses as a function of the method of administration of said composition, the mass of tissue to be treated, such as the tumoral mass, the volume of distribution and dosimetry determined beforehand.
In one embodiment, a radioactive pharmaceutical composition according to the present invention may be administered at a radioactivity from 1 to 40 MBq/kg, particularly 1 to 20 MBq/kg, more particularly 1 to 15 MBq/kg, notably 1 to 10 MBq/kg of body weight in the case of curietherapy or by intratumoral injection.
In one embodiment, a radioactive pharmaceutical composition according to the present invention may be administered at a unit radioactivity from 50 to 3200 MBq, particularly 50 to 1600 MBq, more particularly 50 to 1200 MBq, notably 50 to 800 MBq in the case of curietherapy or by intratumoral injection.
In another embodiment, a radioactive pharmaceutical composition according to the present invention may be administered at a radioactivity from 1 to 50 MBq/kg, particularly 1 to 20 MBq/kg, more particularly 1 to 15 MBq/kg, even more particularly 1 to 10 MBq/kg, notably 1 to 5 MBq/kg of body weight in the case of targeted radiotherapy or by systemic injection.
In another embodiment, a radioactive pharmaceutical composition according to the present invention may be administered at a unit radioactivity from 50 to 4000 MBq, particularly 50 to 1600 MBq, more particularly 50 to 1200 MBq, even more particularly 50 to 800 MBq, notably 50 to 400 MBq in the case of targeted radiotherapy or by systemic injection.

Other advantages may become apparent to a person skilled in the art on reading the following examples, illustrated by the appended figures, given for purposes of illustration.

### BRIEF DESCRIPTION OF THE FIGURES

FIG. 1 shows a procedure for labeling with Tc99m.

FIG. 2 shows a purity assessment of the radiolabeled products by paper chromatography.

FIG. 3 shows planar images obtained with compound APAG9-1 immediately post-injection (A) and 2 hours post-injection (B).

FIG. 4 shows planar images obtained with compound APAG22-1 immediately post-injection (A) and 2 hours post-injection (B).

FIG. 5 shows planar images obtained with compound AP045 immediately post-injection (A) and 2 hours post-injection (B).

FIG. 6 shows planar images obtained with compound AP071 2 hours post-injection (A) and 4 hours post-injection (B).

FIG. 7 shows planar images obtained with compound AP070 1 hour post-injection (A) and 2 hours post-injection (B).

FIG. 8 shows planar images obtained with compound XI labeled with Tc99m, acquired just after intradermal injection (A) and SPECT acquired 1 h after injection (B) of a mouse injected at the end of the two hind paws with 10 microliters-5 MBq.

FIG. 9 shows a diagram listing the % radioactivity ratio relative to the activity injected in different organs measured at 24 hours in a mouse injected at the end of the two hind paws with a total volume of 10 microliters and an activity of 2.8 MBq of the solute described in FIG. 8. The activity that is still present in the feet and the whole of the lower limbs is evidence of complete drainage of the lymphatic bed and of the injection site, and the very high level of urinary elimination of the compound, with a very low hepatic activity, secondary to secondary drainage in the blood circulation.

FIG. 10 shows a planar image obtained with generation 1 compound APAG4 labeled with indium-111 SPECT acquired 3 h after intradermal injection at the end of the two hind paws of a mouse of 10 MBq of solute APAG4 labeled with indium-111. The drainage in the entire lymphatic network as far as the lumbar-aortic stage is clearly visible.

FIG. 11 shows a graph illustrating radiochemical purity assessment of compound AP034-Tc99m.

FIG. 12 shows a SPECT planar image acquired 2 h after intradermal injection at the end of the two hind paws of a mouse of 10 microliters-38 MBq of solute AP034 labeled with Tc99m.

FIG. 13 shows detection of numerous tumoral nodules within the ipsi-lateral popliteal lymph node, in the popliteal lymph node taken 3 weeks after injection of the tumor cells (Example 10).

FIG. 14 illustrates a clack system according to the invention, for example by using a targeting agent V conjugated to an iodinated and/or fluorinated dendrimer DD_{A}.

FIG. 15 schematically shows an example of the use of a “click-clack” system according to the present invention (two-stage protocol).

### EXAMPLES

### Example 1

### Synthesis of the Control Compound (2, APAG0045)

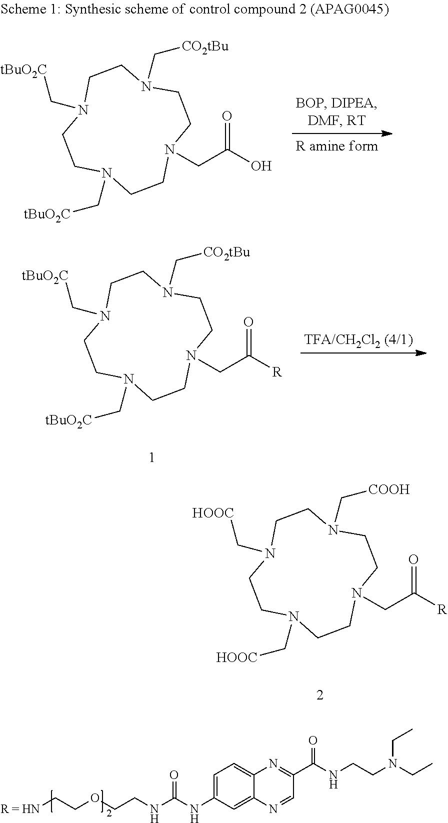

Coupling Reaction to Give Compound 1

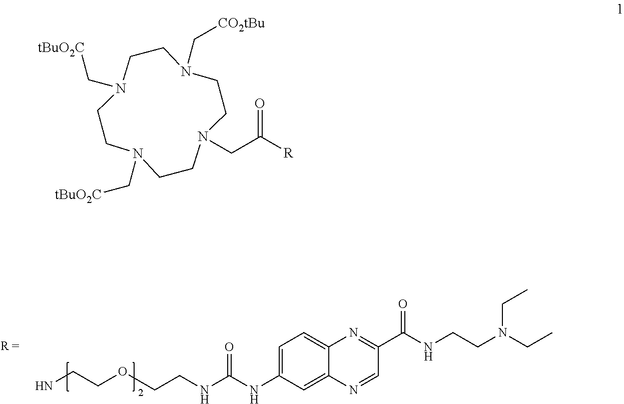

The synthesis of compound 1 was carried out by the method described in Org. Biomol. Chem., 5, 935-944, 2007.

BOP (0.03 g, 0.07 mmol) and DIPEA (0.027 ml, 0.15 mmol) were added to a suspension of DOTA (0.037 g, 0.06 mmol) and of amine R (0.03 g, 0.06 mmol) in DMF (4 ml). The mixture was stirred at room temperature for 48 h, and then the solvent was evaporated under vacuum. The crude product was diluted with ethyl acetate, washed with brine, dried over MgSO₄ and evaporated under reduced pressure. The residue was purified by size exclusion chromatography (CH₂Cl₂/MeOH 50/50) to give the desired compound 1 (83%) in the form of yellow powder.

¹H NMR (300 MHz, CDCl₃) δ (ppm) 1.25-1.49 (m, 33H, 2CH₃, 3 tBu), 1.70-2.70 (bs, 24H, CH₂ DOTA), 2.8-3.55 (m, 20H, CH₂O, CONHCH₂CH₂N(CH₂CH₃)₂), 3.89 (m, 2H, CONHCH₂CH₂N(CH₂CH₃)₂), 7.27 (m, 1H, NH), 7.87 (d, 1H, J=9.2 Hz, Ar-3-H), 8.07 (dd, 1H, J=2.3 Hz, J=9.2 Hz, Ar-4-H), 8.57 (s, 1H, Ar-6-H), 8.58 (m, 2H, NH), 9.43 (s, 1H, Ar-8-H), 10.47 (s, 1H, NH); ¹³C NMR (75 MHz, CDCl₃) δ (ppm) 172.2, 171.8, 164.9, 156.1, 145.7, 144.7, 143.3, 139.7, 136.1, 129.6, 125.5, 120.5, 112.6, 81.8, 73.8, 70.6, 70.4, 70.1, 69.3, 56.1, 55.7, 55.5, 51.2, 47.6, 39.8, 39.3, 28.0, 9.7.

### Final Deprotection to Give Compound 2: First Control Compound

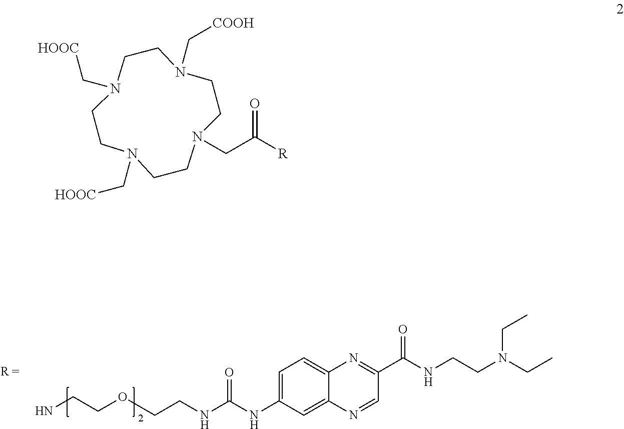

Trifluoroacetic acid TFA (1 ml) was added to a suspension of 1 (0.04 g, 0.04 mmol) in CH₂Cl₂ (9 ml) at 0° C. The solution was brought back to room temperature and stirred for 3 h. Then the solvent was evaporated under vacuum and the residue obtained was purified by size exclusion chromatography (Sephadex LH20. GE Healthcare, CH₂Cl₂/MeOH 50/50) to give the desired compound 2 (50%) in the form of yellow powder. A new solution for deprotection of the esters of compound 1 is to use TMSBr in excess (30 eq. per 1 eq. of compound 1) in CH₂Cl₂ to obtain the desired compound 2.

¹H NMR (300 MHz, MeOD) δ (ppm) 1.34-1.60 (m, 6H, 2CH₃), 2.00-3.80 (m, 44H, CH₂ DOTA, CH₂O, CONHCH₂CH₂N(CH₂CH₃)₂), 7.95 (d, 1H, J=9.2 Hz, Ar-3-H), 8.15 (dd, 1H, J=2.3 Hz, J=9.2 Hz, Ar-4-H), 8.44 (s, 1H, Ar-6-H), 9.45 (s, 1H, Ar-8-H); ¹³C NMR (75 MHz, MeOD) δ (ppm) 167.1, 162.3, 161.9, 157.5, 145.2, 144.6, 142.5, 138.1, 131.4, 126.0, 119.7, 114.0, 71.4, 71.3, 71.2, 71.1, 70.5, 70.3, 55.8, 52.5, 43.7, 40.7, 40.4, 9.7.

### Example 2

### Synthesis of a Compound (8, AP071) Comprising DOTA and Two Melanoma Targeting Agents

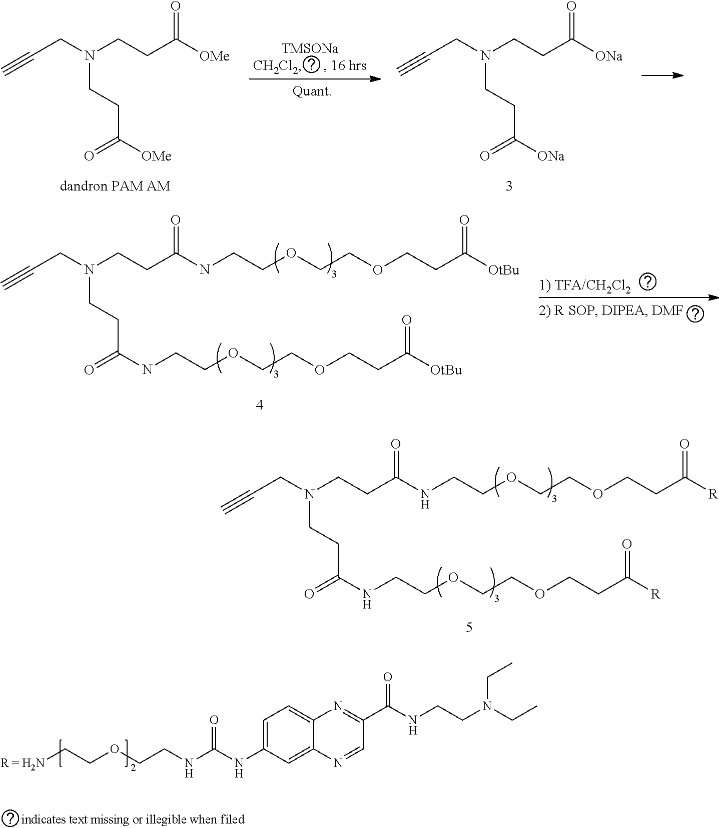

A solution of sodium trimethylsilanolate (1 M) 0.33 mmol) in dichloromethane (66 mL, 66 mmol) was added to a solution of PAMAM dendron (5 g, 22 mmol) in dichloromethane (90 mL). The mixture was stirred for 16 hours at room temperature, after which a gel had formed. The solvent was evaporated under vacuum and the residue was precipitated in ethyl acetate. The solid was precipitated to give compound 3 quantitatively, in the form of a yellow solid.

¹H NMR (300 MHz, D₂O), δ 3.41 (s, 2H), 2.83 (t, J=8.2 Hz, 4H), 2.39 ((t, J=8.2 Hz, 4H),

¹³C NMR (75 MHz, D₂O) δ 181.1, 78.0, 74.4, 49.8, 41.1, 34.9.

MS (MALDI-TOF) m/z calculated for C₉H₁₁NNa₂O₄243.05, obtained 242.288.

EDCI (0.38 g, 1.97 mmol), HOBt (0.05, 0.33 mmol) and DIPEA (0.31 mL, 1.8 mmol) were added to a suspension of compound 3 (0.2 g, 082 mmol) and of amino-dPEG®3-CO2tBu (0.58 g, 1.8 mmol) in acetonitrile (9 mL). The mixture obtained was stirred at room temperature for 16 hours and then the solvent was evaporated under vacuum. The residue was purified by silica gel chromatography (CH₂Cl₂/MeOH 90/10) to give compound 4 (84%) in the form of a yellow oil.

¹H NMR (300 MHz, CDCl₃) δ 3.70 (t, J=6.7 Hz, 4H), 3.66-3.60 (m, 24H), 3.54 (t, J=5.3 Hz, 4H), 3.41 (m, 6H), 2.83 (t, J=6.3 Hz, 4H), 2.49 (t, J=6.7 Hz, 4H), 2.36 (t, J=6.3 Hz, 4H), 2.23 (t, J=2.3 Hz, 1H), 1.44 (s, 18H).

¹³C NMR (75 MHz, CDCl₃) δ 171.9, 170.7, 80.3, 73.7, 70.5, 70.45, 70.42, 70.4, 70.2, 70.1, 69.8, 49.4, 41.4, 39.0, 36.2, 33.7, 28.0.

MS (MALDI-TOF) m/z calculated for C₃₉H₇₁N₃O₁₄ 805.49, obtained [M+H]+=806.551.

A solution of trifluoroacetic acid TFA in CH₂Cl₂ (1/4) was added dropwise to a suspension of compound 4 (0.04 g, 0.05 mmol) in CH₂Cl₂ (5 mL) at 0° C. The resulting solution was stirred for 4 hours at room temperature until there was complete deprotection of the esters. The solvent was evaporated under reduced pressure and the excess of TFA was removed by washing the residue with ether and co-evaporation (3 times). Then the residue was dissolved in DMF (4 mL) in the presence of R (0.06 g, 0.12 mmol), BOP (0.07 g, 0.13 mmol) and DIPEA (0.06 mL, 0.30 mmol). The amine “R” was prepared by the method of J. Molieras et al., Nanoscale, 2013, 5, 1603-1615, Development of gadolinium based nanoparticles having an affinity towards melanin. The mixture obtained was stirred at room temperature for 48 hours and then the solvent was evaporated under vacuum. The residue was purified by size exclusion chromatography (Sephadex, LH, GE Healthcare, CH₂Cl₂/MeOH 50/50) to give compound 5 (87%) in the form of a brown oil.

¹H NMR (300 MHz, CDCl₃) δ (ppm) 1.1 (dd, 12H, J=7 Hz, 4 CH₃), 2.23 (s, 1H, H alkyne), 2.37 (dd, J=6.15 and 6.36 Hz, 4H, 2 CH₂CONH), 2.53 (dd, J=5.7 Hz, 4H, 2CH₂CONH PAMAM), 2.64-2.84 (m, 24H, CONHCH₂CH₂O, CH₂NHCONH, CONHCH₂CH₂N(CH₂CH₃)₂), 3.39-89 (m, 58H, CONHCH₂CH₂N(CH₂CH₃)₂), CH₂O, CH₂N and NCH₂ PAMAM), 6.23 (n, 2H, NH), 7.29 (m, 2H, NH), 7.53 (m, 2H, NH), 7.96 (d, 2H, J=9.2 Hz, Ar-3-H), 8.05 (s, 2H, NH), 8.25 (n, 2H, Ar-4-H), 8.40 (m, 2H, NH), 9.32 (s, 2H, Ar-6-H), 9.50 (s, 2H, Ar-8-H); ¹³C NMR (75 MHz, CDCl₃) δ (ppm) 172.6, 172.4, 164.1, 155.5, 145.1, 143.9, 143.8, 141.0, 136.5, 130.1, 124.5, 113.1, 77.7, 73.8, 70.8, 70.5, 70.4, 70.3, 70.2, 70.1, 70.0, 69.8, 67.1, 51.6, 49.4, 47.3, 41.5, 39.6, 39.5, 39.1, 36.9, 36.8, 33.9, 27.9, 11.2.

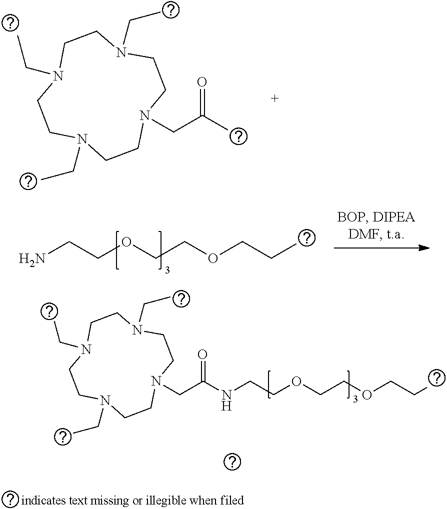

BOP (0.1 g, 0.23 mmol) and DIPEA (0.12 mL, 0.68 mmol) were added to a suspension of DOTA (0.1 g, 0.17 mmol) and amino-dPEG®8-N3 (0.07 g, 0.19 mmol) in DMF (4 mL). The mixture obtained was stirred at room temperature for 96 hours and then the solvent was evaporated under vacuum. The residue was dissolved in CH₂Cl₂, washed with brine, dried over MgSO₄ and evaporated under reduced pressure. The residue was purified by silica gel chromatography (CH₂Cl₂/MeOH 100/0 to 95/5) to give compound 6 (82%) in the form of a colorless oil.

¹H NMR (300 MHz, CDCl₃) δ (ppm) 1.47 (s, 27H, 3 tBu), 1.90-3.2 (bs, 24H, H DOTA), 3.38 (dd, J=5.7 Hz, 2H, CH₂N3), 3.53 (m, 2H, CONHCH₂CH₂O), 3.57-3.71 (m, 28H, CH₂O), 7.27 (m, 1H, NH) ¹³C NMR (75 MHz, CDCl₃) δ (ppm) 172.5, 171.6, 81.8, 70.6, 70.5, 70.4, 70.2, 69.9, 69.4, 56.0, 55.6, 50.7, 39.1, 28.1.

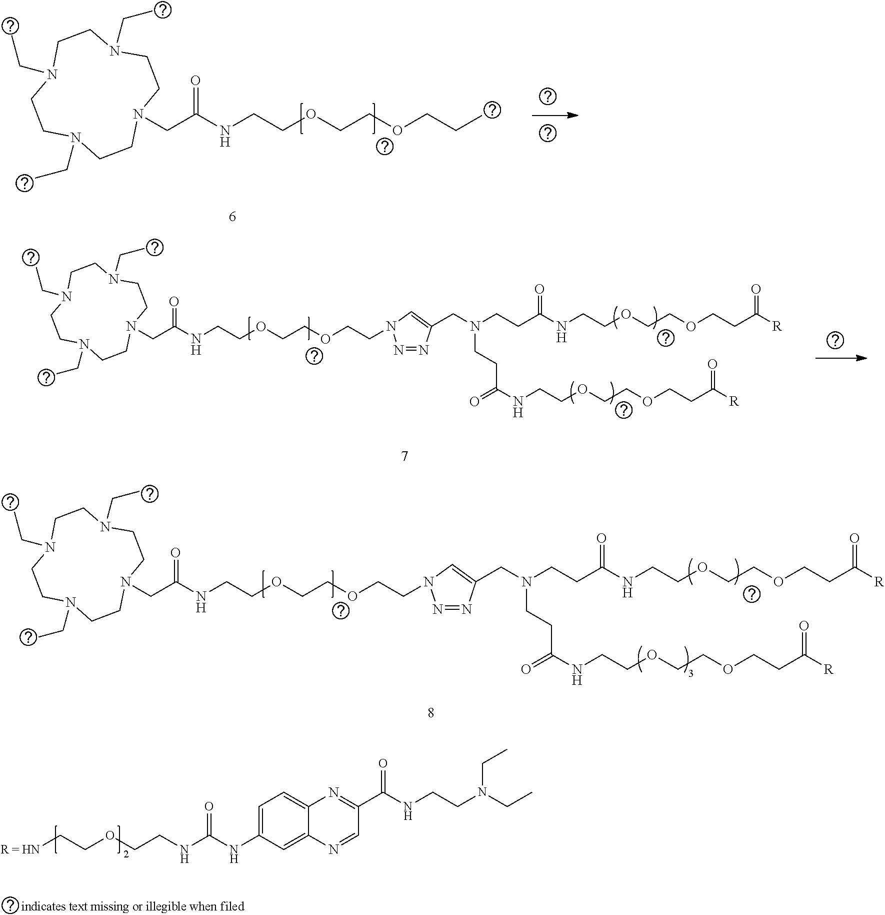

A mixture of azide precursor 6 (0.033 g, 0.035 mmol) and PAMAM propargyl dendron 5 (0.05 g, 0.032 mol) in a THFH₂O mixture (4 ml (1/1)) in the presence of 5 mol % of CuSO₄.5H₂O and of 10 mol % of sodium ascorbate was stirred at room temperature for 16 h. After evaporation of the solvents under vacuum, the residue was dissolved in water (30 mL), a spatula of Chelex100 (Bio-Rad) was added to the solution, and the mixture was stirred for one hour at room temperature. The resin was then filtered and washed with water (2×20 mL). The filtrate was concentrated under reduced pressure and the residue obtained was purified by size exclusion chromatography (Sephadex, LH20, GE Healthcare. CH₂Cl₂/MeOH 50/50) to give compound 7 (46%).

¹H NMR (300 MHz, CDCl₃) δ (ppm) 1.0-1.54 (m, 39H, CH₃, tBu), 2.3-3.85 (m, 152H, CH₂CONH₂, CH₂CONH, PAMAM, CONHCH₂CH₂O, CH₂NHCONH, CONHCH₂CH₂N(CH₂CH₃)₂, CONHCH₂CH₂N(CH₂CH₃)₂), CH₂O, CH₂N and NCH₂ PAMAM, CH₂ DOTA), 4.5 (m, 2H, CH₂ next to triazole ring), 6.76 (m, 2H, NH), 7.14 (m, 2H, NH), 7.45-8.56 (m, 11H, NH, Ar-3-H, Ar-4-H, H triazole ring), 9.32-9.45 (m, 5H, NH, Ar-6-H and Ar-8-H); ¹³C NMR (75 MHz, CDCl₃) δ (ppm) 172.9, 172.6, 164.2, 159.9, 155.7, 145.1, 143.9, 143.6, 140.7, 136.4, 130.0, 124.6, 124.4, 112.9, 81.9, 73.6, 70.5, 70.2, 70.0, 69.6, 67.1, 58.1, 51.4, 50.5, 50.0, 49.3, 47.1, 41.2, 39.6, 39.4, 39.0, 37.2, 36.5, 33.6, 29.6, 27.9, 10.8.

TMSBr (0.047 mL, 0.35 mmol) was added to a suspension of 7 (0.03 g, 0.02 mmol) in a CH₃CN/CH₂Cl₂ mixture (5 mL, 1/1). After 4 h of stirring, the silyl esters were hydrolyzed by adding MeOH (5 mL), the mixture being stirred for 2 hours at RT. The solvent was evaporated under reduced pressure, and the residue obtained was purified by size exclusion chromatography (Sephadex, LH20, GE Healthcare, MeOH) to give compound 8 (50%) in the form of a yellow powder.

¹H NMR (300 MHz, MeOD) δ (ppm) 1.34-1.54 (m, 12H, CH₃), 2.3-3.94 (m, 152H, CH₂CONH, CH₂CONH PAMAM, CONHCH₂CH₂O, CH₂NHCONH, CONHCH₂CH₂N(CH₂CH₃)₂, CONHCH₂CH₂N(CH₂CH₃)₂), CH₂O, CH₂N and NCH₂ PAMAM, CH₂ DOTA), 4.65 (m, 2H, CH₂ next to triazole ring), 7.95 (m, 3H, NH, Ar-3-H, H triazole ring), 8.04 (m, 2H, Ar-4-H), 8.38 (m, 2H, Ar-6-H), 9.42 (m, 2H, Ar-8-H); ¹³C NMR (75 MHz, MeOD) δ (ppm) 173.1, 172.7, 165.6, 155.9, 144.6, 143.8, 143.2, 141.0, 136.6, 129.9, 124.5, 112.5, 70.1, 69.8, 69.7, 69.2, 69.0, 66.8, 66.2, 51.1, 50.7, 50.0, 49.2, 39.3, 39.0, 36.2, 34.3, 33.0, 7.7.

### Example 2

### Synthesis of a Compound (15, AP070) Comprising DOTA and Four Melanoma Targeting Agents

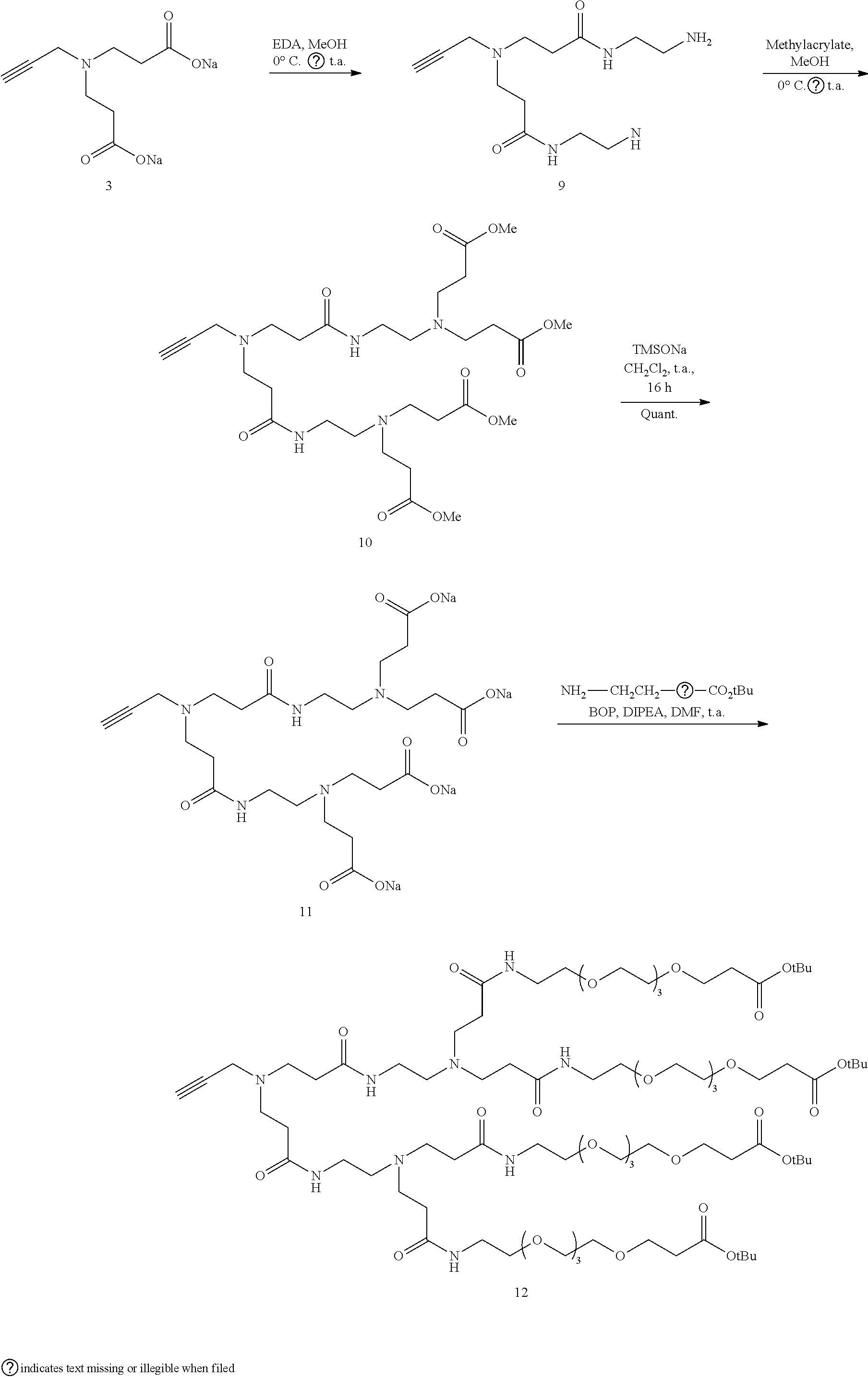

A solution of EDA (76 mL, 113.1 mmol) in MeOH (120 mL) was added dropwise to a solution of 3 (5.35 g, 23.5 mmol) in MeOH (30 mL), in the space of one hour, at 0° C. The reaction mixture was then stirred for 144 hours at room temperature, and then the solvents were evaporated under reduced pressure. The excess EDA was removed by concentration of the residue dissolved in a toluene/MeOH mixture (9/1). Compound 9 was obtained quantitatively in the form of a yellow oil by azeotropic distillation of toluene in the presence of MeOH.

¹H NMR (300 MHz, MeOD) δ (ppm) 2.37 (m, 4H, CH₂NH₂), 2.64 (dd, 1H, J=2.2 Hz, J=2.4 Hz, H alkyne), 2.69-2.75 (m, 4H, 2CH₂CONH), 2.81-2.85 (m, 4H, 2 CONHCH₂), 3.23-3.35 (m, 4H, NCH₂), 3.48 (d 2H, J=2.4 Hz, CH₂ alkyne); ¹³C NMR (75 MHz, MeOD) δ (ppm) 173.2, 77.6, 73.8, 57.8, 49.3, 41.7, 40.9, 40.8, 33.6, 33.5.

A solution of 9 (5.35 g, 23.5 mmol) in MeOH (22 mL) was added dropwise with stirring to a solution of methylacrylate (13 mL, 141.4 mmol) in MeOH (90 mL), in the space of one hour, at 0° C. The reaction mixture was then stirred for 11 days at room temperature, and the volatile compounds were evaporated under reduced pressure. The residue obtained was purified by size exclusion chromatography (SX-8, BioRad, THF 100%) and then by flash chromatography (SiO₂, CH₂Cl₂/MeOH 95/5 to 80/20) to give compound 10 (40%) in the form of a yellow oil.

¹H NMR (300 MHz, CDCl₃) δ (ppm) 2.19 (dd, 1H, J=2.2 Hz, J=2.4 Hz, H alkyne), 2.36-2.46 (m, 12H, 4 CH₂CONH), 2.55 (dd, 4H, J=5.9 Hz, J=5.9 Hz, 2 NHCH₂CH₂N), 2.76 (dd, 8H, J=6.8 Hz, J=6.6 Hz, 4 NCH₂), 2.85 (dd, 4H, J=6.8 Hz, J=6.6 Hz, 2NCH₂ next to CH₂ alkyne), 3.29 (dd, 4H, J=5.7 Hz, J=11.6 Hz, 2 CONHCH₂), 3.48 (d, 2H, J=2.2 Hz, CH₂ alkyne), 3.70 (s, 12H, OMe), 7.09 (m, 2H, NH).

A solution of sodium trimethylsilanolate (1 M) in CH₂Cl₂ (10 mL, 9.6 mmol) was added to a solution of compound 10 (1 g, 1.6 mmol) in CH₂Cl₂ (11 mL). The mixture was stirred for 16 hours at room temperature, after which a precipitate had formed. The mixture was filtered, washed with CH₂Cl₂ and concentrated under vacuum. Compound 11 was obtained quantitatively in the form of a yellow solid.

¹H NMR (300 MHz, D₂O) δ (ppm) 2.12-2.36 (m, 17H, H alkyne, 4 CH₂CONH, 2 NHCH₂CH₂N), 2.53-2.75 (m, 12H, 4 NCH₂, 2 NCH₂ next to CH₂ alkyne), 3.20-3.30 (m, 4H, 2 CONHCH₂), 3.48 (m, 2H, CH₂ alkyne); ¹³C NMR (75 MHz, D₂O) δ (ppm) 181.2, 174.6, 77.7, 51.7, 51.0, 49.5, 48.9, 41.3, 36.6, 34.1, 33.2.

BOP (0.3 g, 0.73 mmol) and DIPEA (0.26 mL, 1.52 mmol) were added to a suspension of 11 (0.1 g, 0.15 mmol) and amino-CH₂CH₂—POE4-CO2tBu (0.21 g, 0.67 mmol) in DMF (6 mL). The reaction mixture was stirred for 96 hours at room temperature, and the solvent was evaporated under reduced pressure. The residue was dissolved in CH₂Cl₂, washed with brine, dried over MgSO₄ and concentrated under reduced pressure. The residue was purified by size exclusion chromatography (Sephadex, LH20. GE Healthcare, CH₂Cl₂/MeOH 50/50) to give compound 12 (88%) in the form of a yellow oil.

¹H NMR (300 MHz, CDCl₃) δ (ppm) 1.45 (s, 36H, 4 tBu), 2.24 (bs., 1H, H alkyne), 2.30-3.00 (m, 24H, 4 CH₂COOtBu, 6 CH₂CONH PAMAM, 2 CONHCH₂CH₂NHCO), 3.10-3.71 (m, 88H, CH₂N and NCH₂ PAMAM, CH₂O, 4 CONHCH₂CH₂O), 3.79 (m, 2H, CH₂ alkyne), 5.30 (m, 2H, NH), 7.20 (m, 4H, NH); ¹³C NMR (75 MHz, CDCl₁) δ (ppm) 173.2, 170.9, 170.7, 80.3, 73.9, 70.4, 70.3, 70.2, 70.0, 69.9, 69.2, 52.7, 50.5, 49.0, 39.9, 39.1, 36.0, 33.1, 27.9.

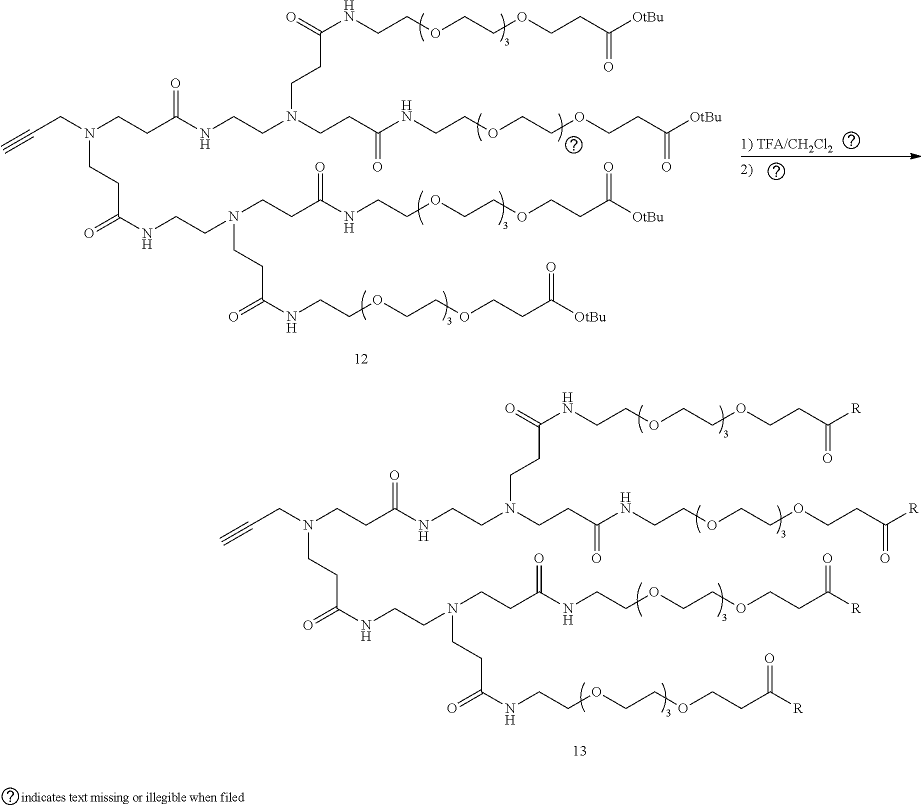

A solution of trifluoroacetic acid TFA in CH₂Cl₂ (5 mL, 1/4) was added dropwise to a suspension of compound 12 (0.05 g, 0.03 mmol) in CH₂Cl₂ (5 mL) at 0° C. The resulting solution was stirred for 4 hours at room temperature and then the volatile compounds were evaporated under reduced pressure. The excess TFA was removed by washing the residue with ether and co-evaporation (3 limes). Then the residue was dissolved in DMF (4 mL) in the presence of R (0.06 g, 0.14 mmol). BOP (0.07 g, 0.15 mmol) and DIPEA (0.06 mL, 0.32 mmol). The mixture obtained was stirred at room temperature for 96 hours and then the solvent was evaporated under vacuum. The residue was purified by size exclusion chromatography (Sephadex, LH20, GE Healthcare, CH₂Cl₂/MeOH 50/50) to give compound 13 (74%) in the form of a brown oil.

¹H NMR (300 MHz, MeOD) δ (ppm) 1.3 (dd, 12H, J=7 Hz, CH₃), 2.41-2.64 (m, 20H, CH₂CONHCH₂CH₂O, CONH CH₂CH₂N, CH₂NHCONH), 2.71 (s, 1H, H alkyne), 2.84 (m, 12H, CH₂CONH PAMAM).) 3.13-3.81 (m, 162H, CH₂ alkyne, CONHCH₂CH₂O, CONHCH₂CH₂N(CH₂CH₃)₂, CH₂O, CH₂N and NCH₂ PAMAM), 7.90 (m, 4H, Ar-3-H), 8.06 (d, 4H, J=8.97 Hz, Ar-4-H), 8.35 (s, 4H, Ar-6-H), 9.38 (s, 4H, Ar-8-H); ¹³C NMR (75 MHz, MeOD) δ (ppm) 177.4, 176.9, 176.6, 168.9, 159.8, 148.4, 147.6, 147.1, 145.1, 140.5, 133.8, 128.4, 116.5, 81.5, 77.8, 74.0, 73.9, 73.8, 73.7, 73.1, 70.8, 56.0, 55.0, 53.6, 53.2, 44.8, 43.3, 42.9, 41.1, 40.2, 39.2, 37.5, 37.0, 33.2, 31.0, 30.8, 12.8.

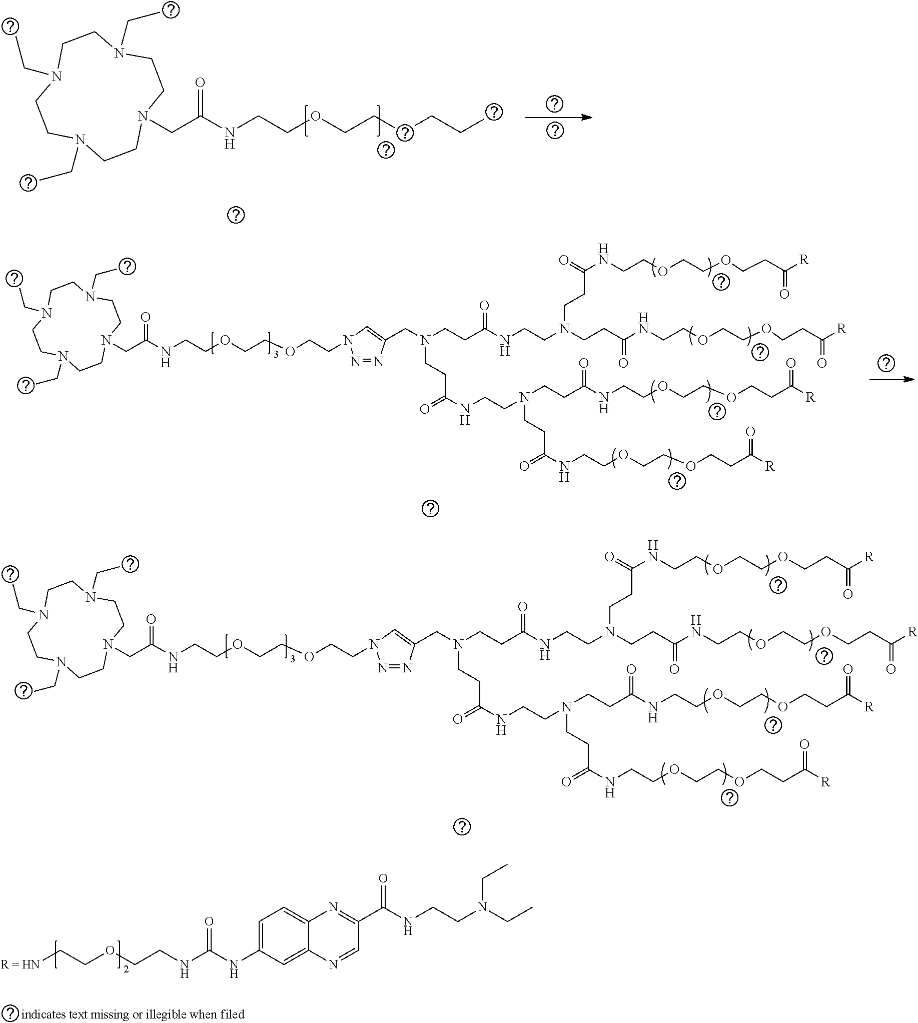

A mixture of azide precursor 6 (0.016 g, 0.016 mmol) and PAMAM propargyl dendron 13 (0.05 g, 0.015 mol) in a THF/H₂O mixture (4 ml (1/1)) in the presence of 5 mol % of CuSO₄.5H₂O and 10 mol % of sodium ascorbate was stirred at room temperature for 16 h. After evaporation of the solvents under vacuum, the residue was dissolved in water (30 mL), a spatula of Chelex100 (Bio-Rad) was added to the solution, and the mixture was stirred for one hour at room temperature. The resin was then filtered and washed with water (2×20 mL). The filtrate was concentrated under reduced pressure and the residue obtained was purified by size exclusion chromatography (Sephadex, LH20, GE Healthcare. MeOH) to give compound 14 (40%).

¹H NMR (300 MHz, MeOD) δ (ppm) 1.01-1.55 (m, 39H, CH₃, tBu), 2.4-3.85 (m, 264H, CH₂CONH, CH₂CONH PAMAM, CONHCH₂CH₂O, CH₂NHCONH, CONHCH₂CH₂N(CH₂CH₃)₂, CONHCH₂CH₂N(CH₂CH₃)₂), CH₂O, CH₂N and NCH₂ PAMAM, CH₂ DOTA), 4.55 (m, 2H, CH₂ next to triazole ring), 7.92 (m, 5H, Ar-3-H, H triazole ring), 8.07 (m, 4H, Ar-4-H), 8.37 (m, 4H, Ar-6-H), 9.40 (m, 4H, Ar-8-H); ¹³C NMR (75 MHz, MeOD) δ (ppm) 173.4, 173.3, 173.2, 173.0, 172.6, 165.1, 161.8, 161.3, 155.9, 144.5, 143.7, 143.2, 141.1, 136.5, 129.9, 124.5, 118.7, 114.9, 112.5, 81.3, 77.3, 73.8, 72.2, 70.1, 70.0, 69.9, 69.8, 69.7, 69.2, 69.1, 67.9, 66.8, 62.4, 55.8, 55.3, 51.0, 49.7, 49.1, 40.9, 39.5, 39.3, 39.0, 38.9, 36.5, 34.9, 34.6, 33.8, 33.0, 31.7, 27.1, 8.9.

TMSBr (0.03 mL, 0.21 mmol) was added to a suspension of 14 (0.03 g, 0.02 mmol) in CH₂Cl₂ (5 mL). After 3 h of stirring, the silyl esters were hydrolyzed by adding MeOH (3 mL), the mixture being stirred for 2 hours at RT. The solvent was evaporated under reduced pressure, and the residue obtained was purified by size exclusion chromatography (Sephadex. LH20, GE Healthcare. CH₂Cl₂/MeOH 50/50) to give compound 15 (50%) in the form of a yellow powder.

¹H NMR (300 MHz, MeOD) δ (ppm) 1.34-1.46 (m, 24H, CH₃), 2.5-3.95 (m, 264H, CH₂CONH, CH₂CONH PAMAM, CONHCH₂CH₂O, CH₂NHCONH, CONHCH₂CH₂N(CH₂CH₃)₂, CONHCH₂CH₂N(CH₂CH₃)₂), CH₂O, CH₂N and NCH₂ PAMAM, CH₂ DOTA), 4.55 (m, 2H, CH₂ next to triazole ring), 7.93 (m, 5H, Ar-3-H, H triazole ring), 8.08 (m, 4H, Ar-4-H), 8.39 (m, 4H, Ar-6-H), 9.43 (m, 4H, Ar-8-H); ¹³C NMR (75 MHz, MeOD) δ (ppm) 172.3, 171.1, 165.6, 155.9, 144.6, 143.8, 143.7, 143.2, 141.0, 136.6, 129.9, 124.5, 112.5, 72.3, 70.9, 70.1, 69.7, 69.2, 69.0, 68.9, 66.8, 66.4, 66.2, 60.8, 51.0, 50.7, 50.2, 41.2, 39.4, 39.0, 36.2, 34.4, 34.3, 31.6, 29.3.

### Example 3

### Synthesis of Compound 40 (APAG22-1) Comprising a Tripodal Chelating Agent and a Melanoma Agent Targeting

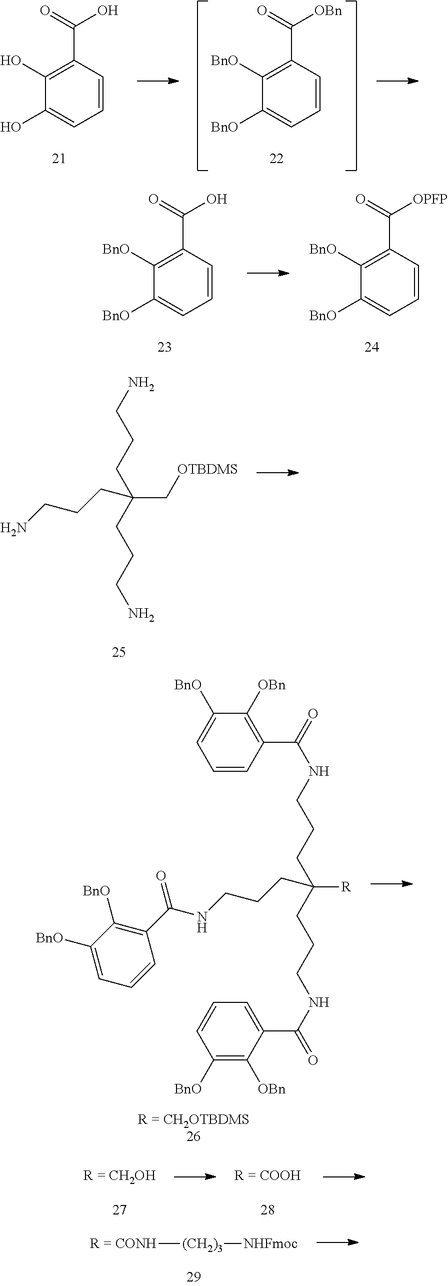

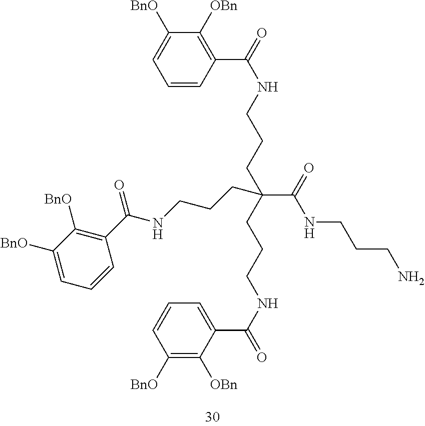 [SMILES: NCCCNC(=O)C(CCCNC(=O)c1cccc(OCc2ccccc2)c1OCc1ccccc1)(CCCNC(=O)c1cccc(OCc2ccccc2)c1OCc1ccccc1)CCCNC(=O)c1cccc(OCc2ccccc2)c1OCc1ccccc1]

A solution of 2,3-dihydrobenzoic acid (21) (9.50 g, 61.6 mmol) and K₂CO₃ (33.20 g, 240.07 mmol) in acetonitrile (140 mL) was heated at 80° C. for 1 h. The reaction mixture was then cooled to room temperature and a solution of benzyl bromide (29.5 mL, 246.8 mmol) in acetonitrile (50 mL) was added dropwise. The reaction mixture obtained was stirred for 16 h at 80° C. and filtered. The filtrate (containing compound 22) was concentrated under reduced pressure and then diluted in EtOH (100 mL). After adding a solution of NaOH (7.50 g, 185.0 mmol) in 20 mL of water, the reaction mixture was refluxed for 4 hours and then concentrated under reduced pressure. The residue was poured into 200 mL of water and hydrochloric acid was added until acidic pH was obtained. The precipitate that formed was filtered, washed with water and then with petroleum ether, and dried under vacuum. Compound 23 was obtained in the form of a white solid (94%) and was used without further purification.

¹H NMR (300 MHz, CDCl₃); δ (ppm) 5.21 (s, 2H, CH₂-Benzyl), 5.26 (s, 2H, CH₂-Benzyl), 7.20 (t, 1H, 3J=8.0 Hz, Ar-5-H), 7.25 (dd, 1H, J=1.8 and 8.0 Hz, Ar-4-H), 7.32 to 7.50 (m, 10H, ArHBenzyl), 7.74 (dd, 1H, J=2.0 and 8.0 Hz, Ar-6-H), 10.95 (s, 1H, COOH); ¹³C NMR (75 MHz, CDCl₃); δ (ppm) 165.58, 151.23, 147.02, 135.87, 134.51, 129.28, 128.81 (J=2.7 Hz), 128.52, 127.74, 125.02, 124.26, 122.94, 118.93, 77.12, 71.49. MALDI: calculated for C₂₁H₁₈NaO₄: 357.12, obtained: 357.11; calculated for C₄₂H₃₆NaO₈: 691.24, obtained: 691.23

DCC (3.70 g, 18.0 mmol, 1.2 eq.) and phenol pentafluoride (3.30 g, 18.0 mmol, 1.2 eq.) were added to a solution of carboxylic acid 23 (5.0 g, 15.0 mmol) in 100 mL of CH₂Cl₂ at 0° C. The reaction mixture was stirred for 4 h at room temperature, concentrated under reduced pressure to reduce the volume of solvent, filtered on Celite, and then concentrated under reduced pressure. The residue was purified by silica gel chromatography (cyclohexane/ethyl acetate (90/10)). Compound 24 was obtained in the form of yellowish oil, which crystallizes under vacuum. (72%).

¹H NMR (300 MHz, CDCl₃): δ (ppm) 5.18 (s, 2H, CH₂-Benzyl), 5.21 (s, 2H, CH₂-Benzyl), 7.18 (t, 1H, 3J=8.0 Hz, Ar-5-H), 7.31 (dd, 1H, J=1.8 and 8.0 Hz, Ar-4-H), 7.33 to 7.50 (m, 10H, ArHBenzyl), 7.66 (dd, 1H, J=1.9 and 8.0 Hz, Ar-6-H); 19F (81 MHz, CDCl₃): δ (ppm) −152.21 (d, 2F. Ar-2.6-F), −158.23 (t, 1F, Ar-3-F), −162.47 (t, 2F, Ar-3,5-F); ¹³C NMR (75 MHz, CDCl₃): δ (ppm) 161.32, 151.03, 149.83, 136.92, 136.17, 128.63 (J=6.6 Hz), 128.22, 128.05, 127.55, 124.17, 123.82, 122.91, 118.92, 75.83, 71.35. MALDI: calculated for C₂₇H₁₇NaF₅O₄: 523.10, obtained: 523.09.

A solution of compound 24 (5.22 g, 10.44 mmol) in freshly distilled dichloromethane (20 mL) was added dropwise to a solution of the triamine derivative 25 (1.08 g, 3.26 mmol) and N,N-diisopropylethylamine (2.30 mL, 14.0 mmol, 4.3 eq.) in freshly distilled dichloromethane (70 mL) stored under nitrogen. The reaction mixture was stirred overnight at room temperature. The organic phase was washed with 1M sodium hydroxide solution (2×40 mL), 1M HCl solution (2×40 mL), brine (2×50 mL) and water (2×50 mL), then dried over MgSO₄, filtered and concentrated under reduced pressure. The residue was purified by silica gel chromatography (dichloromethane/methanol 100 to 99/1) to give compound 26 in the form of colorless oil, which crystallizes under vacuum (71%).

¹H NMR (300 MHz, CDCl₃): δ (ppm) −0.09 (s, 6H, Si(CH₃)2CCH₃)3, 0.77 (s, 9H, Si(CH₃)2CCH₃)3, 0.98 (m, 6H, CH₂CH₂CH₂NH), 1.11 (m, 6H, CH₂CH₂CH₂NH), 3.04 (s, 2H, CH₂OSi), 3.18 (q, 6H, J=6.0 Hz, CH₂CH₂CH₂NH), 5.05 (s, 6H, CH₂-Benzyl), 5.12 (s, 6H, CH₂-Benzyl), 7.10 to 7.15 (m, 6H, Ar-4-H and Ar-5-H), 7.25 to 7.47 (m, 30H, ArHBenzyl), 7.73 (dd, 3H, J=3.0 and 6.6 Hz, Ar-6-H), 7.18 (t, 3H, J=5.3 Hz, CH₂CH₂CH₂NH): ¹³C NMR (75 MHz, CDCl₃): δ (ppm) 164.96, 151.72, 146.82, 136.23 (J=6.6 Hz), 128.61, 128.18, 127.58, 127.47, 124.33, 123.21, 116.82, 76.08, 71.07, 65.92, 41.25, 39.03, 30.91, 25.80, 22.91, 17.02, −5.83. MALDI: calculated for C₈₀H₈₉NaN₃O₁₀Si; 1302.63, obtained: 1302.62.

5.4 mL (5.4 mmol, 3 eq.) of a solution of tetra-n-butylammonium fluoride 1.0 M in THF was added slowly to a solution of compound 26 (2.3 g, 1.80 mmol) in 40 mL of distilled THF, at 0° C. After 3 hours of reflux, the reaction mixture was concentrated under reduced pressure, 50 mL of dichloromethane were then added, the organic phase was washed with brine and then water, dried over MgSO₄, filtered and concentrated under reduced pressure. The oil obtained was purified by silica gel chromatography (dichloromethane/methanol 98/2 to 97/3) to give compound 27 in the form of colorless oil, which crystallizes under vacuum (92%).

¹H NMR (300 MHz, CDCl₃): δ (ppm) 0.98 (m, 6H, CH₂CH₂CH₂NH), 1.15 (m, 6H, CH₂CH₂CH₂NH), 3.06 (s, 2H, CH₂OH), 3.18 (q, 6H, J=6.0 Hz, CH₂CH₂CH₂NH), 5.04 (s, 6H, CH₂-Benzyl), 5.12 (s, 6H, CH₂-Benzyl), 7.05 to 7.12 (m, 6H, Ar-4-H and Ar-5-H), 7.27 to 7.48 (m, 30H, ArHBenzyl), 7.73 (dd, 3H, J=2.8 and 6.3 Hz, Ar-6-H), 7.18 (t, 3H, J=5.3 Hz, CH₂CH₂CH₂NH): ¹³C NMR (75 MHz, CDCl₃): δ (ppm) 165.08, 151.81, 146.80, 136.21, 128.68, 128.19, 127.61, 127.33, 124.35, 123.12, 116.79, 76.24, 71.12, 41.30, 39.02, 30.78, 22.88. MALDI: calculated for C₇₄H₇₅NaN₃O₁₀: 1188.55, obtained: 1188.51.

1.65 mL (3.26 mmol) of a 2M solution of oxalyl chloride in dichloromethane was slowly added to a solution of dimethyl sulfoxide anhydride (0.23 mL, 3.26 mmol) in 2 mL of dry dichloromethane at −60° C. The mixture was then stirred for 15 minutes at the same temperature, then a solution of compound 27 (1.9 g, 1.63 mmol) in dry dichloromethane (10 mL) was added in the space of ten minutes. The resulting solution was stirred for one additional hour and the temperature of the reaction mixture rose to −30° C. Triethylamine (2.3 mL, 16.3 mmol, 10 equiv.) was added and the reaction mixture was brought back to room temperature. Water (50 mL) and dichloromethane (50 mL) were added and the organic phase was washed with brine, dried over MgSO₄, filtered and concentrated under reduced pressure. The aldehyde thus obtained was used in the next step without additional purification. Sulfamic acid (0.32 g, 3.27 mmol, 2 eq.) and sodium chlorite (0.33 g, 3.27 mmol, 2 eq.) were added with stirring to a solution of said aldehyde (1.9 g, 1.63 mmol) in a THF/water mixture (1/1). The reaction mixture was stirred overnight at room temperature. Water (50 mL) and dichloromethane (50 mL) were added and the organic phase was washed with brine, dried over MgSO₄, filtered and concentrated under reduced pressure. The oil obtained was purified by silica gel chromatography (dichloromethane/methanol 99/1 to 98/2) to give compound 28 in the form of colorless oil, which crystallizes under vacuum (83% on both steps).

¹H NMR (300 MHz, CDCl₃): δ (ppm) 1.12 (m, 6H, CH₂CH₂CH₂NH), 1.32 (m, 6H, CH₂CH₂CH₂NH), 3.17 (q, 6H, J=5.5 Hz, CH₂CH₂CH₂NH), 5.01 (s, 6H, CH₂-Benzyl), 5.11 (s, 6H, CH₂-Benzyl), 7.06 to 7.12 (m, 6H, Ar-4-H and Ar-5-H), 7.27 to 7.43 (m, 30H, ArHBenzyl), 7.73 (dd, 3H, J=3.3 and 6.1 Hz, Ar-6-H), 7.18 (t, 3H, J=5.4 Hz, CH₂CH₂CH₂NH); ¹³C NMR (75 MHz, CDCl₃): δ (ppm) 165.02, 151.74, 146.61, 136.20 (J=7.0 Hz), 128.71, 128.18, 127.59, 127.31, 124.12, 123.17, 116.79, 76.26, 71.11, 39.97, 36.96, 31.25, 23.34. MALDI: calculated for C₇₄H₇₃NaN₃O₁₁: 1202.52, obtained: 1202.50.

BOP (0.18 g, 0.39 mmol, 1.3 eq.) was added under argon to a solution of carboxylic acid 28 (0.35 g, 0.3 mmol) in 10 mL of distilled dichloromethane. After 5 minutes, N-Fmoc-1,3-propanediamine (0.12 g, 0.33 mmol, 1.1 eq.) and N,N-diisopropylethylamine (0.17 mL, 1 mmol, 3 eq. per amine) were added. The reaction mixture was stirred overnight at room temperature, 20 mL of dichloromethane was added and the organic phase was washed with brine and then water, dried over MgSO₄, filtered and concentrated under reduced pressure. The residue was purified by silica gel chromatography (dichloromethane/methanol 99/1 to 97/3) to give compound 29 in the form of colorless oil, which crystallizes under vacuum (90%).

¹H NMR (300 MHz, CDCl₃): δ (ppm) 1.18 (m, 6H, CH₂CH₂CH₂NH), 1.31 (m, 6H, CH₂CH₂CH₂NH), 1.52 (m, 2H, NHCH₂CH₂CH₂NHFmoc), 3.06 (m, 2H, NHCH₂CH₂CH₂NHFmoc), 3.17 (m, 8H, CH₂CH₂CH₂NH and NHCH₂CH₂CH₂NHFmoc), 4.15 (t, 1H, J=6.8 Hz, COOCH₂CH), 4.15 (d, 2H, J=7.2 Hz, COOCH₂CH), 5.03 (s, 6H, CH₂-Benzyl), 5.09 (s, 6H, CH₂-Benzyl), 5.71 (t, 1H, J=6.0 Hz, NHCH₂CH₂CH₂NHFmoc), 6.08 (t, 1H, J=5.9 Hz, NHCH₂CH₂CH₂NHFmoc), 7.06 to 7.10 (m, 6H, Ar-4-H and Ar-5-H), 7.21 to 7.48 (m, 34H, ArHBenzyl), 7.57 (d, 2H, J=7.7 Hz, Ar-Fmoc-H), 7.65 to 7.77 (m, 5H, Ar-Fmoc-H and Ar-6-H), 7.91 (t, 3H, J=5.4 Hz, CH₂CH₂CH₂NH); ¹³C NMR (75 MHz, CDCl₃): δ (ppm) 165.31, 151.81, 146.72, 144.02, 141.35, 128.75, 128.70, 128.65, 128.60, 127.62, 127.60, 127.45, 127.04, 125.22, 124.31, 123.19, 119.82, 116.79, 76.38, 71.20, 66.37, 47.81, 47.23, 39.97, 32.08, 23.88. MALDI: calculated for C₉₂H₉₁NaN₅O₁₂: 1480.67, obtained: 1480.63.

Piperidine (0.21 mL, 0.24 mmol, 1 eq.) was added slowly under argon to a solution of compound 29 (0.35 g, 0.24 mmol) in 5 mL of distilled dichloromethane at 0° C. The reaction mixture was stirred for 2 hours at room temperature, 20 mL of dichloromethane was then added, the organic phase was washed with 1 M sodium hydroxide solution and then brine, dried over MgSO₄, filtered and concentrated under reduced pressure. The residue was purified by silica gel chromatography (dichloromethane/methanol 98/2 to 90/10) to give compound 30 in the form of colorless oil, which crystallizes under vacuum (90%).

¹H NMR (300 MHz, CDCl₃): δ (ppm) 1.15 (m, 6H, CH₂CH₂CH₂NH), 1.41 (m, 6H, CH₂CH₂CH₂NH), 1.95 (m, 2H, NHCH₂CH₂CH₂NH₂), 2.95 (m, 2H, NHCH₂CH₂CH₂NH₂), 3.12 (m, 6H, CH₂CH₂CH₂NH), 3.36 (m, 2H, NHCHCH₂CH₂NH₂), 5.04 (s, 6H, CH₂-Benzyl), 5.11 (s, 6H, CH₂-Benzyl), 7.06 to 7.10 (m, 6H, Ar-4-H and Ar-5-H), 7.21 to 7.45 (m, 30H, ArHBenzyl), 7.62 (m, 3H, Ar-6-H), 8.11 (t, 3H, J=5.4 Hz, CH₂CH₂CH₂NH), 8.36 to 8.51 (m 3H, NHCH₂CH₂CH₂NH₂ and NHCH₂CH₂CH₂NH₂); ¹³C NMR (75 MHz, CDCl₃): δ (ppm) 165.23, 151.62, 146.74, 136.22, 128.81, 128.70, 128.65, 128.31, 127.67, 127.18, 124.46, 123.11, 117.02, 76.43, 71.17, 47.96, 40.22, 31.71, 23.96. MALDI: calculated for C₇₇H₈₂NsO₁₀: 1236.60, obtained: 1236.60; C₇₂H₈₁NaN₅O₁₀: 1258.60, obtained: 1258.58; calculated for C₇₇H₈₁KN₅O₁₀: 1274.60, obtained: 1274.56.

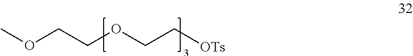

A solution of para-toluenesulfonyl chloride (22.3 g, 105 mmol) in THF (35 mL) was added dropwise to a solution of tetraethylene glycol methyl ether (20.0 g, 96 mmol) and NaOH (6.7 g, 166 mmol) in a THF/H₂O mixture (135 mL/45 ml) at 0° C. After stirring for one hour at 0° C., the reaction mixture was left to return to room temperature and then it was stirred for a further 20 hours. The solution was then poured into 200 ml of brine and the volatile materials were evaporated. The resultant mixture was extracted several times with dichloromethane and the combined organic phases were washed with brine, dried over MgSO₄, filtered and evaporated under reduced pressure. The oily residue was purified by silica gel chromatography with a dichloromethane/methanol mixture (98/2) as eluent. Compound 32 is obtained in the form of a pale yellow oil in 96% yield

¹H NMR (300 MHz, CDCl₃) δ 2.39 (s, 3H, ArCH₃), 3.31 (s, 3H, OCH₃), 3.64 to 3.47 (m, 14H, OCH₂CH₂O), 4.11 to 4.08 (m, 2H, ArSO₂OCH₂), 7.28 (d, J=1.5 Hz, 2H, Ar-3,5-H), 7.73 (d, J=1.5 Hz, 2H, Ar-2,6-H); ¹³C NMR (75 MHz, CDCl₃) δ 21.78, 59.14, 68.80, 69.45, 70.66, 70.73, 70.86, 72.07, 128.10, 129.99, 133.19, 144.96.

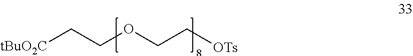

840 mL (6.0 mmol, 3.0 eq.) of triethylamine and 570 mg (3.0 mmol, 1.5 eq.) of para-toluenesulfonyl chloride were added successively to a solution of hydroxy-dPEG™8-t-butyl ester (1.00 g, 2.0 mmol) in 20 ml of dichloromethane at 0° C. After stirring for 40 h at room temperature, the reaction mixture was diluted with 70 ml of dichloromethane. The organic phases were combined, washed with brine, dried over MgSO₄, filtered and concentrated under reduced pressure. The crude product was purified by silica gel chromatography (ethyl acetate/methanol 95/5 to 90/10) to give 33 in the form of a colorless oil, in 70% yield

¹H NMR (300 MHz, CDCl₃): δ (ppm) 1.44 (s, 9H), 2.45 (s, 3H), 2.50 (t, 2H, 3J=6.6 Hz), 3.58-3.73 (m, 32H), 4.16 (t, 2H, 3J=4.9 Hz), 7.34 (2H, AA′ of an AA′BB′ system), 7.81 (2H, BB′ of an AA′BB′ system).

¹³C NMR (75 MHz, CDCl₃): δ (ppm) 21.14, 27.65, 35.84, 66.40, 68.15, 69.00, 70.08, 79.78, 127.46, 129.47, 132.74, 144.32, 170.20. MALDI: calculated for C₁₀H₂₀LiO₅: 227.15, obtained: 227.08; calculated for C₂₆H₄₄LiO₁₂S: 587.27, obtained: 587.13.

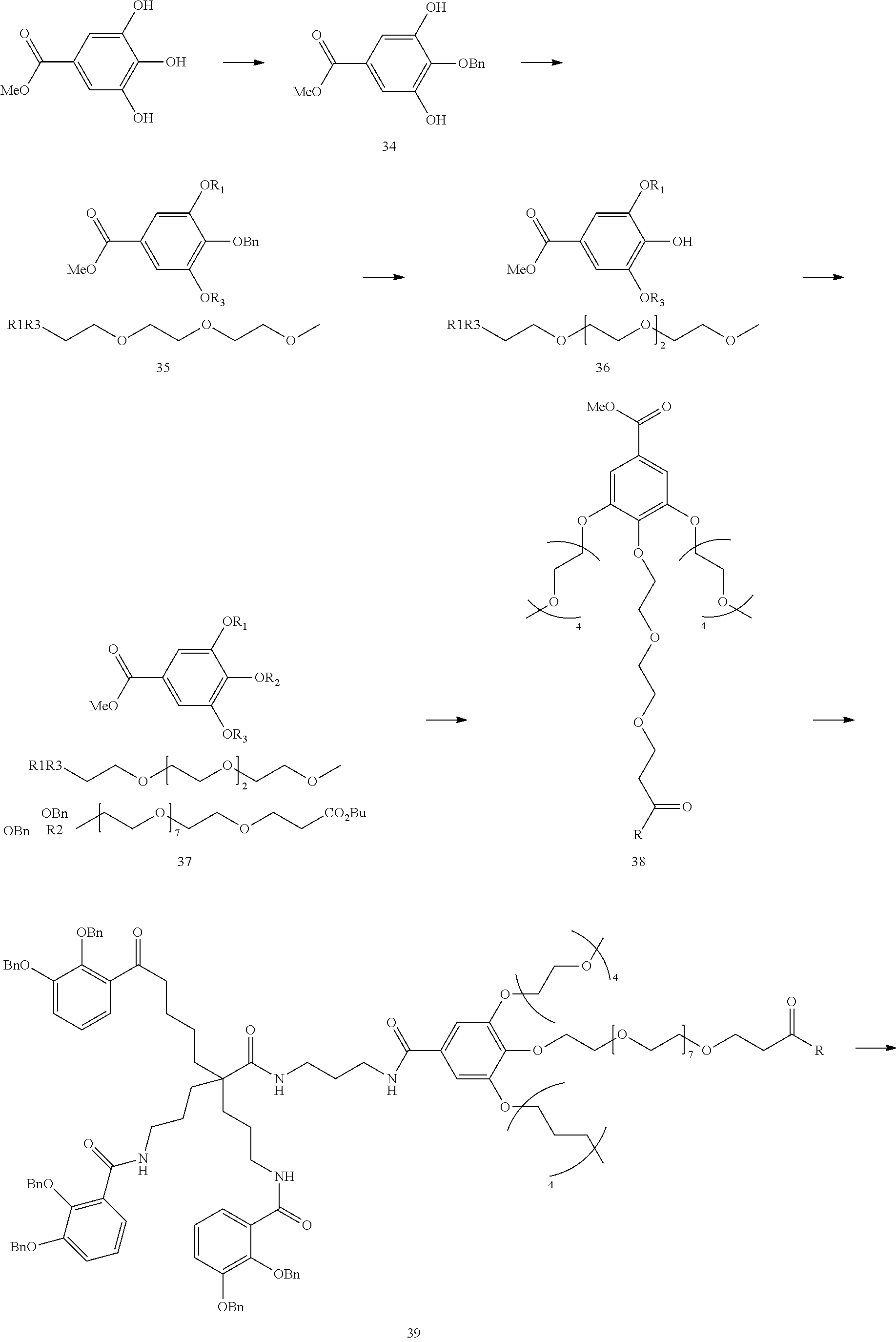

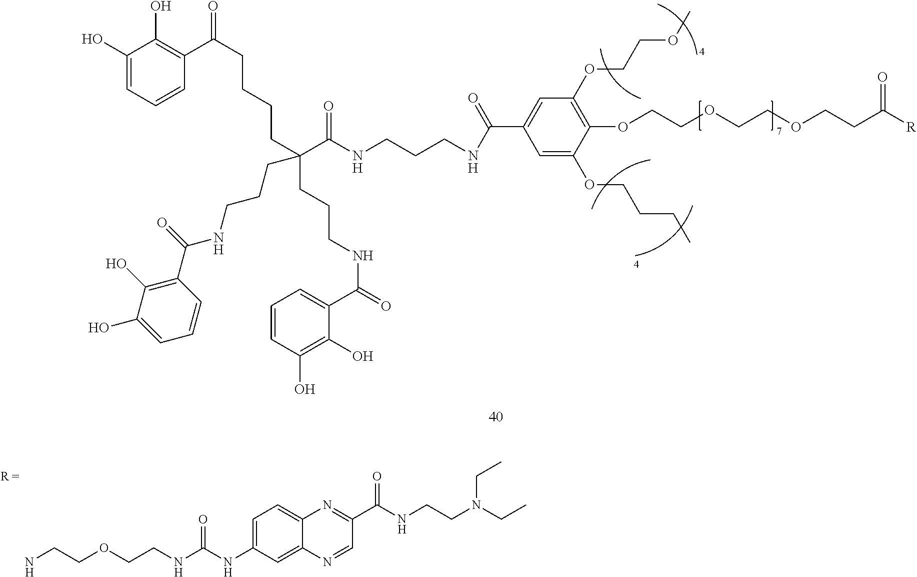

A solution of methyl gallate (20.0 g, 109 mmol). BnBr (14.2 mL, 119 mmol), KHCO₃ (32.4 g, 324 mmol) and KI (0.1 g, 0.60 mmol) in DMF (100 ml) was stirred for 4 days at room temperature. The reaction mixture was then poured into 1 L of water and sulfuric acid was added until neutral pH was obtained. The aqueous phase was then extracted 3 times with 150 ml of dichloromethane. The organic phases were combined, washed three times with 50 mL of brine, dried over MgSO₄, filtered and the volatile substances were evaporated. The solvent was removed by evaporation and the residue was purified by silica gel chromatography, eluting with a CH₂Cl₂/MeOH mixture (98/2) to give a yellow oil. The residue was evaporated several times with dichloromethane. The residue obtained was filtered and washed with petroleum ether to give a white solid 34 in 70% yield.

¹H NMR (300 MHz, CD₃OD) δ 3.83 (s, 3H, COOCH₃), 5.18 (s, 2H, Ar2OCH₂), 7.13 (s, 2H, Ar1-2,6-H), 7.31 (m, 3H, Ar2-3,4,5-H), 7.52 (d, J=7.5 Hz, 2H, Ar2-2,6-H); ¹³C NMR (75 MHz, CD₃OD) δ 51.2, 73.8, 108.8, 125.0, 127.8, 128.0, 128.5, 137.2, 138.2, 150.5, 167.1.

A solution of the diphenol derivative 34 (9.2 g, 33.4 mmol), of the tosylated compound 32 (26.9 g, 74.3 mmol, 2.2 eq.), of K₂CO; (28.0 g, 200 mmol, 6.0 eq.) and of KI (0.6 g, 3.3 mmol, 0.1 eq.) in acetone (600 ml) was stirred for 30 hours at 65° C. The reaction mixture was filtered on Celite and the solvent was evaporated. The crude product obtained was dissolved in dichloromethane (200 mL) and washed twice with a saturated aqueous solution of NaHCO₃ and with brine. After drying over MgSO₄, filtration and evaporation of the solvent, the crude product was purified by silica gel chromatography (dichloromethane/methanol 98/2 to 95/5) to give 35 in the form of a colorless oil, in 75% yield.

¹H NMR (300 MHz, CDCl₃) δ 3.35 (s, 6H, OCH₂CH₂OCH₃), 3.50 to 3.54 (m, 4H, OCH₂CH₂O), 3.60 to 3.67 (m, 16H, OCH₂CH₂O), 3.69 to 3.74 (m, 4H, OCH₂CH₂O), 3.85 to 3.88 (t, J=4.8 Hz, 4H, OCH₂CH₂O), 3.90 (s, 3H, COOCH₃), 4.17 to 4.20 (t, J=4.8 Hz, 4H, Ar1OCH₂), 5.12 (s, 2H, Ar2OCH₂), 7.28 (m, 5H, Ar2-3,4,5-H and Ar1-2.6-H), 7.48 (d, J=7.7 Hz, 2H, Ar2-2,6-H); ¹³C NMR (75 MHz, CDCl₃) δ 52.5, 59.3, 69.2, 70.0, 70.8, 70.9, 71.0, 71.2, 72.3, 74.8, 109.1, 125.3, 127.8, 128.0, 128.2, 138.2, 142.2, 152.5, 166.9. MALDI: calculated for C₃₃H₅₀NaO₁₃: 677.33, obtained: 677.03.

10% Palladium on activated charcoal (0.5 eq.) was added to a solution of the benzylated compound 35 (3.0 g, 4.65 mmol) dissolved in absolute ethanol (50 ml). The mixture was stirred under a hydrogen atmosphere at room temperature for 16 h. The product was filtered on Celite and the solvent was evaporated under reduced pressure. The crude product was purified by silica gel chromatography (dichloromethane/methanol 98/2 to 95/5) to give 36 in the form of a colorless oil in 91% yield.

¹H NMR (300 MHz, CDCl₃) δ 3.37 (s, 6H, OCH₂CH₂OCH₃), 3.50 to 3.56 (m, 4H, OCH₂CH₂O), 3.60 to 3.70 (m, 16H, OCH₂CH₂O), 3.72 to 3.76 (m, 4H, OCH₂CH₂O), 3.85 to 3.88 (t, J=4.8 Hz, 4H, OCH₂CH₂O), 3.89 (s, 3H, COOCH₃), 4.21 (t, J=4.8 Hz, 4H, Ar1OCH₂), 7.26 (s, 2H, Ar1-2,6-H): ¹³C NMR (75 MHz, CDCl₃) δ 52.41, 59.35, 69.18, 70.04, 70.82, 70.88, 71.02, 71.17, 72.30, 109.12, 125.08, 142.11, 152.21, 166.81. MALDI: calculated for C₂₆H₄₄Na₁₃: 587.62, obtained: 587.56.

K₂CO₃ (0.29 g, 2.12 mmol, 4 eq.) was added to an equimolar solution of the phenolic derivative 36 (0.3 g, 0.52 mmol) and the tosylated derivative 33 (0.32 g, 0.52 mmol) in 10 ml of acetone. The reaction mixture was stirred at 60° C., for 24 h. After filtration on Celite, the solvent was evaporated and the residue was diluted in dichloromethane (50 mL). The organic phase was washed twice with a saturated solution of NaHCO₃, then with brine, filtered and concentrated under reduced pressure. The crude product was purified by silica gel chromatography (dichloromethane/methanol 98/2 to 93/7) to give 37 in the form of a pale yellow oil, in 82% yield.

¹H NMR (300 MHz, CDCl₃) δ 1.47 (s, 9H, CH₂COOC(CH₃)3), 2.51 (t, 2H, J=6.5 Hz, CH₂COOC(CH₃)3), 3.36 (s, 6H, OCH₂CH₂OCH₃), 3.50-3.54 (m, 4H, OCH₂CH₂O), 3.60 to 3.72 (m, 54H, OCH₂CH₂O), 3.78 (t, J=5.4 Hz, 2H, OCH₂CH₂O), 3.84 (t, J=4.8 Hz, 4H, OCH₂CH₂O), 3.88 (s, 3H, COOCH₃), 4.15-4.22 (t, J=4.8 Hz, 6H, Ar1OCH₂), 7.31 (s, 2H, Ar1-2,6-H); ¹³C NMR (75 MHz, CDCl₃) δ 28.11, 39.78, 52.39, 59.37, 69.22, 70.03, 70.80, 70.88, 71.03, 71.24, 72.30, 109.19, 125.21, 142.14, 152.25, 166.89, 172.36. MALDI: calculated for C₄₉H₈₈NaO₂₃: 1067.57, obtained: 1067.45.

1 ml (large excess) of trifluoroacetic acid was added dropwise to a solution of compound 37 (0.2 g, 0.19 mmol) in 3 mL of dichloromethane at 0° C. The reaction mixture was stirred for 1 h at room temperature under argon, and then the volatile compounds were evaporated to dryness. The crude product in acidic form was obtained in the form of a colorless oil and was used without further purification. The coupling agent BOP (76 mg, 0.17 mmol, 1.3 eq. per acid) is added under argon to a solution of carboxylic acid thus obtained (0.13 g, 0.13 mmol, 1.1 eq) in 5 mL of distilled dichloromethane. After 5 min, the amine R (55 mg, 0.12 mmol 1.0 eq.) and N,N-diisopropylethylamine (60 μl, 0.36 mmol, 3 eq. per amine) were added. The reaction mixture was stirred overnight at room temperature. 50 ml of dichloromethane was added and the organic phase was washed with 1N sodium hydroxide solution (3×20 ml), brine (2×20 ml) and water (2×20 mL), dried over MgSO₄, filtered and concentrated under reduced pressure. The crude product was purified by column chromatography on neutral alumina (dichloromethane/methanol 99/1 to 98/2) to give 38 in the form of a pale yellow oil, in 82% yield.

¹H NMR (300 MHz, CDCl₃) δ 1.11 (t, 6H, J=7.2 Hz, N(CH₂CH₃)₂), 2.51 (t, 2H, J=5.6 Hz, CH₂CONH), 2.65 (q, 4H, J=7.2 Hz, N(CH₂CH₃)₂), 2.73 (t, 2H, J=5.6 Hz, CH₂N(CH₂CH₃)₂), 3.33 (s, 6H, OCH₂CH₂OCH₃), 3.48 to 3.80 (m, 68H, CONHCH₂ and OCH₂CH₂O), 3.80 to 3.86 (t, J=4.8 Hz, 6H, OCH₂CH₂O), 3.89 (s, 3H, COOCH₃), 4.17 to 4.22 (t, J=4.8 Hz, 6H, Ar1OCH₂), 6.12 (t, 1H, J=4.5 Hz, CH₂NHCONH), 7.21 (t, 1H, J=5.4 Hz, CH₂CONH), 7.25 (s, 2H, Ar1-2,6-H), 7.92 (d, 1H, J=9.2 Hz, Ar2-5-H), 8.03 (d, 1H, J=2.3 Hz, Ar2-8-H), 8.03 (dd, 1H, J=2.3 and 9.2 Hz, Ar2-6-H), 8.35 (t, 1H, J=5.3 Hz, CONHCH₂), 9.19 (s, 1H, CH₂NHCONH), 9.51 (s, 1H, Ar2-2-H); ¹³C NMR (75 MHz, CDCl₃) δ 11.91, 36.95, 37.03, 39.62, 47.03, 51.71, 52.12, 58.93, 67.01, 68.84. 69.51, 69.60, 69.72, 70.02, 70.08, 70.21, 70.25, 70.45, 70.60, 71.82, 72.21, 109.12, 113.18, 124.31, 124.74, 129.97, 136.22, 141.19, 142.23, 143.88 (J=3.9 Hz), 145.01, 152.04, 155.21, 163.86, 166.21, 172.82. MALDI: calculated for C₆₇H₁₁₄N₇O₂₆: 1432.77, obtained: 1432.56, calculated for C₆₇H₁₁₃NaN₇O₂₆: 1454.77, obtained: 1454.53; calculated for C₆₇H₁₁₃KN₇O₂₆: 1470.77, obtained: 1470.53.

Sodium hydroxide (15 mg, 0.3 mmol, 5 eq.) was added to a solution of compound 38 (80 mg, 0.06 mmol) in a 4/1 methanol/water mixture (5 mL). The reaction mixture was stirred for 2 h at 85° C., and was then evaporated to dryness and 30 ml of dichloromethane was added. The organic phase was dried over MgSO₄, filtered and concentrated under reduced pressure. The crude product (carboxylic acid) was obtained in the form of an orange foam and was used without further purification. The coupling agent BOP (28 mg, 0.06 mmol, 1.3 eq. per acid) was added under argon to a solution of carboxylic acid thus obtained (70 mg, 0.05 mmol, 1.1 eq) in 5 mL of distilled dichloromethane. After 5 min, the amine 10 (56 mg, 0.045 mmol, 1.0 eq.) and N,N-diisopropylethylamine (25 μl, 0.135 mmol, 3 eq. per amine) were added. The reaction mixture was stirred overnight at room temperature, 40 ml of dichloromethane was added and the organic phase was washed with 1N sodium hydroxide solution (3×20 ml), brine (2×20 ml) and water (2×20 mL), dried over MgSO₄, filtered and concentrated under reduced pressure. The crude product was purified by column chromatography on neutral alumina (dichloromethane/methanol 95/5 to 90/10) to give 39 in the form of a pale yellow oil, in 59% yield.

¹H NMR (300 MHz, CDCl₃) δ 1.11 (t, 6H, J=7.1 Hz, N(CH₂CH₃)₂), 1.15 (m, 6H, CCH₂CH₂CH₂NH), 1.32 (m, 6H, CCH₂CH₂CH₂NH), 1.62 (m, 2H, NHCH₂CH₂CH₂NHCO), 2.51 (t, 2H, J=5.6 Hz, CH₂CONH), 2.69 (q, 4H, J=7.1 Hz, N(CH₂CH₃)₂), 2.80 (t, 2H, J=5.6 Hz, CH₂N(CH₂CH₃)₂), 3.17 (m, 6H, CCH₂CH₂CH₂NH), 3.31 (s, 6H, OCH₂CH₂OCH₃), 3.34 (m, 2H, NHCH₂CH₂CH₂NH₂), 3.40 to 3.72 (m, 70H, NHCH₂CH₂CH₂NHCO, CONHCH₂ and OCH₂CH₂O), 3.73 to 3.78 (m, 6H, OCH₂CH₂O), 4.08 to 4.17 (m, 6H, Ar1OCH₂), 5.02 (s, 6H, CH₂-Benzyl), 5.09 (s, 6H, CH₂-Benzyl), 6.22 (m, 1H, CH₂NHCOC), 6.51 (t, 1H, J=4.5 Hz, CH₂NHCONH), 7.06 (m, 6H, Ar^{tricatechol}-4-H and Ar^{tricatechol}-5-H), 7.12 (s, 2H, Ar1-2,6-H), 7.21 to 7.42 (m, 31H, CH₂CONH and ArHBenzyl), 7.58 (m, 3H, Ar^{tricatechol}-6-H), 7.71 (m, 1H, Ar1CONH), 7.92 (m, 4H, CH₂CH₂CH₂NH and Ar2-5-H), 8.09 (d, 1H, J=2.3 Hz, Ar2-8-H), 8.18 (dd, 1H, J=2.3 and 9.2 Hz, Ar2-6-H), 8.40 (t, 1H, J=5.3 Hz, CONHCH₂), 9.27 (s, 1H, CH₂NHCONH), 9.47 (s, 1H, Ar2-2-H); ¹³C NMR (75 MHz, CDCl₃) δ 11.48, 24.07, 29.61, 31.94, 36.95. 37.03, 39.71, 40.17, 47.11, 47.94, 51.78, 58.95, 67.04, 68.76, 69.52, 69.60, 69.72, 70.01, 70.09, 70.21, 70.25, 70.45, 70.60, 71.21, 71.83, 72.24, 76.28, 106.91, 113.07, 116.98, 123.01, 124.32, 124.39, 127.16, 127.65, 128.30, 128.65, 128.70, 128.82, 129.61, 130.02, 136.22, 140.86, 141.17, 143.91 (J=4.1 Hz), 145.03, 146.73, 151.61, 152.04, 155.33, 163.94, 165.06, 166.46, 167.22, 172.61, 176.03. MALDI: calculated for C₁₄₃H₁₉₁N₁₂O₃₅; 2636.36, obtained: 2636.29; calculated for C₁₄₃H₁₉₀NaN₁₂O₃₅: 2658.36, obtained: 2658.27; calculated for C₁₄₃H₁₉₁KN₁₂O₃₅: 2674.36, obtained: 2674.21.

20% Palladium hydroxide on charcoal (44 mg, 0.32 mmol, 12 eq.) was added to a solution of the benzyl compound 39 (70 mg, 0.02 mmol) dissolved in absolute ethanol (5 ml). The mixture was stirred under a hydrogen atmosphere at room temperature for 4 h. The product was filtered on Celite and the solvent was evaporated under reduced pressure. The crude product was purified by column chromatography on LH (methanol) to give 40 in the form of an orange foam, in 30% yield.

¹H NMR (300 MHz, CD3OD) δ 1.20 to 1.35 (m, 12H, CCH₂CH₂CH₂NH and N(CH₂CH₃)₂), 1.55 (m, 6H, CCH₂CH₂CH₂NH), 1.71 (m, 6H, CCH₂CH₂CH₂NH), 1.64 (m, 2H, NHCH₂CHCH₂NHCO), 2.48 (m, 2H, CH₂CONH), 3.10 to 3.65 (m, 84H, N(CH₂CH₃)₂, CH₂N(CH₂CH₃)₂, OCH₂CH₂OCH₃, NHCH₂CH₂CH₂NH₂, NHCH₂CH₂CH₂NHCO, CONHCH₂ and OCH₂CH₂O), 3.73 to 3.78 (m, 6H, OCH₂CH₂O), 4.10 to 4.20 (m, 6H, Ar1OCH₂), 6.61 (m, 3H, Ar^{tricatechol}-4-H), 6.92 (m, 3H, Ar^{tricatechol}-5-H), 7.15 (s, 2H, Ar1-2,6-H), 7.37 (m, 3H, Ar^{tricatechol}-6-H), 7.96 (m, 1H, Ar2-5-H), 8.05 (m, 1H, Ar2-8-H), 8.37 (m, 1H, Ar2-6-H), 9.41 (s, 1H, Ar2-2-H); ¹³C NMR (75 MHz, CD3OD) δ 7.92, 23.21, 26.12, 28.82, 28.93, 31.02, 34.21, 35.83, 35.91, 36.52, 38.58, 39.01, 57.19, 66.23, 68.04, 68.81, 68.93, 69.43, 69.60, 69.71, 69.82, 69.89, 71.09, 71.86, 105.88, 112.08, 114.89, 116.82, 117.81, 124.04, 127.18, 127.54, 127.72, 127.85, 128.81, 129.42, 136.18, 140.22, 140.72, 142.84, 143.23, 144.16, 145.51, 147.41, 151.94, 155.36, 165.02, 167.51, 169.77, 172.26, 177.08. MALDI: calculated for C₁₀H₁₅₄N₁₂O₃₅: 2095.06, obtained: 2095.70.

### Example 4

### Synthesis of a Tricatechol Chelating Agent Grafted Directly with a Melanoma Cell Targeting Agent, APAG 1.9

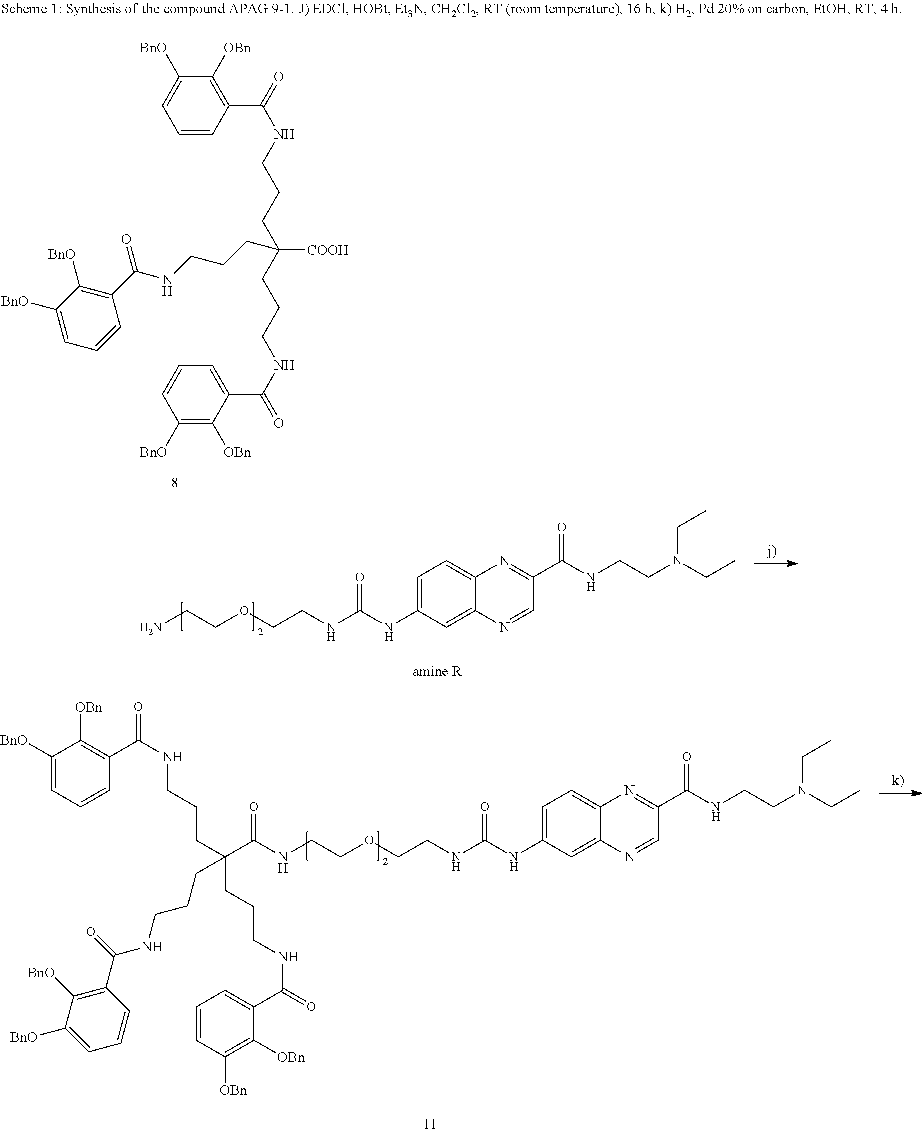

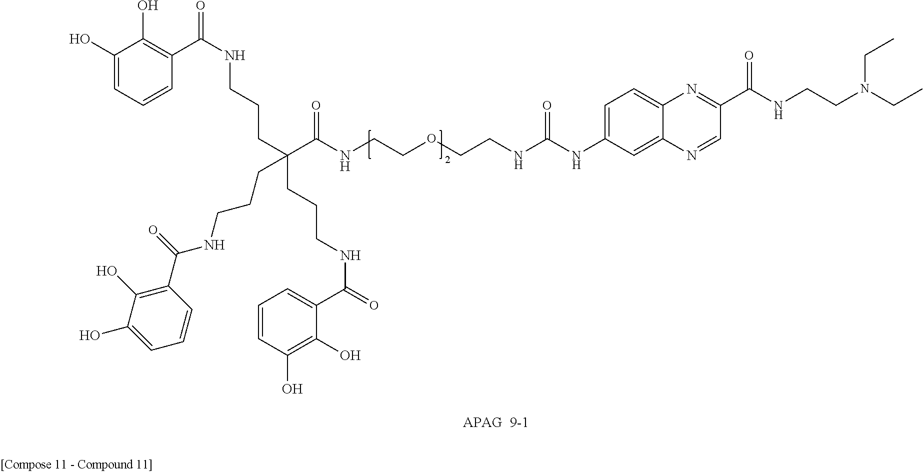

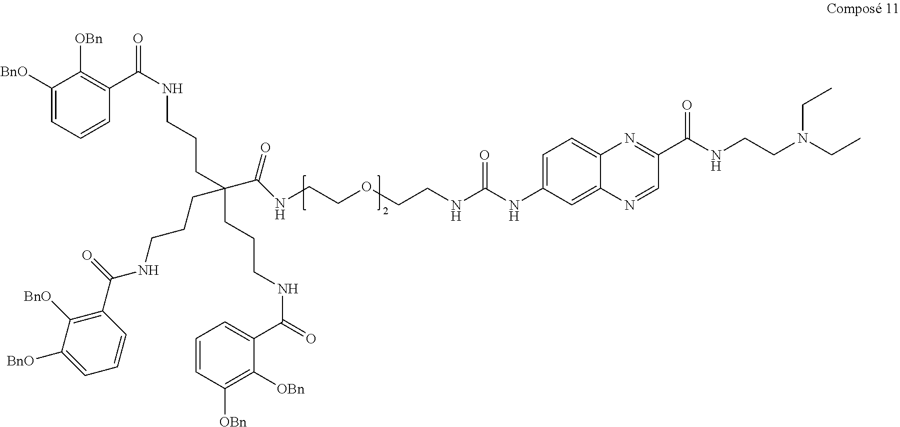

The coupling reagent BOP (0.1 g, 0.22 mmol, 1.3 eq. per acid) was added under argon to a solution of carboxylic acid derivative 8 (0.2 g, 0.17 mmol, 1.0 eq.) in 5 mL of distilled dichloromethane. After 5 min, the amine R (86 mg, 0.18 mmol, 1.1 eq.) and N,N-diisopropylethylamine (90 μl, 0.56 mmol, 3 eq. per amine) were added. The reaction mixture was stirred overnight at room temperature. 40 ml of dichloromethane was added and the organic phase was washed with 1N sodium hydroxide solution (3×20 mL), brine (2×20 ml) and water (2×20 mL), dried over MgSO₄, then filtered and concentrated under reduced pressure. The crude product was purified by silica gel column chromatography (dichloromethane/methanol 98/2-90/10) to give 11 in the form of a yellow solid in 89% yield.

¹H NMR (300 MHz, CDCl₃) δ (ppm) 1.07-1.14 (m, 12H, N(CH₂CH₃)₂) and CCH₂CH₂CH₂NH), 1.31 (m, 6H, CCH₂CH₂CH₂NH), 2.64 (m, 4H, N(CH₂CH₃)₂), 2.73 (m, 2H, CH₂N(CH₂CH₃)₂), 3.17 (m, 6H, CCH₂CH₂CH₂NH), 3.38-3.59 (m, 14H, CONHCH₂ CH₂N(CH₂CH₃)₂, CONHCH₂ and OCH₂CH₂O), 5.05 (s, 6H, CH₂-Benzyl), 5.12 (s, 6H, CH₂-Benzyl), 6.10 (m, 1H, NH), 6.47 (m, 1H, NH), 7.09 (m, 6H, Ar^{tricatechol}-4-H and Ar^{tricatechol}-5-H), 7.21-7.45 (m, 31H, CH₂CONH and ArH_{Benzyl}), 7.66 (m, 3H, Ar^{tricatechol}-6-H), 7.71 (d, 1H, 9.2 Hz, Ar²-3-H), 7.97 (m, 4H, CH₂CH₂CH₂NH and Ar²-4-H), 8.12 (m, 1H, CONH), 8.32 (m, 1H, CONH), 9.07 (s, 1H, Ar²-6-H), 9.47 (s, 1H, Ar²-8-H); ¹³C NMR (75 MHz, CDCl₃) δ (ppm) 11.54, 23.58, 31.82, 39.23, 39.42, 40.03, 47.22, 47.68, 51.35, 69.55, 69.71, 70.21, 70.42, 71.04, 113.21, 116.91, 122.91, 124.47, 124.37, 127.21, 127.43, 128.11, 128.42, 128.71, 130.02, 136.33 (J=3.8 Hz), 143.81, 145.05, 146.71, 151.82, 155.42, 164.02, 165.20, 176.02. MALDI: calculated for C₉₆H₁₀₇N₁₀O₁₄: 1622.79, obtained: 1622.60.

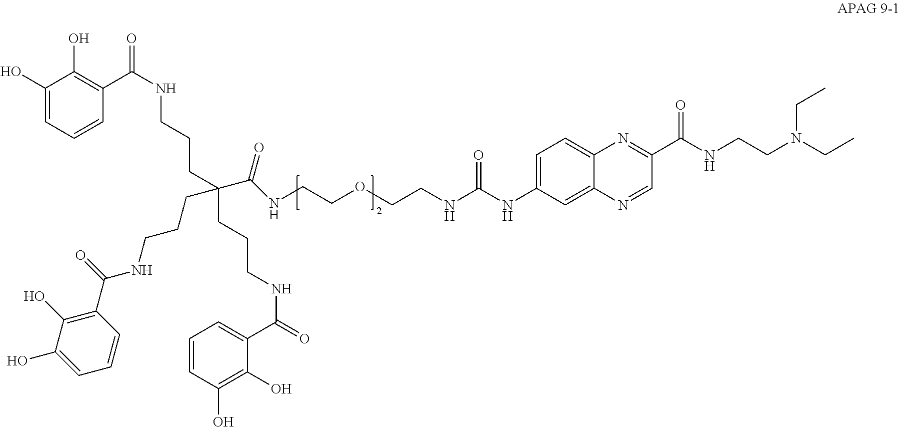

Palladium, 10% on activated charcoal (48 mg, 0.45 mmol, 0.5 eq. per benzyl) was added to a suspension of compound 11 (0.24 g, 0.15 mmol, 1 eq.) dissolved in ethanol (10 mL). The mixture was stirred under a hydrogen atmosphere at room temperature for 4 h. Then the solution was filtered through a Celite stopper, concentrated under vacuum and purified by size exclusion chromatography (GE HealthCare, Sephadex LH20, MeOH) to give the desired compound A (46%) in the form of a yellow solid.

¹H NMR (300 MHz, CD₃OD) δ 1.34-1.65 (m, 18H, CCH₂CH₂CH₂NH, CCH₂CH₂CH₂NH and N(CH₂CH₃)₂), 3.35 to 3.59 (m, 24H, N(CH₂CH₃)₂, CH₂N(CH₂CH₃)₂, CCH₂CH₂CH₂NH and OCH₂CH₂), 3.91 (m, 2H, CONHCH₂CH₂N), 6.69 (dd, 3H, J=7.89 and 7.89 Hz, Ar^{tricatechol}-4-H), 6.92 (m, 3H, Ar^{tricatechol}-5-H), 7.19 (m, 3H, Ar^{tricatechol}-6-H), 7.84 (m, 1H, Ar²-7-H), 7.94 (m, 1H, Ar²-8-H), 8.33 (m, 1H, Ar²-5-H), 9.32 (s, 1H, Ar²-3-H): ¹³C NMR (75 MHz, CD₃OD) δ 7.66, 23.54, 29.32, 31.67, 34.31, 39.09, 39.39, 51.22, 69.28, 69.56, 69.75, 69.91, 112.53, 115.30, 117.25, 118.18, 124.48, 128.19, 129.96, 136.58, 140.81, 143.05, 143.69, 144.47, 145.81, 148.77, 155.96, 165.62, 169.97, 177.76. MALDI: calculated for C₅₄H₇₀N₁₀O₁₄: 1082.51, obtained: 1083.49 [M+H]⁺, 1099.49 [M+OH.]⁺.

### Example 5

### Radiolabeling with Technetium-99m of Compounds Comprising a Chelating Agent of the Following Formula

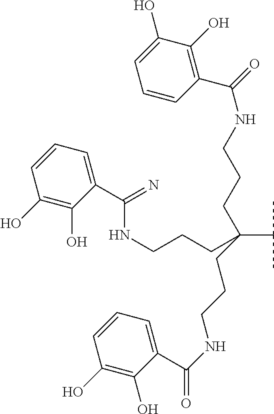

in particular compounds APAG22-1 and APAG9-1.

In a sterile bottle, 2-4 mg of the compound is dissolved in 1 mL of water for injection. 300 μL of a solution of stannous chloride prepared extemporaneously (1 mg·mL⁻¹, 1.31 μg) is added to the bottle. 500 MBq of freshly eluted sodium pertechnetate (Na99mTcO4) is added to the bottle. The mixture is incubated for 20 minutes at room temperature. Then 100 μM of sodium ascorbate is added to the reaction bottle. The pH of the radiolabeled solution is then adjusted to pH=7 with NaOH (FIG. 1).

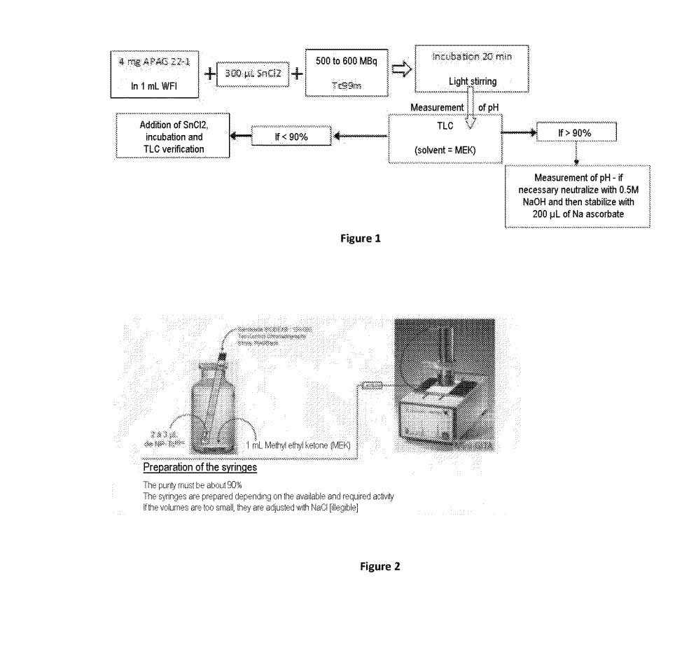

The radiolabeled product is inspected for purity by paper chromatography according to the scheme presented in FIG. 2.

### Example 6

### Synthesis of Compound APAG-53

### General Methods

For synthesis of the intermediate and final products, the reactions were carried out under argon atmosphere. The following solvents were distilled by the drying agents indicated: CH₂Cl₂ (CaH₂), THF (Na), CH₃CN (CaH₂) or dried on 4 of the molecular sieves. All the commercially available reagents were used without additional purification. Column flash chromatography was performed with silica gel (40-63 μm) according to a standard method. Gel permeation chromatography was carried out with Sephadex LH20® (GE Healthcare) according to a gravimetric technique. The nuclear magnetic resonance spectra (¹H, ¹³C) were recorded on a 300 MHz spectrometer. The chemical shifts for the ¹H and ¹³C spectra are recorded in parts per million and are calibrated for the residual solvent peaks (CHCl₃: ¹H 7.26 ppm, ¹³C 77.16 ppm, MeOH: ¹H 3.31 ppm, ¹³C 49.00 ppm and according to the article of Gottlieb et al., JOC, 62, 7512-7515). The multiplicities are indicated by s (singlet), BS (broad singlet), d (doublet), t (triplet), q (quadruplet), quint (quintuplet) and m (multiplet). The coupling constants, J, are given in Hertz. The exact mass was measured on a mass spectrometer coupled with matrix-assisted laser desorption-ionization (MALDI-TOF MS).

The compounds were obtained by the methods in the article by G. Lamanna et al., Biomaterials, 2011, 32, 8562-8573.

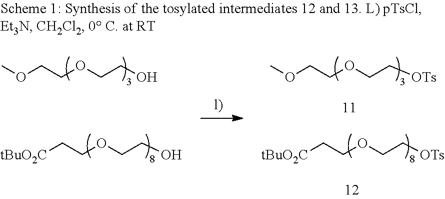

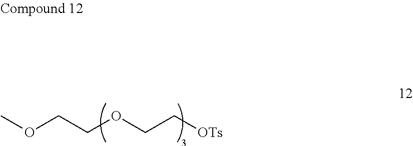

A solution of para-toluene chloride (22.3 g, 105 mmol) in THF (35 mL) was added dropwise to a solution of tetraethylene glycol methyl ether (20.0 g, 96 mmol) and NaOH (6.7 g, 166 mmol) in a THF/H₂O mixture (135 mL/45 mL) at 0° C. After stirring for 1 h at 0° C., the reaction mixture was left to return to room temperature and it was stirred for a further 20 hours. The solution was then poured into 200 ml of brine and the volatile materials were evaporated. The mixture obtained was extracted several times with dichloromethane and the combined organic layers were washed with brine, dried over MgSO₄, filtered and evaporated under reduced pressure. The oil was purified by silica gel column chromatography, eluting with dichloromethane/methanol (98/2). Compound 12 is obtained in the form of a pale yellow oil in 96% yield.

¹H NMR (300 MHz, CDC₃) δ 2.39 (s, 3H, ArCH₃), 3.31 (s, 3H, OCH₃), 3.64 to 3.47 (m, 14H, OCH₂CH₂O), 4.11 to 4.08 (m, 2H, ArSO₂OCH₂), 7.28 (d, J=1.5 Hz, 2H, Ar-3.5-H), 7.73 (d, J=1.5 Hz, 2H, Ar-2,6-H); ¹³C NMR (75 MHz, CDCl₃) δ 21.78, 59.14, 68.80, 69.45, 70.66, 70.73, 70.86, 72.07, 128.10, 129.99, 133.19, 144.96.

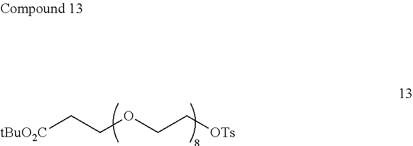

840 mL (6.0 mmol, 3.0 eq.) of NEt3 and 570 mg (3.0 mmol, 1.5 eq.) of paratoluenesulfonyl chloride are added successively to a solution of dPEG™ 8-tert-butyl hydroxy-ester (1.00 g, 2.0 mmol) in 20 ml of CH₂Cl₂ at 0° C. After stirring for 40 h at room temperature, the reaction mixture is diluted with 70 ml of CH₂Cl₂. The organic phases are combined, washed with brine, dried over MgSO₄, filtered and concentrated under reduced pressure. The crude product is purified by silica gel column chromatography (ethyl acetate/methanol 95/5-90/10 of ethyl), obtaining 13 in the form of colorless oil in 70% yield.

¹H NMR (300 MHz, CDCl₃): δ (ppm) 1.44 (s, 9H), 2.45 (s, 3H), 2.50 (t, 2H, ₃J=6.6 Hz), 3.58-3.73 (m, 32H), 4.16 (t, 2H, ₃J=4.9 Hz), 7.34 (2H, AA′ part of an AA′BB′ system), 7.81 (2H, BB′ part of an AA′BB′ system). ¹³C NMR (75 MHz, CDCl₃): δ (ppm) 21.14, 27.65, 35.84, 66.40, 68.15, 69.00, 70.08, 79.78, 127.46, 129.47, 132.74, 144.32, 170.20. MALDI: calculated for Co₁₀H₂₀LiO₅: 227.15, obtained: 227.08; calculated for C₂₆H₄₄LiO₁₂S: 587.27, obtained: 587.13.

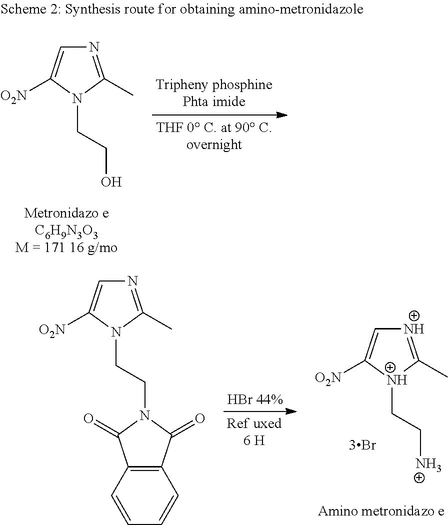 [SMILES: Cc1ncc([N+](=O)[O-])n1CCO ; Cc1ncc([N+](=O)[O-])n1CCN1C(=O)c2ccccc2C1=O ; CC1=[NH+]C=C([N+](=O)[O-])[NH+]1CC[NH3+]]

The metronidazole derivative (amino-metronidazole) was obtained by the methods in the article: M. Bertinaria et al., Drug Dev. Res., 2003, 60, 225-239.

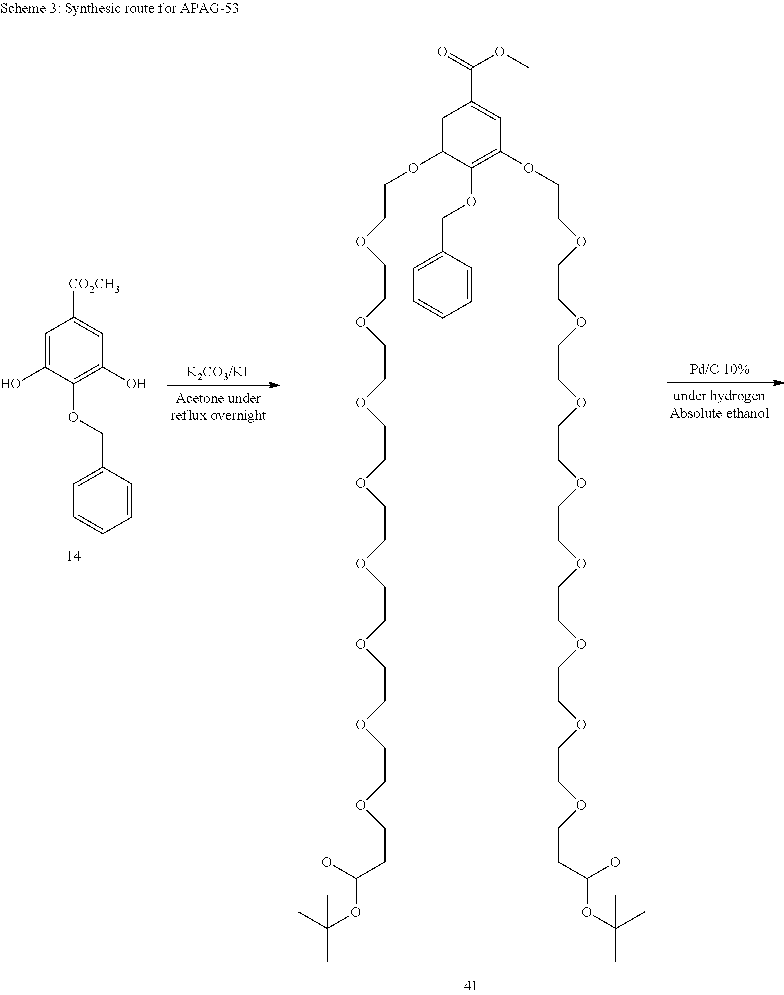 [SMILES: COC(=O)c1cc(O)c(OCc2ccccc2)c(O)c1 ; CCC(C)(C)OC(O)CCOCCOCCOCCOCCOCCOCCOCCOCCOC1CC(C(=O)OC)=CC(OCCOCCOCCOCCOCCOCCOCCOCCOCCC(O)OC(C)(C)C)=C1OCc1ccccc1]

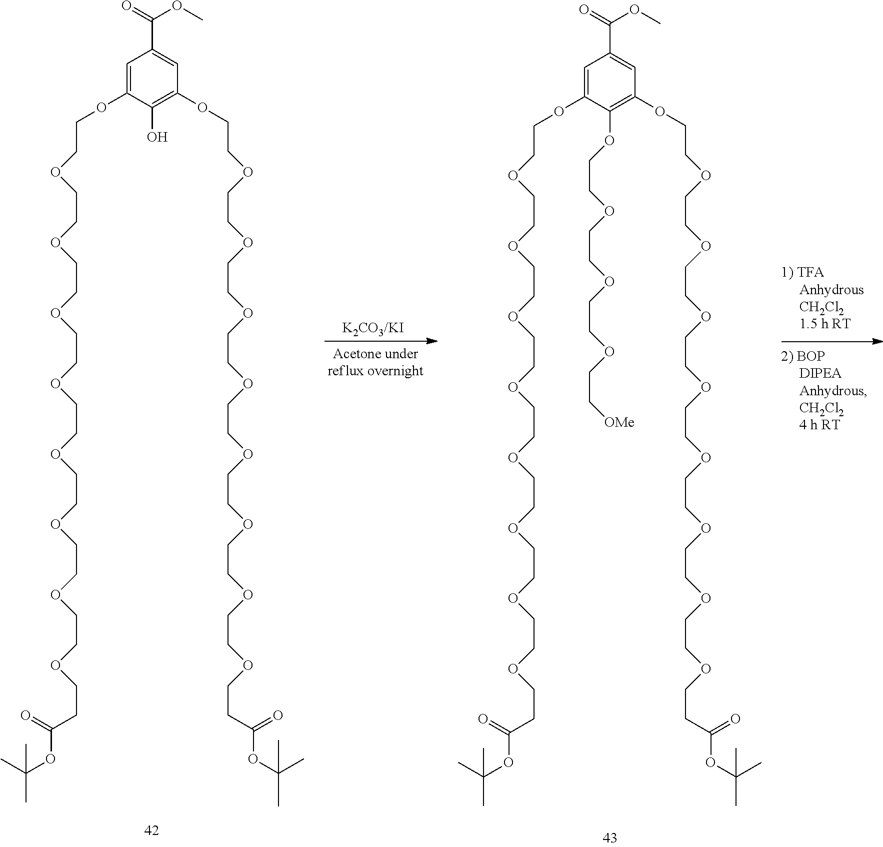 [SMILES: COC(=O)c1cc(OCCOCCOCCOCCOCCOCCOCCOCCOCCC(=O)OC(C)(C)C)c(O)c(OCCOCCOCCOCCOCCOCCOCCOCCOCCC(=O)OC(C)(C)C)c1 ; COCCOCCOCCOCCOc1c(OCCOCCOCCOCCOCCOCCOCCOCCOCCC(=O)OC(C)(C)C)cc(C(=O)OC)cc1OCCOCCOCCOCCOCCOCCOCCOCCOCCC(=O)OC(C)(C)C]

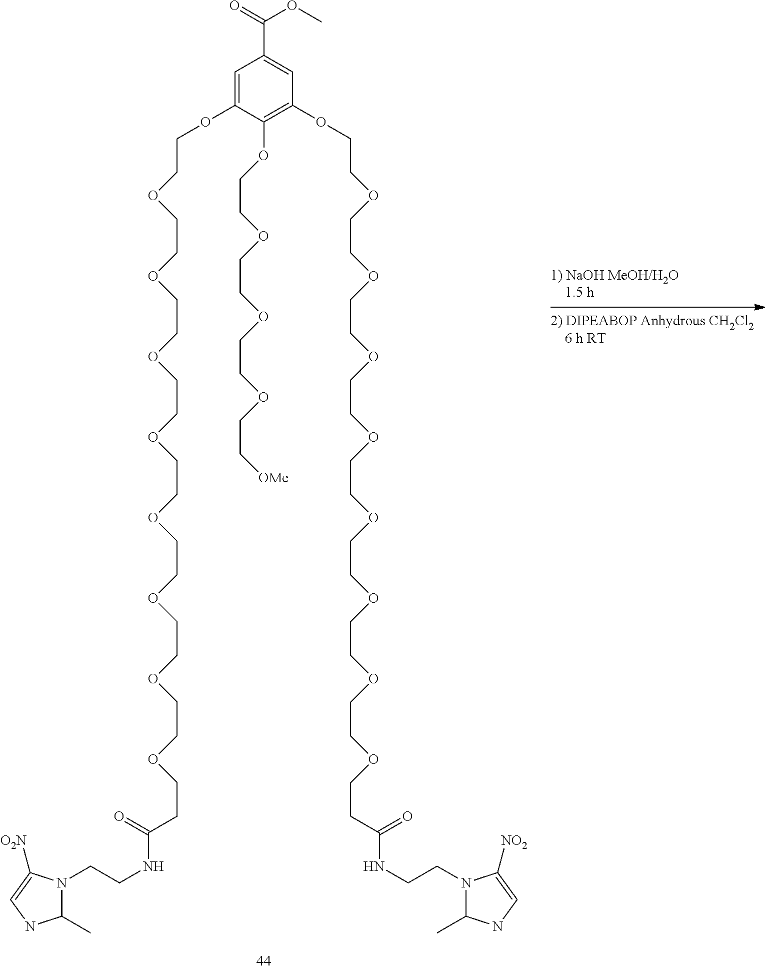 [SMILES: COCCOCCOCCOCCOc1c(OCCOCCOCCOCCOCCOCCOCCOCCOCCC(=O)NCCN2C([N+](=O)[O-])=CNC2C)cc(C(=O)OC)cc1OCCOCCOCCOCCOCCOCCOCCOCCOCCC(=O)NCCN1C([N+](=O)[O-])=CNC1C]

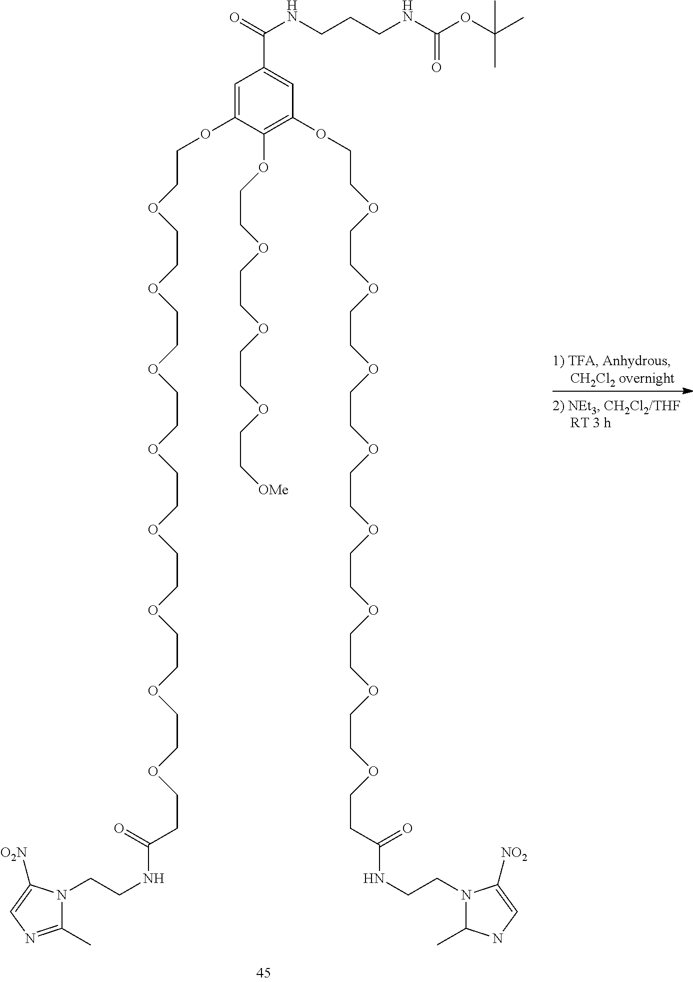 [SMILES: COCCOCCOCCOCCOc1c(OCCOCCOCCOCCOCCOCCOCCOCCOCCC(=O)NCCN2C([N+](=O)[O-])=CNC2C)cc(C(=O)NCCCNC(=O)OC(C)(C)C)cc1OCCOCCOCCOCCOCCOCCOCCOCCOCCC(=O)NCCn1c([N+](=O)[O-])cnc1C]

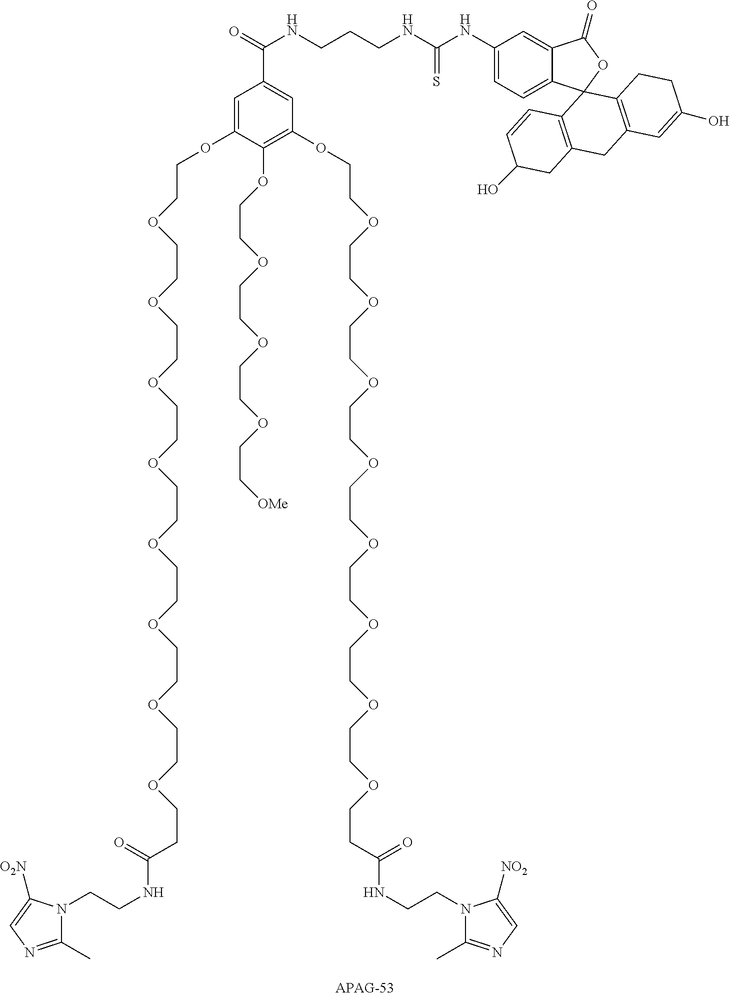 [SMILES: COCCOCCOCCOCCOc1c(OCCOCCOCCOCCOCCOCCOCCOCCOCCC(=O)NCCn2c([N+](=O)[O-])cnc2C)cc(C(=O)NCCCNC(=S)Nc2ccc3c(c2)C(=O)OC32C3=C(CC4=C2CCC(O)=C4)CC(O)C=C3)cc1OCCOCCOCCOCCOCCOCCOCCOCCOCCC(=O)NCCn1c([N+](=O)[O-])cnc1C]

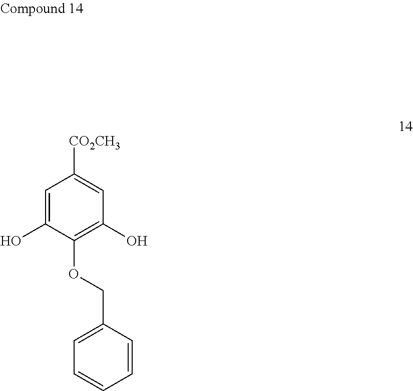 [SMILES: COC(=O)c1cc(O)c(OCc2ccccc2)c(O)c1]

A solution of methyl gallate (20.0 g, 109 mmol), BnBr (14.2 mL, 119 mmol), KHCO₃ (32.4 g, 324 mmol) and KI (0.1 g, 0.60 mmol) in DMF (100 ml) is stirred for 4 days at room temperature. The reaction mixture is poured into 1 L of water and sulfuric acid is added, until neutral pH is obtained. The aqueous layer is then extracted 3 times with 150 ml of CH₂Cl₂. The organic phases are combined, washed three times with 50 mL of brine, dried over MgSO₄, filtered and the volatiles are evaporated. The solvent is removed by evaporation and the residue is purified by silica gel column chromatography, eluting with CH₂Cl₂/MeOH (98/2) to give a yellow oil. The crude material is evaporated several times with dichloromethane. The residue obtained is filtered and washed with petroleum ether, giving 14 in the form of white solid at 70% yield.

¹H NMR (300 MHz, CD₃OD) δ 3.83 (s, 3H, COOCH₃), 5.18 (s, 2H, Ar²OCH), 7.13 (s, 2H, Ar¹-2,6-H), 7.31 (m, 3H, Ar²-3,4,5-H), 7.52 (d, J=7.5 Hz, 2H, Ar²-2,6-H); ¹³C NMR (75 MHz, CD₃OD) δ 51.2, 73.8, 108.8, 125.0, 127.8, 128.0, 128.5, 137.2, 138.2, 150.5, 167.1.

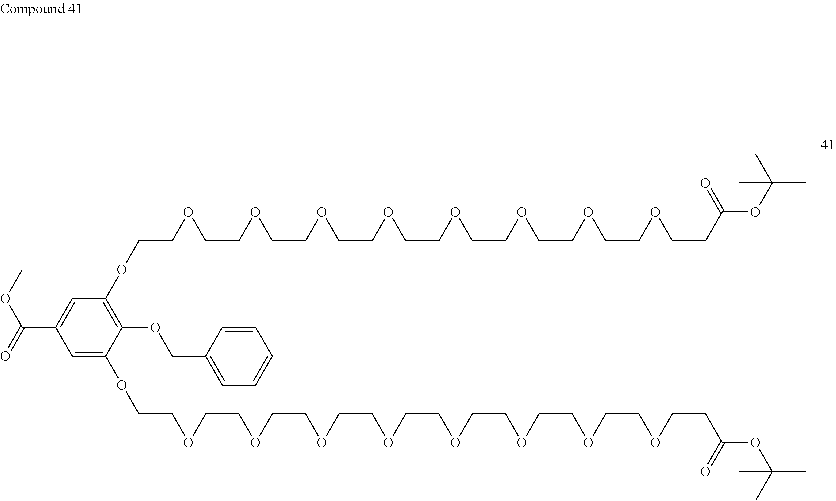 [SMILES: COC(=O)c1cc(OCCOCCOCCOCCOCCOCCOCCOCCOCCC(=O)OC(C)(C)C)c(OCc2ccccc2)c(OCCOCCOCCOCCOCCOCCOCCOCCOCCC(=O)OC(C)(C)C)c1]

A solution of diphenol derivative 14 (0.3 g, 1.08 mmol), the tosylated compound 13 (1.5 g, 2.4 mmol, 2.2 eq.), K₂CO; (0.9 g, 6.6 mmol, 6.0 eq.) and KI (17 mg, 0.11 mmol, 0.1 eq.) in acetone (30 mL) was stirred overnight at 65° C. The reaction mixture was filtered on Celite and the solvent was evaporated. The crude product obtained was diluted in dichloromethane (50 ml) and washed twice with aqueous saturated solution of NaHCO₃ and with brine. After drying over MgSO₄, filtration and evaporation of the solvent, the crude product was purified by silica gel column chromatography (dichloromethane/methanol 98/2-97/3) to give 41 in the form of colorless oil in 63% yield.

¹H NMR (300 MHz, CDCl₃) δ 1.42 (s, 18H, CH₂COOC(CH₃)₃), 2.51 (t, J=6.5 Hz, 4H, CH₂COOC(CH₃)₃), 3.55 to 3.72 (m, 60H, OCH₂CH₂O), 3.86 (t, J=5.0 Hz, 4H, OCH₂CH₂O), 3.89 (s, 3H, COOCH₃), 4.17 (t, J=4.8 Hz, 4H, ArOCH₂CH₂), 5.08 (s, 2H, ArOCH₂-Benzyl), 7.27 to 7.35 (m, 5H, Ar^{Benzyl}-3,4,5-H and Ar-2,6-H), 7.49 (d, J=7.7 Hz, 2H, Ar^{Benzyl}-2,6-H); ¹³C NMR (75 MHz, CDCl₃) δ 28.0, 36.2, 52.1, 66.8, 68.6, 69.5, 70.3, 70.45, 70.5, 70.55, 70.8, 74.8, 80.4, 108.4, 125.1, 127.8, 128.1, 128.35, 137.7, 141.9, 152.25, 166.5, 170.9.

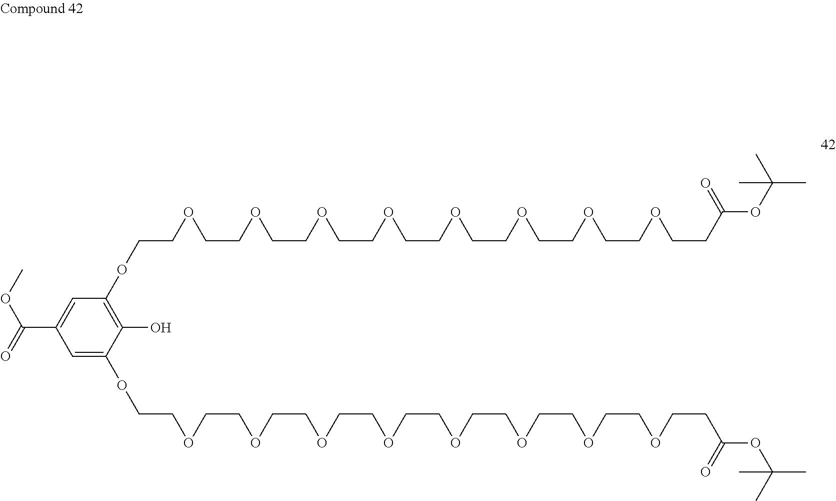 [SMILES: COC(=O)c1cc(OCCOCCOCCOCCOCCOCCOCCOCCOCCC(=O)OC(C)(C)C)c(O)c(OCCOCCOCCOCCOCCOCCOCCOCCOCCC(=O)OC(C)(C)C)c1]

Palladium activated on charcoal at 10% (2 eq.) was added to a solution of the benzyl compound 41 (0.85 g, 0.7 mmol) dissolved in absolute ethanol (15 ml). The mixture was stirred under a hydrogen atmosphere at room temperature for 16 h. The product was filtered through a Celite stopper and the solvent was evaporated under reduced pressure. The crude product was purified by silica gel column chromatography (dichloromethane/methanol 98/2-95/5) to give 42 in the form of colorless oil in 98% yield.

¹H NMR (300 MHz, CDCl₃) δ 1.41 (s, 18H, CH₂COOC(CH)₃), 2.49 (t, J=6.6 Hz, 4H, CH₂COOC(CH₃)₃), 3.55 to 3.70 (m, 60H, OCH₂CH₂O), 3.85 to 3.90 (m, 7H, OCH₂CH₂O and COOCH₃), 4.21 (t, J=5.2 Hz, 4H, ArOCH₂CH₂), 7.34 (s, 2H, Ar-2,6-H); ¹³C NMR (75 MHz, CDCl₃) δ 28.0, 36.2, 52.0, 66.9, 69.4, 70.35, 70.5, 70.55, 70.7, 80.5, 110.4, 120.4, 142.1, 146.2, 166.7, 170.9.

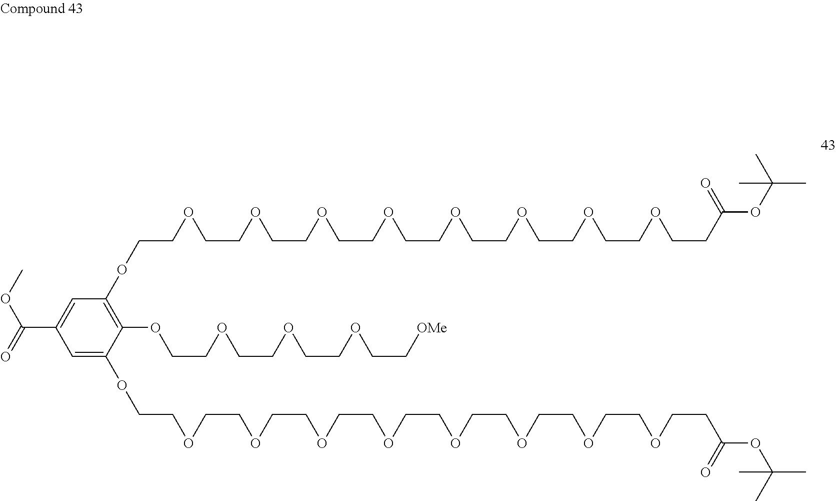 [SMILES: COCCOCCOCCOCCOc1c(OCCOCCOCCOCCOCCOCCOCCOCCOCCC(=O)OC(C)(C)C)cc(C(=O)OC)cc1OCCOCCOCCOCCOCCOCCOCCOCCOCCC(=O)OC(C)(C)C]

K₂CO₃ (0.27 g, 2 mmol, 3 eq.) was added to an equimolar solution of phenolic derivative (42) (0.75 g, 0.66 mmol) and of tosylated derivative 12 (0.27 g, 0.66 mmol) in 10 ml of acetone. The reaction mixture was stirred at 60° C. overnight. After filtration on Celite, the solvent was evaporated and the residue was diluted in dichloromethane (50 mL). The organic layer was washed twice with a saturated solution of NaHCO₃, then with brine, filtered and concentrated under reduced pressure. The crude product was purified by silica gel column chromatography (dichloromethane/methanol 98/2-95/5) to give 43 in the form of pale yellow oil at 75% yield.

¹H NMR (300 MHz, CDCl₃) δ 1.42 (s, 18H, CH₂COOC(CH₃)₃), 2.49 (t, J=6.5 Hz, 4H, CH₂COOC(CH₃)₃), 3.36 (s, 3H, OCH₂CH₂OCH₃), 3.50-3.54 (m, 2H, OCH₂CH₂O), 3.60 to 3.70 (m, 70H, OCH₂CH₂O), 3.78 (t, J=4.8 Hz, 2H, OCH₂CH₂O), 3.83 to 3.88 (m, 7H, OCH₂CH₂O and COOCH₃), 4.15 to 4.23 (m, 6H, ArOCH₂CH₂), 7.28 (s, 2H, Ar-2.6-H); ¹³C NMR (75 MHz, CDCl₃) δ 28.1, 36.2, 52.1, 59.0, 66.9, 68.8, 69.4, 70.35, 70.5, 70.55, 70.65, 70.8, 71.9, 72.4, 80.4, 109.0, 125.0, 142.5, 152.1, 166.6, 170.9. MALDI: calculated for C₆₃H₁₁₄NaO₂₉: 1357.74, obtained: 1357.80, calculated for C₆₃H₁₁₄KO₂₉: 1373.74, obtained: 1373.78.

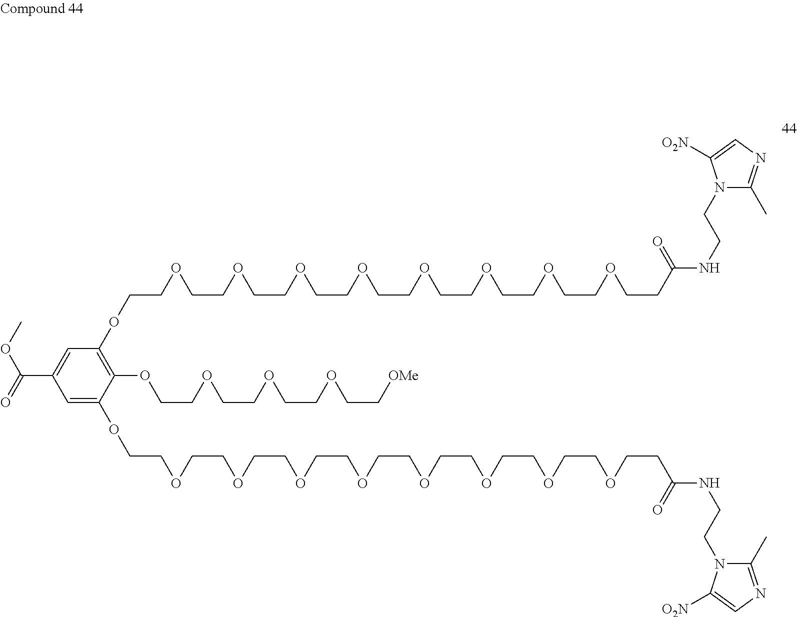 [SMILES: COCCOCCOCCOCCOc1c(OCCOCCOCCOCCOCCOCCOCCOCCOCCC(=O)NCCn2c([N+](=O)[O-])cnc2C)cc(C(=O)OC)cc1OCCOCCOCCOCCOCCOCCOCCOCCOCCC(=O)NCCn1c([N+](=O)[O-])cnc1C]

2 ml (large excess) of trifluoroacetic acid was added dropwise to a solution of compound 43 (0.16 g, 0.12 mmol) in 4 ml of CH₂Cl₂ at 0° C. The reaction mixture was stirred for 1 h at room temperature under argon, and then the volatile substances were evaporated to dryness. The crude product in the form of acid was obtained in the form of a colorless oil and was used without further purification. The coupling reagent BOP (0.13 g, 0.3 mmol, 2.6 eq. per acid) was added under argon to a solution of carboxylic acid derivative (0.14 g, 0.11 mmol, 1 eq) in 5 mL of distilled dichloromethane. After 30 min, amino-metronidazole (0.12 g, 0.25 mmol, 2.2 eq.) and N,N-diisopropylethylamine (180 pl, 1 mmol, 4 eq. per amine) were added. The reaction mixture was stirred for 6 h at room temperature. 50 ml of dichloromethane was added and the organic layer was washed with 1N sodium hydroxide solution (3×20 ml) and brine (3×20 mL), dried over MgSO₄, filtered and concentrated under reduced pressure. The crude product was purified by column chromatography on LH (dichloromethane) to give 44 as a pale brown oil at 75% yield.

¹H NMR (300 MHz, CDCl₃) δ 2.44 (t, J=5.5 Hz, 4H, CH₂CONH), 2.53 (s, 6H, CH₃), 3.32 (s, 3H, OCH₂CH₂OCH₃), 3.60 to 3.72 (m, 76H, OCH₂CH₂O and NHCH₂CH₂N), 3.78 (t, J=4.9 Hz, 2H, OCH₂CH₂O), 3.83 to 3.89 (m, 7H, OCH₂CH₂O and COOCH₃), 4.15 to 4.21 (m, 6H, ArOCH₂CH₂), 4.46 (t, J=6.3 Hz, 4H, NHCH₂CH₂N), 7.18 (m, 2H, NHCH₂CH₂N), 7.28 (s, 2H, Ar-2,6-H), 7.96 (s, 2H, Ar^{Nitro}-H); ¹³C NMR (75 MHz, CDCl₃) δ 14.1, 36.4, 39.0, 45.2, 52.1, 59.0, 66.8, 68.8, 69.5, 70.1 (J=6.6 Hz), 70.35, 70.45, 70.5, 70.55, 70.6, 70.75, 71.9, 72.3, 108.9, 125.0, 132.7, 142.5, 152.1, 166.5, 172.8. MALDI: calculated for C₆₇H₁₁₇N₈O₂₇: 1469.66, obtained: 1469.90, calculated for C₆₇H₁₁₅N₈O₂₉: 1498.66, obtained: 1498.89, calculated for C₆₇H₁₁₃NaN₈O₃₁: 1549.66, obtained: 1549.87.

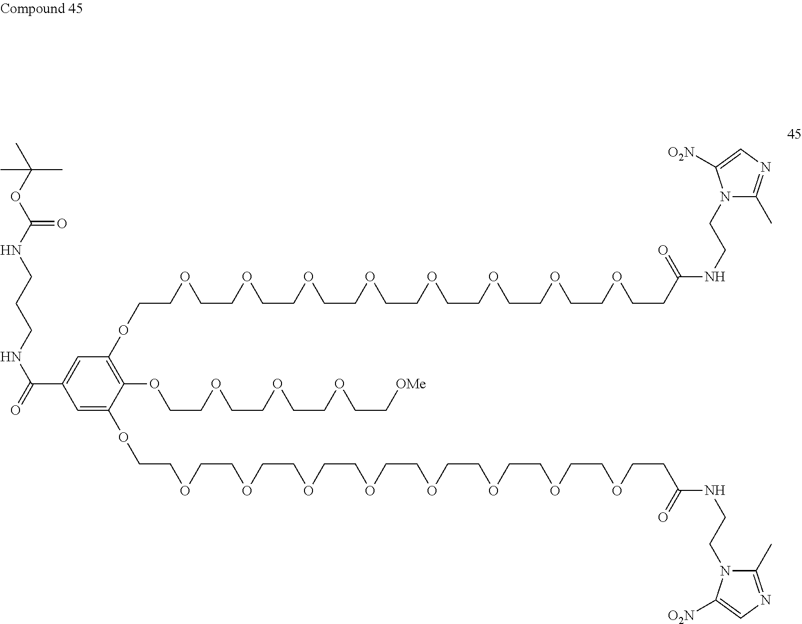 [SMILES: COCCOCCOCCOCCOc1c(OCCOCCOCCOCCOCCOCCOCCOCCOCCC(=O)NCCn2c([N+](=O)[O-])cnc2C)cc(C(=O)NCCCNC(=O)OC(C)(C)C)cc1OCCOCCOCCOCCOCCOCCOCCOCCOCCC(=O)NCCn1c([N+](=O)[O-])cnc1C]

Sodium hydroxide (18 mg, 0.45 mmol, 5 eq.) was added to a solution of compound 44 (0.14 g, 0.09 mmol) in a 3/1 methanol/water mixture (4 ml). The reaction mixture was stirred for 2 h at 85° C., and stopped. The mixture was evaporated to dryness and 30 ml of dichloromethane was added. The organic phase was dried over MgSO₄, filtered and concentrated under reduced pressure. The crude product in the form of acid was obtained in the form of a red foam and was used without further purification. The coupling reagent BOP (38 mg, 0.09 mmol, 1.3 eq. per acid) was added under argon to a solution of carboxylic acid derivative (0.1 g, 0.07 mmol, 1 eq) in 7 ml of distilled dichloromethane. After 30 min. 1,3-mono-NHBoc-propylamine (14 mg, 0.08 mmol, 1.2 eq.) and N,N-diisopropylethylamine (30 μl, 0.16 mmol, 2 eq. per amine) were added. The reaction mixture was stirred for 6 h at room temperature. 40 ml of dichloromethane was added and the organic layer was washed with 1N sodium hydroxide solution (3×20 mL), brine (3×20 ml) and water (20 mL), dried over MgSO₄, filtered and concentrated under reduced pressure. The crude product was purified by column chromatography on LH (dichloromethane) to give 45 in the form of a pale brown oil at 64% yield.

¹H NMR (300 MHz, CDCl₃) δ 1.41 (s, 9H, NHCOOC(CH₃)₃), 1.67 (m, J=5.1 Hz, 2H, NHCH₂CH₂CH₂NH), 2.39 (t, J=5.6 Hz, 4H, CH₂CONH), 2.49 (s, 6H, CH₃), 3.18 (m, 2H, NHCH₂CH₂CH₂NH), 3.32 (s, 3H, OCH₂CH₂OCH₃), 3.41 (m, 2H, NHCH₂CH₂CH₂NH), 3.60 to 3.72 (m, 76H, OCH₂CH₂O and NHCH₂CH₂N), 3.78 (t, J=4.9 Hz, 2H, OCH₂CH₂O), 3.82 (t, J=4.7 Hz, 4H, OCH₂CH₂O), 4.12 to 4.20 (m, 6H, ArOCH₂CH₂), 4.42 (t, J=6.3 Hz, 4H, NHCH₂CH₂N), 5.12 (t, J=5.1 Hz, 1H, NHCH₂CH₂CH₂NH), 7.12 to 7.18 (m, 4H, Ar-2.6-H and NHCH₂CH₂N), 7.51 (m, 1H, NHCH₂CH₂CH₂NH), 7.91 (s, 2H, Ar^{Nitro}-H); ¹³C NMR (75 MHz, CDCl₃) δ 14.1, 28.2, 36.4, 39.1, 45.1, 59.0, 66.8, 68.8, 69.65, 70.1 (J=5.0 Hz), 70.35, 70.45, 70.5, 70.55, 70.6, 70.7, 71.9, 72.2, 79.2, 106.8, 129.7, 133.2, 152.2, 166.6, 172.8. MALDI: calculated for C₆₉H₁₁₉N₁₈O₃₀: 1569.86, obtained: 1569.97, calculated for C₇₄H₁₃₁N₈O₂₈: 1610.86, obtained: 1611.06, calculated for C₇₄H₁₂₉N₈O₃₀: 1640.86, obtained: 1641.05, calculated for C₇₄H₁₂₇NaN₁₀O₃₂: 1691.86, obtained: 1692.0.

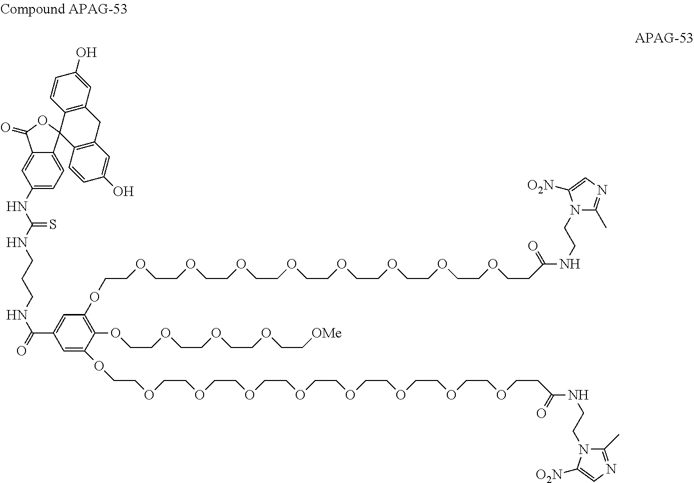 [SMILES: COCCOCCOCCOCCOc1c(OCCOCCOCCOCCOCCOCCOCCOCCOCCC(=O)NCCn2c([N+](=O)[O-])cnc2C)cc(C(=O)NCCCNC(=S)Nc2ccc3c(c2)C(=O)OC32c3ccc(O)cc3Cc3cc(O)ccc32)cc1OCCOCCOCCOCCOCCOCCOCCOCCOCCC(=O)NCCn1c([N+](=O)[O-])cnc1C]

Trifluoroacetic acid (110 pl, 1.26 mmol, 30 eq.) was added dropwise to a solution of compound 45 (70 mg, 0.04 mmol) in 4 ml of CH₂Cl₂ at 0° C. The reaction mixture was stirred overnight at room temperature under argon, and then the volatile substances were evaporated to dryness. The crude product in the form of acid was obtained in the form of a brown oil and was used without further purification. Triethylamine (30 μL, 0.23 mmol, 6 eq.) was added under argon to a solution of amine derivative (0.08 g, 0.04 mmol, 1 eq) in a dichloromethane/tetrahydrofuran mixture (3/5). After 30 min, fluorescein isothiocyanate FITC (26 mg, 0.04 mmol, 1.1 eq.) was added. The reaction mixture was stirred for 3 h at room temperature and the volatiles were evaporated to dryness. The crude product was purified by column chromatography on LH (once with methanol and once again with dichloromethane) to give APAG-53 in the form of an orange foam in 49% yield.

¹H NMR (300 MHz, MeOD) δ 1.98 (m, 2H, NHCH₂CH₂CH₂NH), 2.41 (t, J=5.8 Hz, 4H, CH₂CONH), 2.53 (s, 6H, CH₃), 3.38 to 3.42 (m, 5H, NHCH₂CH₂CH₂NH and OCH₂CH₂OCH₃), 3.50 (m, 2H, NHCH₂CH₂CH₂NH), 3.58 to 3.85 (m, 78H, OCH₂CH₂O and NHCH₂CH₂N), 3.88 (t, J=4.8 Hz, 4H, OCH₂CH₂O), 4.20 to 4.26 (m, 6H, ArOCH₂CH₂), 4.51 (t, J=6.0 Hz, 4H, NHCH₂CH₂N), 6.52 (dd, J=2.3 and 8.7 Hz, 2H, Ar^{FITC}-H), 6.72 (d, J=2.3 Hz, 2H, Ar^{FITC}-H), 6.79 (d, J=8.7 Hz, 2H, Ar^{FITC}-H), 7.23 (s, 4H, Ar-2.6-H), 7.28 (d, J=8.3 Hz, 1H, Ar^{FITC}-H), 7.79 (m, 1H, Ar^{FITC}-H), 7.98 (s, 2H, Ar^{Nitro}-H), 8.18 (m, 1H, Ar^{FITC}-H). ¹³C NMR (75 MHz, MeOD) δ 28.2, 35.5, 37.9, 44.9, 57.2, 66.0, 68.1, 68.9, 70.1, 70.35, 70.45, 70.5, 70.55, 70.6, 70.7, 71.0, 71.8, 79.2, 101.8, 105.9, 109.6, 111.95, 118.8, 124.0, 128.5, 131.2, 137.2, 140.1, 150.6, 151.8, 152.1, 159.3, 167.1, 169.0, 172.6, 180.4. MALDI: calculated for C₉₁H₁₃₅N₁₁SO₃₂: 1927.15, obtained: 1927.0.

### Example 5

### Dinitro Derivatives

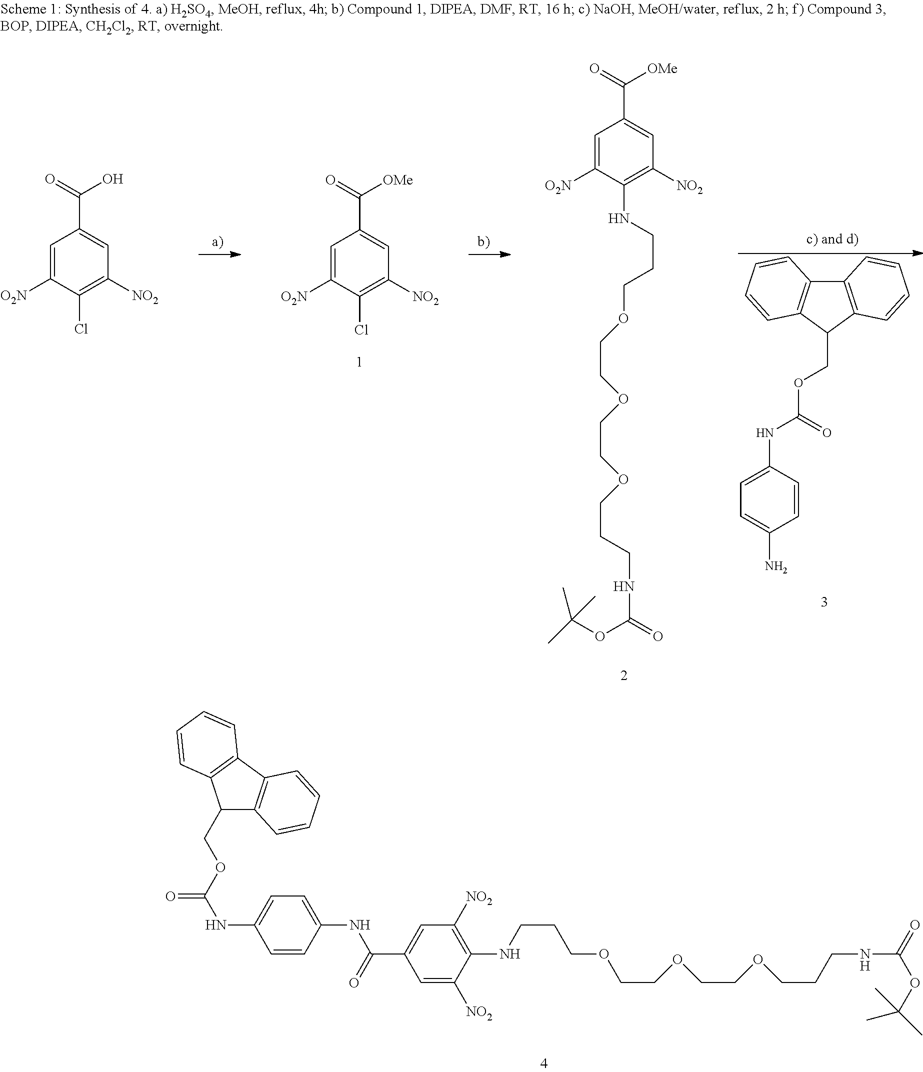 [SMILES: O=C(O)c1cc([N+](=O)[O-])c(Cl)c([N+](=O)[O-])c1 ; COC(=O)c1cc([N+](=O)[O-])c(Cl)c([N+](=O)[O-])c1 ; COC(=O)c1cc([N+](=O)[O-])c(NCCCOCCOCCOCCCNC(=O)OC(C)(C)C)c([N+](=O)[O-])c1 ; Nc1ccc(NC(=O)OCC2c3ccccc3-c3ccccc32)cc1 ; CC(C)(C)OC(=O)NCCCOCCOCCOCCCNc1c([N+](=O)[O-])cc(C(=O)Nc2ccc(NC(=O)OCC3c4ccccc4-c4ccccc43)cc2)cc1[N+](=O)[O-]]

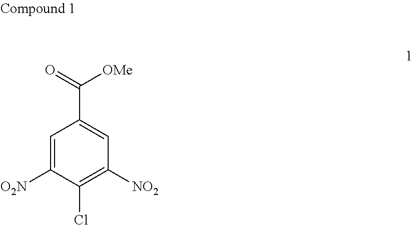 [SMILES: COC(=O)c1cc([N+](=O)[O-])c(Cl)c([N+](=O)[O-])c1]

Concentrated sulfuric acid (1 ml) was added to a solution of 4-chloro-3,5-dinitrobenzoic (2.0 g, 8.13 mmol) dissolved in methanol (10 ml). The mixture was stirred for 4 h under reflux and put in ice for 1 h. The crude product was filtered, washed with petroleum ether (3×20 ml) and dried under vacuum to obtain 1 in the form of white solid in 98% yield.

¹H NMR (300 MHz, CDCl₃) δ 4.02 (s, 3H, COOCH₃), 8.61 (s, 2H, ArH); ¹³C NMR (75 MHz, CDCl₃) δ 52.5, 125.3, 132.6, 137.1, 142.1, 163.8.

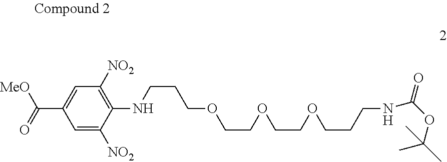 [SMILES: COC(=O)c1cc([N+](=O)[O-])c(NCCCOCCOCCOCCCNC(=O)OC(C)(C)C)c([N+](=O)[O-])c1]

N,N-diisopropylethylamine (0.28 ml, 1.56 mmol, 0.5 eq. per amine) was added under argon to a solution of mono-Nt-Boc-amido-dPEG™3-amine (1.0 g, 3.12 mmol, 1.1 eq) in 10 ml of distilled dichloromethane. After 30 min. compound 1 (0.74 g, 2.84 mmol, 1.0 eq.) was added. The reaction mixture was stirred overnight at room temperature. 100 ml of brine was added and the aqueous phase was extracted with ethyl ether (3×40 ml). The organic layer was washed with 1N hydrochloric acid solution (2×40 ml) and brine (2×40 mL), dried over MgSO₄, filtered and concentrated under reduced pressure. The crude product was purified by silica gel column chromatography (dichloromethane/methanol 100 to 98.5/1.5) to give 2 in the form of a pale yellow oil in 92% yield.

¹H NMR (300 MHz, CDCl₃) δ 1.42 (s, 9H, NHCOOC(CH₃)₃), 1.73 (m, J=6.1 Hz, 2H, CH₂CH₂CH₂NHCOO), 1.94 (m, J=6.2 Hz, 2H, ArH—NHCH₂CH₂CH₂), 3.10 to 3.18 (m, 4H, NHCH₂CH₂CH₂), 3.50 to 3.70 (m, 12H, OCH₂CH₂O and OCH₂CH₂CH₂), 3.95 (s, 3H, COOCH₃), 4.94 (m, 1H, NHCOOC(CH₃)₃), 8.75 (s, 2H, ArH) 9.25 (m, 1H, ArH—NHCH₂): ¹³C NMR (75 MHz, CDCl₃) δ 38.5, 46.3, 52.6, 69.6, 70.15, 70.2, 70.4, 70.5, 70.8, 78.8, 115.3, 133.0, 136.9, 142.0, 155.9, 163.8.

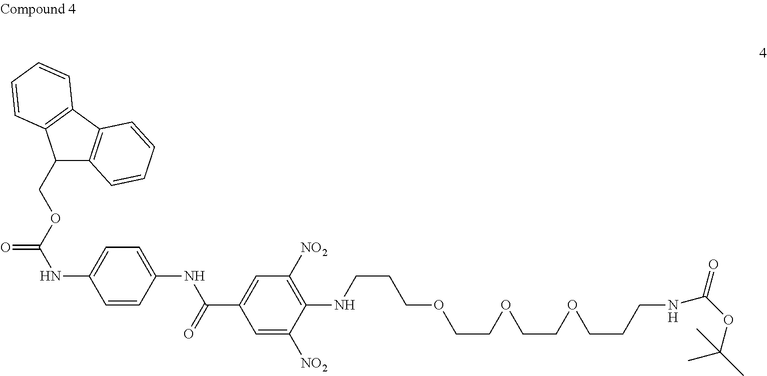 [SMILES: CC(C)(C)OC(=O)NCCCOCCOCCOCCCNc1c([N+](=O)[O-])cc(C(=O)Nc2ccc(NC(=O)OCC3c4ccccc4-c4ccccc43)cc2)cc1[N+](=O)[O-]]

Sodium hydroxide (51 mg, 1.8 mmol, 5 eq.) was added to a solution of compound 2 (0.2 g, 0.36 mmol) in a 4/1 methanol/water mixture (5 ml). The reaction mixture was stirred for 2 h at 85° C. and stopped. The mixture was evaporated to dryness and 30 ml of dichloromethane was added. The organic phase was dried over MgSO₄, filtered and concentrated under reduced pressure. The crude product in the form of acid was obtained in the form of an orange foam and was used without further purification. The coupling reagent BOP (0.13 g, 0.29 mmol, 1.3 eq. per acid) was added under argon to a solution of carboxylic acid derivative (0.12 g, 0.23 mmol, 1.0 eq) in 5 mL of distilled dichloromethane. After 30 min, aniline derivative 3 (WO 2009037229) (76 mg, 0.25 mmol, 1.1 eq.) diluted in 0.5 ml of N,N′-dimethylformamide and N,N-diisopropylethylamine (90 pi, 0.5 mmol, 2 eq. per amine) was added. The reaction mixture was stirred overnight at room temperature. 40 ml of dichloromethane was added and the organic phase was washed with 1N hydrochloric acid solution (2×20 ml), brine (2×20 ml) and water (1×20 mL), dried over MgSO₄, filtered and concentrated under reduced pressure. The crude product was purified by column chromatography on neutral alumina gel (dichloromethane/methanol 99.5-0.5 to 99/1) to give 4 in the form of a pale yellow oil in 62% yield. Compound 4 may be used as a synthon to form a radiolabelable moiety or group R according to the invention.

¹H NMR (300 MHz, DMSO-d₆) δ 1.33 (s, 9H, NHCOOC(CH₃)₃), 1.52 (m, J=6.6 Hz, 2H, CH₂CH₂CH₂NHCOO), 1.84 (m, J=6.0 Hz, 2H, ArH—NHCH₂CH₁CH₂), 2.91 (q, J=6.6 Hz, 2H, CH₂CH₂CH₂NHCOO), 3.03 (q, J=6.0 Hz, 2H, ArH—NHCH₂CH₂CH₂), 3.40 to 3.55 (m, 12H, OCH₂CH₂O and OCH₂CH₂CH₂), 4.28 (t, J=6.6 Hz, 2H, CH₂CH), 4.47 (d, J=6.6 Hz, 2H, CH₂CH), 6.71 (t, J=5.2 Hz, 1H, NHCOOC(CH₃)₃), 7.30 to 7.50 (m, 6H, Ar^{Fmoc}H and Ar²H), 7.62 (d, J=8.6 Hz, 2H, Ar²H), 7.73 (d, J=7.4 Hz, 2H, Ar^{Fmoc}H), 7.91 (d, J=7.4 Hz, 2H, Ar^{Fmoc}H), 8.72 (t, J=4.8 Hz, 1H, ArH—NHCH₂), 8.88 (s, 2H, Ar¹H), 9.71 (s, 1H, Ar²H—NHCOO), 8.72 (t, J=4.8 Hz, 1H, Ar²H—NH—Ar¹H); ¹³C NMR (75 MHz, DMSO-d₆) δ 30.1, 30.8, 31.7, 39.1, 47.4, 48.5, 67.4, 69.8, 70.8, 71.4, 71.45, 71.5, 71.55, 79.1, 120.4, 121.2, 121.9, 122.9, 127.0, 128.9, 129.5, 133.0, 135.2, 136.9, 138.7, 142.7, 145.7, 155.2, 157.3, 163.0.

### Example 7

### Indium-111 Radiolabeling of Compounds Comprising a DOTA Chelating Agent, in Particular Compounds AP070 and AP071

In a sterile bottle, 2 mg of the compound is dissolved in 1 mL of water for injection, 0.3 mL of citrate buffer 50 mM (pH=5.5) is added to the bottle. Indium 111 chloride (185 to 370 MBq in 0.5 mL) is then added to the aforementioned bottle. The solution is incubated for 1 hour at room temperature on a shaker. After incubation, the solution is purified on a Sephadex PD-10 column (GE) packed beforehand with the 50 mM citrate buffer solution and then eluted with this same buffer. The various eluates are recovered in 5-mL tubes. Thin-layer chromatography (ITLC-SG) is performed on all of the eluates obtained. The solutions having radiochemical purity above 98% are mixed, filtered (0.22 μm) and are used for the tests in vivo.
The purity of the radiolabeled product is tested by paper chromatography according to the scheme presented in FIG. 2.

### Example 8

### In Vivo Results

### 8.1 Animal Model Used:

C57B16 mice bearing xenografts of murine melanoma (B16FO line) obtained by subcutaneous injection of 300 000 cells suspended in 100 μl of RPMI culture medium are injected subcutaneously in the back. The experiment is performed when the tumors are palpable. i.e. about 10 days post-25 graft. All the experiments (tumoral graft, injection and imaging) are carried out under isoflurane/oxygen (2.5%/2.5%) gas anesthesia.

### 8.2. Injection of the Radiolabeled Solutions:

Injection of 50 to 100 microliters of the radiolabeled solution, with a maximum activity of 20 MBQ, by direct intravenous injection (caudal vein). The activity of the empty syringe after injection+compress is also measured for calculating the actual activity injected (actual Ac injected=Ac normalized full syringe—normalized activity (empty syringe and compress). Normalization consists in calculating the theoretical activity at one and the same time chosen as reference (for example the time of injection). The measurements are performed on a regularly calibrated activity meter for medical use (Medisystem©).

### 8.3 Imaging Protocol:

Scintigraphic imaging is carried out using a dedicated small animal micro-5 imager SPECT-CT (BIOSCAN™). A planar scintigraphic image is obtained immediately after injection to verify absence of intracaudal stasis. If there is stasis, the mouse will be sacrificed at late times (24 or 48 h). Static scintigraphic images may be recorded at 2 h, 4 h, 24 h and 48 h (Imager micro-SPECT-CT BIOSCAN©).

### 8.4 Quantitative Study of Tumoral Fixation and of the Biodistribution of the Radiolabeled Compound:

This is done ex-vivo by gamma counting of the various samples, using a gamma well counter for medical use, regularly calibrated for the isotopes used (Tc-99m and In-111). The animals are euthanized according to the protocols allowed by the animal ethics committee at 2 h, 4 h, 24 h or 48 h. The main organs are removed (brain, spleen, kidneys, lungs, heart, alimentary canal, liver) as well as a fragment of skin, muscle, bone and a blood sample and the whole tumor. Each sample is weighed and its gamma activity is counted in a sample-changing well counter for dedicated medical use, calibrated for the radioelements used. The animal's carcass is also weighed after removing the tail; these samples are incinerated and their gamma activity is also measured. The gamma activity of the samples containing all the collected urine and the feces is also measured. The activity of each sample is normalized (relative to the time of injection), and the ratio of the activity of each sample is calculated relative to the total activity injected (=the sum of all of the activities measured except that of the tail), expressed in % per gram of tissue, for calculating the ratio of the activity present in the tumor relative to the main organs (liver, kidneys, lungs and blood). The percentage of the activity eliminated is calculated.

The mean values of all of the data for all of the mice whose data are exploitable are calculated, as well as after eliminating the animals with urinary excretion that is too quick (>50% at 2 h or 75% at 4 h, as well as the mice with tumors >2 g, as the existence of necrosis may distort the calculation of the ratio of fixation in the tumor).

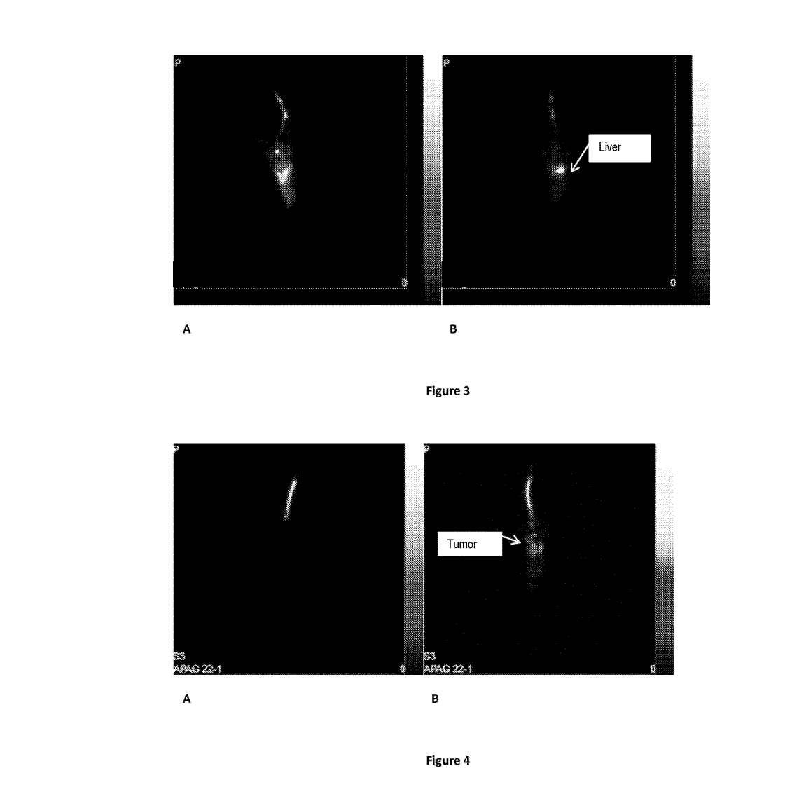

The biodistribution data of the various dendrimer compounds coupled to the IFC01102 ligand (also called amine R) and to a chelating agent are compared with those of the ligand alone coupled to the DOTA chelating agent (compound AP045) and radiolabeled with the same radioelement.
8.5 Results with Compound APAG9-1 (Radiolabeled with Tc99m)
The radiochemical purity is above 95%, according to the chromatographic profile obtained as described above (example 5).
12 animals were injected.
The average activity injected is 3.5±2.4 MBq.
2 animals were euthanized at 2 h, 4 at 24 h and 48 h post-injection.
Examples of planar image immediately post-injection and 2 hours post-injection are illustrated in FIG. 3.

The quantitative data on biodistribution obtained on 10 animals are presented in the following table:

| | | Time | | | | | | | |
|---|---|---|---|---|---|---|---|---|---|
| | | | | 4 h | | | | | |
| | | 2 h post-iv | | post-IV | | 24 h post-IV | | 48 post-IV | |
| | | # Animals | | | | | | | |
| | | | | 0 | | | | | |
| | | 2 | | Mean | | 4 | | 4 | |
| | | Mean value | SD | value | SD | Mean value | SD | Mean value | SD |
| | Tumor weight | 3.91E−01 | | | | 1.59E−01 | 8.19E−01 | 3.91E−01 | 3.33E−01 |
| | Tumor (g/Al) | 2.08E+00 | | | | 1.35E+00 | 4.25E−01 | 1.02E+00 | 3.10E−01 |
| Ratio (g)/Al | Tu/Liver | 4.91E−02 | | | | 2.88E−01 | 5.42E−01 | 7.89E−02 | 1.22E−01 |
| | Tu/Lungs | 6.61E−01 | | | | 9.57E−01 | 5.29E−01 | 6.40E−01 | 2.73E−01 |
| | TU/kidneys | 2.16E−01 | | | | 3.85E−01 | 2.64E−01 | 2.44E−01 | 8.23E−02 |
| | Tu/blood | 2.16E−01 | | | | 2.19E+00 | 7.86E−01 | 1.25E+00 | 7.61E−01 |
| | Total | 2.70E+01 | | | | 2.4E+01 | 2.1E+00 | 3.10E+01 | 5.92E+00 |
| | Elimination | | | | | | | | |
| | (%/Al) | | | | | | | | |

8.6 Results with Compound APAG22-1 (Radiolabeled with Tc99m)
The radiochemical purity is above 99%, according to the chromatographic profile obtained as described above (example 5).

16 animals were injected.

The average activity injected is 4.16±1.31 MBq
3 animals were euthanized at 2 h, 4 at 4 h, 24 h and 48 h post-injection.
Examples of planar image immediately post-injection and 2 hours post-injection are illustrated in FIG. 4.

The quantitative data on biodistribution obtained on 15 animals are presented in the following table:

| | | Time | | | | | | | |
|---|---|---|---|---|---|---|---|---|---|
| | | 2 h post-iv | | 4 h post-IV | | 24 h post-IV | | 48 post-IV | |
| | | # Animals | | | | | | | |
| | | 3 | | 4 | | 4 | | 4 | |
| | | Mean value | SD | Mean value | SD | Mean value | SD | Mean value | SD |
| | Tumor weight | 1.17E+00 | 2.56E−01 | 7.63E−01 | 4.73E−01 | 8.58E+00 | 8.58E+00 | 1.57E+00 | 1.58E+00 |
| Ratio (g)/Al | Tumor (g/Al %) | 2.98E+00 | 1.17E+00 | 2.14E+00 | 4.19E−01 | 1.19E+00 | 1.62E−01 | 6.32E−01 | 2.23E−01 |
| | Tu/Liver | 3.06E−01 | 3.17E−02 | 1.90E−01 | 5.18E−02 | 1.20E−01 | 8.85E−02 | 1.17E−01 | 3.29E−02 |
| | Tu/Lungs | 5.44E−01 | 4.19E−01 | 9.45E−01 | 7.77E−02 | 2.26E+00 | 4.05E−01 | 2.22E+00 | 2.34E−01 |
| | TU/kidneys | 3.36E−01 | 4.89E−01 | 1.09E−01 | 1.52E−01 | 2.11E−02 | 1.26E−02 | 1.74E−02 | 3.14E−03 |
| | Tu/blood | 3.69E−01 | 7.16E−01 | 4.62E−01 | 2.77E−01 | 9.00E+00 | 9.92E+00 | 3.80E+00 | 1.96E−01 |
| | Total Elimination | 2.59E+01 | 2.06E+01 | 3.58E+01 | 3.06E+00 | 6.45E+01 | 3.41E+00 | 7.36E+01 | 2.63E+00 |
| | (%/Al) | | | | | | | | |

A second series of data is presented, after eliminating the results for the mice with urinary secretion that was too quick or a tumor that was too large, as described above in the method:

| | | Time | | | | | | | |
|---|---|---|---|---|---|---|---|---|---|
| | | 2 h post-iv | | 4 h post-IV | | 24 h post-IV | | 48 post-IV | |
| | | # Animals | | | | | | | |
| | | 2 | | 4 | | 3 | | 3 | |
| | | Mean value | SD | Mean value | SD | Mean value | SD | Mean value | SD |
| | Tumor weight | 1.02E+00 | ND | 7.63E−01 | 4.73E−01 | 8.58E+00 | 1.85E−01 | 7.78E−01 | 7.04E−01 |
| | Tumor (g/Al %) | 3.50E+00 | ND | 2.14E+00 | 4.19E−01 | 1.19E+00 | 1.62E−01 | 6.68E−01 | 2.58E−01 |
| Ratio (g)/Al | Tu/Liver | 3.15E−01 | ND | 1.90E−01 | 5.18E−02 | 1.20E−01 | 8.85E−02 | 1.17E−01 | 3.29E−02 |
| | Tu/Lungs | 4.17E−01 | ND | 9.45E−01 | 7.77E−02 | 2.26E+00 | 4.05E−01 | 2.22E+00 | 2.34E−01 |
| | TU/kidneys | 4.75E−01 | ND | 1.09E−01 | 1.52E−01 | 2.11E−02 | 2.11E−02 | 1.74E−02 | 3.14E−00 |
| | Tu/blood | 1.10E+00 | ND | 4.62E−01 | 2.77E−01 | 9.00E+00 | 9.00E+00 | 3.80E+00 | 1.96E−01 |
| | Total Elimination | 1.46E+01 | ND | 3.58E+01 | 3.06E+00 | 5.45E+01 | 3.41E+00 | 7.32E+01 | 3.10E+00 |
| | (%/Al) | | | | | | | | |

The maximum tumoral uptake is observed 2 hours after IV injection, with the highest ratios relative to the liver, lungs, kidneys and blood.
Comparing with the biodistribution of the ligand alone (AP045), it can be seen that elimination is far greater at 24 hours and 48 hours, and that the Tumor to Liver ratio is much higher, providing evidence of far lower nonspecific uptake in the liver. At two hours post-IV, the uptake rate in the tumor is higher.
8.7 Results with Compound AP045 Radiolabeled with Indium-111
2 series of experiments were conducted.
The radiochemical purity is above 99%, according to the chromatographic profile obtained as described above (example 6).
17 animals were injected.
The average activity injected is 2.45±1.5 MBq
4 animals were euthanized at 4 h, 7 at 24 h, 6 at 48 h post-injection.
Examples of planar image immediately post-injection and 2 hours post-injection are illustrated in FIG. 5.
The quantitative data on biodistribution obtained on 17 animals are presented in the following table:

| Time | | | |
|---|---|---|---|
| 2 h post-iv | 4 h post-IV | 24 h post-IV | 48 post-IV |
| # Animals | | | |
| 2 | 4 | 7 | 6 |

| | | Mean value | SD | Mean value | SD | Mean value | SD | Mean value | SD |
|---|---|---|---|---|---|---|---|---|---|
| | Tumor weight | | | 2.44E+00 | 4.22E−01 | 2.36E+00 | 8.19E−01 | 4.12E+00 | 1.66E+00 |
| | Tumor (g/Al %) | | | 1.44E+00 | 5.99E−01 | 1.77E+00 | 4.15E−01 | 1.19E+00 | 3.72E−01 |
| Ratio (g)/Al | Tu/Liver | | | 4.97E−02 | 1.09E−02 | 1.31E+00 | 3.74E−01 | 7.14E−02 | 4.20E−02 |
| | Tu/Lungs | | | 1.92E+00 | 8.65E−01 | 3.55E+00 | 6.42E−01 | 2.97E+00 | 5.07E−01 |
| | TU/kidneys | | | 2.45E−01 | 1.03E−01 | 2.93E−01 | 6.48E−02 | 4.06E−01 | 1.23E−01 |
| | Tu/blood | | | 1.12E+01 | 6.01E+00 | ND | ND | 9.95E+00 | 4.18E+00 |
| | Total | | | 6.38E+01 | 1.04E+01 | 90.3262579 | 1.81685483 | 7.23E+01 | 1.39E+00 |
| | Elimination (% | | | | | | | | |
| | del ‘Al’) | | | | | | | | |

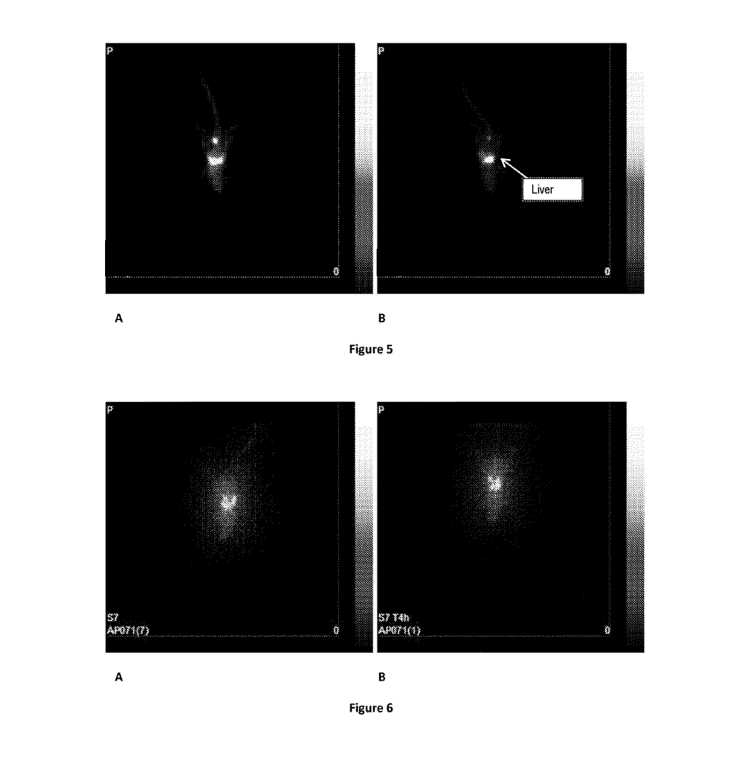

The maximum tumoral uptake is observed 24 hours after IV injection with a ratio relative to the liver greater than 1; in contrast, the activity in the kidneys is still high with an unfavorable tumor/kidney ratio.
8.8 Results with Compound AP071 (Radiolabeled with Indium-111)
The radiochemical purity is above 95, according to the chromatographic profile obtained as described above (example 6).
16 animals were injected. 1 mouse died 30 minutes after injection (cause unknown, but probably connected with the anesthesia).
The average activity injected is 3.14±0.45 MBq
2 animals were euthanized at 2 h, 3 at 4 h, and 4 at 24 h and 48 h post-injection.
Examples of planar image 2 hours post-injection and 4 hours post-injection are illustrated in FIG. 6.
The quantitative data on biodistribution obtained on 13 animals are presented in the following table:

| | | Time | | | | | | | |
|---|---|---|---|---|---|---|---|---|---|
| | | 2 h post-iv | | 4 h post-IV | | 24 h post-IV | | 48 post-IV | |
| | | # Animals | | | | | | | |
| | | 2 | | 3 | | 4 | | 4 | |
| | | Mean value | SD | Mean value | SD | Mean value | SD | Mean value | SD |
| | Tumor weight | 1.41E−02 | | 2.57E−02 | 2.10E−02 | 9.10E−02 | 7.83E−02 | 3.40E−01 | 1.30E−01 |
| | Tumor (g/Al %) | 4.42E+00 | | 1.62E+01 | 2.01E+01 | 3.07E+00 | 1.40E+00 | 3.40E−01 | 1.30E−01 |
| Ratio (g)/Al | Tu/Liver | 1.38E−01 | | 2.09E+00 | 2.48E+00 | 6.98E−01 | 4.68E−01 | 4.79E−01 | 7.69E−02 |
| | Tu/Lungs | 2.43E−01 | | 6.20E+00 | 7.97E+00 | 2.88E+00 | 1.56E+00 | 2.85E+00 | 6.70E−01 |
| | TU/kidneys | 2.41E−02 | | 1.12E+00 | 1.18E+00 | 4.61E−01 | 2.20E−01 | 3.15E−01 | 6.75E−02 |
| | Tu/blood | 8.69E−01 | | 3.92E+01 | 4.64E+01 | 9.46E+00 | 6.31E+00 | 1.17E+01 | 6.03E+00 |
| | % Elimination/Al | 6.0E+01 | | 7.3E+01 | 6.2E+00 | 8.5E+01 | 4.2E+00 | 8.5E+01 | 1.1E+00 |

A second series of data is presented, after eliminating the results for two mice with urinary excretion that was too quick (>50% at 2 h, >75% at 4 h):

| | | Time | | | | | |
|---|---|---|---|---|---|---|---|
| | | 2 h post-iv | 4 h post-IV | 24 h post-IV | | 48 post-IV | |
| | | # Animals | | | | | |
| | | | 2 | 4 | | 4 | |
| | | 1 | Mean value | Mean value | SD | Mean value | SD |
| | Tumor weight | 6.00E−02 | 1.50E−02 | 9.10E−02 | 7.83E−02 | 3.40E−01 | 1.30E−01 |
| | Tumor (g/Al %) | 6.57E+00 | 2.25E+01 | 3.07E+00 | 1.40E+00 | 3.40E−01 | 1.30E−01 |
| Ratio (g)/Al | Tu/Liver | 4.89E−01 | 2.80E+00 | 6.98E−01 | 4.68E−01 | 4.79E−01 | 7.69E−02 |
| | Tu/Lungs | 9.54E−01 | 8.36E+00 | 2.88E+00 | 1.56E+00 | 2.86E+00 | 6.70E−01 |
| | TU/kidneys | 2.25E−01 | 1.39E+00 | 4.61E−01 | 2.20E−01 | 3.15E−01 | 6.75E−02 |
| | Tu/blood | 2.73E+00 | 7.20E+01 | 9.46E+00 | 6.31E+00 | 1.17E+01 | 6.03E+00 |
| | % Elimination/Al | 5.1E+01 | 6.9E+01 | 8.5E+01 | 4.2E+00 | 8.5E+01 | 1.1E+00 |

The tumoral uptake 4 hours after IV injection is very high, with high ratios relative to the liver, lungs and blood.
Comparing with the biodistribution of the ligand alone (AP045), it can be seen that the tumoral uptake is far higher, while there are still high nonspecific fixations and rapid elimination.
8.9 Results with Compound AP070 (Radiolabeled with Indium-111)
The radiochemical purity is above 95%, according to the chromatographic profile obtained as described above (example 6).
5 animals were injected in a first experiment and were euthanized at 2 h, 4 h or 24 h; 16 animals were injected in a second experiment, under hydric restriction (no access to drink 30 minutes before injection and up to 3 hours post-injection) and were euthanized at 2 h, 4 h, 48 h.
Examples of planar image 1 hour post-injection and 2 hours post-injection are illustrated in FIG. 7.
The quantitative data on biodistribution collected from 20 animals having low urinary excretion spontaneously (<50% or 75% at 2 h or 4 h respectively) or under experimental conditions of hydric restriction are presented in the following table:

| | | Time | | | | | | | |
|---|---|---|---|---|---|---|---|---|---|
| | | 2 h post-iv | | 4 h post-IV | | 24 h post-IV | | 48 post-IV | |
| | | # Animals | | | | | | | |
| | | 6 | | 5 | | 5 | | 4 | |
| | | Mean value | SD | Mean value | SD | Mean value | SD | Mean value | SD |
| | Tumor weight | 6.3E−01 | 2.1E+00 | 6.65E−01 | 1.22E+00 | 6.56E−02 | 7.05E−02 | 1.36E−01 | 6.10E−02 |
| | Tumor (g/Al %) | 5.9E+00 | 1.6E+00 | 1.27E+01 | 1.53E+00 | 3.69E+00 | 3.12E+00 | 3.60E+00 | 1.24E+00 |
| Ratio (g)/Al | Tu/Liver | 1.4E+00 | 3.4E−01 | 1.28E+00 | 1.86E−01 | 4.06E−01 | 5.55E−01 | 1.59E+00 | 5.03E−02 |
| | Tu/Lungs | 2.1E+00 | 4.4E−01 | 2.93E+00 | 6.48E−01 | 3.52E+00 | 2.54E+00 | 6.28E+00 | 3.38E−01 |
| | TU/kidneys | 3.6E−01 | 1.1E−01 | 3.96E−01 | 6.01E−02 | 2.58E−01 | 1.83E−01 | 5.23E−01 | 2.51E−02 |
| | Tu/blood | 1.9E+00 | 5.7E−01 | 3.07E+00 | 2.57E+00 | 2.00E+01 | ND | 1.74E+01 | 3.34E+00 |
| | Total | 4.8E+01 | 2.5E+01 | 2.6E+01 | 1.5E+01 | 6.8E+01 | 1.6E+01 | 8.4E+01 | 4.7E+00 |
| | Elimination | | | | | | | | |

The tumoral uptake 4 hours after IV injection is high, with high ratios relative to the liver, lungs and blood.
Comparing with the biodistribution of the ligand alone (AP045), it can be seen that the tumoral uptake is far higher, and there are still high nonspecific fixations and rapid elimination. However, relative to the compound AP071, it can be seen that the tumoral uptake is lower, notably at 4 hours, with a less favorable tumor/liver ratio.

### Example 9

### Characterization of the Sentinel Lymph Node

### 9.1 Imaging Protocol:

Scintigraphic imaging is performed using a dedicated small animal micro-5 imager SPECT-CT (BIOSCAN™). A planar scintigraphic image is obtained immediately after injection to verify absence of intracaudal stasis. Static scintigraphic images may be recorded at 2 h, 4 h, 24 h and 48 h (Imager micro-SPECT-CT BIOSCAN©).

### 9.2 Preclinical Protocol:

Intradermal injection of 1 to 20 microliters of a suspension of radiolabeled dendrimers with a hypodermic syringe in the pad of the hindpaw of a nude mouse.
A whole-body planar scintigraphic image of the mouse is obtained 5 minutes after injection to verify absence of passage of the compound through the blood.
Acquisition of tomographic images (SPECT) every 30 minutes to monitor the lymphatic drainage of the compound and determine the time of disappearance of the signal in the popliteal lymph node.
9.3.a Injection of Compound X1 Labeled with Tc99m (Generation 2 Molecule)

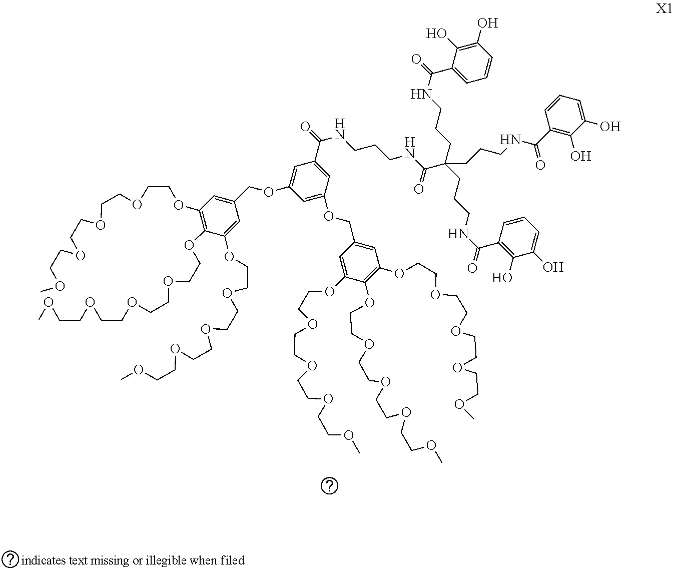 [SMILES: COCCOCCOCCOCCOc1cc(COc2cc(OCc3cc(OCCOCCOCCOCCOC)c(OCCOCCOCCOCCOC)c(OCCOCCOCCOCCOC)c3)cc(C(=O)NCCCNC(=O)C(CCCNC(=O)c3cccc(O)c3O)(CCCNC(=O)c3cccc(O)c3O)CCCNC(=O)c3cccc(O)c3O)c2)cc(OCCOCCOCCOCCOC)c1OCCOCCOCCOCCOC]

Compound X1 is synthesized by a method similar to the synthesis of the compound hereunder published in WO 2008/043911, simply by changing the length of the PEG chains.

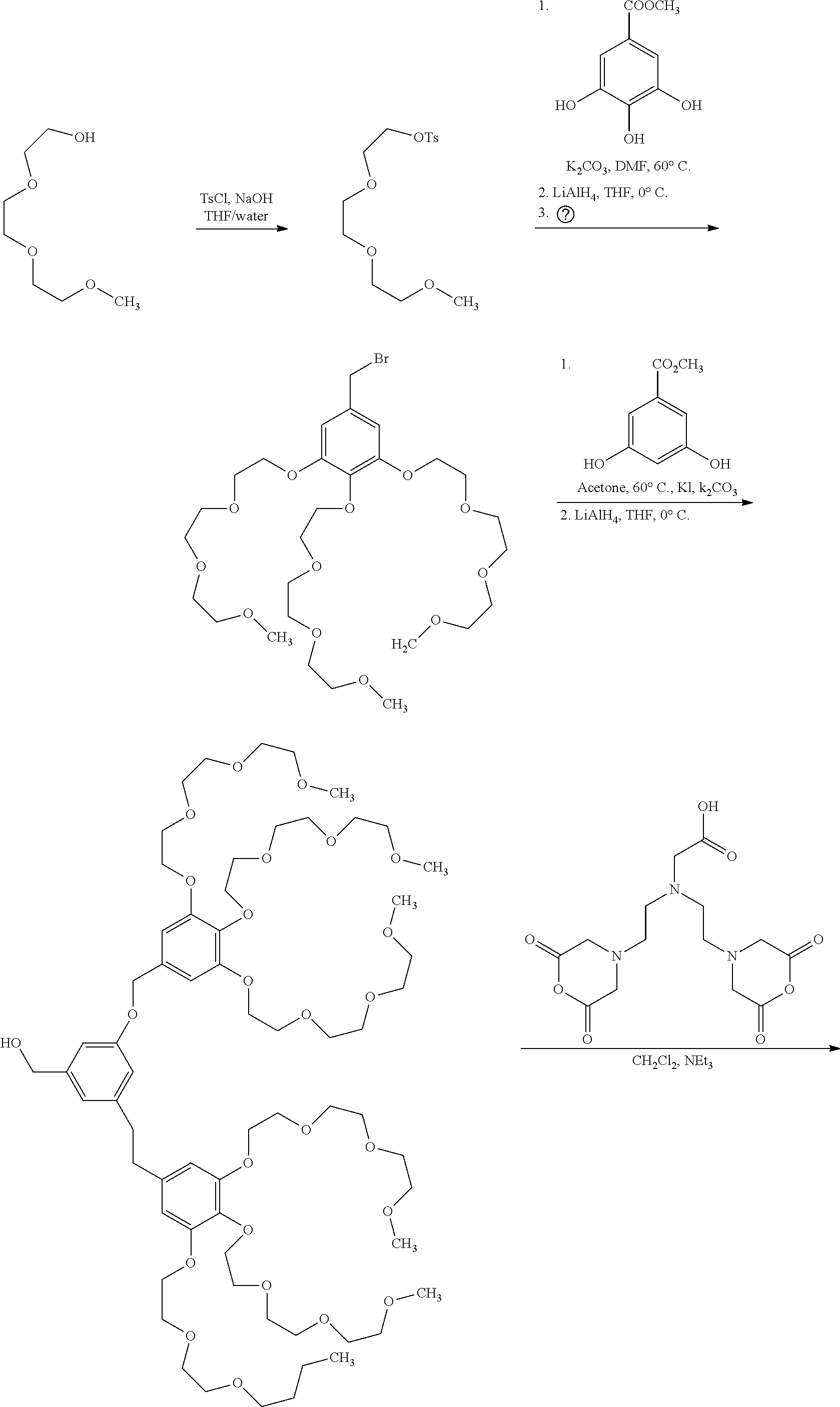

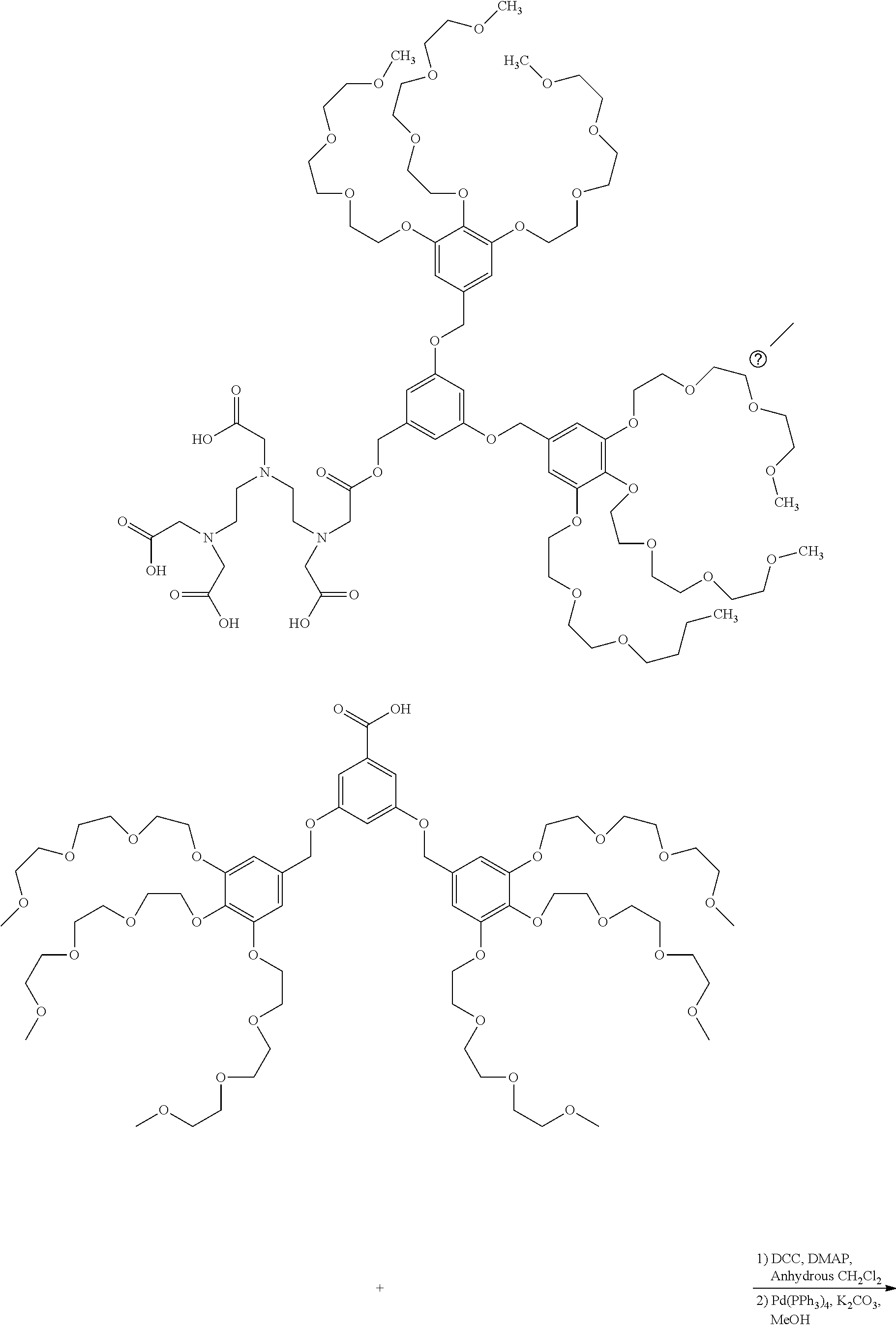

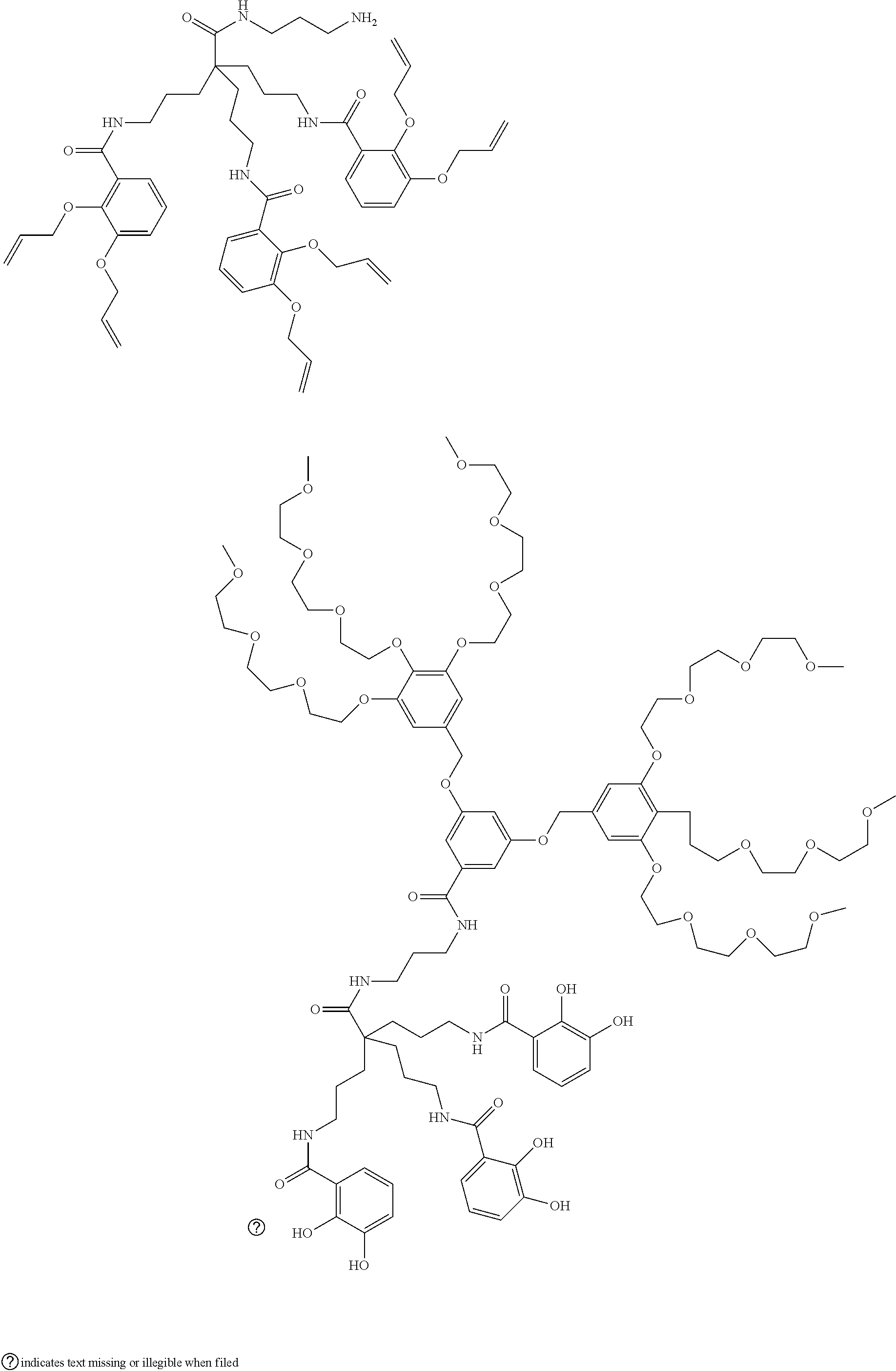 [SMILES: C=CCOc1cccc(C(=O)NCCCC(CCCNC(=O)c2cccc(OCC=C)c2OCC=C)(CCCNC(=O)c2cccc(OCC=C)c2OCC=C)C(=O)NCCCN)c1OCC=C ; COCCOCCOCCCc1c(OCCOCCOCCOC)cc(COc2cc(OCc3cc(OCCOCCOCCOC)c(OCCOCCOCCOC)c(OCCOCCOCCOC)c3)cc(C(=O)NCCCNC(=O)C(CCCNC(=O)c3cccc(O)c3O)(CCCNC(=O)c3cccc(O)c3O)CCCNC(=O)c3cccc(O)c3O)c2)cc1OCCOCCOCCOC]

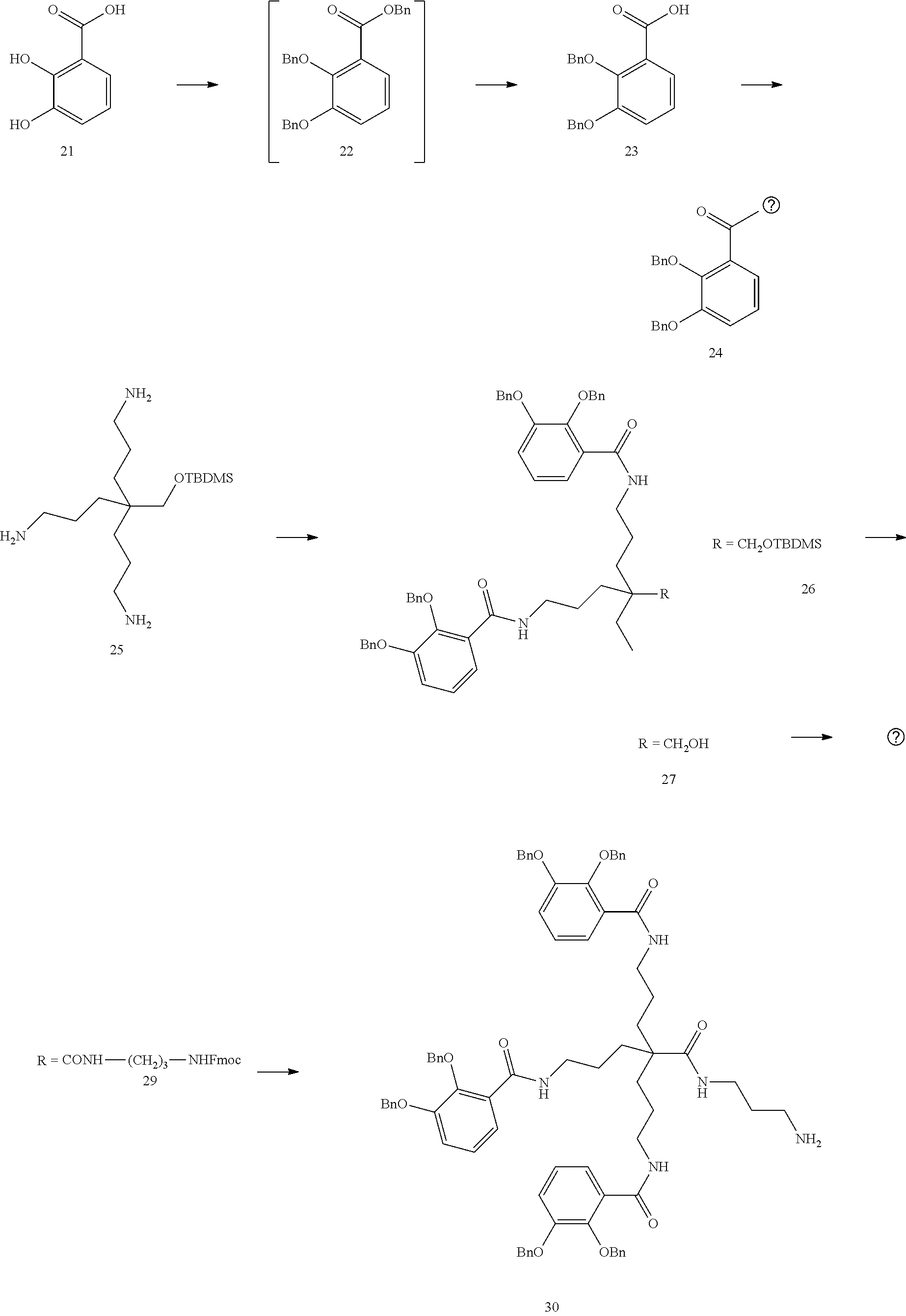

The synthesis of compound 30 is detailed in Example 3.

9.3.b Injection of Compound APAG4 (Generation 1) Labeled with Indium-111

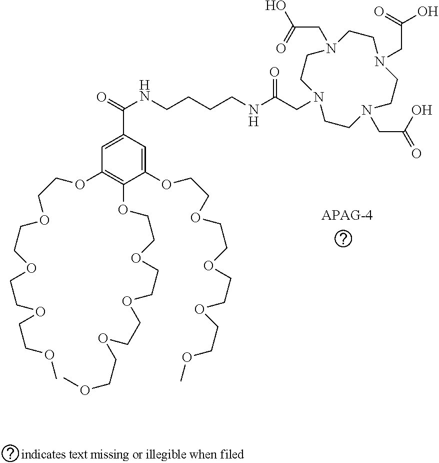 [SMILES: COCCOCCOCCOCCOc1cc(C(=O)NCCCCNC(=O)CN2CCN(CC(=O)O)CCN(CC(=O)O)CCN(CC(=O)O)CC2)cc(OCCOCCOCCOCCOC)c1OCCOCCOCCOCCOC]

9.3.c Intradermal Injection of Compound AP034 Labeled with Tc-99m

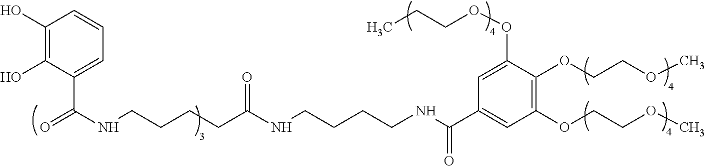

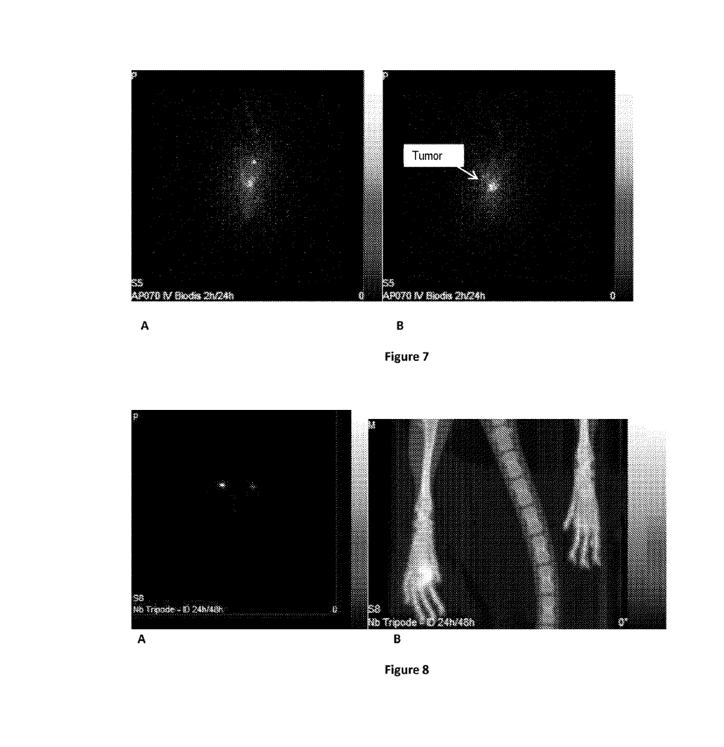

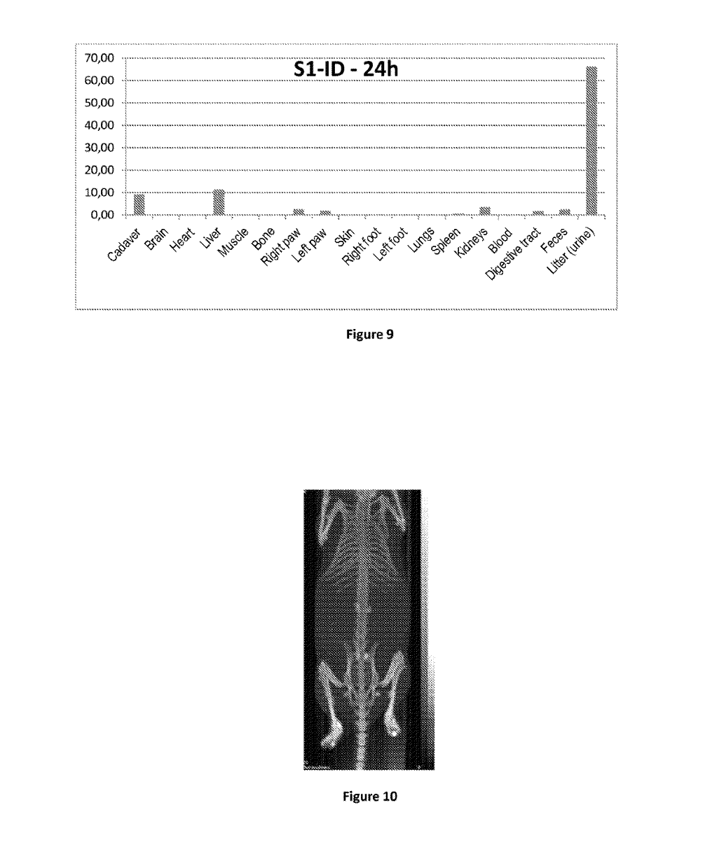

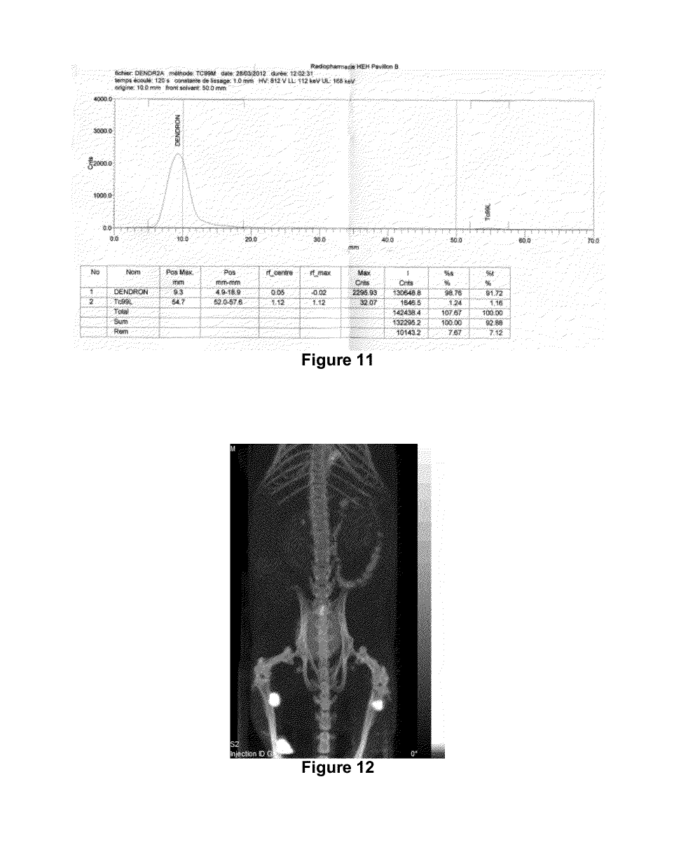

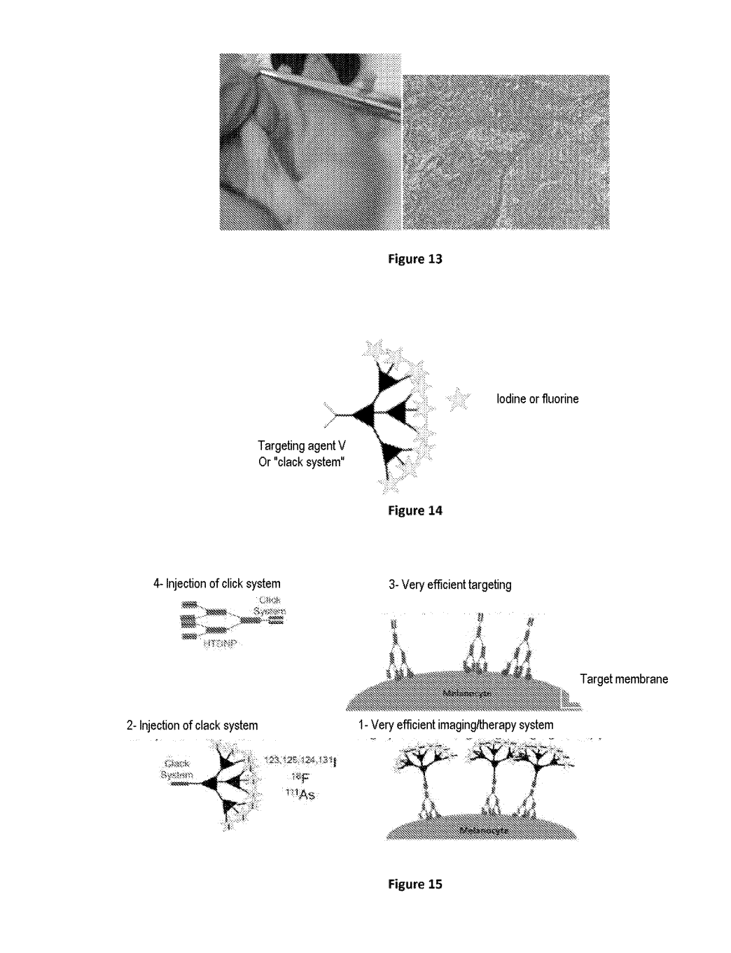

The PEG-tosylated and gallate-PEG-OMe chains required for obtaining compound APO 34 were obtained following the procedures in the article G. Lamanna et al., Biomaterials, 2011, 32, 8562-8573. After saponification of the ester of gallate-PEG-OMe, the pale yellow oil was dissolved in dry CH₂Cl₂ with the catecholamine chelating agent tamine 12 obtained in the article A Bertin et al., New J Chem., 2010, 34, 267-275) for a peptide coupling reaction in the presence of EDCI, DMAP and DIPEA. After 48 h, the residue is purified by silica gel column chromatography (CH₂Cl₂/MeOH 95:5-90/10) to give a pale yellow oil (65%). Then, final deallylation using Pd (PPh3) 4 and NaBH₄ min dry THF gives the compound APO34 “Catechol chelating agent-gallate-PEG-OMe” in the form of yellow oil with a 58% yield.
Compounds X1 and APO34 were to be radiolabeled according to the protocol detailed in example 5.
Compound APAG4 may be radiolabeled according to the protocol detailed in example 7. The results after injection for these three compounds are illustrated in FIGS. 8 to 12.

### Example 10

### Characterization of the Tumoral Sentinel Lymph Node

In the model of tumoral sentinel lymph node, obtained by intradermal injection of 10⁶ tumor cells (human melanoma line A375), compounds G1 or G2 of example 9 functionalized with the targeting agents V according to the invention and in particular:
the nucleolin membrane ligands:

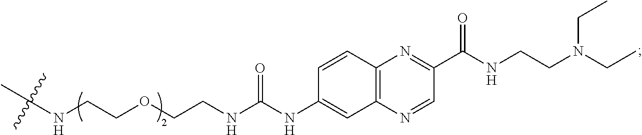

and/or
alpha-MSH ligands (HB-19):

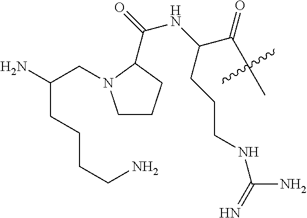 [SMILES: *C(=O)C(CCCNC(=N)N)NC(=O)C1CCCN1CC(N)CCCCN]

will make it possible to distinguish nontumoral sentinel lymph nodes (no fixation lasting for several hours after injection at the level of the popliteal lymph node) from the tumoral lymph nodes (persistence of fixation), whether or not a two-stage protocol is used.
For example, the following compounds may be used for a one-stage protocol:

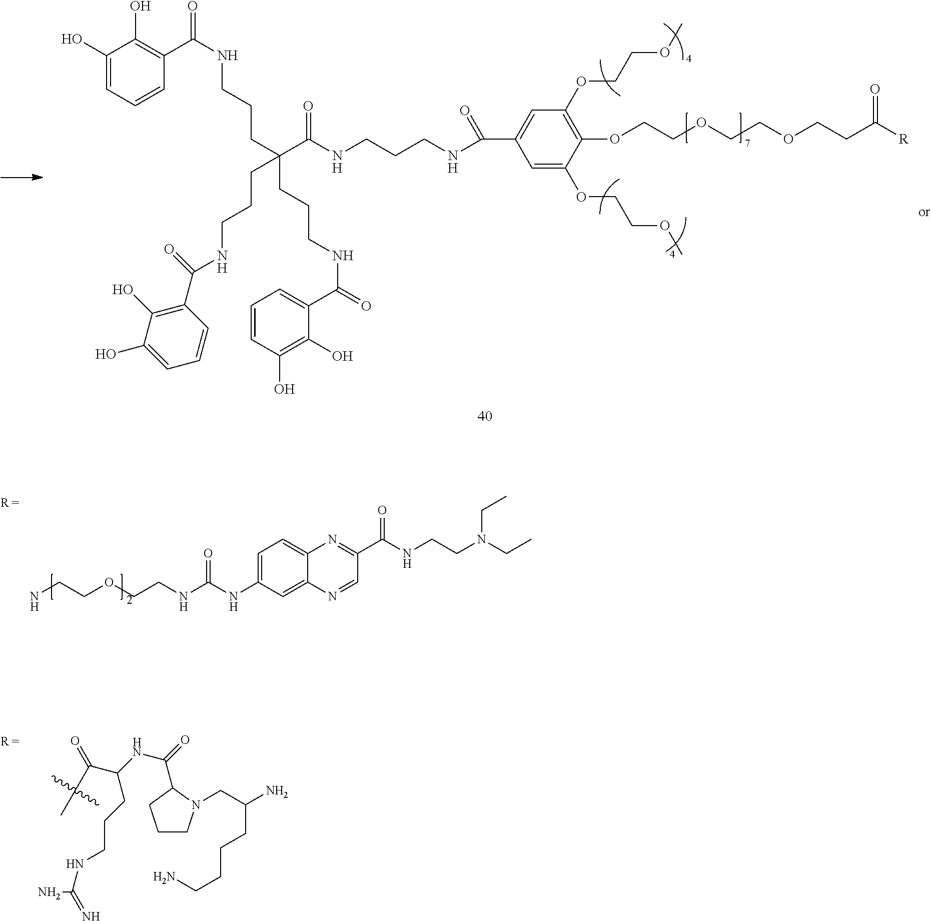

The one-stage protocol may be described as comprising the injection of the radioactive solutes. The two-stage protocol may be described as comprising the injection first of the compound that is not radiolabeled but comprises a “clack”, and secondly, of a radiolabeled compound comprising a “click”.

### LIST OF REFERENCES

[1] S. E. Stiriba, H. Frey et R. Haag, Angew. Chem. Int. Ed., 2002, 41, 1329-1334;
[2] M. J. Cloninger, Curr. Opin. Chem. Biol., 2002, 6, 742-748;
[3]R. Duncan et L. Izzo, Adv. Drug Delivery Rev., 2005, 57, 2215-2237;
[4]C. C. Lee, J. A. MacKay, J. M. J. Frdchet et F. C. Szoka, Nat Biotechnol., 2005, 23, 1517-1526;
[5]O. Rolland, C-O. Turrin. A-M. Caminade, J-P. Majoral, New J. Chem., 2009, 33, 1809-1824
[6] E. C. Wiener, M. W. Brechbiel, H. Brothers, R. L. Magin, O. A. Gansow, D. A. Tomalia et P. C. Lauterbur, Magn. Reson. Med., 1994, 31, 1
[7]E. C. Wiener, F. P. Auteri, J. W. Chen, M. W. Brechbiel, O. A. Gansow, D. S. Schneider, R L. Belford, R. B. Clarkson et P. C. Lauterbur. J. Am. Chem. Soc., 1996, 118, 7774
[8]V. S. Vexler, O. Clement, H. Schmitt-Willich et R. C. Brasch, J. Magn. Reson. Imaging, 1994, 4, 381,
[9] T. S. Desser, D. L. Rubin, H. H. Muller, F. Qing, S. Khodor, G. Zanazzi, S. W. Young, D. L. Ladd, J. A. Wellons et K. E. Kellar, J. Magn. Reson. Imaging, 1994, 4, 467
[10] L. H. Bryant, Jr, M. W. Brechbiel, C. Wu, J. W. Bulte, V. Herynek et J. A. Frank, J. Magn. Reson. Imaging, 1999, 9, 348
[11] E. Toth, D. Pubanz, S. Vauthey, L. Helm et A. E. Merbach, Chem. Eur. J., 1996, 2, 1607
[12] J. Rudovsky, P. Hermann, M. Botta, S. Aime et I. Lukes, Chem. Commun., 2005, 2390
[13] H. Kobayashi, S. Kawamoto, S.-K. Jo, H. L. Bryant, Jr, M. W. Brechbiel, Jr et R. A. Star, Bioconjugate Chem., 2003, 14, 388
[14] S. Langereis, Q. G. de Lussanet, M. H. P. van Genderen, W. H. Backes et E. W. Meijer, Macromolecules, 2004, 37, 3084
[15]C. Fink, F. Kiessling, M. Bock. M. P. Lichy, B. Misselwitz, P. Peschke, N. E. Fusenig, R. Grobholz et S. Delorme, J. Magn. Reson. Imaging, 2003, 18, 59;
[16] G. M. Nicolle. E. Toth, H. Schmitt-Willich, B. Raduchel et A. E. Merbach. Chem. Eur. J, 2002, 8, 1040;
[17] G. Adam, J. Neuerburg, E. Spuntrup, A. Muhler, K. Scherer et R. W. Gunther, J. Magn. Reson. Imaging, 1994, 4, 462;
[18]G. Adam, J. Neuerburg, E. Spuntrup, A. Muhler, K. Scherer et R. W. Gunther, Magn. Reson. Med, 1994, 32, 622;
[19] H. C. Schwickert, T. P. Roberts, A. Muhler, M. Stiskal, F. Demsar et R. C. Brasch. Eur. J Radiol., 1995, 20, 144;
[20] H. C. Roberts, M. Saeed, T. P. Roberts, A. Muhler, D. M. Shames, J. S. Mann, M. Stiskal, F. Demsar et R. C. Brasch, J. Magn. Reson. Imaging, 1997, 7, 331
[21] D. Artemov, J. Cell. Biochem., 2003, 90, 518
[22]H. Stephan, H. Spies, B. Johannsen, K. Gloe, M. Gorka. F. Vögtle, Eur., J. Inorg. Chem., 2001, 2957-2963
[23] M. Subbarayan, S. J. Shetty, T. S. Srivastava, O. P. D. Noronha. A. M. Samuel, H. Mukhtar, Biochem. and Biophys. Res. Commun., 2001, 281, 32-36
[24] M. C. Parrott, S. R. Benhabbour, C. Saab, J. A. Lemon, S. Parker, J. F. Valliant, A. Adronov, J. Am. Chem. Soc., 2009, 131, 2906-2916
[25] Sefah. Kwame. et al. “Molecular recognition of acute myeloid leukemia using aptamers.” Leukemia, 23 (2009); 235-244
[26] Maisonial et al. J. Med. Chem. 2011, 54, 2745,
[27] Rbah-Vidal et al. Eur. J. Med. Mol. Imaging 2012, 39, 1449.
[28] Vivier et al. Eur. J. Med. Chem. 2011, 46, 5705
[29] WO2009/095872
[30] Soundararajan et al. Cancer Res. 2008, 68, 2358
[31] A. Bertin et al., New J. Chem., 2010, 34, 267-275
[32] G. Lamanna et al., Biomaterials, 2011, 32, 8562-8573

1. A compound of Formula I
T-L₁-D-R-L₂V)ₙ (I)
wherein:
T is a chelating agent, a fluorochrome or a recognition agent [symbol] [symbol]
wherein T represents:
a chelating agent of formula: 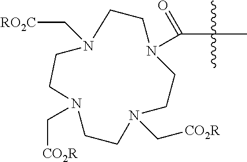
where each occurrence of R represents independently H or a protective group such as t-Bu;
a chelating agent of formula: 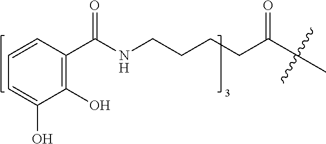
except in the case when D is a gallate dendrimer and V is a DOPA targeting agent comprising the structure of formula 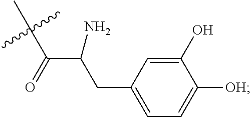 [SMILES: *C(=O)C(N)Cc1ccc(O)c(O)c1]
a fluorescein fluorochrome of formula: 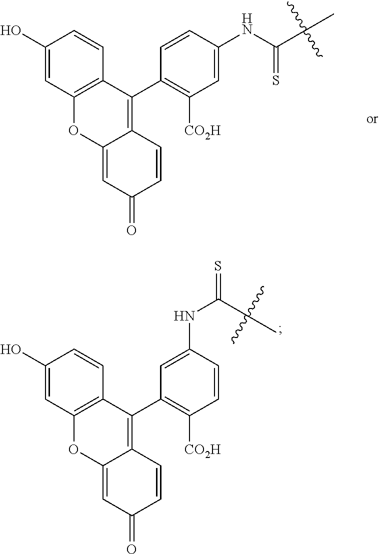 [SMILES: *C(=S)Nc1ccc(-c2c3ccc(=O)cc-3oc3cc(O)ccc23)c(C(=O)O)c1 ; *C(=S)Nc1ccc(C(=O)O)c(-c2c3ccc(=O)cc-3oc3cc(O)ccc23)c1]
a recognition agent selected from cyclodextrin, adamantane, biotin, avidin, or streptavidin;
L₁ represents Ø (i.e. a covalent bond), or a spacer —C(═O)NH—, —NHC(═O)—, —NHC(═S)NH— or triazolyl of formula: 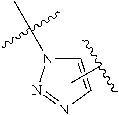
wherein one of the points of attachment is bound to T and the other to D directly or via a heteroalkyl chain of formula: 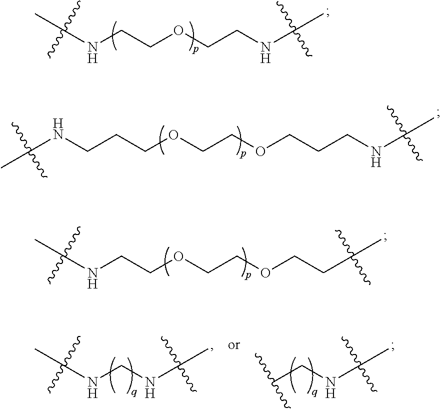
wherein p is an integer from 1 to 10, and q is an integer from 1 to 12;
D is a PAMAM; gallate or aspartate dendrimer of formula (DD): 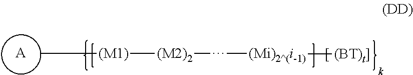
wherein:
(a) A is the dendrimer nucleus, of multivalence k, where:
k represents the number of dendrons and is an integer ranging from 2 to 8;
A is a synthon of structure: 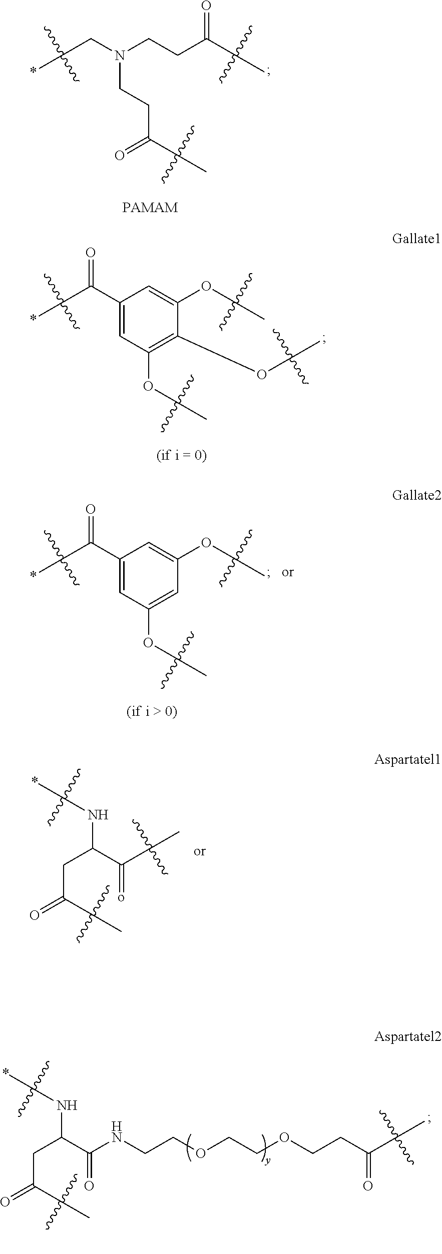
where * indicates the point of attachment to the spacer L₁; and y represents an integer from 1 to 10;
(b) Mi is a monomer of generation i, where:
i is an integer ranging from 0 to g, g being the generation number of the dendrimer,
when i=0, Mi is absent, and the terminal branch TB is then bound directly to the nucleus A;
when i>0, Mi represents: 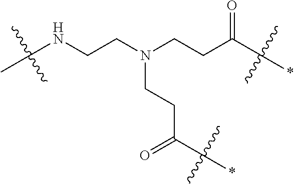
when D is PAMAM; 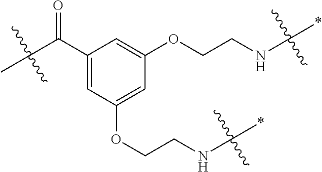
when D is gallate; 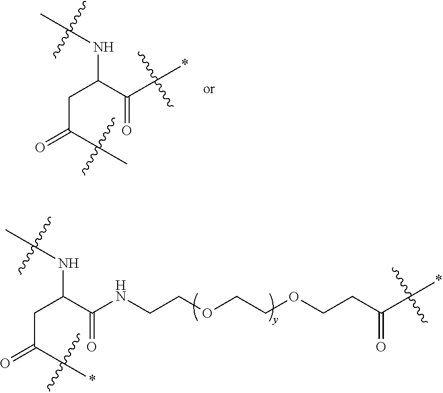
when D is aspartate
where the symbol * denotes the point of attachment of the monomer Mi to the monomer of higher generation; and y represents an integer from 1 to 10;
(c) TB is the terminal branch, and t is the number of terminal units from the monomer of lower generation, where:
t is an integer ranging from 1 to 3;
TB is a radical selected from the group comprising a hydrogen, an —NH₂ group, an —OH group, a C₁ to C₆ alkyl, a polyethylene glycol chain of formula 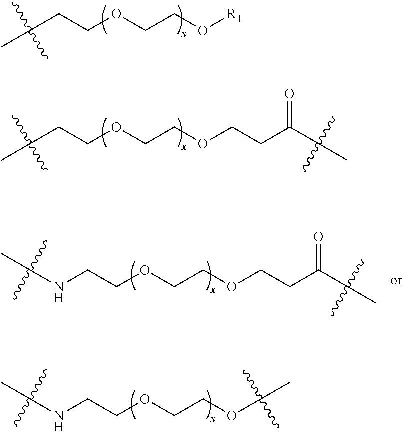
wherein each occurrence of x is independently an integer from 1 to 10;
R₁ represents C₁₋₆alkyl or —C(═O)OR₂ where R₂ represents C₁₋₆alkyl;
and at least one occurrence of TB is covalently bound to a targeting agent V;
R is Ø (i.e. a covalent bond), or represents a radiolabelable moiety or a group such as L-thyroxine or dinitrobenzoate;
L₂ represents Ø (i.e. a covalent bond), or a heteroalkyl chain of formula: 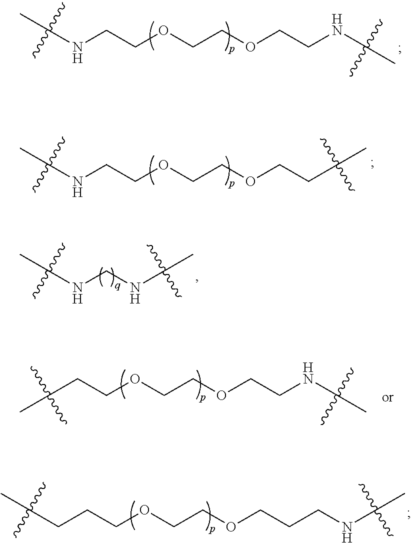
wherein p is an integer from 1 to 10, and q is an integer from 1 to 12;
V is a targeting agent selected from: 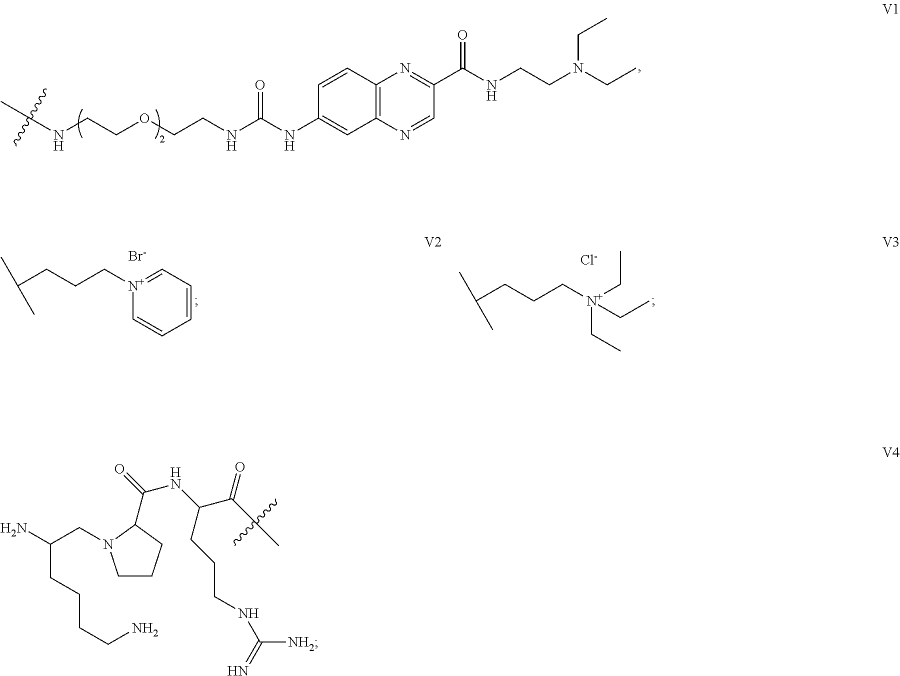
a nidazole derivative (misonidazole (MISO) or metronidazole (METRO)),
preferably an amino-metronidazole of formula 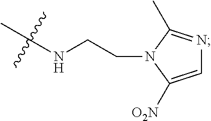 [SMILES: *NCCn1c([N+](=O)[O-])cnc1C]
L-DOPA, or a derivative thereof;
a ligand of the Human Epidermal Growth Factor Receptor 2 (HER2);
a specific antibody targeting the PSMA receptor; and
n is an integer greater than or equal to 1, and represents the number of targeting agents attached to the compound of formula I;
provided that when D is a gallate dendrimer, V does not comprise a DOPA structure of formula 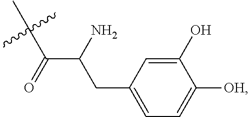 [SMILES: *C(=O)C(N)Cc1ccc(O)c(O)c1]
and T does not comprise a catechol structure of the following formula:

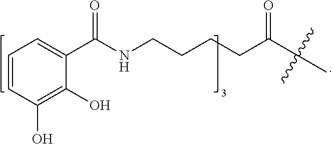

2. Compound of claim 1, wherein L₂ is a bond, and the compound has the following formula II:
T-L₁-D-RV)ₙ (II)
wherein:
T, L₁, D, V and n are as defined in claim 1; and
R is a radiolabelable moiety selected from: 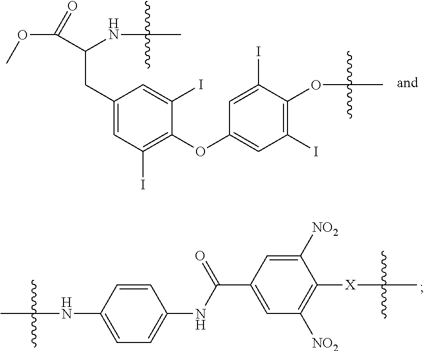
where X represents O or NH.

3. Compound of claim 1, wherein R and L₂ each represents a bond, and the compound has the following formula III:
T-L₁-DV)ₙ (III)
wherein T, L₁, D, V and n are as defined in claim 1.

4. (canceled)

5. Compound of claim 1, wherein: [symbol]
a) L₁ may represent a covalent bond, or a spacer —C(═O)NH—, —NHC(═O)—, —NHC(═S)NH—, or triazolyl of formula: 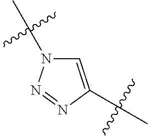 [SMILES: *c1cn(*)nn1] [symbol]
b) L₁ may advantageously represent a covalent bond, or a triazolyl spacer of formula: 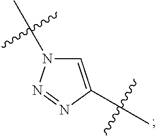 [SMILES: *c1cn(*)nn1] [symbol]
c) L₁ as defined at a) or b) may advantageously be bound to T via a heteroalkyl chain of formula: 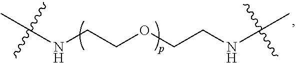
wherein p is an integer from 1 to 10; [symbol]
d) L₁ as defined at a) or b) may advantageously be bound to T via a heteroalkyl chain of formula: 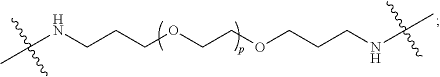
wherein p is an integer from 1 to 10; [symbol]
e) L₁ as defined at a) or b) may advantageously be bound to T via a heteroalkyl chain of formula: 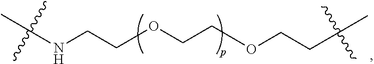
wherein p is an integer from 1 to 10; [symbol]
f) L₁ as defined at a) or b) may advantageously be bound to T via a heteroalkyl chain of formula: 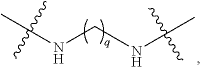
wherein q is an integer from 1 to 12; [symbol]
g) L₁ as defined at a) or b) may advantageously be bound to T via a heteroalkyl chain of formula: 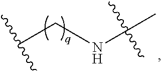
wherein q is an integer from 1 to 12.

6. Compound of claim 1, wherein: [symbol]
D may represent a PAMAM dendrimer of formula (DD) wherein:
A is a synthon of structure 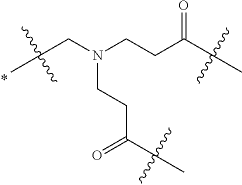 [SMILES: *CN(CCC(*)=O)CCC(*)=O]
where * indicates the point of attachment to the spacer L₁;
(b) Mi is a monomer of generation i, where:
i is an integer ranging from 0 to 3; and when i=0, Mi is absent, and the terminal branch TB is then bound directly to the nucleus A;
when i>0, Mi represents: 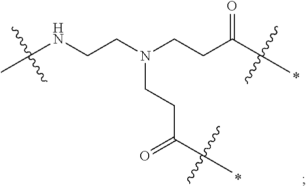 [SMILES: *NCCN(CCC(*)=O)CCC(*)=O]
where the symbol * denotes the point of attachment of the monomer Mi to the monomer of higher generation;
(c) TB is the terminal branch, and t is the number of terminal units from the monomer of lower generation, where:
t is an integer ranging from 1 to 2;
TB is a radical selected from the group comprising a hydrogen, an —NH₂ group, a —COOH group, an —OH group, a C₁ to C₆ alkyl, a polyethylene glycol chain of formula 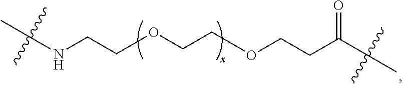
wherein each occurrence of x is independently an integer from 1 to 10; and at least one occurrence of TB is covalently bound to a targeting agent V or a radiolabelable moiety R; the occurrences of TB not bound to V are terminated with H, or a C₁ to C₆ alkyl such as methyl or ethyl; [symbol]
D may represent a gallate dendrimer of formula (DD) wherein:
A is a synthon of structure 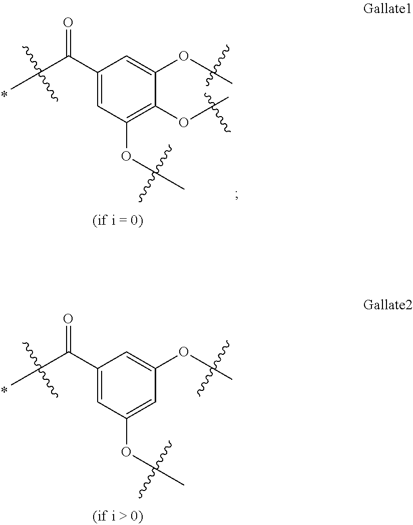 [SMILES: *Oc1cc(C(*)=O)cc(O*)c1O* ; *Oc1cc(O*)cc(C(*)=O)c1]
where * indicates the point of attachment to the spacer L₁;
(b) Mi is a monomer of generation i, where:
i is an integer ranging from 0 to 3; and when i=0, Mi is absent, and the terminal branch TB is then bound directly to the nucleus A;
when i>0, Mi represents: 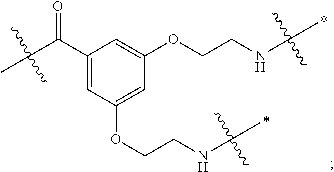 [SMILES: *NCCOc1cc(OCCN*)cc(C(*)=O)c1]
where the symbol * denotes the point of attachment of the monomer Mi to the monomer of higher generation;
(c) TB is the terminal branch, and t is the number of terminal units from the monomer of lower generation, where:
t is an integer ranging from 1 to 3;
TB is a radical selected from the group comprising a hydrogen, an —NH₂ group, an —OH group, a C₁ to C₆ alkyl, a polyethylene glycol chain of formula 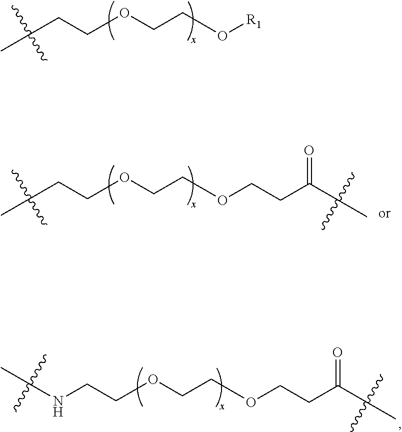
wherein each occurrence of x is independently an integer from 1 to 10;
R₁ represents C₁₋₆alkyl or —C(═O)OR₂ where R₂ represents C₁₋₆alkyl;
and at least one occurrence of TB is covalently bound to a targeting agent V or a radiolabelable moiety R; the occurrences of TB not bound to V are terminated by a C₁ to C₆ alkyl such as methyl or ethyl; [symbol]
D may represent an aspartate dendrimer of formula (DD) wherein:
A is a synthon of structure 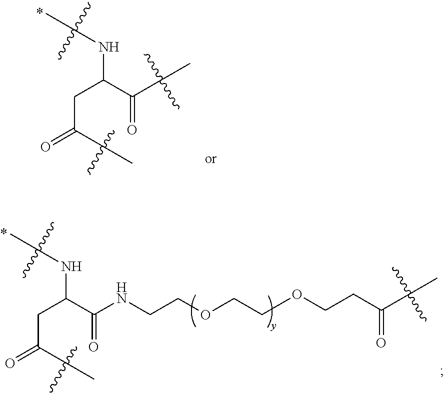
where * indicates the point of attachment to the spacer L₁; and y represents an integer from 1 to 10;
(b) Mi is a monomer of generation i, where:
i is an integer ranging from 0 to 3; and when i=0, Mi is absent, and the terminal branch TB is then bound directly to the nucleus A;
when i>0, Mi represents: 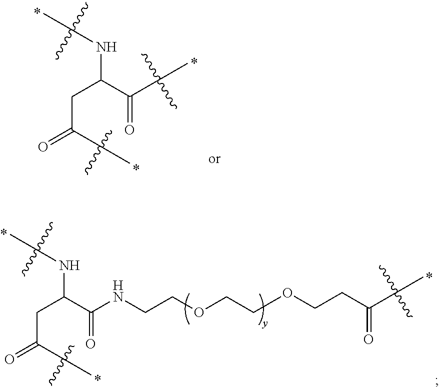
where the symbol * denotes the point of attachment of the monomer Mi to the monomer of higher generation; and y represents an integer from 1 to 10;
c) TB is the terminal branch, and t is the number of terminal units from the monomer of lower generation, where:
t is an integer ranging from 1 to 2;
TB is a radical selected from the group comprising a hydrogen, an —NH₂ group, an —OH group, a C₁ to C₆ alkyl, a polyethylene glycol chain of formula 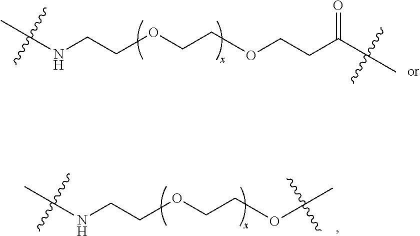
wherein each occurrence of x is independently an integer from 1 to 10; and at least one occurrence of TB is covalently bound to a targeting agent V or a radiolabelable moiety R; the occurrences of TB not bound to V are terminated with H, or a C₁ to C₆ alkyl such as methyl or ethyl.

7. Compound of claim 1, wherein: [symbol]
R represents a covalent bond: [symbol]
R represents a labelable moiety such as L-thyroxine of formula: 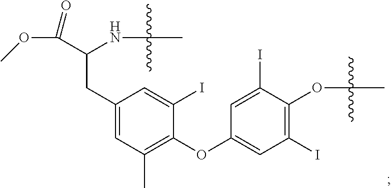 [SMILES: *NC(Cc1cc(C)c(Oc2cc(I)c(O*)c(I)c2)c(I)c1)C(=O)OC]
or [symbol]
R represents a labelable moiety such as the dinitrobenzoate of formula: 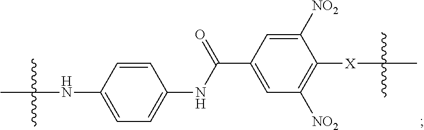
where X represents O or NH.

8. Compound of claim 1, wherein: [symbol]
L₂ represents a covalent bond; [symbol]
L₂ represents a heteroalkyl chain of formula: 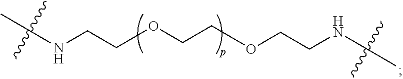
wherein p is an integer from 1 to 10; [symbol]
L₂ represents a heteroalkyl chain of formula: 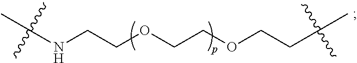
wherein p is an integer from 1 to 12; [symbol]
L₂ represents a heteroalkyl chain of formula: 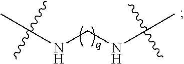
wherein q is an integer from 1 to 10; [symbol]
L₂ represents a heteroalkyl chain of formula: 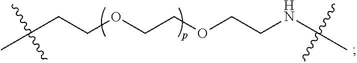
wherein p is an integer from 1 to 10; [symbol]
L₂ represents a heteroalkyl chain of formula: 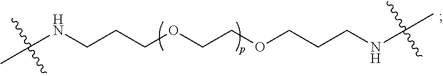
wherein p is an integer from 1 to 10; [symbol]
L₂ represents a heteroalkyl chain of formula: 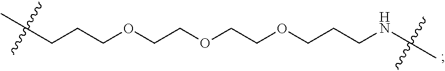 [SMILES: *CCCOCCOCCOCCCN*]
when R represents a labelable moiety such as the dinitrobenzoate of formula: 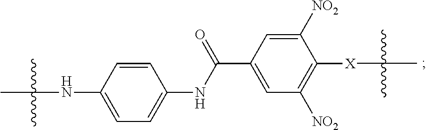
where X represents NH, to form a structure of formula:

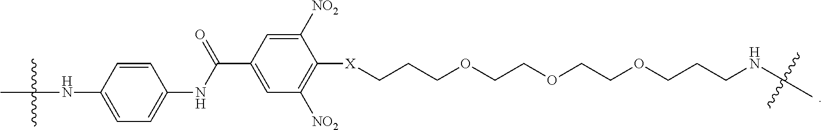

9. Compound of claim 1, wherein: [symbol]
V represents a melanoma targeting agent of formula: 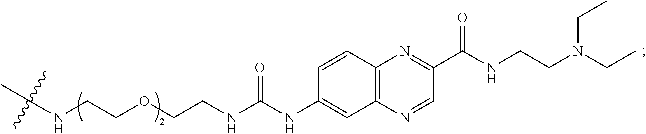 [symbol]
V represents a tripeptide targeting agent of formula: 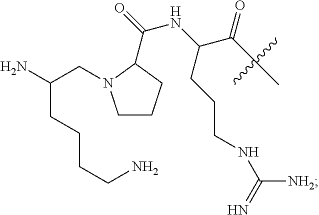 [SMILES: *C(=O)C(CCCNC(=N)N)NC(=O)C1CCCN1CC(N)CCCCN] [symbol]
V represents an amino-metronidazolyl targeting agent of formula:

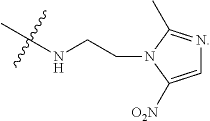 [SMILES: *NCCn1c([N+](=O)[O-])cnc1C]

10. Compound of claim 1, having one of the following structures: 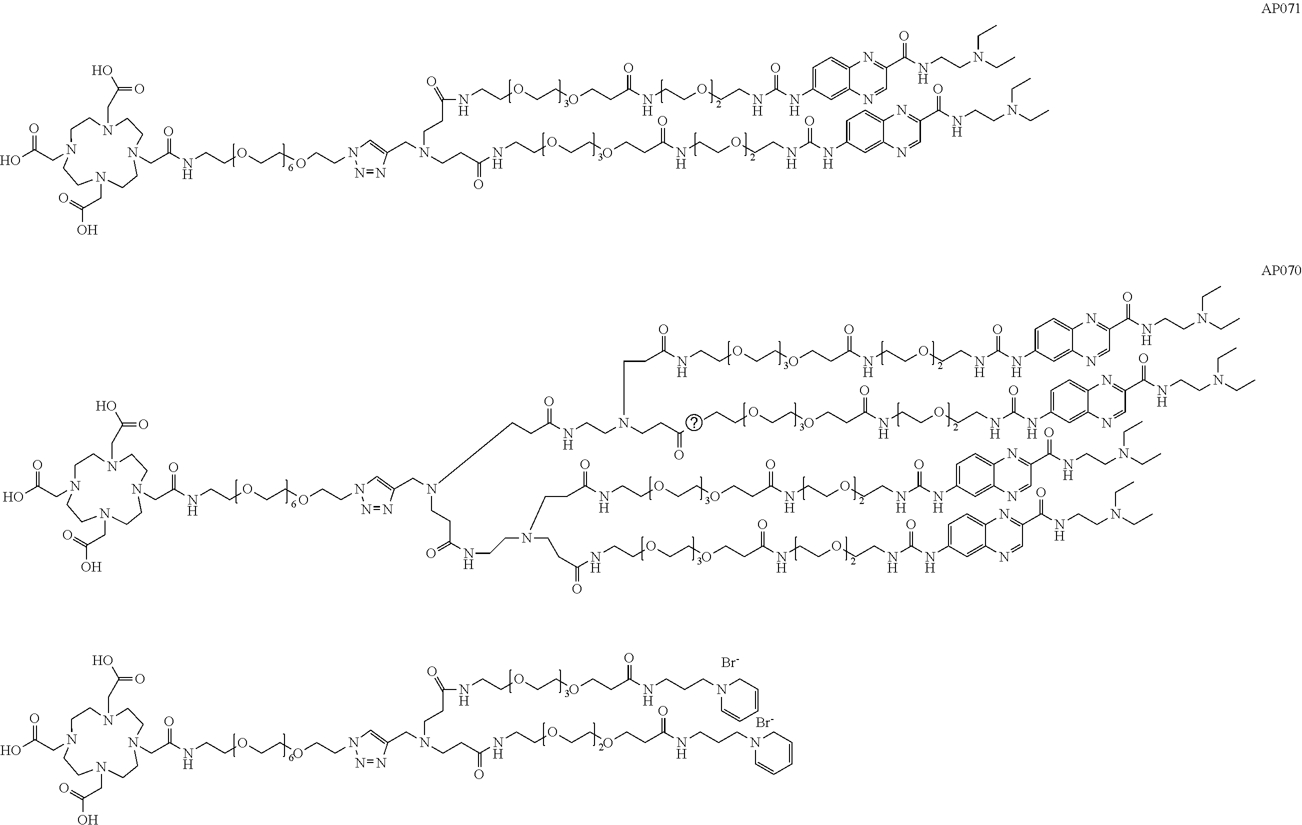 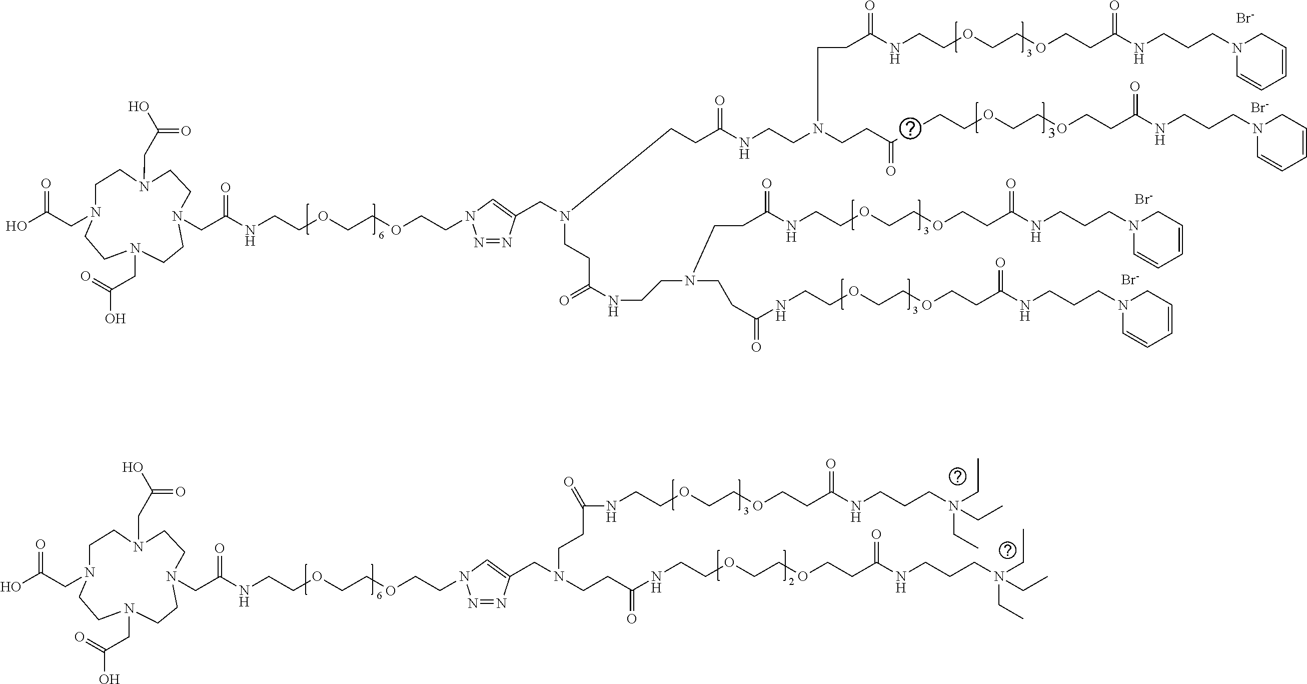 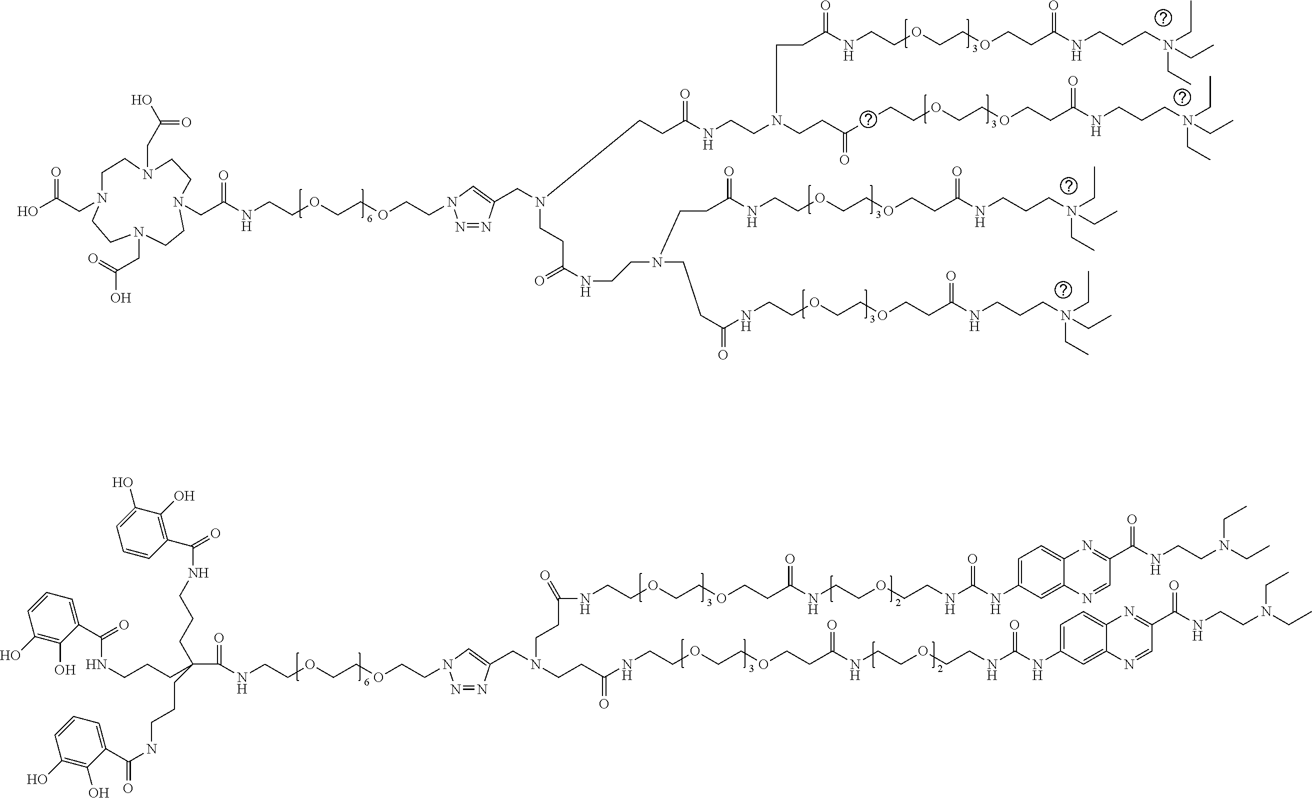 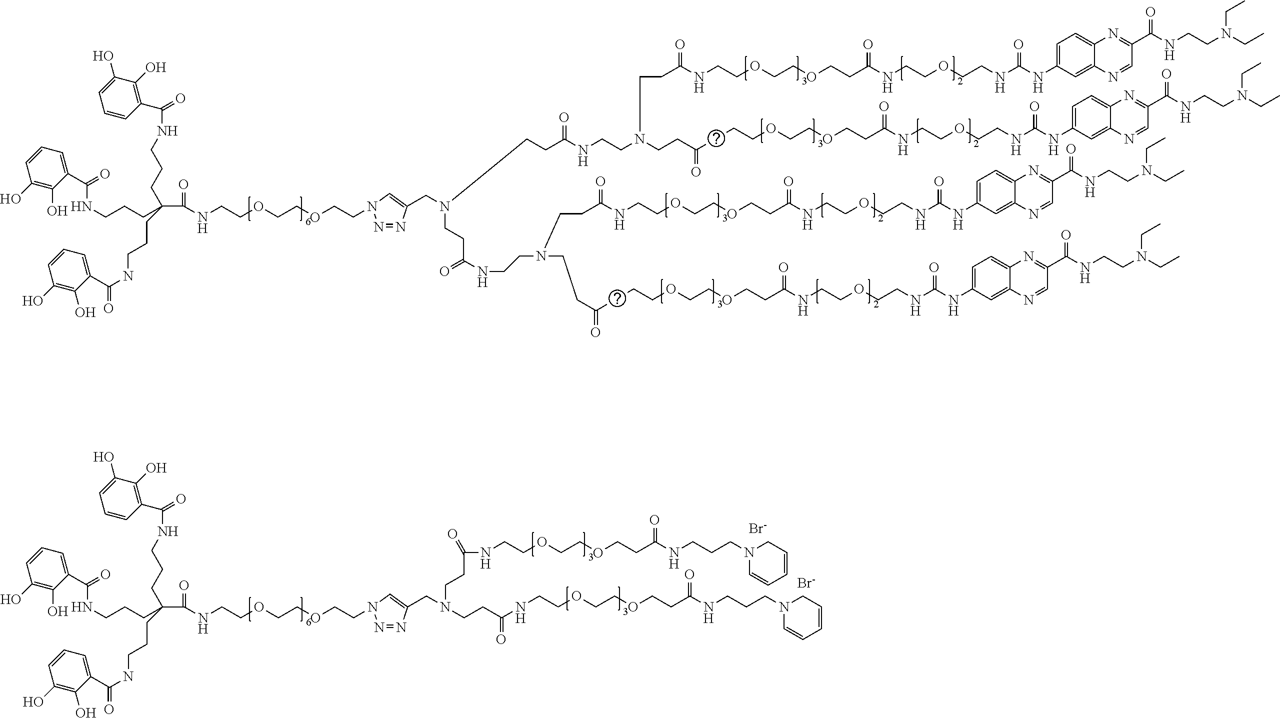 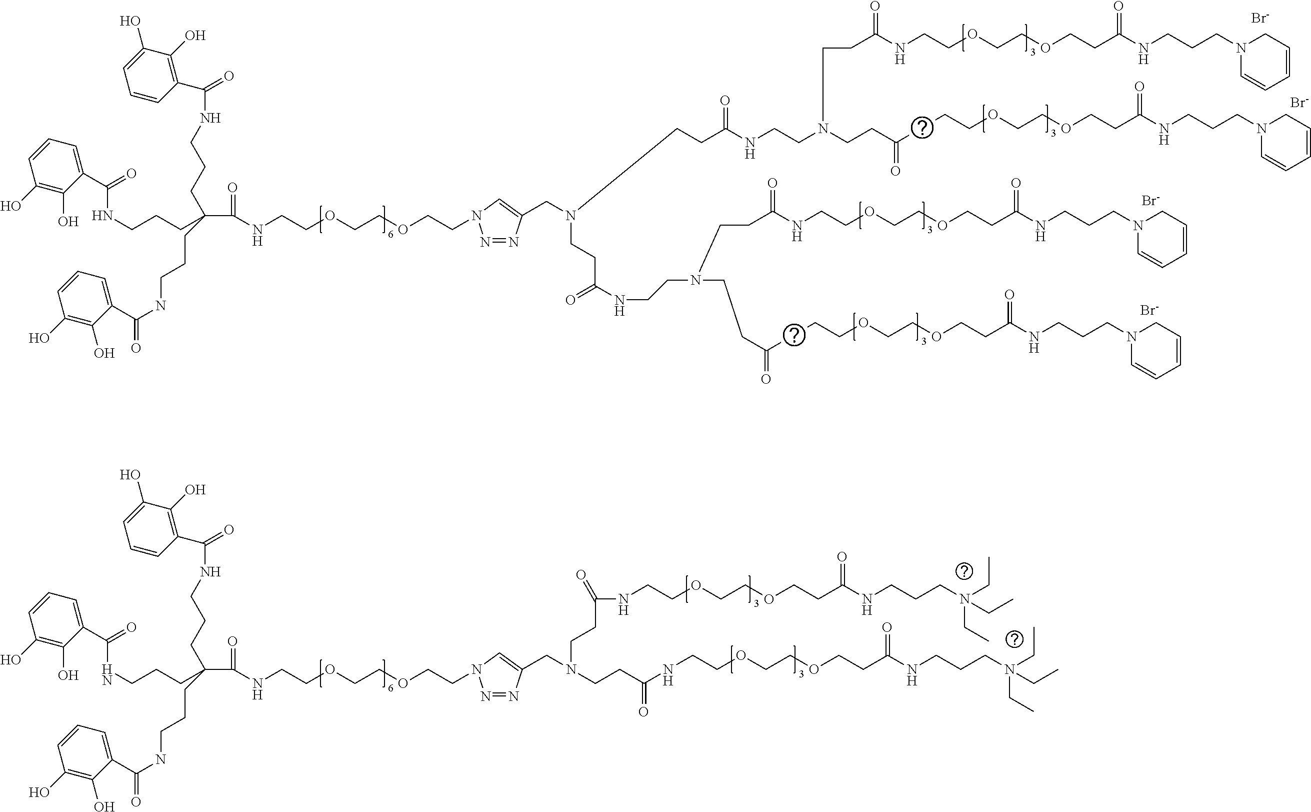 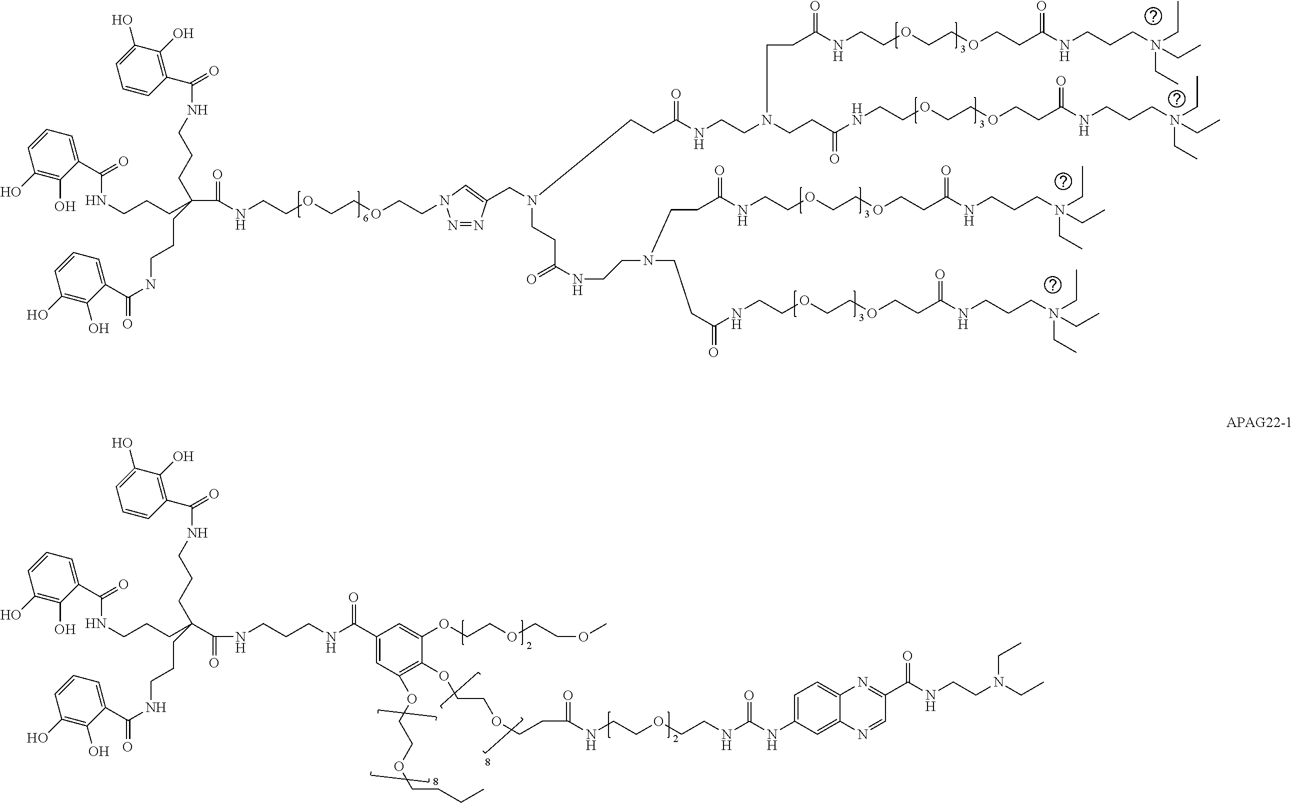 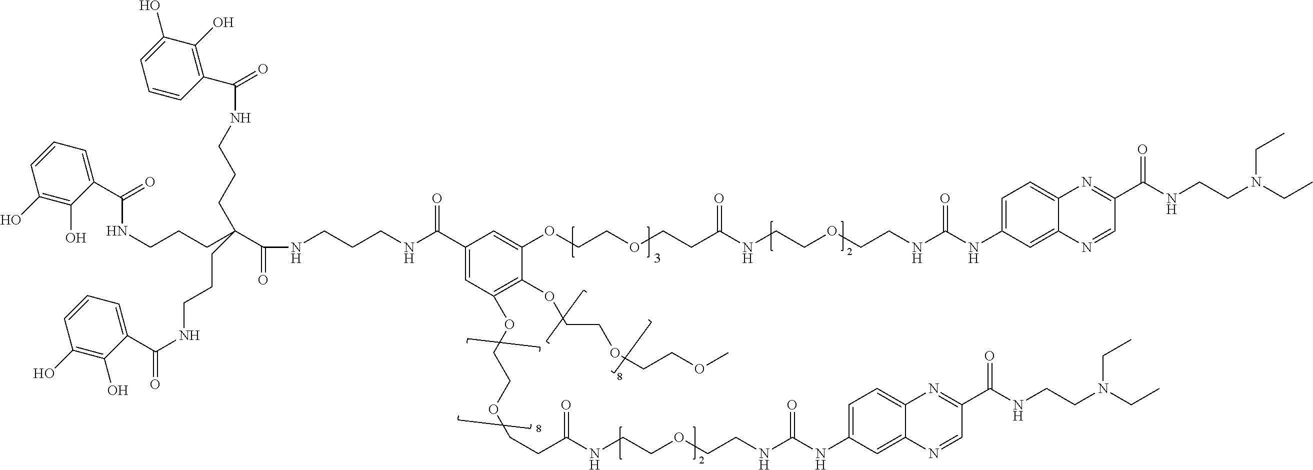 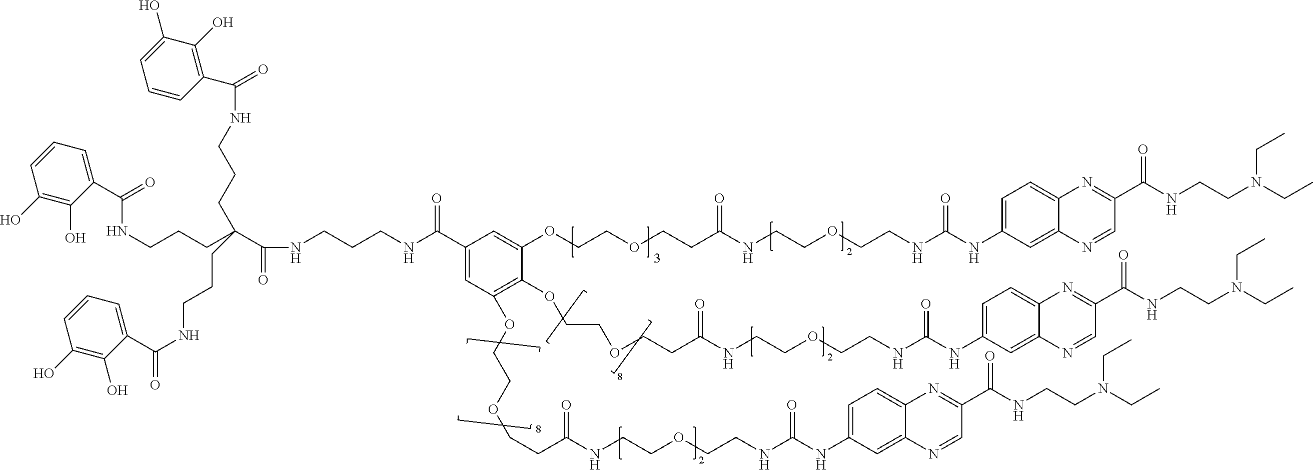 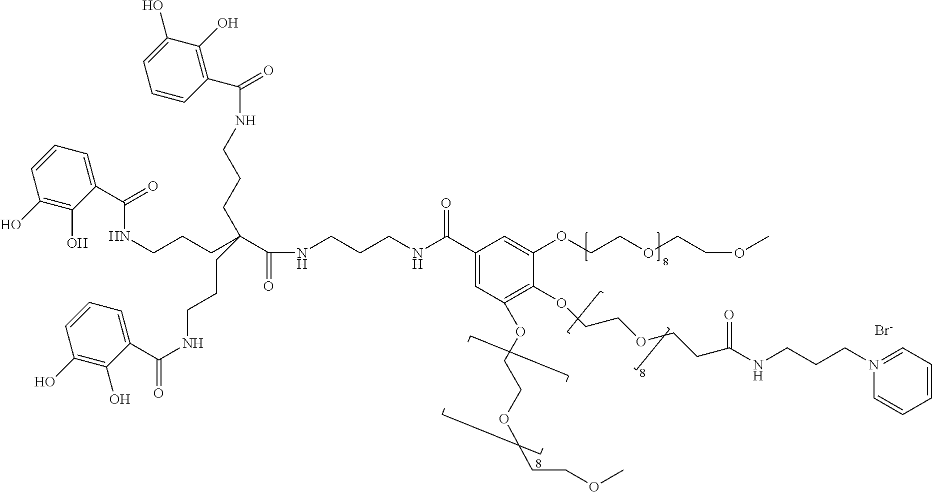 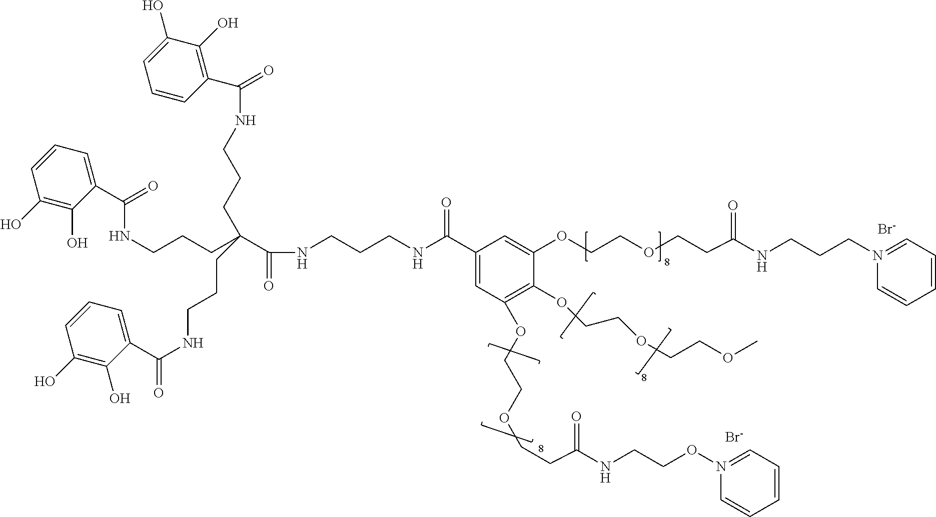 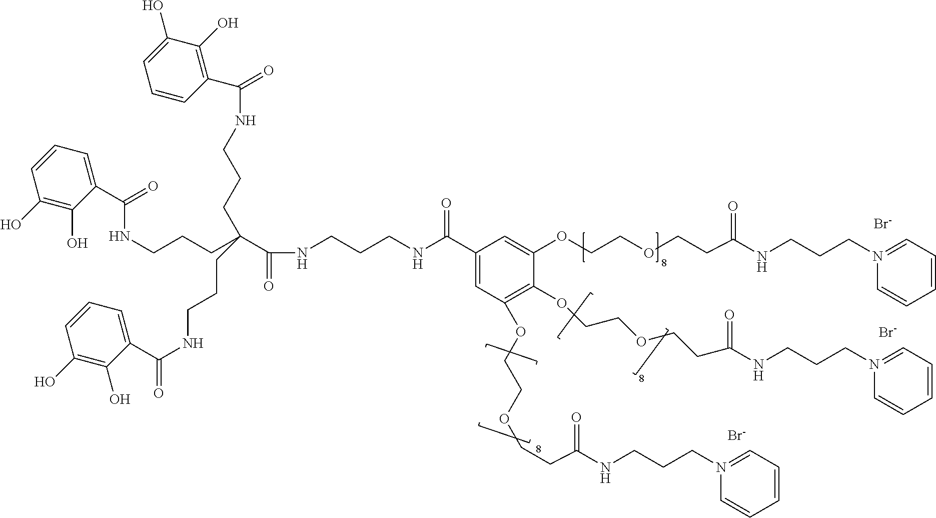 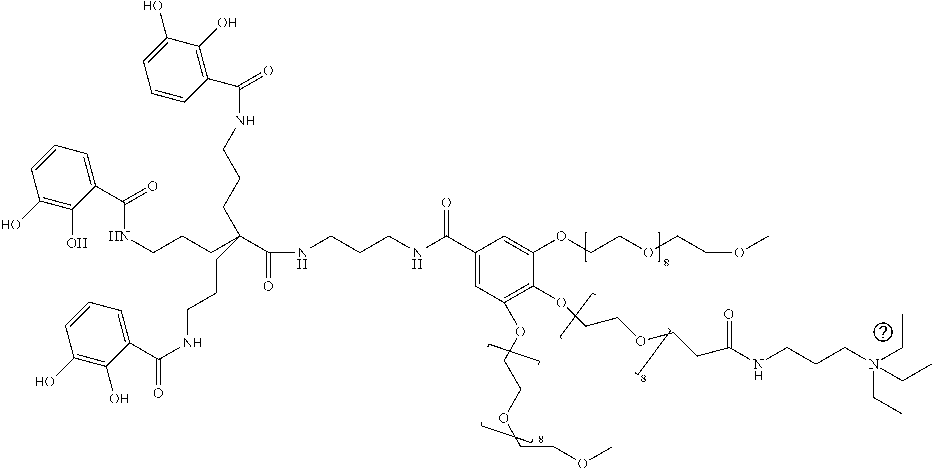 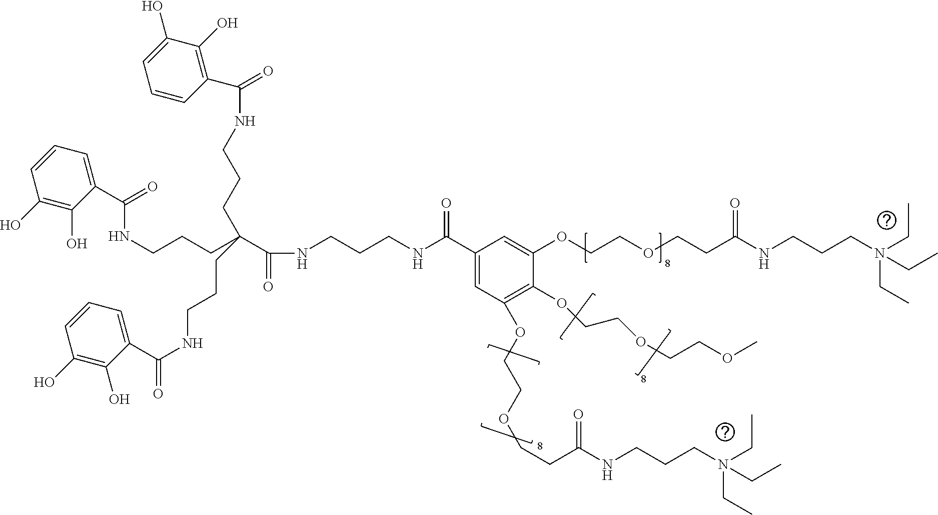 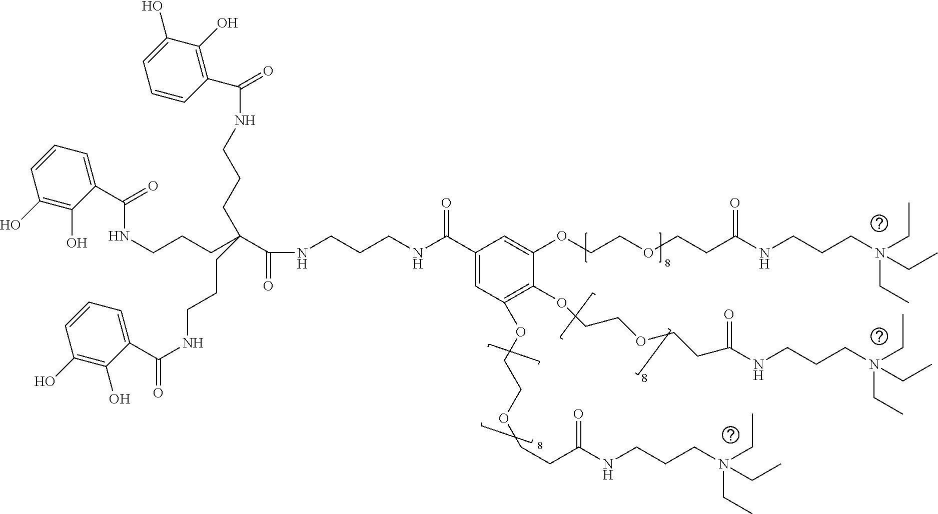 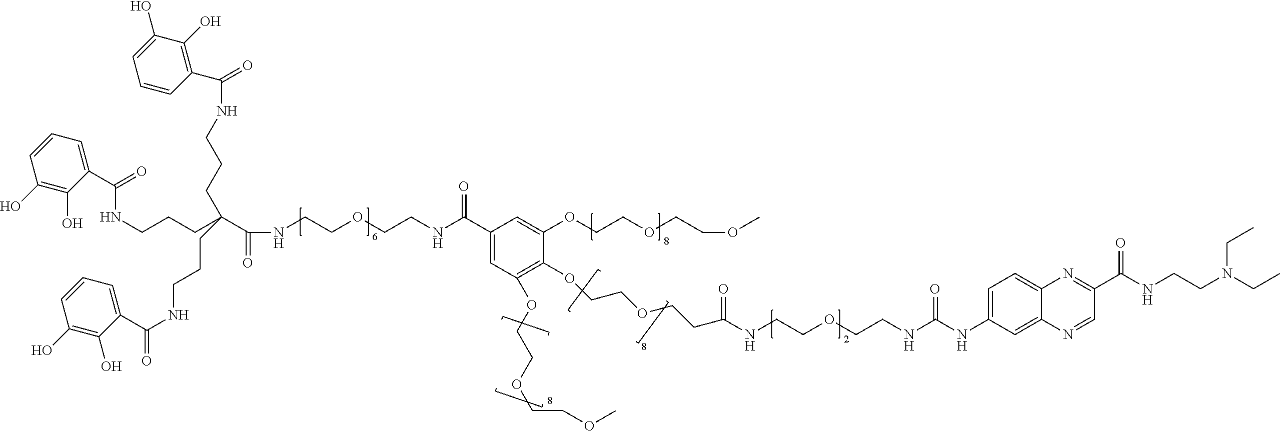 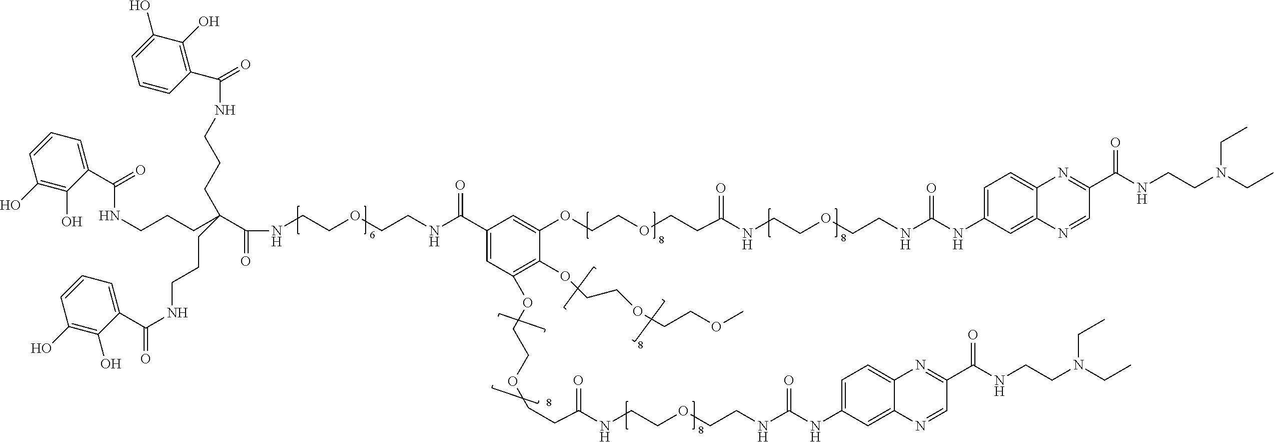 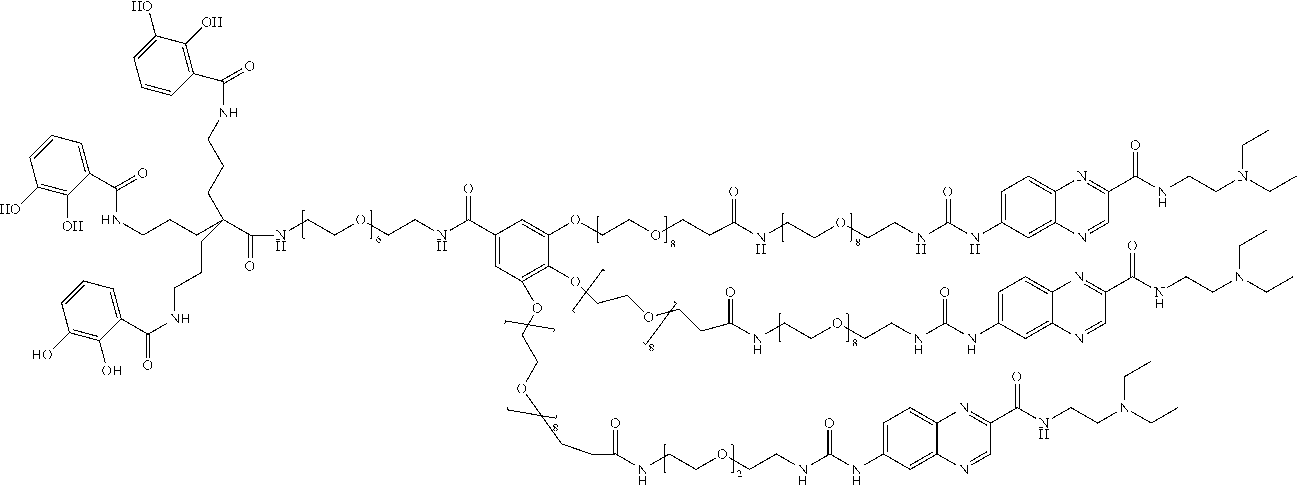 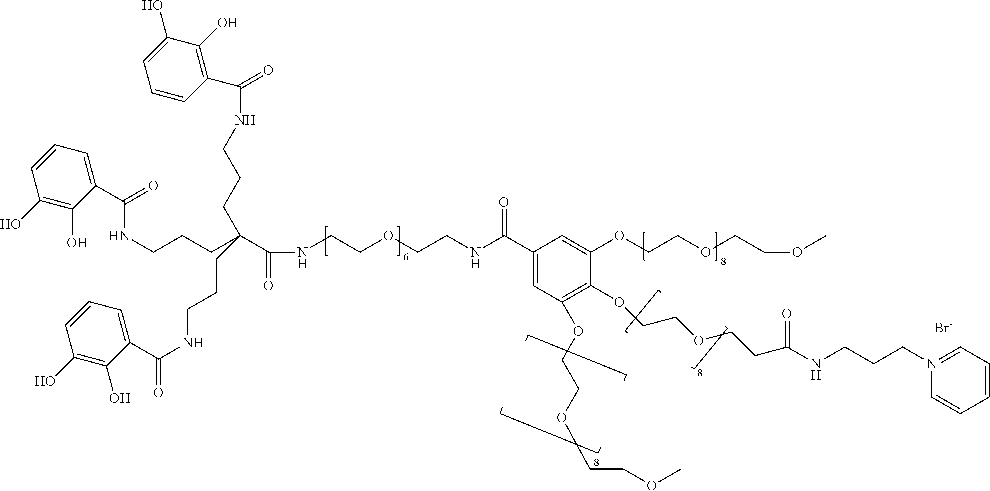 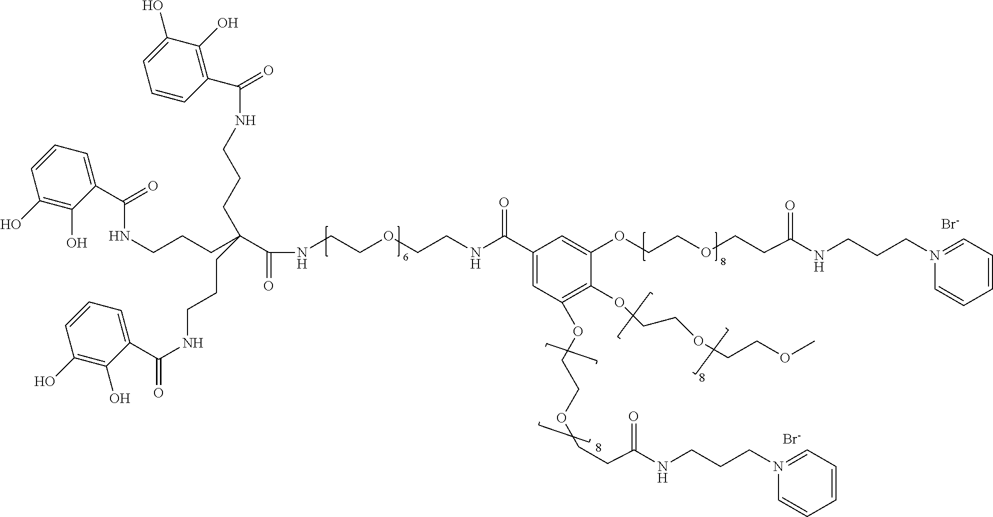 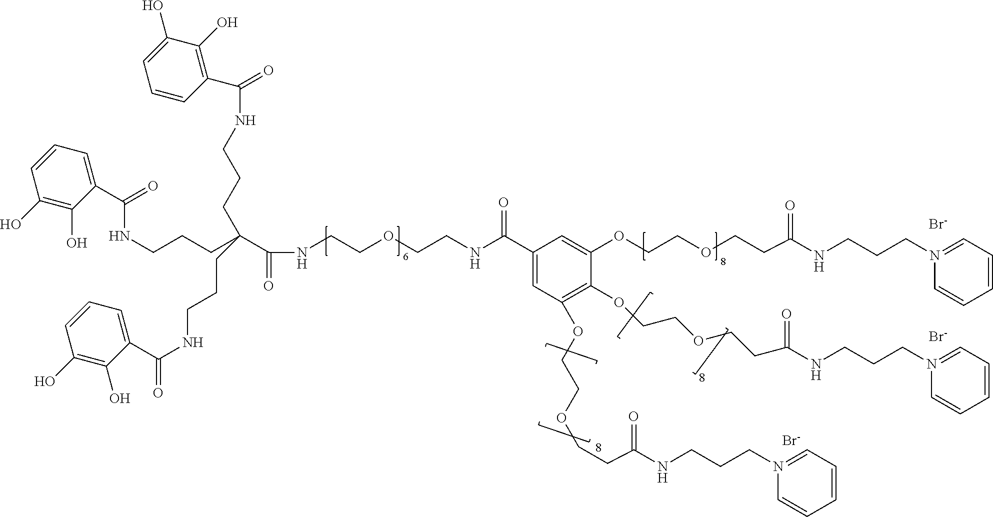 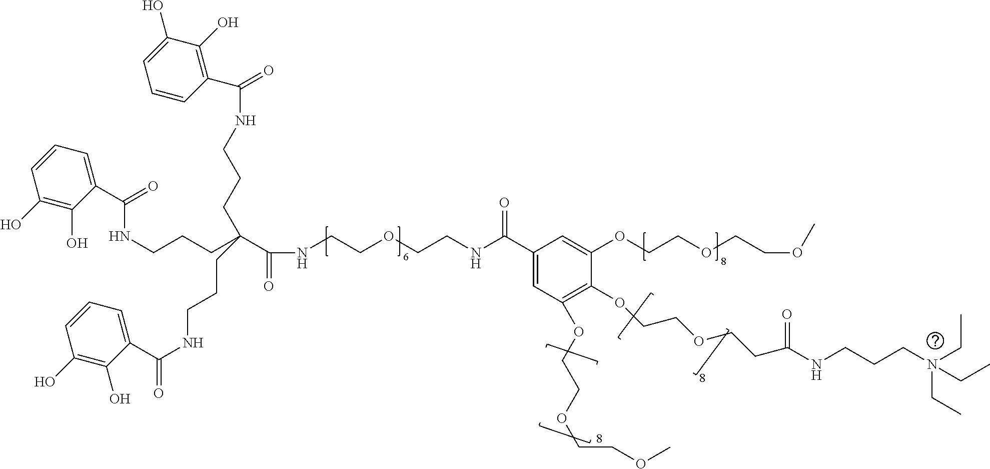 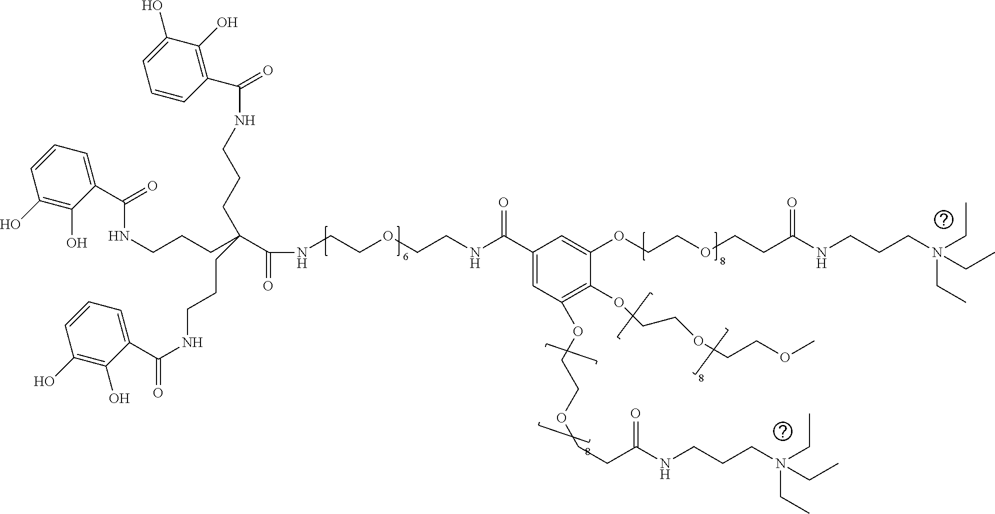 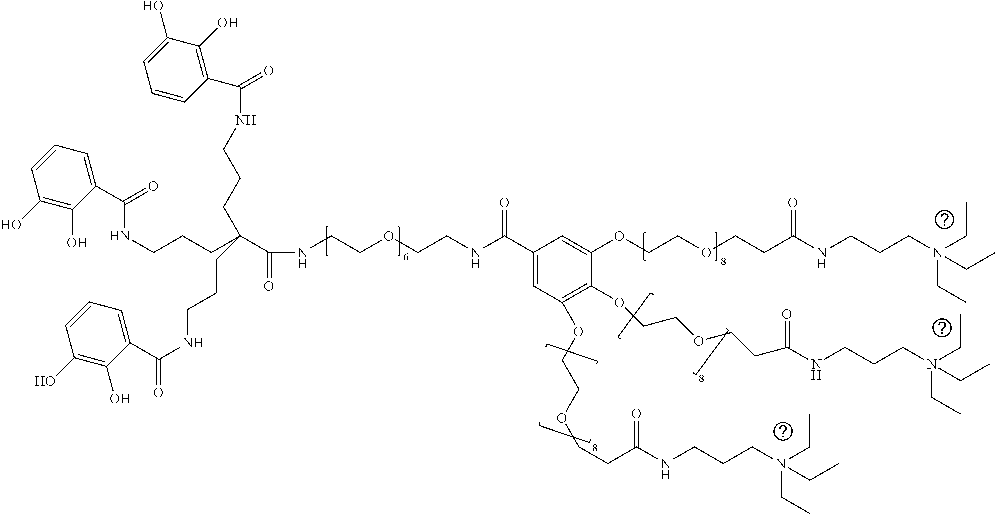 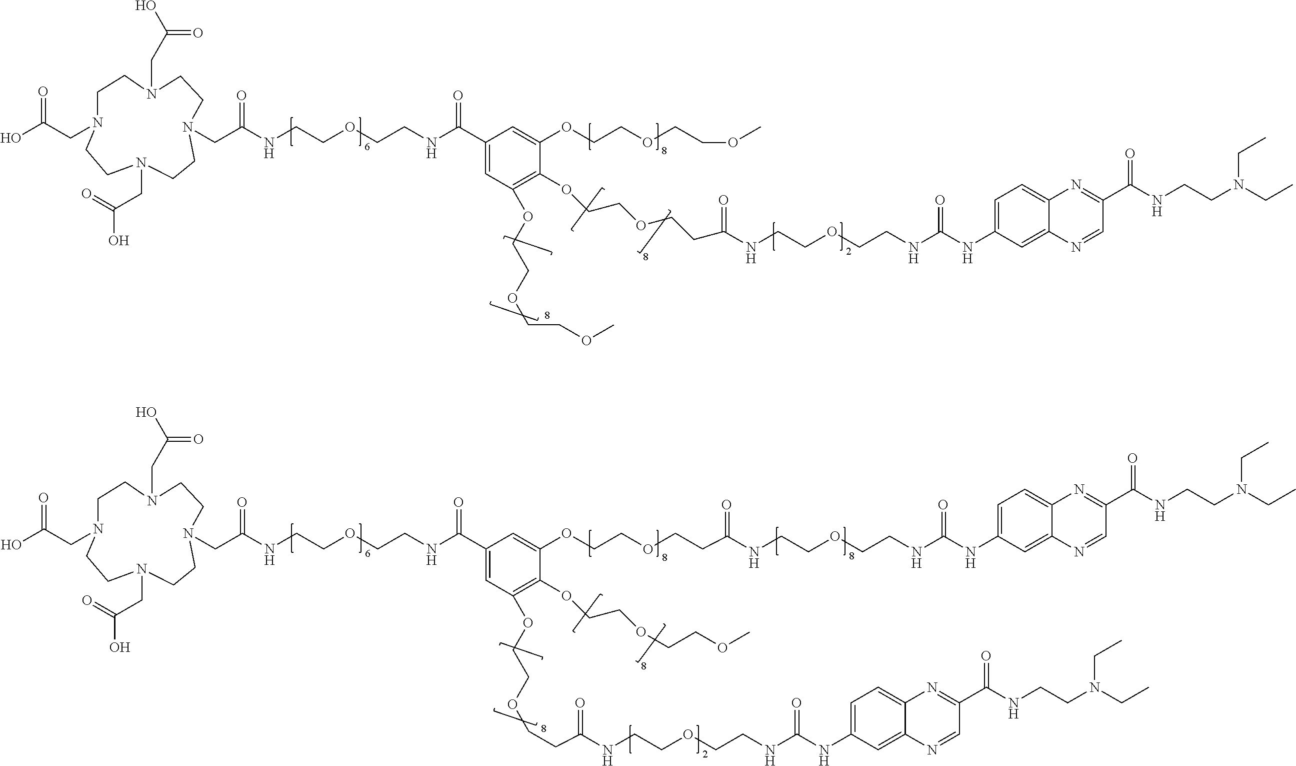 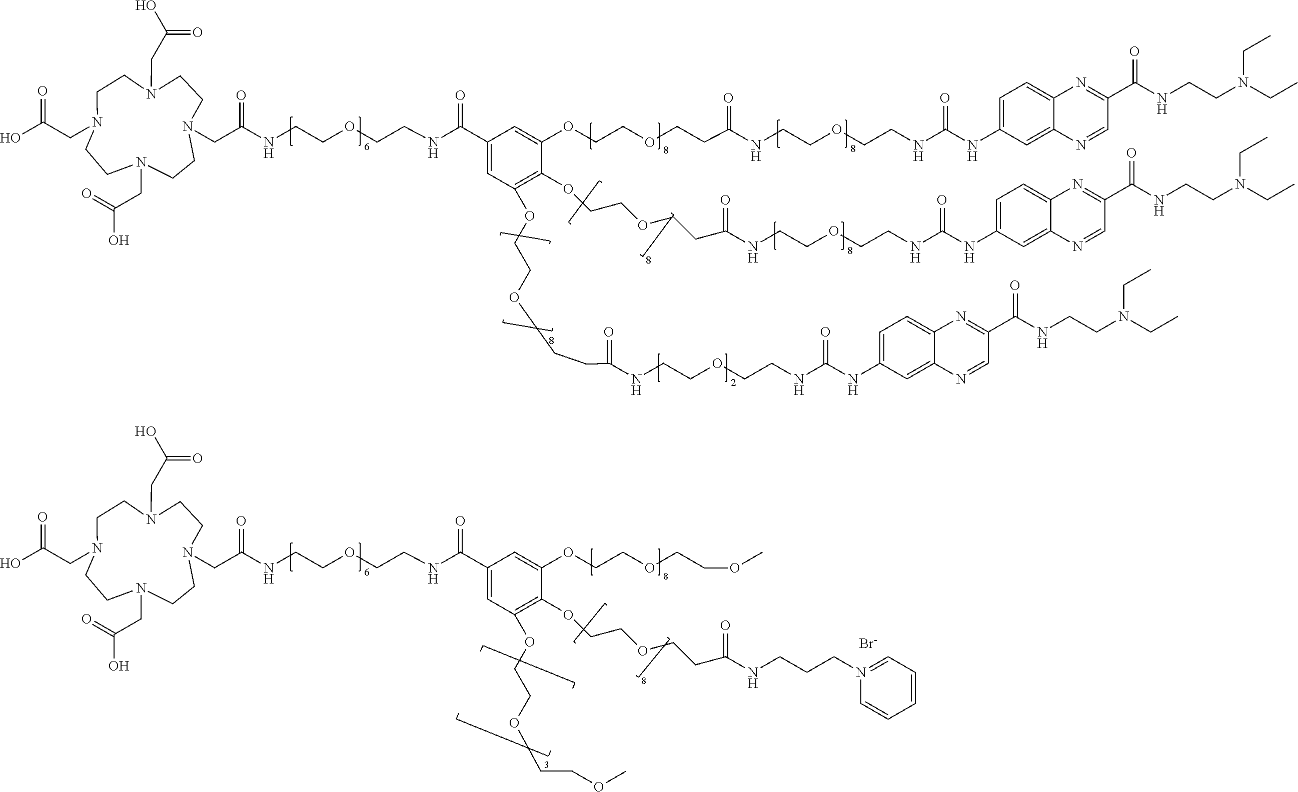 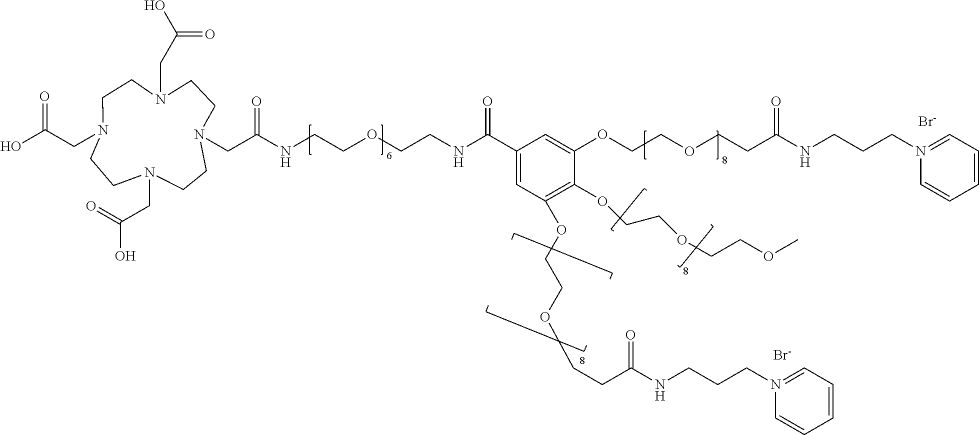 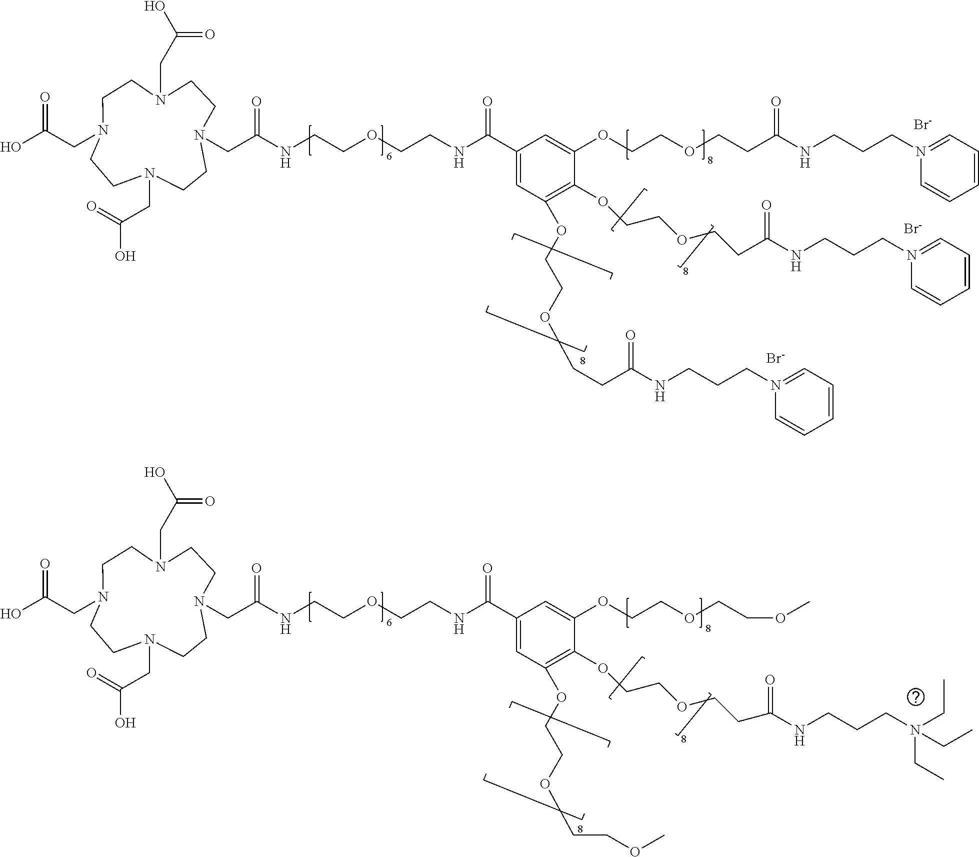 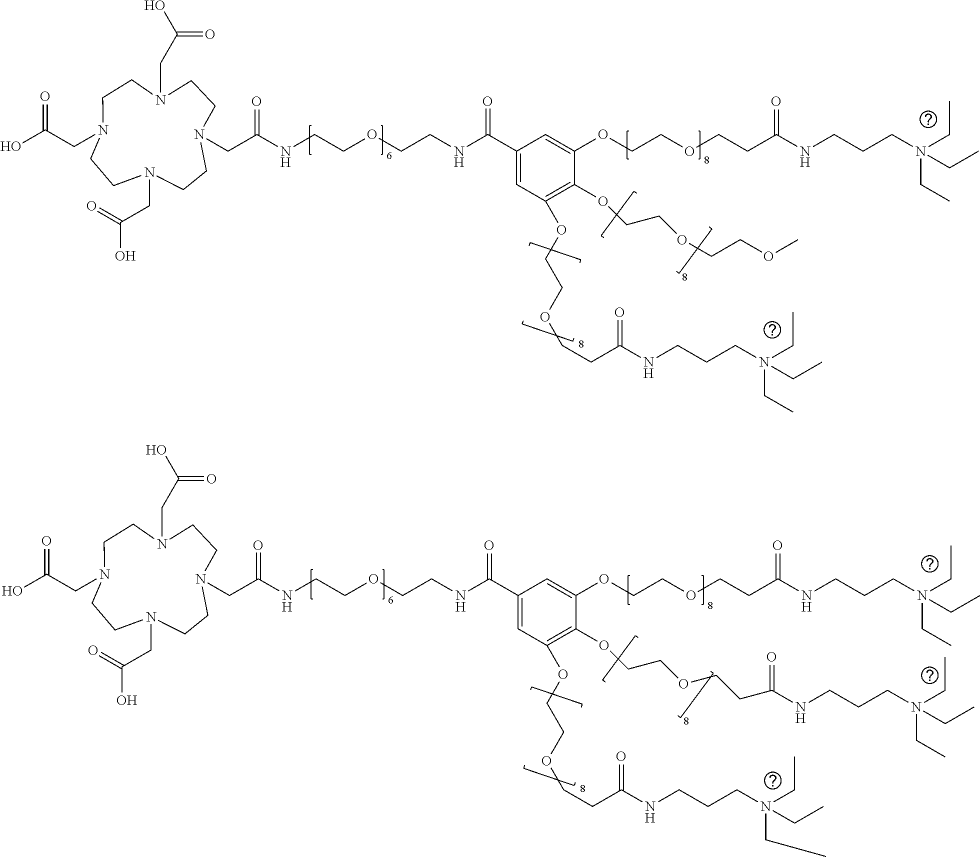 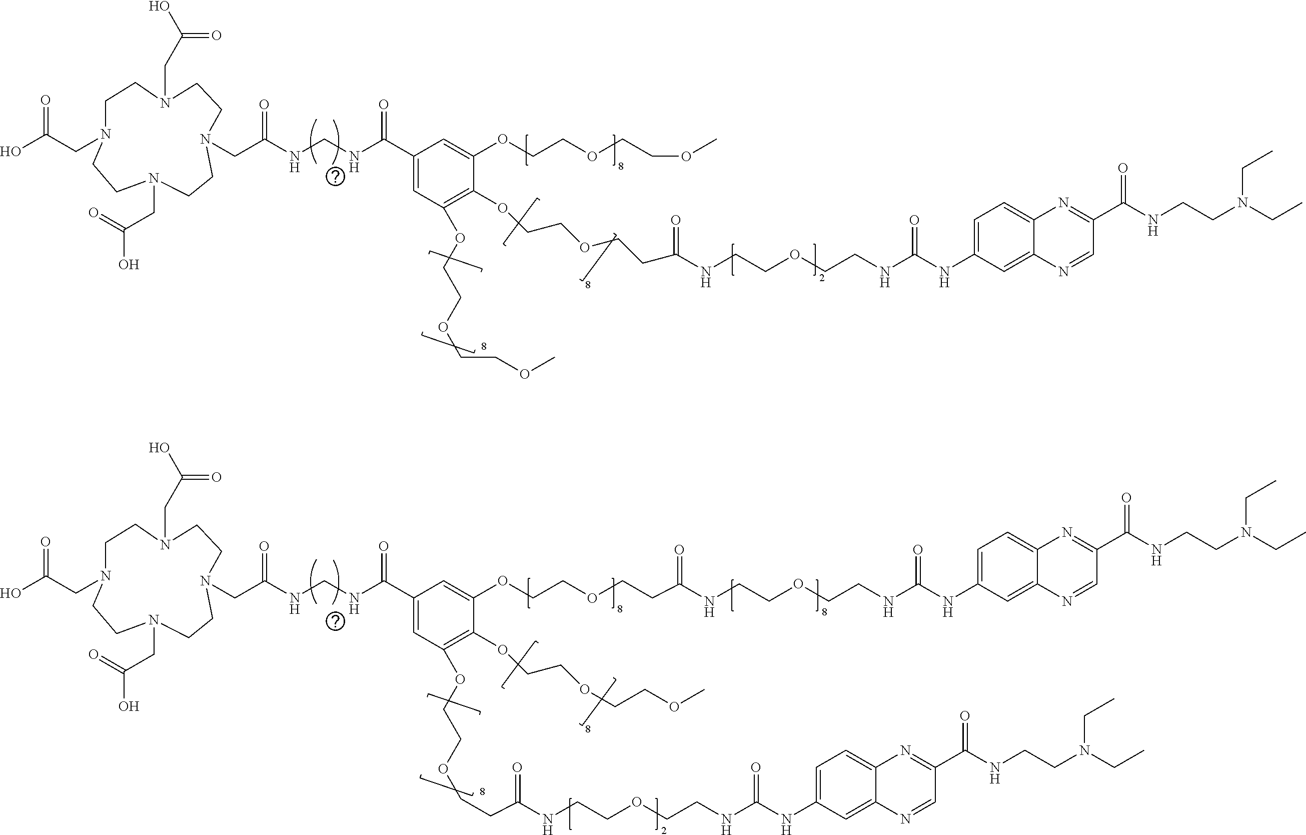 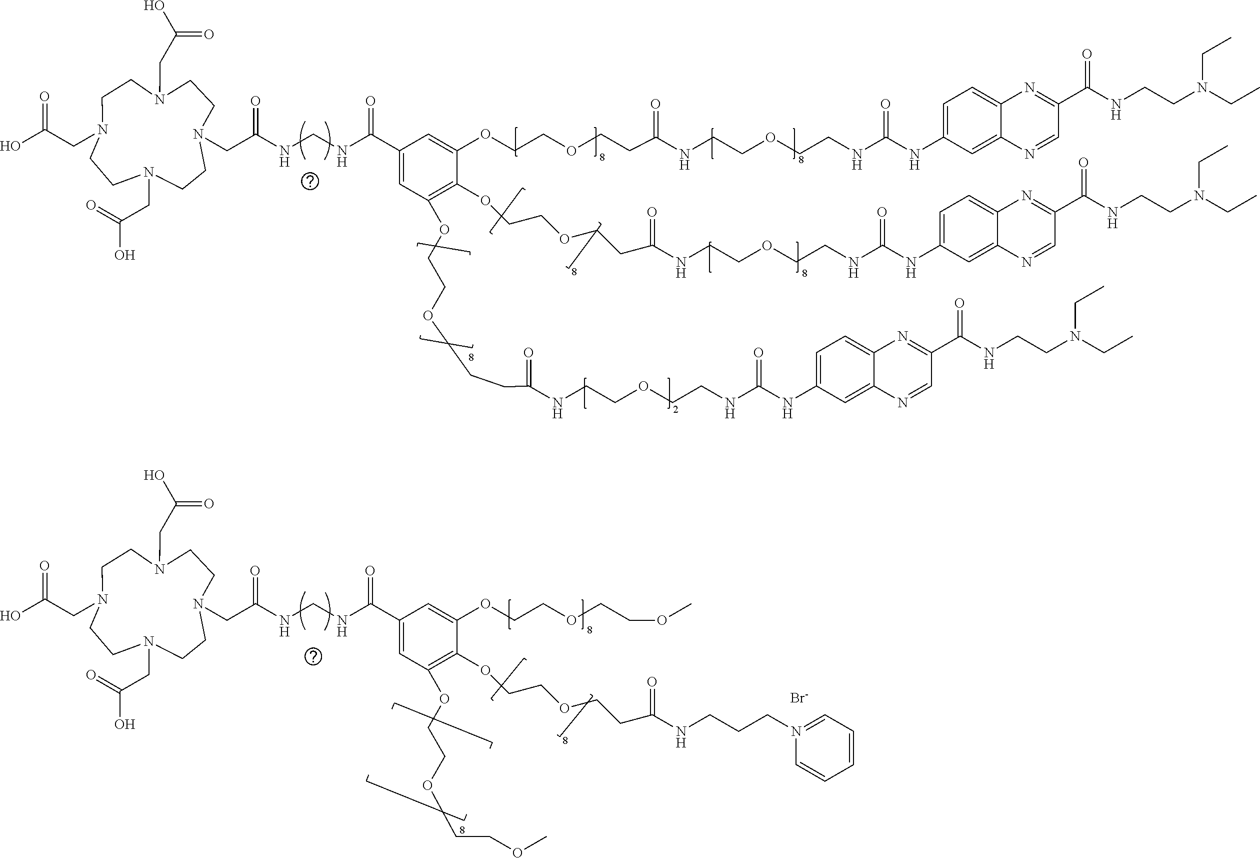 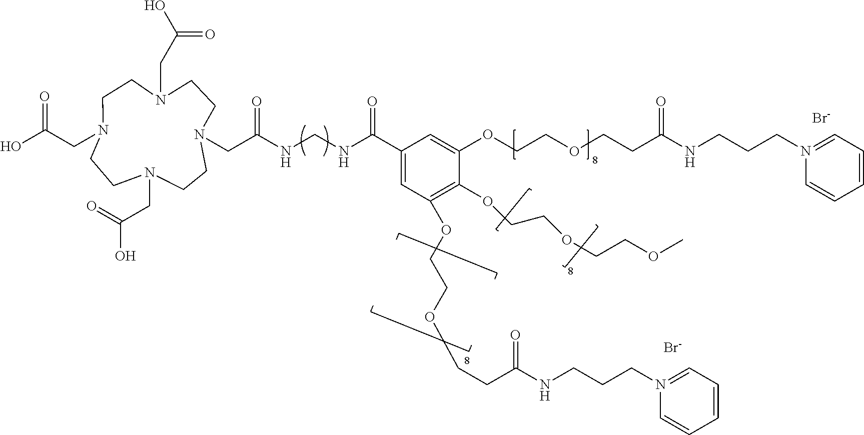 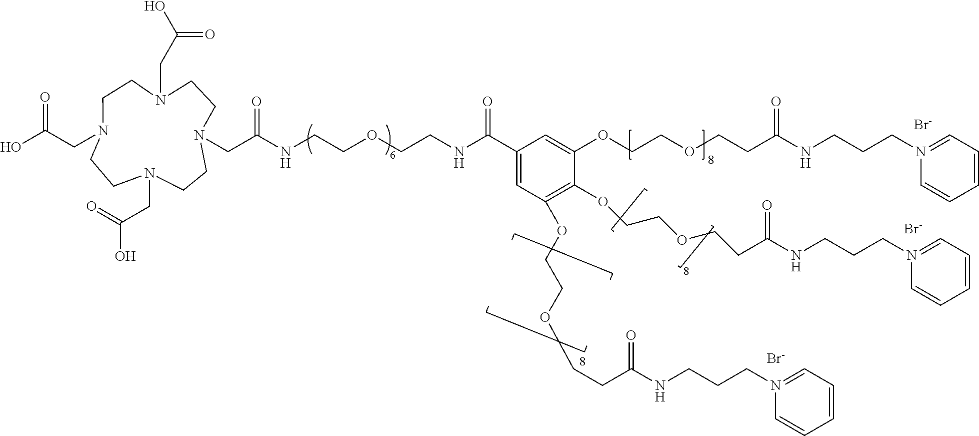 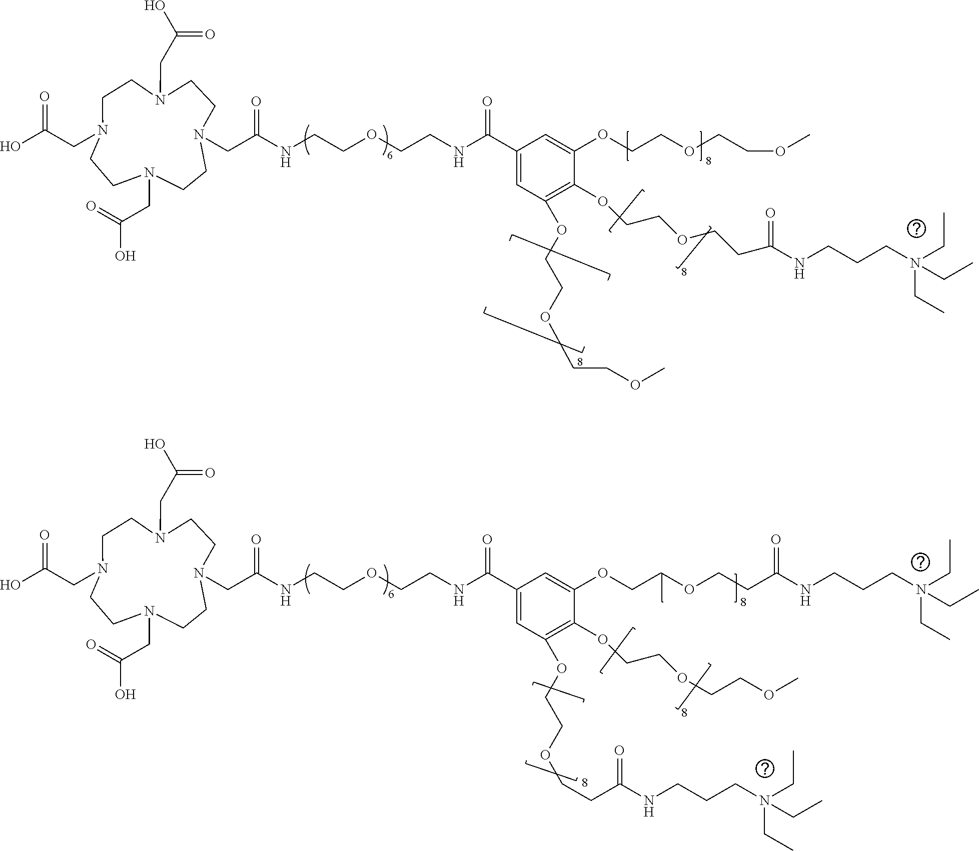 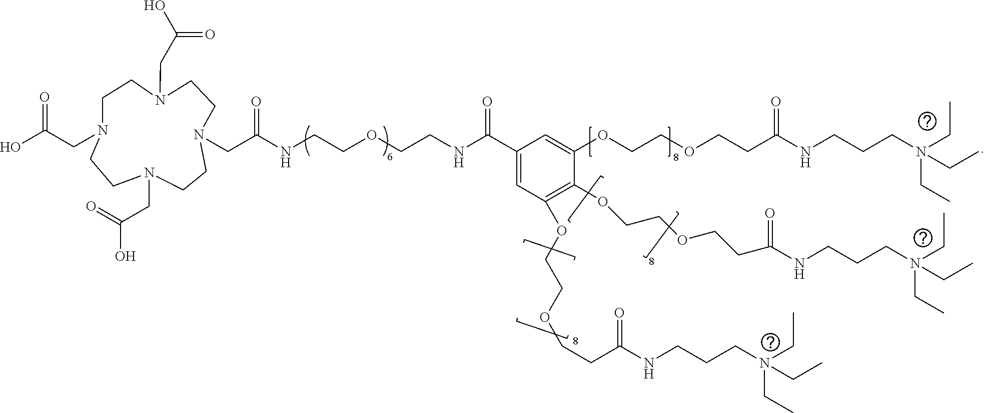
as well as the compounds comprising gallate units depicted above, where the spacer of formula —NH(CH₂CH₂O)₆CH₂CH₂NH— between the chelating agent and the dendrimer is replaced by the spacer of formula:

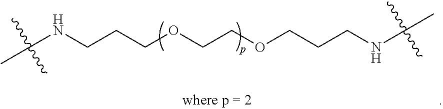

11. A method for the diagnosis of cancers, notably the diagnosis of micrometastases, comprising administering to a subject in need thereof a combination product comprising:
(i) a compound of claim 1; and
(ii) a compound corresponding to formula IV:
T′-L₁-R IV
wherein
T′ represents: [symbol]
biotin, when group T of the compound of claim 1 is avidin or streptavidin, or [symbol]
avidin or streptavidin, when group T of the compound of any one of claims 1 to 9 is biotin, [symbol]
adamantane, when group T of the compound of any one of claims 1 to 9 is a cyclodextrin, or [symbol]
a cyclodextrin, when group T of the compound of any one of claims 1 to 9 is adamantane,
L₁ is a heteroalkyl chain of structure: 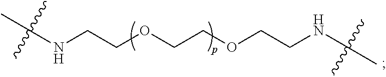
wherein p is an integer from 1 to 10;
or else T′ is absent and L₁ corresponds to one of the following azide structures: 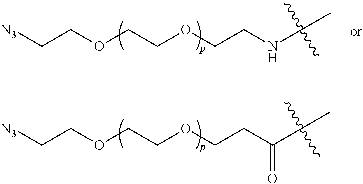
wherein p is an integer from 1 to 10; preferably from 1 to 8; preferably 3 or 7;
R is a labelable moiety such as L-thyroxine or dinitrobenzoate: 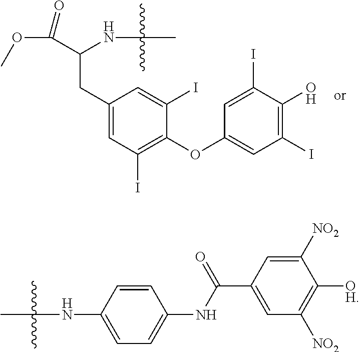 [SMILES: *NC(Cc1cc(I)c(Oc2cc(I)c(O)c(I)c2)c(I)c1)C(=O)OC ; *Nc1ccc(NC(=O)c2cc([N+](=O)[O-])c(O)c([N+](=O)[O-])c2)cc1]
wherein the administration of compounds (i) and (ii) is carried out separately or spread over time.

12. A pharmaceutical or diagnostic composition comprising a compound of claim 1, and a pharmaceutically acceptable vehicle.

13. A method for:
detecting tumors, notably in the form of micrometastases, and/or of cells in a particular metabolic state, notably hypoxic cells or apoptotic cells;
detecting tumors by manganese-enhanced magnetic resonance imaging;
detecting tumors by gamma scintigraphy (GSc) or by single-photon emission tomography (SPET);
detecting tumors by positron emission tomography (PET);
characterizing the sentinel lymph node;
the radiotherapeutic treatment of tumors, notably in the form of micrometastases; and/or
curietherapy or internal or targeted, or vectorized radiotherapy;
comprising administering to a subject in need thereof a compound of claim 1.

14. The method of claim 13 for characterizing the sentinel lymph node.

15. The method of claim 14, wherein in the compound, the targeting agent V is a nucleolin membrane ligand or an alpha-MSH ligand (HB-19).

16. The method of claim 14, wherein the compound has the structure: [chemical structure]
